(19) United States
(12) Patent Application Publication
(10) Pub. No.: US 2020/0297725 A1
(43) Pub. Date: Sep. 24, 2020

(54) ALLOSTERIC BCR-ABL PROTEOLYSIS TARGETING CHIMERIC COMPOUNDS

(71) Applicant: YALE UNIVERSITY, New Haven, CT (US)

(72) Inventors: Craig M. CREWS, New Haven, CT (US); George BURSLEM, Sandwich (GB)

(21) Appl. No.: 16/825,878

(22) Filed: Mar. 20, 2020

Related U.S. Application Data
(60) Provisional application No. 62/822,594, filed on Mar. 22, 2019.
(60) Provisional application No. 62/824,154, filed on Mar. 26, 2019.

(51) Int. Cl. A61K 31/506; C07D 417/14; C07D 403/12; C07D 403/14; A61K 31/4439

(52) U.S. Cl. CPC A61K 31/506; C07D 417/14; A61K 31/4439; C07D 403/14; C07D 403/12

(57) ABSTRACT

The present invention includes novel compounds and methods for preventing or treating diseases associated with and/or caused by overexpression and/or uncontrolled activation of a tyrosine kinase in a subject in need thereof. In certain embodiments, the compounds of the present invention include an allosteric tyrosine kinase inhibitor, a linker, and a ubiquitin ligase binder. The methods of the present invention include administering to the subject an pharmaceutically effective amount of at least one compound of the invention.

### CROSS-REFERENCE TO RELATED APPLICATIONS

This application claims priority to U.S. Provisional Patent Application Ser. No. 62/822,594 entitled “ALLOSTERIC BCR-Abl PROTEOLYSIS TARGETING CHIMERIC COMPOUNDS,” filed Mar. 22, 2019, and to U.S. Provisional Patent Application Ser. No. 62/824,154 entitled “ALLOSTERIC BCR-Abl PROTEOLYSIS TARGETING CHIMERIC COMPOUNDS,” filed Mar. 26, 2019, the disclosures of which are incorporated herein by reference in their entirety.

### STATEMENT REGARDING FEDERALLY SPONSORED RESEARCH

This invention was made with government support under CA197589 and CA212229 awarded by National Institutes of Health. The government has certain rights in the invention.

### BACKGROUND

The current inhibitor-based drug paradigm not only limits drug targets to those proteins with a tractable active site, but also requires high dosing in order to achieve adequate IC₉₀ concentrations for therapeutic efficacy. To circumvent these issues, alternative therapeutic strategies have been employed to specifically knock down target proteins. While genetic techniques such as RNAi, and CRISPR/Cas9 can significantly reduce protein levels, the pharmacokinetic properties (i.e., metabolic stability and tissue distribution) associated with these approaches have so far limited their development as clinical agents.

The pathologic fusion protein BCR-ABL is a constitutively active tyrosine kinase that drives uncontrolled cell proliferation, resulting in chronic myelogenous leukemia (CML). With the advent of tyrosine kinase inhibitors (TKIs) targeting BCR-ABL, CML has become a chronic but manageable disease. For example, imatinib mesylate, the first TKI developed against BCR-ABL, binds competitively at the ATP-binding site of c-ABL and inhibits both c-ABL and the oncogenic fusion protein BCR-ABL. Second generation TKIs (such as dasatinib and bosutinib) were subsequently developed to treat CML patients with acquired resistance to imatinib. Despite the remarkable success of BCR-ABL TKIs, all CML patients must remain on treatment for life because the TKIs are not curative due to persistent leukemic stem cells (LSCs).

Chronic exposure to BCR-ABL TKIs can lead to resistance mutations in patient populations, which can reduce the efficacy of these compounds over time. For example, the T315I mutation at the gatekeeper residue in the ATP-binding site of BCR-ABL is common in advanced phases of CML and is one of the main causes of resistance, disrupting important contact points between the inhibitors and the enzyme. While BCR-ABL TKIs target the catalytic site of BCR-ABL, BCR-ABL also possesses an allosteric site that can be targeted for potent and selective inhibition. Allosteric tyrosine kinasae inhibitors (ATKIs) of BCR-ABL have different resistance (mutation) profiles than catalytic inhibitors, and can thus be useful in treating patient populations with resistance to standard BCR-ABL TKI therapy.

There is thus an unmet need in the art for compositions and methods to inhibit c-ABL and/or BCR-ABL in a cell with a compound that includes an allosteric tyrosine kinase inhibitor (ATKI). In certain embodiments, such methods can be used to treat and/or prevent CML in a mammal. The present invention addresses this need.

### BRIEF SUMMARY OF THE INVENTION

In some embodiments, the a compound of Formula (I) is provided. The compound of Formula (I) has the structure:

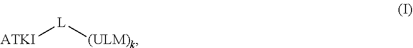

wherein:
ATKI is an allosteric tyrosine kinase inhibitor,
L is a linker,
each ULM is independently a ubiquitin ligase binder, and
k is an integer ranging from 1 to 4,
ATKI is covalently linked to L and wherein each ULM is covalently linked to L;
or a salt, enantiomer, stereoisomer, solvate, polymorph or N-oxide thereof.

In various embodiments, the compounds of Formula (I) can advantageously bind to an allosteric pocket or region of a tyrosine kinase. In some embodiments, the compounds of Formula (I) are useful in methods of treating or preventing a disease or disorder associated with overexpression and/or uncontrolled activation of c-Abl and/or BCR-ABL. The allosteric binding mode of the compounds of Formula (I) can, in some embodiments, advantageously result in binding and ubiquitination of kinases and/or proteins that have developed resistance (and hence reduced efficacy) to ATP-competitive tyrosine kinase inhibitors.

### BRIEF DESCRIPTION OF THE FIGURES

The drawings illustrate generally, by way of example, but not by way of limitation, various embodiments of the present invention.

FIG. 1 illustrates cell proliferation in Ba/F3 parental and BCR-Abl transformed Ba/F3 cells, in accordance with various embodiments. Imatinib (a catalytic site inhibitor of BCR-Abl) is compared with allosteric inhibitors Compound 10 and Compound 14.

FIGS. 2A-2B illustrate BCR-Abl PROTACs function in CML patient samples, in accordance with various embodiments. FIG. 2A illustrates immunoblotting of CML patient stem cells (CD34+CD38−) treated with Compound 10 (also designated C10) or Compound 14 (also designated C14). FIG. 2B illustrates apoptosis induction in normal and CML bone marrow samples.

FIGS. 3A-3C illustrate the in vivo characterization of Compound 15, in accordance with various embodiments. FIG. 3A shows tumor volumes over time. Animals were placed into groups with equal tumor volumes on day 0. Treatment began on day 4. FIG. 3B shows tumor volumes prior to treatment. FIG. 3C shows final tumor volumes collected on day 6 prior to euthanization.

FIGS. 4A-4C illustrate the development of an allosteric BCR-ABL1 bifunctional compound. FIG. 4A shows the X-ray Crystal Structure of GNF-2 bound to the myristate pocket of Abl (PDB ID: 3K5V). FIG. 4B shows structures of GNF-2 and GNF-5. FIG. 4C illustrates a schematic of GNF-5 to bifunctional compound conversion and optimization.

FIGS. 5A-5F illustrate various embodiments of bifunctional compounds inhibiting and degrading BCR-ABL1 via the proteasome in CML cell lines. FIG. 5A shows dose response of GMB-475 (Compound 10) in K562 cells by immunoblot after 18 hours. FIG. 5B shows K562 sensitivity to PROTACs assessed by cell proliferation assay. FIG. 5C shows the time course of degradation in Ba/F3 BCR-ABL1 cells. FIG. 5D shows the effect of PROTACs on Ba/F3 BCR-ABL1 cell proliferation. FIG. 5E illustrates the degradation mechanism interrogation by immunoblot in K562 cells following 8 hour treatment. FIG. 5F shows the quantification of BCR-ABL1 protein levels, from panel FIG. 5E by densitometry.

FIGS. 6A-6E illustrate various embodiments of Compound 10-mediated degradation enhances efficacy of ATP-competitive TKIs and retains potency against imatinib resistant point mutations. FIG. 6A shows IC₅₀ values for single agents and combinations in Ba/F3 BCR-ABL1 cells. FIG. 6B shows the effects of immunoblot of overnight co-treatment with ponatinib in Ba/F3 BCR-ABL1 cells. FIGS. 6C and 6D illustrate the effects of imatinib and PROTACs on cell proliferation in Ba/F3 cells expressing mutant BCR-ABL1. FIG. 6E shows a summary of IC₅₀ values for PROTAC compounds and imatinib in Ba/F3 cell lines.

FIGS. 7A-7B illustrates an embodiment where combined inhibition and degradation of BCR-ABL1 by GMB-475 (Compound 10) reduces scaffolding of downstream interactors. FIG. 7A shows the immunoblot analysis of downstream signaling and scaffolding proteins in K562 cells treated with 2.5 μM PROTAC or diastereomer. FIG. 7B shows the differences in pSTAT5, pGAB2, and pSHC between stimulated and unstimulated K562 cells.

FIGS. 8A-8D illustrate an embodiment showing how GMB-475 (Compound 10) reduces cell viability, induces apoptosis, and degrades BCR-ABL1 in primary CML patient stem/progenitor cells. FIG. 8A shows the cell viability dose response curves for CD34+ cells (patient 1) treated with PROTAC or diastereomer. FIG. 8B shows the effects of Annexin V staining healthy donor or CML primary CD34+ cells (patient 4). FIG. 8C shows the effects of Annexin V staining in sorted progenitor (CD34+/CD38+) and stem (CD34+/CD38−) CML cells (patient 1) by Guava Nexin assay. FIG. 8D is an embodiment showing BCR-ABL1 degradation in CML CD34+/CD38− cells (patient 1) treated overnight with GMB-475 (Compound 10) or diastereomer.

FIGS. 9A-9B illustrate enhanced inhibition and protein degradation of BCR-ABL1 by GMB-475 (Compound 10). FIG. 9A illustrates immunoblot analysis in K562 cells of precursor compound GMB-101 (Compound 1) with doses ranging from 0.25 μM to 20 μM in duplicate for 18 hours. FIG. 9B shows immunoblot analysis of expanded dose range (0.001 μM to 30 μM, in duplicate) of GMB-475 (Compound 10) in K562 cells for 18 hours.

FIGS. 10A-10D illustrate validation of degredation properties of PROTAC and assessment of toxicity in vitro. FIG. 10A shows the degradation of BCR-ABL1 after 18 h incubation with GMB-651 (Compound 14) in K562. FIG. 10B shows the degradation of BCR-ABL1 after 18 h incubation with GMB-651 (Compound 14) in Ba/F3 BCR-ABL1 WT. FIG. 10C shows the activity of GMB-475 (Compound 10) confirmed by immunoblot validation of RPPA analysis. FIG. 10D shows the results of a cell proliferation assay in Ba/F3 parental cells, showing no toxicity to either GMB-475 (Compound 10) or GMB-651 (Compound 14) up to highest tested concentration.

FIGS. 11A-11D shows how embodiments of ATP-competitive TKIs with PROTAC demonstrate selective efficacy against imatinib resistant point mutations. FIG. 11A shows a comparison of IC50 values for imatinib in parental and BCR-ABL1 T315I Ba/F3 cells treated with both PROTAC and diastereomer control in combination with imatinib. FIG. 11B shows immunoblot analysis of Ba/F3 BCR-ABL1 T315I cells treated with ponatinib in combination with PROTACs for 18 h. FIGS. 11C and 11D show immunoblot analysis of Ba/F3 BCR-ABL1 point mutants treated with GMB-475 (Compound 10) or GMB-651 (Compound 14). Ba/F3 BCR-ABL1 T315I cells were treated for various durations up to 24 h with 2.5 μM of each PROTAC. Ba/F3 BCR-ABL1 G250E cells were treated for 18 h at a range of concentrations (0.25 μM-10 μM).

FIGS. 12A-12D illustrate the effects of other compounds on protein levels in K562 cells. FIG. 12A shows a schematic of Y177 scaffolding roles. FIG. 12B shows immunoblot analysis of downstream signaling and scaffolding proteins in K562 cells treated with 1 μM imatinib. FIG. 12C shows a structure of a VHL Ligand. FIG. 12D shows immunoblot analysis of VHL protein in K562 cells treated with 2.5 μM VHL ligand.

FIGS. 13A-13F show embodiments of PROTAC (compounds of Formula I) efficacy assessed in primary CML patient samples. FIG. 13A illustrates patient sample protocol schema for CD34 MACS column selection and FACS analysis for selection of all primary patient samples. FIG. 13B illustrates flow cytometry gating for sorting CD34+/CD38+ and CD34+/CD38− populations for patient 1. FIG. 13C shows cell proliferation assay results for patient 1 CD34+ cells treated with imatinib (see also FIG. 8A). FIG. 13D shows an Annexin V analysis using ApoScreen Annexin V-FITC for newly diagnosed CML patient cells (patient 4) and normal human CD34+ bone marrow cells treated with imatinib for 48 h (see FIG. 8B). FIG. 13E shows the cell viability dose response curves for CD34+ cells from patient 2 treated with PROTAC or diastereomer. FIG. 13F shows Annexin V bulk CD34+ CML cells from patient 3 after 96 h analyzed by Guava Nexin assay.

FIGS. 14A and 14B show (14A) exemplary bifunctional compounds GMB-475 (Compound 10) and GMB-805 (Compound 19) derived from BRC-Abl inhibitors GNF-5 and Abl-001, respectively, and (14B) associated immunoblots comparing their activity in K562 cells.

FIGS. 15A-15C show co-crystal structures of Abl with GNF-2 [PDB ID: 3K5V] (15A), Abl001 with asciminib [PDB ID: 5MO4] (15B), or overlay of the two allosteric binders (15C).

FIGS. 16A and 16B show characterization data for GMB-805 (Compound 19). FIG. 16A is a dose response in K562 cells treated with the indicated doses for 24 hours. FIG. 16B illustrates the antiproliferative activity of GMB-805 (Compound 19) in K562 cells.

FIG. 17 shows co-treatment experiments with a compound of Formula (I). K562 Cells were treated with 1 μM Abl-001 or GMB-805 (Compound 19) for 8 hours in the presence of the indicated compounds.

FIG. 18 shows characterization of GMB-805. K562 cells were treated with the indicated compounds and concentrations for 24 hours.

FIG. 19 shows a pharmacokinetic profile of GMB-805 (Compound 19) in mouse.

FIGS. 20A and 20B show in vivo data for Compound 19 in a K562 xenograft model. FIG. 20A shows data of GMB-805 (Compound 19) treated animals. FIG. 20B shows data of vehicle treated animals.

FIGS. 21A and 21B show weight loss data for GMB-805 (Compound 19) treated animals. Treatment with GMB-805 (Compound 19) induced no weight loss.

FIG. 22 shows shows chromatographic data for N-(4-(chlorodifluoromethoxy)phenyl)-6-((2-(2-(((S)-1-((2S,4R)-4-hydroxy-2-((4-(4-methyl-1,2,3-thiadiazol-5-yl)benzyl)carbamoyl)pyrrolidin-1-yl)-3,3-dimethyl-1-oxobutan-2-yl)amino)-2-oxoethoxy)ethyl)amino)-5-(1H-pyrazol-5-yl)nicotinamide.

FIG. 23 shows chromatographic data for N-(4-(chlorodifluoromethoxy)phenyl)-6-((2-(2-(((R)-1-((2R,4 S)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethyl)amino)-5-(1H-pyrazol-1-yl)nicotinamide.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

The compounds described herein are, in various embodiments, bifunctional small-molecule compounds that were unexpectedly discovered to be capable of efficiently degrading certain cancer-related tyrosine kinases in a cellular environment. These compounds are in one aspect based on proteolysis targeting chimera bifunctional protein degrader compounds. Each end of the bifunctional compounds described herein is capable of binding to a specific cellular target. One end of the compound can bind to a ubiquitin ligase, while the other end engages the target tyrosine kinase. In various embodiments, the tyrosine kinase binding moiety described herein binds to an allosteric site on the tyrosine kinase instead of at their catalytic (ATP binding) site. Allosteric binding can occur at a site on the tyrosine kinase which is distinct from the ATP binding site. Inhibition of tyrosine kinase function can result, without being bound by theory, from changes in the kinase conformation, from direct competition with protein substrates, and/or from binding in the myristate pocket of the tyrosine kinase.

In various embodiments, the allosteric tyrosine kinases inhibitor (ATKI) moieties described herein bind to the myristate pocket of BCL-Abl. In various embodiments, the ubiquitin ligase is an E3 ubiquitin ligase. The ubiquitin ligase can be, without limitation, a Von Hippel Lindau (VHL) E3 ubiquitin ligase, MDM2 E3 ubiquitin ligase, Inhibitor of Apoptosis Protein (IAP) E3 ubiquitin ligase, and/or a Cereblon (CRBN) E3 ligase. Ternary complex formation can take place when the compounds described herein bind to the tyrosine kinase and the ubiquitin ligase, thus bringing the recruited ligase into close proximity with the tyrosine kinase. Such a binding event leads to the ubiquitination of the tyrosine kinase of interest and its subsequent degradation by proteasomes.

In various embodiments, the compounds described herein can be used to treat diseases associated with overexpression and/or uncontrolled activation of certain tyrosine kinases. The compounds described herein can also be used to treat a cancer that is associated with and/or caused by an oncogenic tyrosine kinase.

The present description provides compounds comprising a ligand, e.g., a small molecule ligand (i.e., having a molecular weight that is lower than about 2,000, 1,000, 500, or 200 Daltons), which is capable of binding to a ubiquitin ligase, such as, but not limited to, VHL or Cereblon. The compounds also comprise a moiety that is capable of binding to a target protein, in such a way that the target protein is placed in proximity to the ubiquitin ligase to effect degradation (and/or inhibition) of that protein. In certain embodiments, “small molecule” means, in addition to the above, that the molecule is non-peptidyl, i.e., it is not generally considered a peptide, e.g., comprises fewer than 4, 3, or 2 amino acids. In accordance with the present description, the ULM and/or PROTAC molecules can be a small molecule.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type might be abnormal for a different cell or tissue type.

A disease or disorder is “alleviated” if the severity of a symptom of the disease or disorder, the frequency with which such a symptom is experienced by a patient, or both, is reduced.

The terms “cancer” refers to the physiological condition in a subject typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small cell lung cancer, non-small cell lung cancer (“NSCLC”), vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, as well as head and neck cancer.

In yet other embodiments, the cancer is at least one selected from the group consisting of ALL, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, Philadelphia chromosome positive CML, lymphoma, leukemia, multiple myeloma myeloproliferative diseases, large B cell lymphoma, and B cell Lymphoma. Without wishing to be limited by any theory, in about 10% of patients with acute lymphocytic leukemia, patients carry a 9;22 translocation cytogenetically indistinguishable from the Philadelphia chromosome of CML.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Eₘₐₓ) achieved within an assay.

As used herein, the term “L” or “Linker” refers to the linker.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds of the invention include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body.

Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(═O)N(R)₂, CN, CF₃, OCF₃, R, C(═O), methylenedioxy, ethylenedioxy, N(R)₂, SR, S(═O)R, S(═O)₂R, S(═O)₂N(R)₂, SO₃R, C(═O)R, C(═O)C(═O)R, C(═O)CH₂C(═O)R, C(═S)R, C(═O)OR, OC(═O)R, C(═O)N(R)₂, OC(═O)N(R)₂, C(═S)N(R)₂, (CH₂)₀₋₂N(R)C(═O)R, (CH₂)₀₋₂N(R)N(R)₂, N(R)N(R)C(═O)R, N(R)N(R)C(═O)OR, N(R)N(R)C(═O)N(R)₂, N(R)S(═O)₂R, N(R)S(═O)₂N(R)₂, N(R)C(═O)OR, N(R)C(═O)R, N(R)C(═S)R, N(R)C(═O)N(R)₂, N(R)C(═S)N(R)₂, N(C(═O)R)—C(═O)R, N(OR)R, C(═NH)N(R)₂, C(═O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(═O)N(R)₂, CN, NO, NO₂, ONO₂, azido, CF₃, OCF₃, R, O (oxo), S (thiono), C(═O), S(═O), methylenedioxy, ethylenedioxy, N(R)₂, SR, S(═O)R, S(═O)₂R, S(═O)₂N(R)₂, SO₃R, C(═O)R, C(═O)C(═O)R, C(═O)CH₂C(═O)R, C(═S)R, C(═O)OR, OC(═O)R, C(═O)N(R)₂, OC(═O)N(R)₂, C(═S)N(R)₂, (CH₂)₀₋₂N(R)C(═O)R, (CH₂)₀₋₂N(R)N(R)₂, N(R)N(R)C(═O)R, N(R)N(R)C(═O)OR, N(R)N(R)C(═O)N(R)₂, N(R)S(═O)₂R, N(R)S(═O)₂N(R)₂, N(R)C(═O)OR, N(R)C(═O)R, N(R)C(═S)R, N(R)C(═O)N(R)₂, N(R)C(═S)N(R)₂, N(C(═O)R)—C(═O)R, N(OR)R, C(═NH)N(R)₂, C(═O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C═C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group. Illustrative examples of cycloalkyl groups include, but are not limited to, the following moieties:

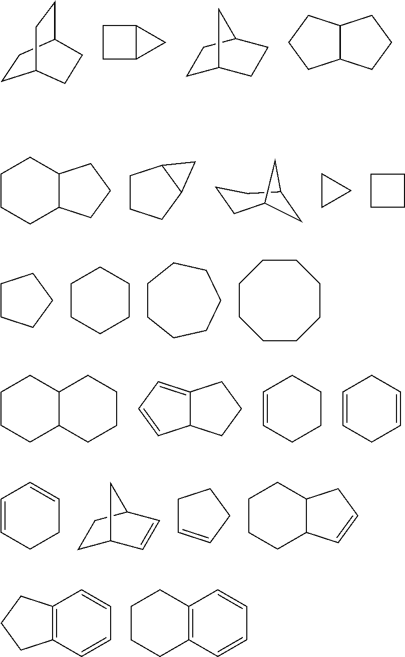

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups.

Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group.

The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein. Non-limiting examples of heterocycloalkyl groups include:

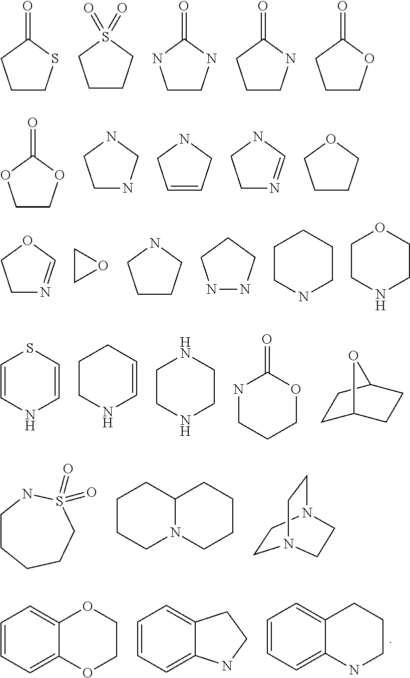

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein. Non-limiting examples of heteroaryl groups include the following moieties:

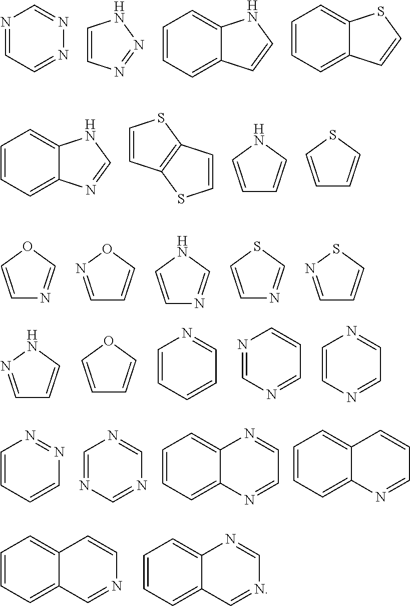

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃, wherein each R is independently selected, and protonated forms of each, except for —NR₃, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxycyclohexyl)ethyl, 2-(2,3-epoxycylopentyl)ethyl, 2-(4-methyl-3,4-epoxycyclohexyl)propyl, 2-(3,4-epoxy-3-methylcylohexyl)-2-methylethyl, and 5,6-epoxyhexyl.

The term “monovalent” as used herein refers to a substituent connecting via a single bond to a substituted molecule. When a substituent is monovalent, such as, for example, F or Cl, it is bonded to the atom it is substituting by a single bond.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

As used herein, the term “PTM” refers to a protein targeting moiety, which is a moiety that can bind to a protein of interest. The term PTM can also refer to an ATKI as defined here.

Compounds

The compounds described herein can be synthesized using techniques well-known in the art of organic synthesis. The starting materials and intermediates required for the synthesis can be obtained from commercial sources or synthesized according to methods known to those skilled in the art. A general procedure for making certain compounds described herein can be found in U.S. Patent Application Publication No. US20140356322, which is hereby incorporated by reference in its entirety.

In various embodiments, a compound of Formula (I), or a salt, enantiomer, stereoisomer, solvate, polymorph or N-oxide thereof, is provided.

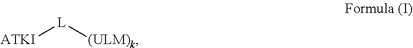

In the compound of Formula (I), ATKI is an allosteric tyrosine kinase inhibitor, L is a linker, ULM is a ubiquitin ligase binder, and k is an integer ranging from 1 to 4. The ATKI moiety is covalently bonded to L, and the ULM moiety is covalently bonded to L. In various embodiments, ATKI is capable of binding to the allosteric site of a tyrosine kinase such as c-ABL and/or BCR-ABL. Binding of the compound of Formula (I) through the ATKI moiety to a tyrosine kinase such as c-ABL and/or BCR-ABL results in the ubiquitination of tyrosine kinase c-ABL and/or BCR-ABL by a ubiquitin ligase. In various embodiments, the ubiquitin ligase is brought into close proximity to the tyrosine kinase by binding to the ULM moiety in the compound of Formula (I), thereby enabling ubiquitination of the tyrosine kinase. In various embodiments, upon binding of the compound of Formula (I) simultaneously to a tyrosine kinase and a ubiquitin ligase, the tyrosine kinase is ubiquitinated by the ubiquitin ligase.

In any of the aspects or embodiments described herein, the ULM has an affinity (IC₅₀) for its respective target protein of less than about 500 μM, 450 μM, 400 μM, 350 μM, 300 μM, 25 μM, 200 μM, 150 μM, 100 μM, 50 μM, 10 μM, 0.10 μM, 0.01 μM, 0.001 μM, 0.1 nM, 0.01 nM, 0.001 nM, or less. The determination of the IC₅₀ can be performed using methods well known to those of skill in the art in view of the present disclosure.

Allosteric Tyrosine Kinase Inhibitor (ATKI)

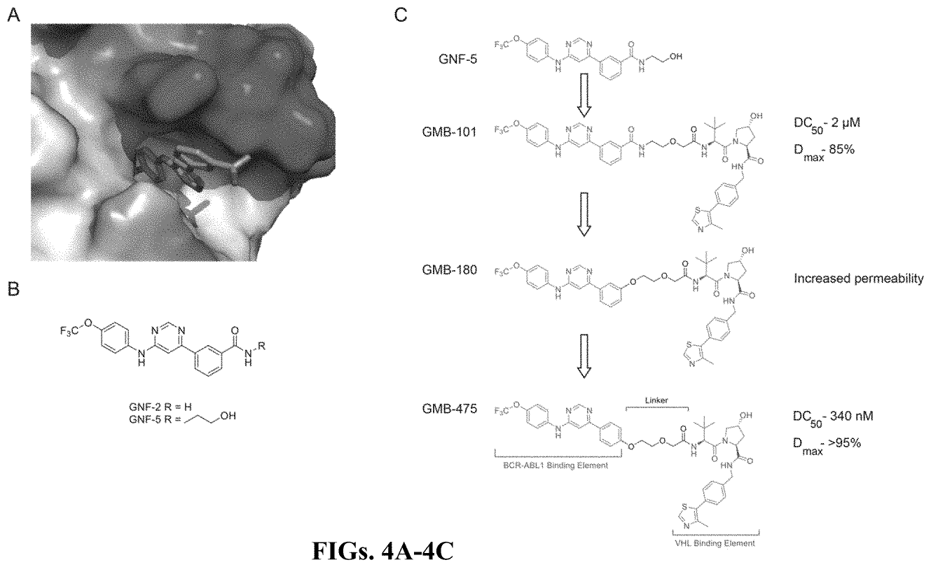

An allosteric tyrosine kinase inhibitor (ATKI) moiety, as described herein, can bind to and inhibit a tyrosine kinase, or a subunit thereof, at an allosteric site of the tyrosine kinase rather than at an ATP-binding site. In various embodiments, the ATKI can bind to and inhibit c-ABL, BCR-ABL, and/or any combinations thereof. In various embodiments, the ATKI can bind to and inhibit c-ABL and BCR-ABL. The ATKI can, in various embodiments, binds to an allosteric site on c-ABL and inhibits c-ABL. The ATKI can bind to an allosteric site on BCR-ABL and inhibit BCR-ABL. In various embodiments, the ATKI binds to an allosteric site on at least one of c-ABL and BCR-ABL, and inhibits at least one of c-ABL and BCR-ABL. In various embodiments, the ATKI is selected from the group consisting of GNF-2, GNF-5, and asciminib, or any combinations thereof. GNF-2 and GNF-5 bind at the membrane tethering myristate binding pocket present on ABL1/BCR-ABL1 (FIG. 4A). Inspection of the crystal structure of the GNF-2/ABL1 complex (PDB ID:3K5V) revealed solvent exposed regions suitable for linker attachment and therefore PROTAC conversion.

In various embodiments, an ATKI moiety can be GNF-2, a fragment thereof, or a substituted analog thereof. GNF-2 is also known as 3-[6-[[4-(trifluoromethoxy)phenyl]amino]-4-pyrimidinyl]-benzamide, or a salt or solvate thereof, and has the following structure:

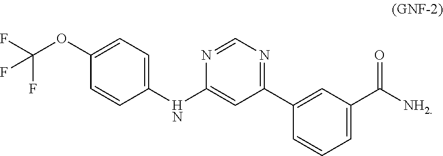 [SMILES: NC(=O)c1cccc(-c2cc(Nc3ccc(OC(F)(F)F)cc3)ncn2)c1]

In various embodiments, linker L can be bonded to any open valence on GNF-2, such that any C—H, N—H, or O—H bond can be replaced by a C-L, N-L, or O-L bond, respectively. In any aspect or embodiment described herein, the ATKI is represented by:

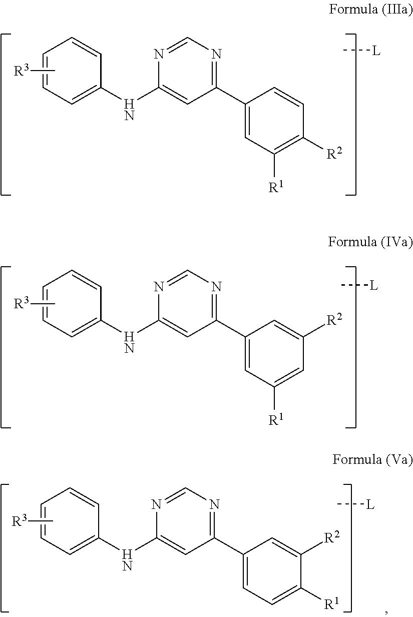

wherein -----L denotes that the linker may be attached to the ATKI moiety via any open valence on the moiety, and each of R¹, R², and R³ is as described herein. For example, any CH, NH, or OH bond of Formula (IIIa)-Formula (Va) can be replaced with C-L, N-L, O-L bond, respectively.

In various embodiments and without limitation, L can be bonded to any of the following positions on GNF-2 or its analogs:

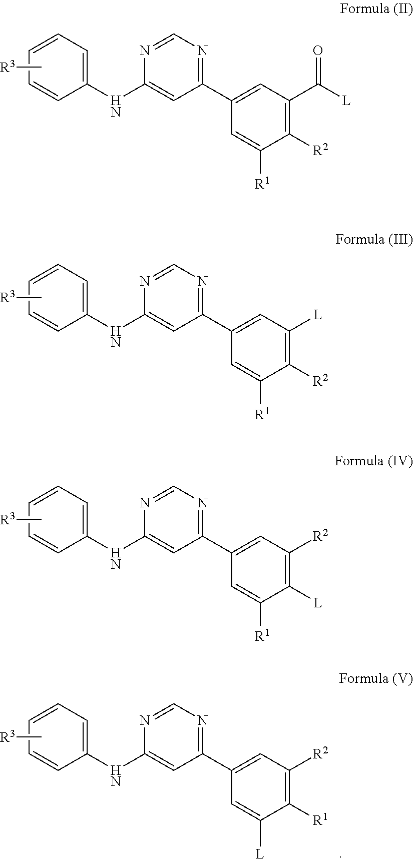

In various embodiments, in the compounds of Formula (II) to Formula (IV) and Formula (IIIa) to Formula (Va), the R³ substituent is not in the para position, such that the phenyl ring bearing the R³ substituent is substituted at the meta or ortho position as follows:

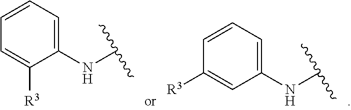

In various embodiments and without limitation, the ATKI moiety can have the structure of Formula (VI) or Formula (VIa).

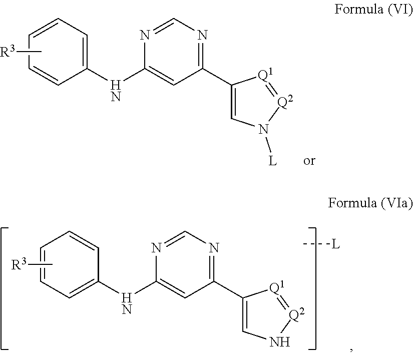

wherein -----L denotes that the linker may be attached to the ATKI moiety via any open valence on the moiety, and each of Q¹, Q², and R³ is as described herein. For example, any CH, NH, or OH bond of Formula (VIa) can be replaced with C-L, N-L, O-L bond, respectively.

In the compounds of Formula (II)-Formula (VI) and Formula (IIIa)-Formula (VIa),

R¹ is independently H, halogen, optionally substituted C₁₋₆ alkyl, or optionally substituted C₁₋₆ alkoxy;

R² is independently H, halogen, an optionally substituted acyl, an optionally substituted amide, optionally substituted C₁₋₆ alkyl, optionally substituted C₁₋₆ alkoxy,

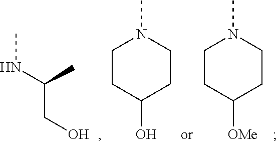

R³ is independently H, halogen, optionally substituted C₁₋₆ alkyl, optionally substituted C₁₋₆ alkoxy, OCF₃, OCFH₂, OCF₂H, OCFCl₂, or OCF₂Cl; and Q¹ and Q² are each independently CH or N.

In various embodiments, the ATKI has the structure of Formula (II) or Formula (IIIa), wherein R¹ and R² are both H. In various embodiments, the ATKI has the structure of Formula (IV) or Formula (IVa), wherein R¹ and R² are both H. In various embodiments, the ATKI has the structure of Formula (VI) or Formula (VIa), wherein Q¹ is CH and Q² is N.

In various embodiments, an ATKI moiety can be GNF-5, a fragment thereof, or a substituted analog thereof. GNF-5 is also known as N-(2-hydroxyethyl)-3-[6-[[4-(trifluoromethoxy) phenyl]amino]-4-pyrimidinyl]benzamide, or a salt or solvate thereof, and has the following structure:

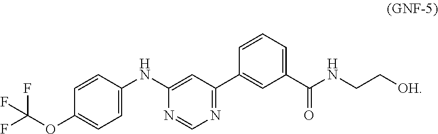 [SMILES: O=C(NCCO)c1cccc(-c2cc(Nc3ccc(OC(F)(F)F)cc3)ncn2)c1]

In various embodiments, linker L can be bonded to any open valence on GNF-5 such that any C—H, N—H, or O—H bond can be replaced by a C-L, N-L, or O-L bond, respectively. In any aspect or embodiment described herein, the ATKI is represented by:

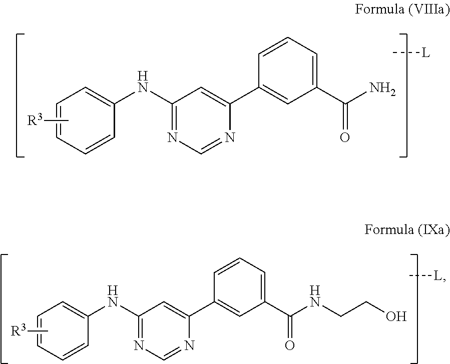

wherein -----L denotes that the linker may be attached to the ATKI moiety via any open valence on the moiety, and R³ is as described herein. For example, any CH, NH, or OH bond of Formula (VIIIa) and Formula (IXa) can be replaced with C-L, N-L, O-L bond, respectively.

In various embodiments and without limitation, L can be bonded to any of the following positions on GNF-5 or its analogs:

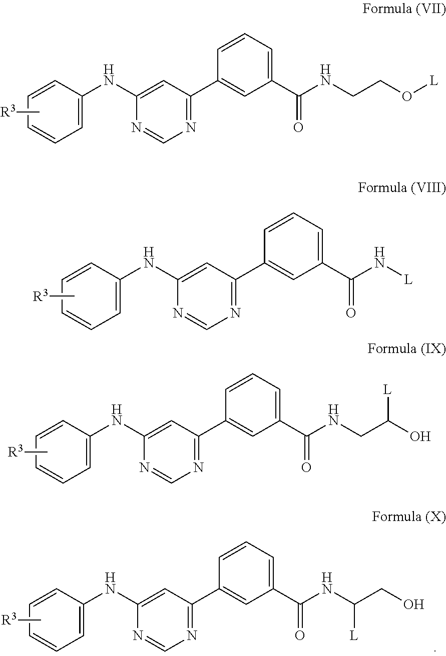

In various embodiments, in the compounds of Formula (VII) to Formula (X), Formula (VIIIa). and Formula (IXa), the R³ substituent is not in the para position, such that the phenyl ring bearing the R³ substituent is substituted at the meta or ortho position as follows:

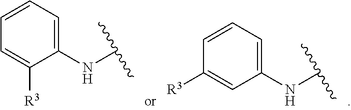

In the compounds of Formula (IX) and Formula (X), although no absolute stereochemistry is indicated at the C-L bond, racemic mixtures and individual (R)- and (S)-enantiomers at the C-L stereocenter of the ATKI are contemplated.

In various embodiments, an TKI moiety can be asciminib, a fragment thereof, or a substituted analog thereof. Asciminib is also known as N-[4-[chloro(difluoro)methoxy]phenyl]-6-[(3R)-3-hydroxypyrrolidin-1-yl]-5-(1H-pyrazol-5-yl)pyridine-3-carboxamide, or a salt or solvate thereof, and has the following structure:

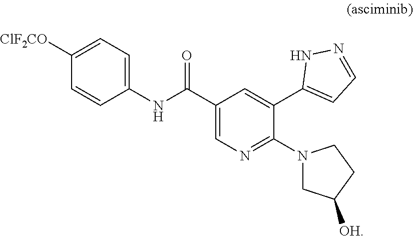

In various embodiments, linker L can be bonded to any open valence on asciminib, such that any C—H, N—H, or O—H bond can be replaced by a C-L, N-L, or O-L bond, respectively. In any aspect or embodiment described herein, the ATKI is represented by:

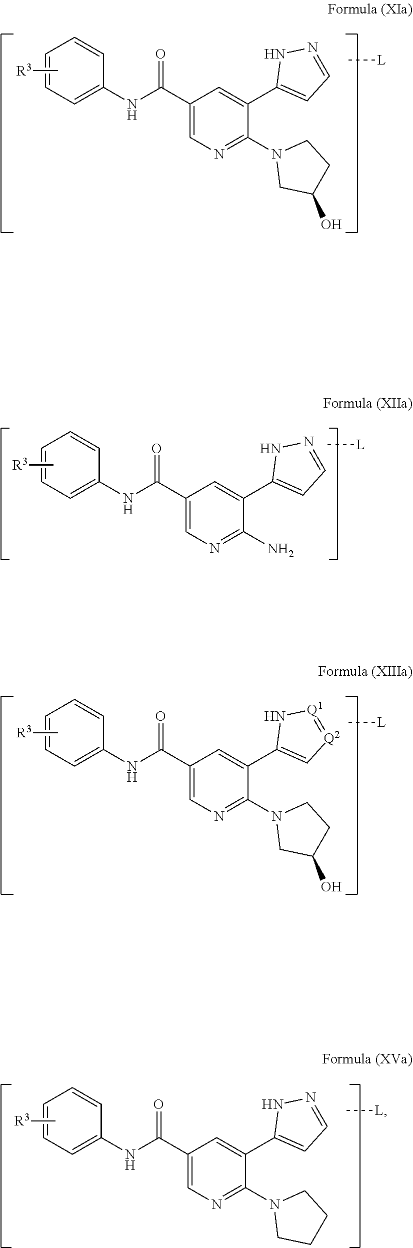

wherein: -----L denotes that the linker may be attached to the ATKI moiety via any open valence on the moiety, and each of Q¹, Q², and R³ is as described herein. For example, any CH, NH, or OH bond of Formula (XIa)-Formula (XIIIa), and Formula (XVa) can be replaced with C-L, N-L, O-L bond, respectively.

In various embodiments and without limitation, L can be bonded to any of the following positions on asciminib or its analogs:

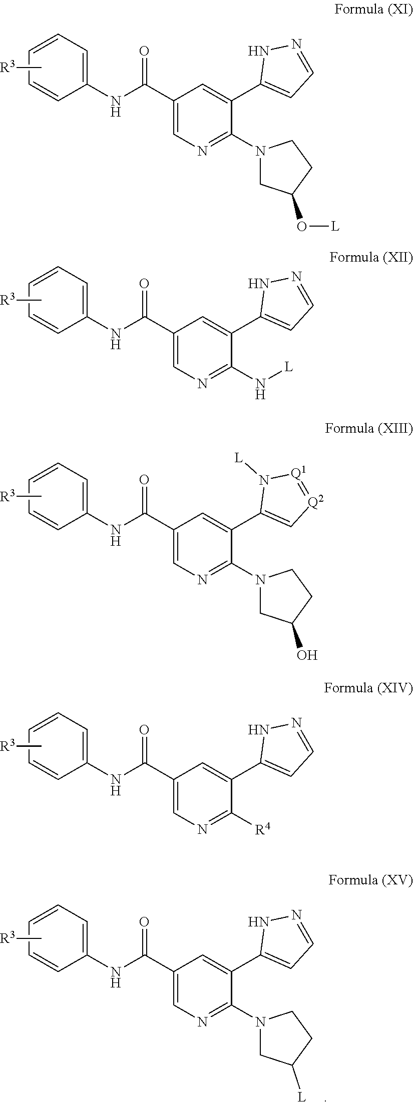

Variable R⁴ is halogen, optionally substituted C₁₋₆ alkyl, optionally substituted C₁₋₆ alkoxy

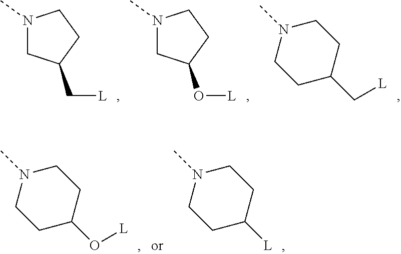

In the compound of Formula (XV), although no absolute stereochemistry is indicated at the C-L bond, racemic mixtures and individual (R)- and (S)-enantiomers at the C-L stereocenter of the ATKI are contemplated.

Linker (L)

A suitable linker in the compounds of Formula (I) is covalently bonded to the ATKI moiety, and is further covalently bonded to at least one ubiquitin ligase binding moiety (ULM).

In various embodiments, the ubiquitin ligase is an E3 ubiquitin ligase. The E3 ubiquitin ligase can be, in various embodiments, Von Hippel Lindau (VHL) E3 ubiquitin ligase, Inhibitor of Apoptosis Protein (IAP) E3 ubiquitin ligase, and/or Cereblon (CRBN) E3 ligase.

In certain embodiments, the linker of the present invention corresponds to formula —(CH₂)ₘ₁—X⁴—((CH₂)_{m2′}—X⁵)ₘ₂—(CH₂)ₘ₃—X⁶—, wherein the ATKI is covalently bonded to —(CH₂)ₘ₁, and the ULM is covalently bonded to X⁶. Alternatively, —(CH₂)ₘ₁ is covalently bonded to the ULM, and X⁶ is covalently bonded to the ATKI moiety. Each m1, m2, m2′, and m3 is independently 0, 1, 2, 3, 4, 5, 6, 7, 8, 9, or 10; each X⁴ and X⁵ is independently absent (a bond), O, S, or N—R²⁰; each X⁶ is independently absent (a bond), C(═O), NHC(═O), C(═S), C(═NR²⁰), O, S, or N—R²⁰, wherein each R²⁰ is independently selected from the group consisting of hydrogen, optionally substituted C₁-C₆ alkyl, optionally substituted aryl, optionally substituted heteroaryl, optionally substituted C₃-C₈ cycloalkyl, and optionally substituted C₃-C₈ cycloheteroalkyl. When X⁶ is NHC(═O), the ATKI or ULM can be covalently bonded to the nitrogen atom or to the carbon atom in C(═O). In various embodiments, linker L is a bond.

In various embodiments, the linker L corresponds to formula —(CH₂)ₘ₁—O—(CH₂—CH₂—O)ₘ₂—(CH₂)ₘ₃—C(O)—, wherein the ATKI moiety is covalently bonded to —(CH₂)ₘ₁, and the ULM is covalently bonded to C(O)—. Alternatively, —(CH₂)ₘ₁ is covalently bonded to the ULM, and C(O)— is covalently bonded to the ATKI moiety. Each m1, m2, and m3 is defined elsewhere herein.

Linker L can correspond to formula —(CHR²¹)ₘ₁—O—(CHR²²—CHR²³—O)ₘ₂—(CHR²⁴)ₘ₃—C(O)—, wherein the ATKI moiety is covalently bonded to —(CH₂)ₘ₁, and the ULM is covalently bonded to C(O)—. Alternatively, —(CH₂)ₘ₁ is covalently bonded to the ULM, and C(O)— is covalently bonded to the ATKI moiety. Each m1, m2, and m3 is defined elsewhere herein; each R²¹, R²², R²³, and R²⁴ is independently selected from the group consisting of hydrogen, optionally substituted C₁-C₆ alkyl, optionally substituted aryl, optionally substituted heteroaryl, optionally substituted C₃-C₈ cycloalkyl, and optionally substituted C₃-C₈ cycloheteroalkyl.

In various embodiments, in linker L m1 is 0, m2′ is 2, m2 is 1 or 2, m3 is 1, and X⁴, X⁵, and X⁶ are O. In various embodiments, in linker L m1 is 2, m2′ is 2, m2 is 1, m3 is 1, and X⁴, X⁵, and X⁶ are O. In various embodiments, in linker L m1 is 2, m2′ is 2, m2 is 3, m3 is 1, and X⁴, X⁵, and X⁶ are O.

In various embodiments, linker L is a polyethylene glycol chain ranging in size from about 1 to about 12 ethylene glycol units, from about 1 to about 10 ethylene glycol units, from about 2 to about 6 ethylene glycol units, from about 2 to about 5 ethylene glycol units, or from about 2 to about 4 ethylene glycol units. In various embodiments, linker L is one ethylene glycol unit.

In yet other embodiments, the linker L corresponds to

-(D-CON-D)ₘ₁- (II),

wherein each D is independently a bond (absent), or —(CH₂)ₘ₁—Y—C(O)—Y—(CH₂)ₘ₁—; wherein m1 is defined elsewhere herein; Y is O, S or N—R⁵; CON is a bond (absent), an optionally substituted C₃-C₈ cycloheteroalkyl, piperazinyl or a group selected from the group consisting of the following chemical structures:

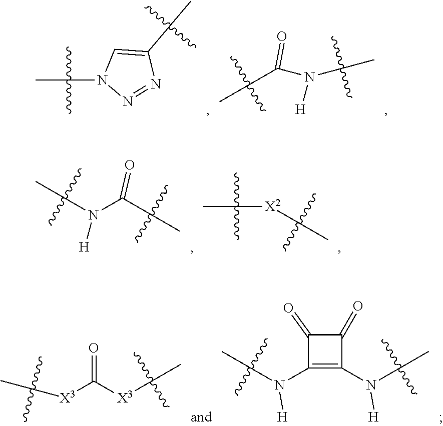

wherein X² is O, S, NR⁵, ( ), S(O)₂, —S(O)₂O, —OS(O)₂, or OS(O)₂O; X³ is O, S, CHR⁵, NR⁵; and R⁵ is H or a C₁-C₃ alkyl group optionally substituted with one or two hydroxyl groups.

The linker L described herein is covalently bonded to the ATKI and ULM, through an amide, ester, thioester, keto group, carbamate (urethane) or ether group. As described herein, the linking position can be at any chemically stable position on the ATKI moiety and the ULM moiety.

In any aspect or embodiment described herein, the linker L is a bond or a chemical linker group represented by the formula -(A^{L})_{q}-, wherein A is a chemical moiety and q is an integer from 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100), and wherein L is covalently bound to the ATKI and the ULM, and provides for sufficient binding of the ATKI to the protein target and the ULM to an E3 ubiquitin ligase to result in target protein ubiquitination.

In any aspect or embodiment described herein, the linker group L is -(A^{L})_{q}-, wherein:
(A^{L})_{q} is a group which is connected to at least one of a ULM (such as a CLM or a VLM), PTM moiety, or a combination thereof;
q of the linker is an integer greater than or equal to 1 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100);
each A^{L} is independently selected from the group consisting of, a bond, CR^{L1}R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4} NR^{L3}C(═NCN), NR^{L3}C(═CNO₂)NR^{L4}, C₃₋₁₁cycloalkyl optionally substituted with 0-6 R^{L1} and/or R^{L2} groups, C₅₋₁₃ spirocycloalkyl optionally substituted with 0-9 R^{L1} and/or R^{L2} groups, C₃₋₁₁heterocyclyl optionally substituted with 0-6 R^{L1} and/or R^{L2} groups, C₅₋₁₃ spiroheterocyclyl optionally substituted with 0-8 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form cycloalkyl and/or heterocyclyl moiety, optionally substituted with 0-4 R^{L5} groups; and R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₃₋₈cycloalkyl, SC₃₋₈cycloalkyl, NHC₃₋₈cycloalkyl, N(C₃₋₈cycloalkyl)₂, N(C₃₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈ alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NH SO₂NH(C₁₋₈alkyl), NH SO₂N(C₁₋₈alkyl)₂, NH SO₂NH₂.

In any aspect or embodiment described herein, the unit A^{L} of linker (L) comprises a group represented by a general structure selected from the group consisting of:
—NR(CH₂)ₙ-(lower alkyl)-, —NR(CH₂)ₙ-(lower alkoxyl)-, —NR(CH₂)ₙ-(lower alkoxyl)-OCH₂—, —NR(CH₂)ₙ-(lower alkoxyl)-(lower alkyl)-OCH₂—, —NR(CH₂)ₙ-(cycloalkyl)-(lower alkyl)-OCH₂—, —NR(CH₂)ₙ-(hetero cycloalkyl)-, —NR(CH₂CH₂O)ₙ-(lower alkyl)-O—CH₂—, —NR(CH₂CH₂O)ₙ-(hetero cycloalkyl)-O—CH₂—, —NR(CH₂CH₂O)ₙ-Aryl-O—CH₂—, —NR(CH₂CH₂O)ₙ-(hetero aryl)-O—CH₂—, NR(CH₂CH₂O)ₙ-(cyclo alkyl)-O-(hetero aryl)-O—CH₂—, —NR(CH₂CH₂O)ₙ-(cyclo alkyl)-O-Aryl-O—CH₂—, —NR(CH₂CH₂O)ₙ-(lower alkyl)-NH-Aryl-O—CH₂—, —NR(CH₂CH₂O)ₙ-(lower alkyl)-O-Aryl-CH₂, —NR(CH₂CH₂O)ₙ-cycloalkyl-O-Aryl-, —NR(CH₂CH₂O)ₙ-cycloalkyl-O-(heteroaryl)l-, —NR(CH₂CH₂)ₙ-(cycloalkyl)-O-(heterocyclyl)-CH₂, —NR(CH₂CH₂)ₙ-(heterocyclyl)-(heterocyclyl)-CH₂, N(R1R2)-(heterocyclyl)-CH₂; where
n of the linker can be 0 to 10;
R of the linker can be H, lower alkyl; and
R1 and R2 of the linker can form a ring with the connecting N.

In any aspect or embodiment described herein, the linker (L) includes an optionally substituted C₁-C₁₀₀ alkyl (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, C₅₀, C₅₁, C₅₂, C₅₃, C₅₄, C₅₅, C₅₆, C₅₇, C₅₈, C₅₉, C₆₀, C₆₁, C₆₂, C₆₃, C₆₄, C₆₅, C₆₆, C₆₇, C₆₈, C₆₉, C₇₀, C₇₁, C₇₂, C₇₃, C₇₄, C₇₅, C₇₆, C₇₇, C₇₈, C₇₉, C₈₀, C₈₁, C₈₂, C₈₃, C₈₄, C₈₅, C₈₆, C₈₇, C₈₈, C₈₉, C₉₀, C₉₁, C₉₂, C₉₃, C₉₄, C₉₅, C₉₆, C₉₇, C₉₈, C₉₉, or C₁₀₀ alkyl), wherein each carbon is optionally substituted with (1) a heteroatom selected from N, S, P, or Si atoms that has an appropriate number of hydrogens, substitutions, or both to complete valency, (2) an optionally substituted cycloalkyl or bicyclic cycloalkly, (3) an optionally substituted heterocyloalkyl or bicyclic heterocyloalkyl, (4) an optionally substituted aryl or bicyclic aryl, or (5) optionally substituted heteroaryl or bicyclic heteroaryl. In any aspect or embodiment described herein, the linker (L) does not have heteroatom-heteroatom bonding (e.g., no heteroatoms are covalently linker or adjacently located).

In any aspect or embodiment describe herein, the linker (L) includes an optionally substituted C₁-C₁₀₀ alkyl (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, C₅₀, C₅₁, C₅₂, C₅₃, C₅₄, C₅₅, C₅₆, C₅₇, C₅₈, C₅₉, C₆₀, C₆₁, C₆₂, C₆₃, C₆₄, C₆₅, C₆₆, C₆₇, C₆₈, C₆₉, C₇₀, C₇₁, C₇₂, C₇₃, C₇₄, C₇₅, C₇₆, C₇₇, C₇₈, C₇₉, C₈₀, C₈₁, C₈₂, C₈₃, C₈₄, C₈₅, C₈₆, C₈₇, C₈₈, C₈₉, C₉₀, C₉₁, C₉₂, C₉₃, C₉₄, C₉₅, C₉₆, C₉₇, C₉₈, C₉₉, or C₁₀₀ alkyl), wherein:
each carbon is optionally substituted with CR^{L1}R^{L2}, O, S, SO, SO₂, NR^{L3}, SO₂NR^{L3}SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, —P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₅₋₁₃ spirocycloalkyl optionally substituted with 0-9 R^{L1} and/or R^{L2} groups, C₃₋₁₁ heterocyclyl optionally substituted with 0-6 R^{L1} and/or R^{L2} groups, C₅₋₁₃ spiroheterocyclyl optionally substituted with 0-8 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form cycloalkyl and/or heterocyclyl moiety, optionally substituted with 0-4 R^{L5} groups; and
R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₃₋₈cycloalkyl, SC₃₋₈cycloalkyl, NHC₃₋₈cycloalkyl, N(C₃₋₈cycloalkyl)₂, N(C₃₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NH SO₂NH(C₁₋₈alkyl), NH SO₂N(C₁₋₈alkyl)₂, NH SO₂NH₂.

In any aspect or embodiment described herein, the linker (L) includes about 1 to about 50 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) alkylene glycol units that are optionally substituted, wherein carbon or oxygen may be substituted with a heteroatom selected from N, S, P, or Si atoms with an appropriate number of hydrogens to complete valency. For example, in any aspect or embodiment described herein, the linker (L) has a chemical structure selected from:

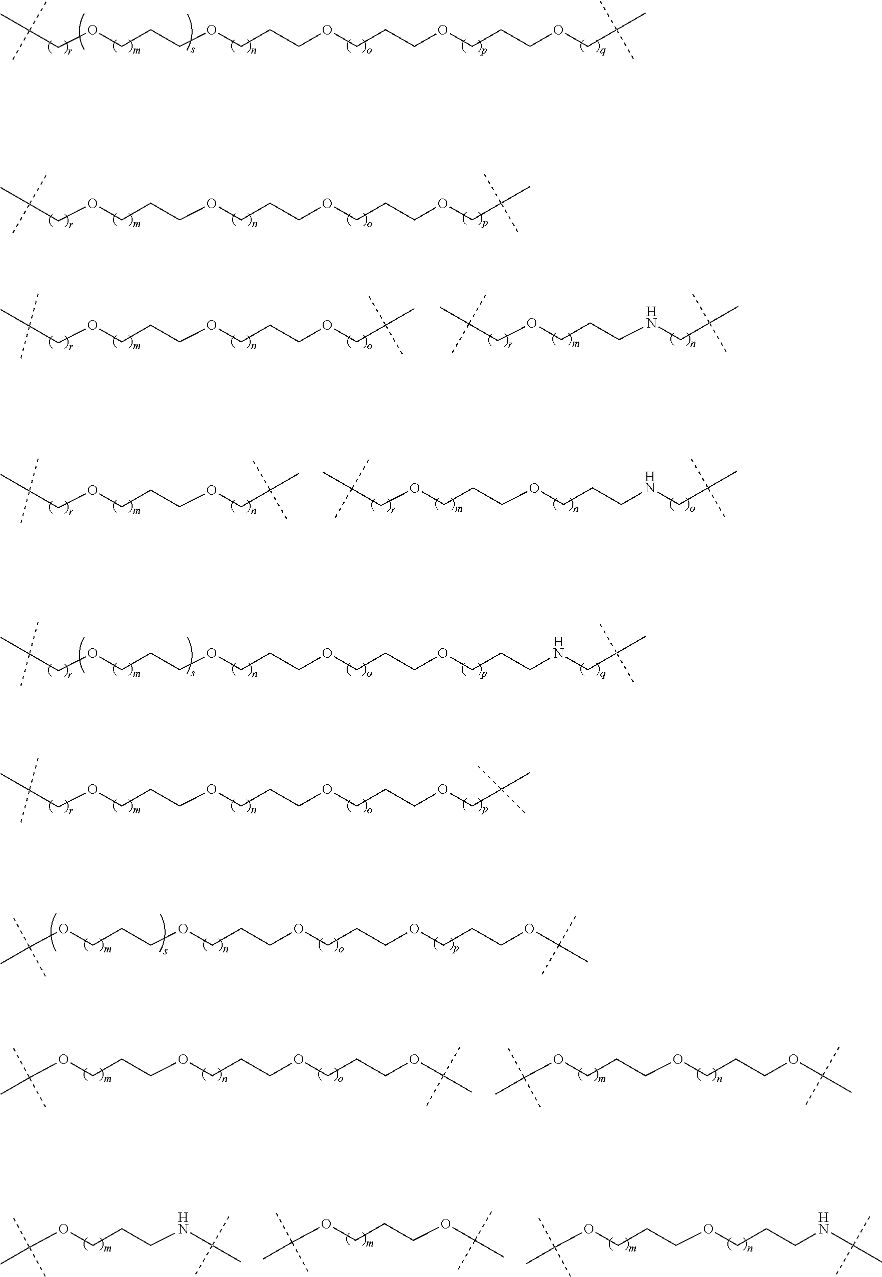

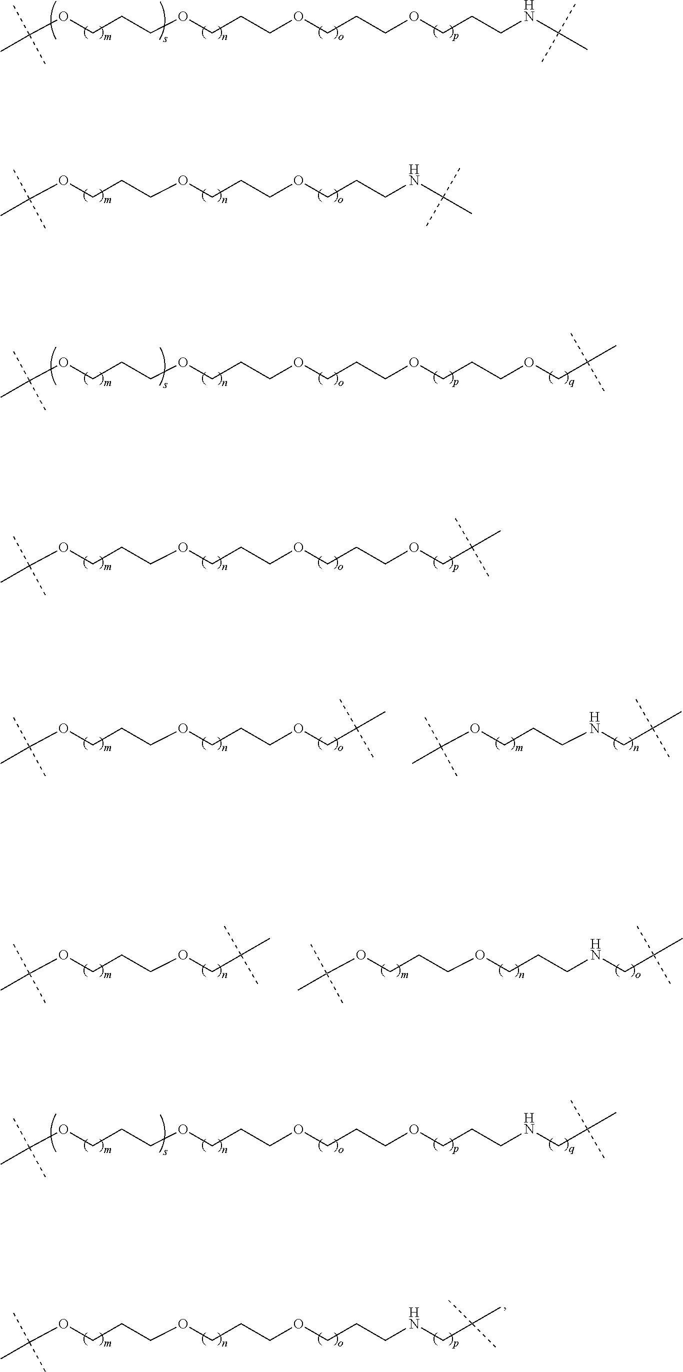

wherein carbon or oxygen may be substituted with a heteroatom selected from N, S, P, or Si atoms with an appropriate number of hydrogens to complete valency, and m, n, o, p, q, r, and s are independently selected from 0, 1, 2, 3, 4, 5, 6, 7, 8, 9, 10, 11, 12, 13, 14, 15, 16, 17, 18, 19, and 20.

Ubiquitin Ligase Moiety (ULM)

A ubiquitin ligase binder (ULM) moiety of compounds described herein binds to a ubiquitin ligase. In various embodiments, the ubiquitin ligase is an E3 ubiquitin ligase. In various embodiments, the E3 ubiquitin ligase is a Von Hippel Lindau (VHL) E3 ubiquitin ligase, an MDM2 E3 ubiquitin ligase, or a Cereblon (CRBN) E3 ubiquitin ligase.

### Von Hippel-Lindau E3 Ubiquitin Ligase Binding Moieties

In various embodiments, ULM corresponds to Formula (XVI):

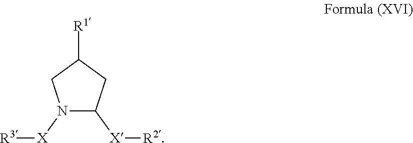

In Formula (XVI), R^{1′} is a group selected from the group consisting of OH, an optionally substituted C₁-C₆ alkyl, an optionally substituted —(CH₂)ₙOH, an optionally substituted —(CH₂)ₙSH, an optionally substituted (CH₂)ₙ—O—(C₁-C₆)alkyl, an optionally substituted (CH₂)ₙ—X⁷—(C₁-C₆)alkyl, an optionally substituted —(CH₂)ₙCOOH, an optionally substituted —(CH₂)ₙC(═O)—(C₁-C₆ alkyl), an optionally substituted —(CH₂)ₙNHC(═O)—R⁶, an optionally substituted —(CH₂)ₙC(═O)—NR⁶R⁷, an optionally substituted —(CH₂)ₙOC(═O)—NR⁶R⁷, —(CH₂O)ₙH, an optionally substituted —(CH₂)ₙOC(═O)—(C₁-C₆ alkyl), an optionally substituted —(CH₂)ₙC(═O)—O—(C₁-C₆ alkyl), an optionally substituted —(CH₂O)ₙCOOH, an optionally substituted —(OCH₂)ₙO—(C₁-C₆ alkyl), an optionally substituted —(CH₂O)ₙC(═O)—(C₁-C₆ alkyl), an optionally substituted —(OCH₂)ₙNHC(═O)—R⁶, an optionally substituted —(CH₂O)ₙC(═O)—NR⁶R⁷, —(CH₂CH₂O)ₙH, an optionally substituted —(CH₂CH₂O)ₙCOOH, an optionally substituted —(OCH₂CH₂)ₙO—(C₁-C₆ alkyl), an optionally substituted —(CH₂CH₂O)ₙC(═O)—(C₁-C₆ alkyl), an optionally substituted —(OCH₂CH₂)ₙNHC(═O)—R⁶, an optionally substituted —(CH₂CH₂O)ₙC(═O)—NR⁶R⁷, an optionally substituted —S(═O)₂R^{S}, S(═O)R⁵, NO₂, CN, and halogen.

Variables R⁶ and R⁷ are each independently H or C₁-C₆ alkyl which may be optionally substituted with one or two hydroxyl groups or up to three halogen groups.

Variable R⁵ is C₁-C₆ alkyl, optionally substituted aryl, optionally substituted heteroaryl or optionally substituted heterocycle or —(CH₂)ₘNR⁶R⁷.

Variables X and X′ are each independently C═O, C═S, —S(═O), S(═O)₂.

Variable X⁷ is an optionally substituted epoxide moiety.

In Formula (XVI), R^{2′} is a group selected from the group consisting of optionally substituted —(CH₂)ₙ—(C═O)ᵤ(NR₁)ᵥ(SO₂)_{w}—C₁-C₆ alkyl, an optionally substituted —(CH₂)ₙ—(C═O)ᵤ(NR⁶)ᵥ(SO₂)_{w}NR^{1N}R^{2N}, an optionally substituted —(CH₂)ₙ—(C═O)ᵤ(NR⁶)ᵥ(SO₂)_{w}-Aryl, an optionally substituted —(CH₂)ₙ—(C═O)ᵤ(NR⁶)ᵥ(SO₂)_{w}-Heteroaryl, an optionally substituted —(CH₂)ₙ—(C═O)ᵥNR⁶(SO₂)_{w}-Heterocycle, an optionally substituted —NR²⁵—(CH₂)ₙ—C(O)ᵤ(NR₁)ᵥ(SO₂)_{w}—C₁-C₆ alkyl, an optionally substituted —NR²⁵—(CH₂)ₙ—C(O)ᵤ(NR⁶)ᵥ(SO₂)_{w}—NR^{1N}R^{2N}, an optionally substituted —NR²⁵—(CH₂)ₙ—C(O)ᵤ(NR⁶)ᵥ(SO₂)_{w}—NR⁶C(O)R^{N}, an optionally substituted —NR²⁵—(CH₂)ₙ—(C═O)ᵤ(NR⁶)ᵥ(SO₂)_{w}-Aryl, an optionally substituted —NR²⁵—(CH₂)ₙ—(C═O)ᵤ(NR⁶)ᵥ(SO₂)_{w}-Heteroaryl or an optionally substituted —NR²⁵—(CH₂)ₙ—(C═O)ᵥNR⁶(SO₂)_{w}-Heterocycle, an optionally substituted —X^{R2′}—C₁-C₆ alkyl; an optionally substituted —X^{R2′}-Aryl, an optionally substituted —X^{R2′}-Heteroaryl, and an optionally substituted —X^{R2′}-Heterocycle;

In Formula (XVI), R^{3′} is a group selected from the group consisting of an optionally substituted C₁-C₆ alkyl, an optionally substituted —(CH₂)ₙ—C(O)ᵤ(NR⁶)ᵥ(SO₂)_{w}—C₁-C₆ alkyl, an optionally substituted —(CH₂)ₙ—C(O)ᵤ(NR⁶)ᵥ(SO₂)_{w}—NR^{1N}R^{2N}, an optionally substituted —(CH₂)ₙ—C(O)ᵤ(NR⁶)ᵥ(SO₂)_{w}—NR⁶C(O)R^{1N}, an optionally substituted —(CH₂)ₙ—C(O)ᵤ(NR⁶)ᵥ(SO₂)_{w}—C(O)NR⁶R⁷, an optionally substituted —(CH₂)ₙ—C(O)ᵤ(NR⁶)ᵥ(SO₂)_{w}-Aryl, an optionally substituted —(CH₂)ₙ—C(O)ᵤ(NR⁶)ᵥ(SO₂)_{w}-Heteroaryl, an optionally substituted —(CH₂)ₙ—C(O)ᵤ(NR⁶)ᵥ(SO₂)_{w}-Heterocycle, an optionally substituted —NR²⁵—(CH₂)ₙ—C(O)ᵤ(NR⁶) (SO₂)_{w}—C₁-C₆ alkyl, an optionally substituted —NR²⁵—(CH₂)ₙ—C(O)ᵤ(NR⁶)(SO₂)_{w}—NR^{1N}R^{2N}, an optionally substituted —NR²⁵—(CH₂)ₙ—C(O)ᵤ(NR⁶)ᵥ(SO₂)_{w}—NR⁶C(O)R^{1N}, an optionally substituted —NR²⁵—(CH₂)ₙ—C(O)ᵤ(NR⁶)ᵥ(SO₂)_{w}-Aryl, an optionally substituted —NR²⁵—(CH₂)ₙ—C(O)ᵤ(NR⁶)ᵥ(SO₂)_{w}-Heteroaryl, an optionally substituted —NR^{1′}—(CH₂)ₙ—C(O)ᵤ(NR⁶)ᵥ(SO₂)_{w}-Heterocycle, an optionally substituted —O—(CH₂)ₙ—(C═O)ᵤ(NR⁶)ᵥ(SO₂)_{w}—C₁-C₆ alkyl, an optionally substituted —O—(CH₂)ₙ—(C═O)ᵤ(NR⁶)(SO₂)_{w}—NR^{1N}R^{2N}, an optionally substituted —O—(CH₂)ₙ—(C═O)ᵤ(NR⁶)ᵥ(SO₂)_{w}—NR⁶C(O)R^{1N}, an optionally substituted —O—(CH₂)ₙ—(C═O)ᵤ(NR⁶)ᵥ(SO₂)_{w}-Aryl, an optionally substituted —O—(CH₂)ₙ—(C═O)ᵤ(NR⁶)ᵥ(SO₂)_{w}-Heteroaryl, an optionally substituted —O—(CH₂)ₙ—(C═O)ᵤ(NR⁶)ᵥ(SO₂)_{w}-Heterocycle, —(CH₂)ₙ—(V)ₙ, —(CH₂)ₙ—(V)_{n′}—C₁-C₆ alkyl, an optionally substituted —(CH₂)ₙ—(V)_{n′}—(CH₂)ₙ—(V)ₙ-Aryl, an optionally substituted —(CH₂)ₙ—(V)ₙ, —(CH₂)ₙ—(V)ₙ—Heteroaryl, an optionally substituted —(CH₂)ₙ—(V)ₙ, —(CH₂)ₙ—(V)_{n′}-Heterocycle, an optionally substituted —(CH₂)ₙ—N(R⁶)(C═O)ₘ—(V)_{n′}—C₁-C₆ alkyl, an optionally substituted —(CH₂)ₙ—N(R⁶)(C═O)_{m′}—(V)_{n′}-Aryl, an optionally substituted —(CH₂)ₙ—N(R⁶)(C═O)_{m′}—(V)_{n′}-Heteroaryl, an optionally substituted —(CH₂)ₙ—N(R⁶)(C═O)ₘ—(V)_{n′}-Heterocycle, an optionally substituted —X^{R3′}—C₁-C₆ alkyl group; an optionally substituted —X^{R3′}-Aryl group; an optionally substituted —X^{R3′}-Heteroaryl group; and an optionally substituted —X^{R3′}-Heterocycle group.

Variables R^{1N} and R^{2N} are each independently H, C₁-C₆ alkyl which is optionally substituted with one or two hydroxyl groups and up to three halogen groups or an optionally substituted —(CH₂)ₙ-Aryl, —(CH₂)ₙ-Heteroaryl or —(CH₂)ₙ-Heterocycle group;

Variable V is O, S or NR⁶.

Variable R²⁵ is independently H or C₁-C₃ alkyl.

X^{R2′} and X^{R3′} are each independently an optionally substituted —CH₂)ₙ—, —CH₂)ₙ—CH(X^{v})═CH(X^{v})-(cis or trans), —CH₂)ₙ—CH—CH—, —(CH₂CH₂O)ₙ— or a C₃-C₆ cycloalkyl, where X^{v} is H, a halo or optionally substituted C₁-C₃ alkyl.

Each m is independently 0, 1, 2, 3, 4, 5, 6. Each m′ is independently 0 or 1. Each n is independently 0, 1, 2, 3, 4, 5, 6. Each n′ is independently 0 or 1. Each u is independently 0 or 1.

Each v is independently 0 or 1. Each w is independently 0 or 1.

In various embodiments, any one or more of R^{1′}, R^{2′}, R^{3′}, X and X′ of ULM group is modified to be covalently bonded to the ATKI group through a linker L.

In various embodiments, the ULM corresponds to Formula (XVII) or (XVIII):

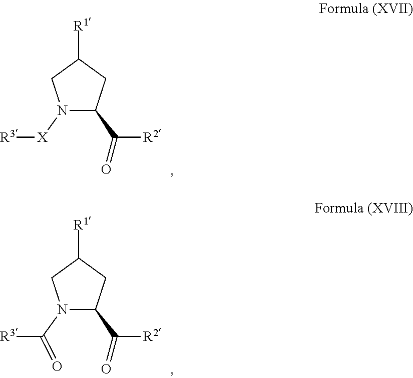

In various embodiments, in Formulas (XVI), (XVII), and (XVIII), R^{1′} is a hydroxyl group or a group that can be metabolized to a hydroxyl or carboxylic group. Exemplary R^{1′} groups include —(CH₂)ₙOH, —(CH₂)ₙ—O—(C₁-C₆)alkyl, —(CH₂)ₙCOOH, —(CH₂O)ₙH, an optionally substituted —(CH₂)ₙOC(O)—(C₁-C₆ alkyl), or an optionally substituted —(CH₂)ₙC(O)—O—(C₁-C₆ alkyl), wherein n is defined above.

In various embodiments, in Formulas (XVI), (XVII), (XVIII), R^{2′} and R^{3′} are each independently selected from the group consisting of an optionally substituted —NR₂₆-T-Aryl, an optionally substituted —NR²⁶-T-Heteroaryl or an optionally substituted —NR²⁶-T-Heterocycle, wherein R²⁶ is H or CH₃, and T is a group selected from the group consisting of —(CH₂)ₙ—, —(CH₂O)ₙ—, —(OCH₂)ₙ—, —(CH₂CH₂O)ₙ—, and —(OCH₂CH₂)ₙ—, wherein each one of the methylene groups may be optionally substituted with one or two substituents, selected from the group consisting of halogen, an amino acid, and C₁-C₃ alkyl; wherein n is defined above.

In various embodiments, in Formulas (XVI), (XVII), (XVIII), R^{2′} or R^{3′} is —NR²⁶-T-Ar¹, wherein the Ar¹ is phenyl or naphthyl optionally substituted with a group selected from the group consisting of a linker group L to which is attached a ATKI moiety, a halogen, an amine, monoalkyl- or dialkyl amine (preferably, dimethylamine), OH, COOH, C₁-C₆ alkyl, CF₃, OMe, OCF₃, NO₂, CN, an optionally substituted phenyl, an optionally substituted naphthyl, and an optionally substituted heteroaryl. Suitable heteroaryl includes an optionally substituted isoxazole, an optionally substituted oxazole, an optionally substituted thiazole, an optionally substituted isothiazole, an optionally substituted pyrrole, an optionally substituted imidazole, an optionally substituted benzimidazole, an optionally substituted oximidazole, an optionally substituted diazole, an optionally substituted triazole, an optionally substituted pyridine or an oxapyridine, an optionally substituted furan, an optionally substituted benzofuran, an optionally substituted dihydrobenzofuran, an optionally substituted indole, indolizine, azaindolizine, an optionally substituted quinoline, and an optionally substituted group selected from the group consisting of the chemical structures:

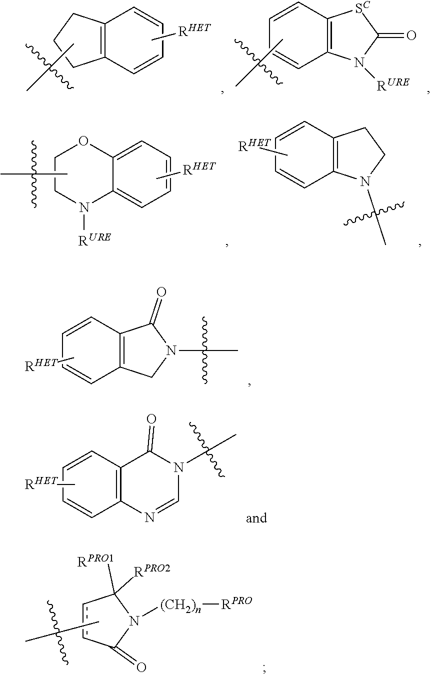

wherein S^{c} is CHR^{SS}, NR^{URE}, or O; R^{HET} is H, CN, NO₂, halo, optionally substituted C₁-C₆ alkyl, optionally substituted O(C₁-C₆ alkyl) or an optionally substituted acetylenic group —C≡C—Rₐ, wherein Rₐ is H or C₁-C₆ alkyl; R^{SS} is H, CN, NO₂, halo, optionally substituted C₁-C₆ alkyl, optionally substituted O—(C₁-C₆ alkyl or optionally substituted —C(O)(C₁-C₆ alkyl); R^{URE} is H, C₁-C₆ alkyl or —C(O)(C₁-C₆ alkyl), wherein the alkyl group is optionally substituted with one or two hydroxyl groups, up to three halogens, an optionally substituted phenyl group, an optionally substituted heteroaryl, or an optionally substituted heterocycle, preferably for example piperidine, morpholine, pyrrolidine, tetrahydrofuran; R^{PRO} is H, optionally substituted C₁-C₆ alkyl, an optionally substituted aryl, an optionally substituted heteroaryl or an optionally substituted heterocyclic group selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, benzofuran, indole, indolizine, and azaindolizine; R^{PRO1} and R^{PRO2} are each independently H, optionally substituted C₁-C₃ alkyl or together form a keto group; n is defined above.

In various embodiments, in Formulas (XVI), (XVII), and (XVIII), R^{2′} or R^{3′} is an optionally substituted —NR²⁶-T-Ar² group, wherein the Ar² group is selected from the group consisting of quinoline, indole, indolizine, azaindolizine, benzofuran, isoxazole, thiazole, isothiazole, thiophene, pyridine, imidazole, pyrrole, diazole, triazole, tetrazole, oximidazole, and a group selected from the group consisting of the following chemical structures:

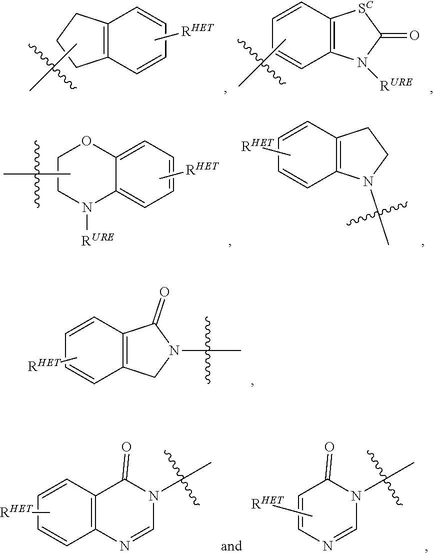

wherein S^{c}, R^{HET}, and R^{URE} are defined elsewhere herein; Y^{C} is N or C—R^{YC}; R^{YC} is H, OH, CN, NO₂, halo, optionally substituted C₁-C₆ alkyl, optionally substituted O(C₁-C₆ alkyl), or an optionally substituted acetylenic group —C≡C—R^{a}; R^{a} is H or C₁-C₆ alkyl.

In yet other embodiments of the Formulas (XVI), (XVII), and (XVIII), R^{2′} or R^{3′} is an optionally substituted —NR²⁶-T-HET¹, wherein the HET¹ is selected from the group consisting of tetrahydrofuran, tetrahydrothiene, tetrahydroquinoline, piperidine, piperazine, pyrrollidine, morpholine, oxane and thiane. The HET¹ is optionally substituted by a group selected from the group consisting of the following chemical structures:

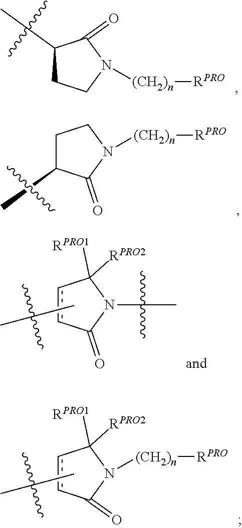

wherein n, R^{PRO}, R^{PRO1}, R^{HET} and R^{PRO2} are defined elsewhere herein.

In various embodiments, in Formulas (XVI), (XVII), and (XVIII), R^{2′} or R^{3′} is optionally substituted —(CH₂)ₙ—(V)_{n′}—(CH₂)ₙ—(V)_{n′}—R^{S3′}, optionally substituted —(CH₂)ₙ—N(R²⁶)(C═O)_{m′}—(V)_{n′}—R^{S3′}, optionally substituted —X^{R3′}—C₁-C₁₀ alkyl, optionally substituted —X^{R3′}—Ar³, optionally substituted —X^{R3′}-HET, optionally substituted —X^{R3′}—Ar³-HET or optionally substituted —X^{R3′}-HET-Ar³, wherein R^{S3′} is optionally substituted C₁-C₁₀ alkyl, optionally substituted Ar³ or HET; R²⁶ is defined elsewhere herein; V is O, S or NR^{1′}; X^{R3′} is —(CH₂)ₙ—, —(CH₂CH₂O)ₙ—, —CH₂)ₙ—CH(X^{V})═CH(X^{V})— (cis or trans), —CH₂)ₙ—CH—CH—, or a C₃-C₆ cycloalkyl group, all optionally substituted; wherein X^{v} is H, a halo or a C₁-C₃ alkyl group which is optionally substituted with one or two hydroxyl groups or up to three halogen groups; Ar³ is an optionally substituted phenyl or napthyl group; and HET is an optionally substituted oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, benzofuran, indole, indolizine, azaindolizine, quinoline, or a group selected from the group consisting of the following chemical structures:

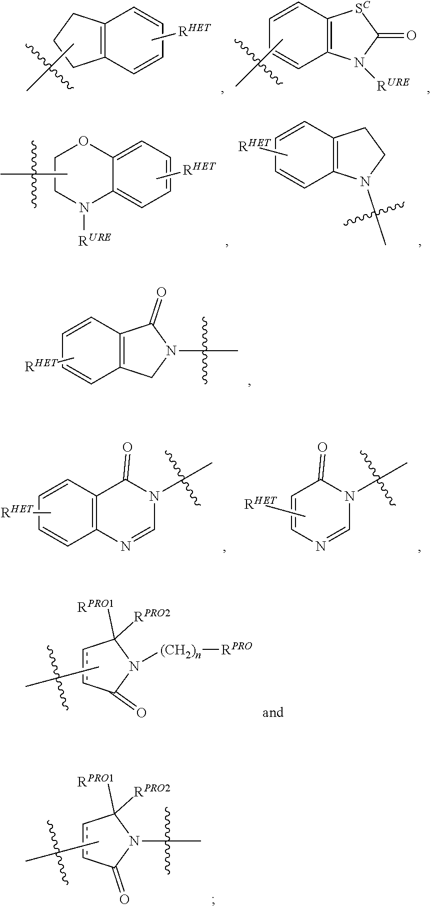

wherein n, v, n′, m′, S^{c}, R^{HET}, R^{URE}, C, R^{PRO1} and R^{PRO2} are defined elsewhere herein.

In various embodiments, in Formulas (XVI), (XVII), and (XVIII), R^{2′} or R^{3′} is an optionally substituted —NR²⁶—X^{R2′}—C₁-C₁₀ alkyl, —NR²⁶—X^{R2′}—Ar³, an optionally substituted —NR²⁶—X^{R2′}-HET, an optionally substituted —NR²⁶—X^{R2′}—Ar³-HET, or an optionally substituted —NR²⁶—X^{R2′}-HET-Ar³, X^{R2′} is an optionally substituted —CH₂)ₙ—, —CH₂)ₙ—CH(X^{v})═CH(X^{v})-(cis or trans), —CH₂)ₙ—CH—CH—, —(CH₂CH₂O)ₙ— or C₃-C₆ cycloalkyl; wherein X^{v} is H, a halo or a C₁-C₃ alkyl group which is optionally substituted with one or two hydroxyl groups or up to three halogen groups; wherein HET, Ar³, and R²⁶ are defined elsewhere herein.

In yet other embodiments, in Formulas (XVI), (XVII), and (XVIII), R^{2′} or R^{3′} is —(CH₂)ₙ—Ar¹, —(CH₂CH₂O)ₙ—Ar¹, —(CH₂)ₙ-HET or —(CH₂CH₂O)ₙ—HET; wherein n, Ar¹, and HET are defined elsewhere herein.

In various embodiments, ULM corresponds to Formula (XIX):

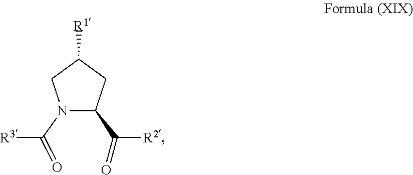

wherein in Formula (XIX), R¹ is OH or a group which is metabolized in a patient or subject to OH; R^{2′} is —NH—CH₂—Ar⁴-HET¹; R³ is —CHR^{CR3′}—NH—C(O)—R^{3P1} or —CHR^{CR3′}—R^{3P2}; wherein RC^{R3′} is C₁-C₄ alkyl, preferably methyl, isopropyl or tert-butyl; R^{3P1} is C₁-C₃ alkyl, optionally substituted oxetane, —CH₂OCH₃, —CH₂CH₂OCH₃, morpholino, or

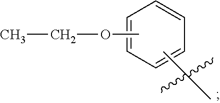

is a

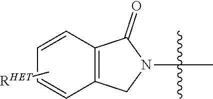

group, wherein Ar⁴ is phenyl; HET¹ is an optionally substituted thiazole or isothiazole; and R^{HET} is H or halo.

In various embodiments, the ULM has the structure of Formula (XX) or Formula (XXI):

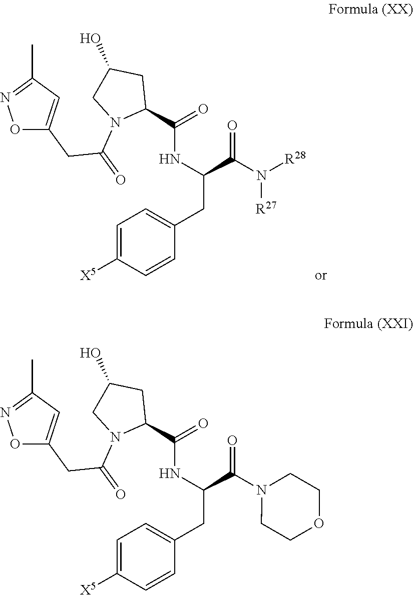

wherein X⁵ is Cl, F, C₁-C₃ alkyl or heterocycle; R²⁷ and R²⁸ are each independently H, C₁-C₃ alkyl.

In any aspect or embodiment described herein, ULM is VLM and comprises a chemical structure selected from the group ULM-a:

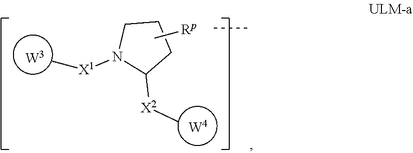

wherein:
[symbol] indicates the attachment of at least one PTM, another ULM or VLM or MLM or ILM or CLM (i.e., ULM′ or VLM′ or CLM′ or ILM′ or MLM′), or a chemical linker moiety coupling at least one PTM, a ULM′ or a VLM′ or a CLM′ or a ILM′ or a MLM′ to the other end of the linker;
X¹, X² of Formula ULM-a are each independently selected from the group of a bond, O, NR^{Y3}, CR^{Y3}R^{Y4}, C═O, C═S, SO, and SO₂; R^{Y3}, R^{Y4} of Formula ULM-a are each independently selected from the group of H, linear or branched C₁₋₆ alkyl, optionally substituted by 1 or more halo, optionally substituted C₁₋₆ alkoxyl (e.g., optionally substituted by 0-3 R^{P} groups); R^{P} of Formula ULM-a is 0, 1, 2, or 3 groups, each independently selected from the group H, halo, —OH, C₁₋₃ alkyl, C═O;
W³ of Formula ULM-a is selected from the group of an optionally substituted T, an optionally substituted -T-N(R^{1a}R^{1b})X³, optionally substituted -T-N(R^{1a}R^{1b}), optionally substituted -T-Aryl, an optionally substituted -T-Heteroaryl, an optionally substituted T-biheteroaryl, an optionally substituted -T-Heterocycle, an optionally substituted -T-biheterocycle, an optionally substituted —NR¹-T-Aryl, an optionally substituted —NR¹-T-Heteroaryl, or an optionally substituted —NR¹-T-Heterocycle;
X³ of Formula ULM-a is C═O, R¹, R^{1a}, R^{1b};
each of R¹, R^{1a}, R^{1b} is independently selected from the group consisting of H, linear or branched C₁-C₆ alkyl group optionally substituted by 1 or more halo or —OH groups, R^{Y3}C═O, R^{Y3}C═S, R^{Y3}SO, R^{Y3}SO₂, N(R^{Y3}R^{Y4})C═O, N(R^{Y3}R^{Y4})C═S, N(R^{Y3}R^{Y4})SO, and N(R^{Y3}R^{Y4})SO₂;
T of Formula ULM-a is selected from the group of an optionally substituted alkyl, —(CH₂)ₙ— group, —(CH₂)ₙ—O—C₁-C₆ alkyl which is optionally substituted, linear, branched, or —(CH₂)ₙ—O-heterocyclyl which is optionally substituted, wherein each one of the methylene groups is optionally substituted with one or two substituents selected from the group of halogen, methyl, optionally substituted alkoxy, a linear or branched C₁-C₆ alkyl group optionally substituted by 1 or more halogen, C(O) NR¹R^{1a}, or NR¹R^{1a} or R¹ and R^{1a} are joined to form an optionally substituted heterocycle, or —OH groups or an amino acid side chain optionally substituted;
W⁴ of Formula ULM-a is an optionally substituted —NR¹-T-Aryl wherein the aryl group may be optionally substituted with an optionally substituted 5-6 membered heteroaryl or an optionally substituted aryl, an optionally substituted —NR¹-T-Heteroaryl group with an optionally substituted aryl or an optionally substituted heteroaryl, or an optionally substituted —NR¹-T-Heterocycle, where —NR¹ is covalently bonded to X² and R¹ is H or CH₃, preferably H; and
n is 0 to 6, often 0, 1, 2, or 3, preferably 0 or 1.

In any of the embodiments described herein, T is selected from the group of an optionally substituted alkyl, —(CH₂)ₙ— group, wherein each one of the methylene groups is optionally substituted with one or two substituents selected from the group of halogen, methyl, optionally substituted alkoxy, a linear or branched C₁-C₆ alkyl group optionally substituted by 1 or more halogen, C(O) NR¹R^{1a}, or NR¹R^{1a} or R¹ and R^{1a} are joined to form an optionally substituted heterocycle, or —OH groups or an amino acid side chain optionally substituted; and n is 0 to 6, often 0, 1, 2, or 3, preferably 0 or 1.

In any aspect or embodiment described herein, W⁴ of Formula ULM-a is

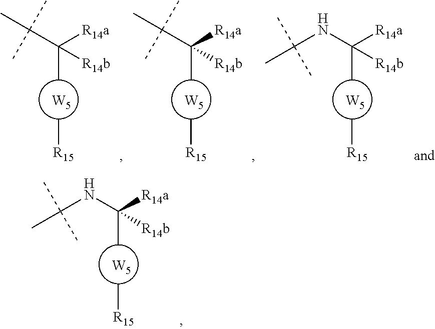

wherein:
W⁵ is optionally substituted (e.g., W⁵ is an optionally substituted phenyl, an optionally substituted napthyl, or an optionally substituted 5-10 membered heteroaryl; e.g., W⁵ is optionally substituted with one or more [such as 1, 2, 3, 4, or 5] halo, CN, optionally substituted alkyl, optionally substituted haloalkyl, optionally substituted alkoxy, hydroxy, or optionally substituted haloalkoxy), and R₁₄ₐ, R_{14b}, are each independently selected from the group of H, haloalkyl (e.g., fluoalkyl), optionally substituted alkyl (e.g., C₁-C₆ alkyl), optionally substituted alkoxy, optionally substituted hydroxyl alkyl, optionally substituted alkylamine, optionally substituted heterolkyl, optionally substituted alkyl-heterocycloalkyl, optionally substituted alkoxy-heterocycloalkyl, COR₂₆, CONR₂₇ₐR_{27b}, NHCOR₂₆, or NHCH₃COR₂₆; and the other of R₁₄ₐ and R_{14b} is H; or R₁₄ₐ, R_{14b}, together with the carbon atom to which they are attached, form an optionally substituted 3 to 5 membered cycloalkyl, heterocycloalkyl, spirocycloalkyl or spiroheterocyclyl, wherein the spiroheterocyclyl is not epoxide or aziridine.

In any of the aspect or embodiments described herein, W⁵ of Formula ULM-a is selected from the group of an optionally substituted phenyl, an optionally substituted napthyl, or an optionally substituted 5-10 membered heteroaryl (e.g., W⁵ is optionally substituted with one or more [such as 1, 2, 3, 4, or 5] halo, CN, optionally substituted alkyl, optionally substituted haloalkyl, optionally substituted alkoxy, hydroxy, or optionally substituted haloalkoxy), R₁₅ of Formula ULM-a is selected from the group of H, halogen, CN, OH, NO₂, N R₁₄ₐR_{14b}, OR₁₄ₐ, CONR₁₄ₐR_{14b}, NR₁₄ₐCOR_{14b}, SO₂NR₁₄ₐR_{14b}, NR₁₄ₐSO₂R_{14b}, optionally substituted alkyl, optionally substituted haloalkyl, optionally substituted haloalkoxy; optionally substituted aryl, optionally substituted heteroaryl, optionally substituted cycloalkyl, or optionally substituted cycloheteroalkyl.

In any aspect or embodiment described herein, W⁴ substituents for use in the present disclosure also include specifically (and without limitation to the specific compound disclosed) the W⁴ substituents which are found in the identified compounds disclosed herein. Each of these W⁴ substituents may be used in conjunction with any number of W³ substituents which are also disclosed herein.

In any aspect or embodiment described herein, ULM-a, is optionally substituted by 0-3 R^{P} groups in the pyrrolidine moiety. Each R^{P} is independently H, halo, —OH, C1-3alkyl, C═O.

In any aspect or embodiment described herein, the W³, W⁴ of Formula ULM-a can independently be covalently coupled to a linker which is attached one or more PTM groups.

and wherein the dashed line indicates the site of attachment of at least one PTM, another ULM (ULM′) or a chemical linker moiety coupling at least one PTM or a ULM′ or both to ULM.

In any aspect or embodiment described herein, ULM is VHL and is represented by the structure:

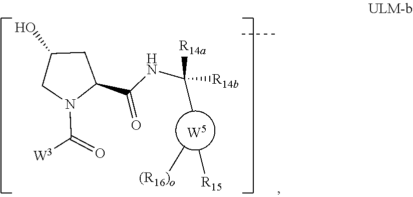

wherein:
W³ of Formula ULM-b is selected from the group of an optionally substituted aryl, optionally substituted heteroaryl, or

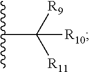

R₉ and R₁₀ of Formula ULM-b are independently hydrogen, optionally substituted alkyl, optionally substituted cycloalkyl, optionally substituted hydroxyalkyl, optionally substituted heteroaryl, or haloalkyl, or R₉, R₁₀, and the carbon atom to which they are attached form an optionally substituted cycloalkyl;
R₁₁ of Formula ULM-b is selected from the group of an optionally substituted heterocyclyl, optionally substituted alkoxy, optionally substituted heteroaryl, optionally substituted aryl,

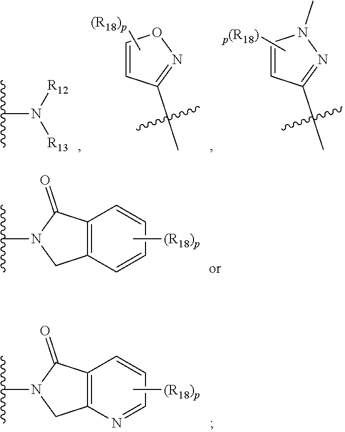

R₁₂ of Formula ULM-b is selected from the group of H or optionally substituted alkyl;
R₁₃ of Formula ULM-b is selected from the group of H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R₁₄ₐ, R_{14b} of Formula ULM-b, are each independently selected from the group of H, haloalkyl (e.g. fluoroalkyl), optionally substituted alkyl (e.g., C1-C6 alkyl), optionally substitute alkoxy, aminomethyl, alkylaminomethyl, alkoxymethyl, optionally substituted hydroxyl alkyl, optionally substituted alkylamine, optionally substituted heterolkyl, optionally substituted alkyl-heterocycloalkyl, optionally substituted alkoxy-heterocycloalkyl, CONR₂₇ₐR_{27b}, CH₂NHCOR₂₆, or (CH₂)N(CH3)COR₂₆; and the other of R₁₄ₐ and R_{14b} is H; or R₁₄ₐ, R_{14b}, together with the carbon atom to which they are attached, form an optionally substituted 3 to 6 membered cycloalkyl, heterocycloalky, spirocycloalkyl or spiroheterocyclyl, wherein the spiroheterocyclyl is not epoxide or aziridine;
W⁵ of Formula ULM-b is selected from the group of an optionally substituted phenyl or an optionally substituted 5-10 membered heteroaryl (e.g., W⁵ is optionally substituted with one or more [such as 1, 2, 3, 4, or 5] halo, CN, optionally substituted alkyl, optionally substituted haloalkyl, optionally substituted alkoxy, hydroxy, or optionally substituted haloalkoxy),
R₁₅ of Formula ULM-b is selected from the group of H, halogen, CN, OH, NO₂, N R₁₄ₐR_{14b}, OR₁₄ₐ, CONR₁₄ₐR_{14b}, NR₁₄ₐCOR_{14b}, SO₂NR₁₄ₐR_{14b}, NR₁₄ₐ SO₂R_{14b}, optionally substituted alkyl, optionally substituted haloalkyl, optionally substituted haloalkoxy, optionally substituted aryl, optionally substituted heteroaryl, optionally substituted cycloalkyl, or optionally substituted cycloheteroalkyl;
each R₁₆ of Formula ULM-b is independently selected from the group of H, CN, halo, optionally substituted alkyl, optionally substituted haloalkyl, hydroxy, or optionally substituted haloalkoxy;
o of Formula ULM-b is 0, 1, 2, 3, or 4;
R₁₈ of Formula ULM-b is independently selected from the group of H, halo, optionally substituted alkoxy, cyano, optionally substituted alkyl, haloalkyl, haloalkoxy or a linker; and
p of Formula ULM-b is 0, 1, 2, 3, or 4, and wherein the dashed line indicates the site of attachment of at least one PTM, another ULM (ULM′) or a chemical linker moiety coupling at least one PTM or a ULM′ or both to ULM.

In any aspect or embodiment described herein, R₁₅ of Formula ULM-b is selected from the group of H, halogen, CN, OH, NO₂, NR₂₇ₐR_{27b}, OR₂₇ₐ, CONR₂₇ₐR_{27b}, NR₂₇ₐCOR_{27b}, SO₂NR₂₇ₐR_{27b}, NR₂₇ₐ SO₂R_{27b}, optionally substituted alkyl, optionally substituted haloalkyl, optionally substituted haloalkoxy, optionally substituted aryl, optionally substituted heteroaryl, optionally substituted cycloalkyl, or optionally substituted heterocyclyl, wherein each R₂₆ is independently selected from H, optionally substituted alkyl or NR₂₇ₐR_{27b}; and each R₂₇ₐ and R_{27b} is independently H, optionally substituted alkyl, or R₂₇ₐ and R_{27b} together with the nitrogen atom to which they are attached form a 4-6 membered heterocyclyl.

In any aspect or embodiment described herein, R₁₅ of Formula ULM-b is

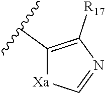

wherein R₁₇ is H, halo, optionally substituted C₃₋₆cycloalkyl, optionally substituted C₁₋₆alkyl, optionally substituted C₁₋₆alkenyl, and C₁₋₆haloalkyl; and Xa is S or O.

In any aspect or embodiments described herein, R₁₇ of Formula ULM-b is selected from the group methyl, ethyl, isopropyl, and cyclopropyl.

In any aspect or embodiments described herein, R₁₅ of Formula ULM-b is selected from the group consisting of:

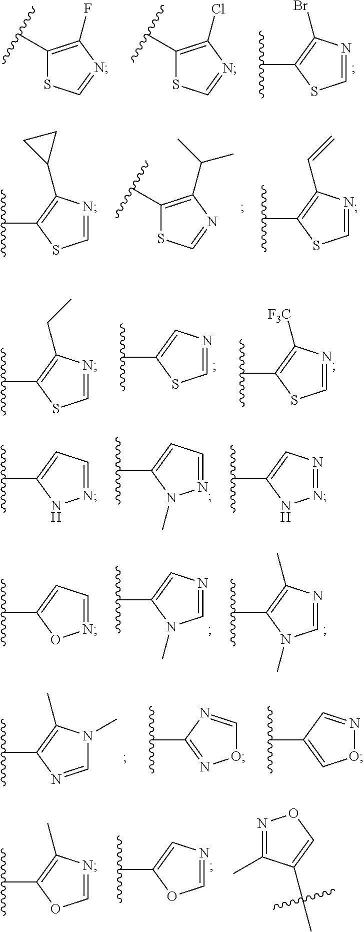

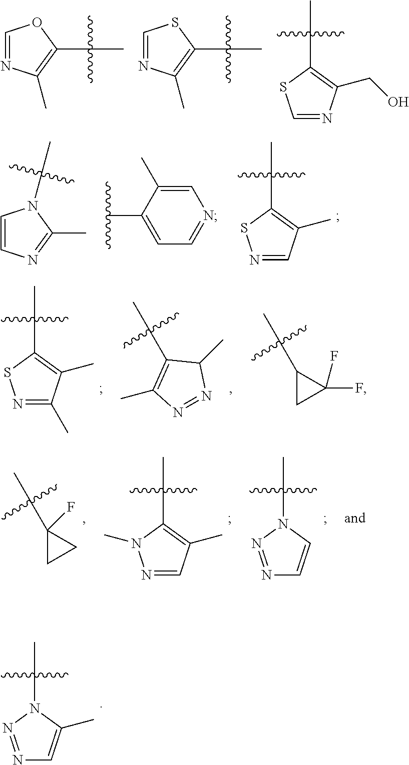

In any aspect or embodiments described herein, R₁₁ of Formula ULM-b is selected from the group consisting of:

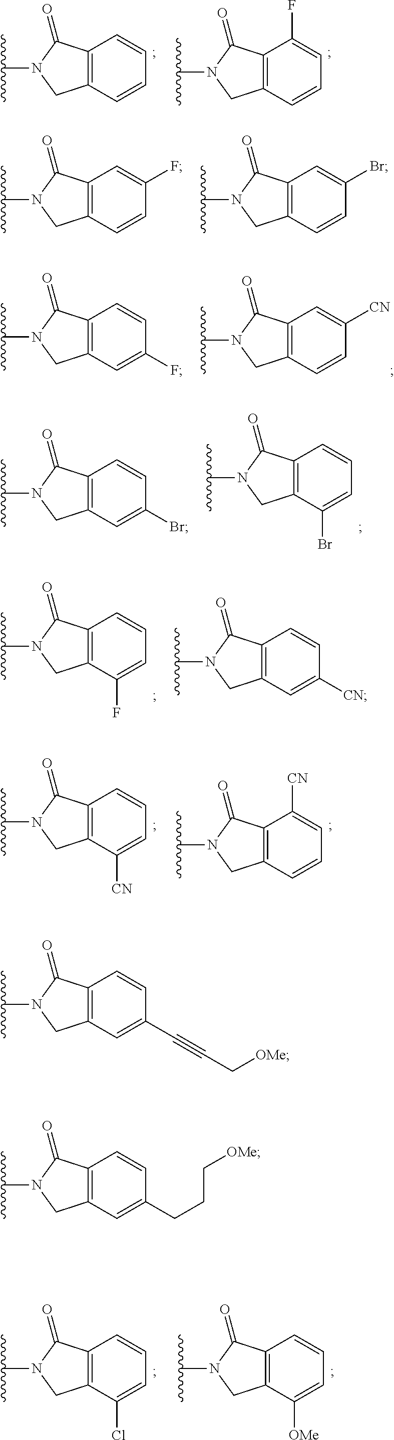

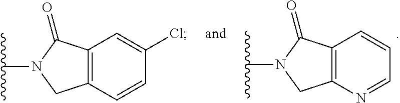 [SMILES: *N1Cc2ccc(Cl)cc2C1=O ; *N1Cc2ncccc2C1=O]

In any aspect or embodiments described herein, R₁₄ₐ, R_{14b} of Formula ULM-b, are each independently selected from the group of H, optionally substituted haloalkyl, optionally substituted alkyl, optionally substituted alkoxy, optionally substituted hydroxyl alkyl, optionally substituted alkylamine, optionally substituted heterolkyl, optionally substituted alkyl-heterocycloalkyl, optionally substituted alkoxy-heterocycloalkyl, CH₂OR₃₀, CH₂NHR₃₀, CH₂NCH₃R₃₀, CONR₂₇ₐR_{27b}, CH₂CONR₂₇ₐR_{27b}, CH₂NHCOR₂₆, or CH₂NCH₃COR₂₆; and the other of R₁₄ₐ and R_{14b} is H; or R₁₄ₐ, R_{14b}, together with the carbon atom to which they are attached, form an optionally substituted 3- to 6-membered cycloalkyl, heterocycloalkyl, spirocycloalkyl or spiroheterocyclyl, wherein the spiroheterocyclyl is not epoxide or aziridine, the said spirocycloalkyl or spiroheterocycloalkyl itself being optionally substituted with an alkyl, a haloalkyl, or —COR₃₃ where R₃₃ is an alkyl or a haloalkyl,

wherein R₃₀ is selected from H, alkyl, alkynylalkyl, cycloalkyl, heterocycloalkyl, cycloalkylalkyl, heterocycloalkylalkyl, arylalkyl or heteroarylalkyl further optionally substituted; R₂₆ and R₂₇ are as described above.

In any aspect or embodiments described herein, R₁₅ of Formula ULM-b is selected from H, halogen, CN, OH, NO₂, NR₂₇ₐR_{27b}, OR₂₇ₐ, CONR₂₇ₐR_{27b}, NR₂₇ₐCOR_{27b}, SO₂NR₂₇ₐR_{27b}, NR₂₇ₐ SO₂R_{27b}, optionally substituted alkyl, optionally substituted haloalkyl (e.g. optionally substituted fluoroalkyl), optionally substituted haloalkoxy, optionally substituted aryl, optionally substituted heteroaryl, optionally substituted cycloalkyl, or optionally substituted heterocyclyl wherein optional substitution of the said aryl, heteroaryl, cycloalkyl and heterocycloalkyl includes CH₂OR₃₀, CH₂NHR₃₀, CH₂NCH₃R₃₀, CONR₂₇ₐR_{27b}, CH₂CONR₂₇ₐR_{27b}, CH₂NHCOR₂₆, CH₂NCH₃COR₂₆ or

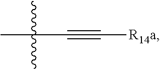

wherein R₂₆, R₂₇, R₃₀ and R₁₄ₐ are as described above.

In any aspect or embodiments described herein, R₁₄ₐ, R_{14b} of Formula ULM-b, are each independently selected from the group of H, optionally substituted haloalkyl, optionally substituted alkyl, CH₂OR₃₀, CH₂NHR₃₀, CH₂NCH₃R₃₀, CONR₂₇ₐR_{27b}, CH₂CONR₂₇ₐR_{27b}, CH₂NHCOR₂₆, or CH₂NCH₃COR₂₆; and the other of R₁₄ₐ and R_{14b} is H; or R₁₄ₐ, R_{14b}, together with the carbon atom to which they are attached, form an optionally substituted 3- to 6-membered spirocycloalkyl or spiroheterocyclyl, wherein the spiroheterocyclyl is not epoxide or aziridine, the said spirocycloalkyl or spiroheterocycloalkyl itself being optionally substituted with an alkyl, a haloalkyl, or —COR₃₃ where R₃₃ is an alkyl or a haloalkyl, wherein R₃₀ is selected from H, alkyl, alkynylalkyl, cycloalkyl, heterocycloalkyl, cycloalkylalkyl, heterocycloalkylalkyl, arylalkyl or heteroarylalkyl further optionally substituted;

R₁₅ of Formula ULM-b is selected from H, halogen, CN, OH, NO₂, NR₂₇ₐR_{27b}, OR₂₇ₐ, CONR₂₇ₐR_{27b}, NR₂₇ₐCOR_{27b}, SO₂NR₂₇ₐR_{27b}, NR₂₇ₐ SO₂R_{27b}, optionally substituted alkyl, optionally substituted haloalkyl, optionally substituted haloalkoxy, optionally substituted aryl, optionally substituted heteroaryl, optionally substituted cycloalkyl, or optionally substituted heterocyclyl wherein optional substitution of the said aryl, heteroaryl, cycloalkyl and heterocycloalkyl includes CH₂OR₃₀ CH₂NHR₃₀ CH₂NCH₃R₃₀, CONR₂₇ₐR_{27b}, CH₂CONR₂₇ₐR_{27b}, CH₂NHCOR₂₆, CH₂NCH₃COR₂₆ or

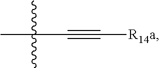

wherein R₂₆, R₂₇, R₃₀ and R₁₄ₐ are as described above.

In any aspect or embodiments described herein, ULM has a chemical structure selected from the group of:

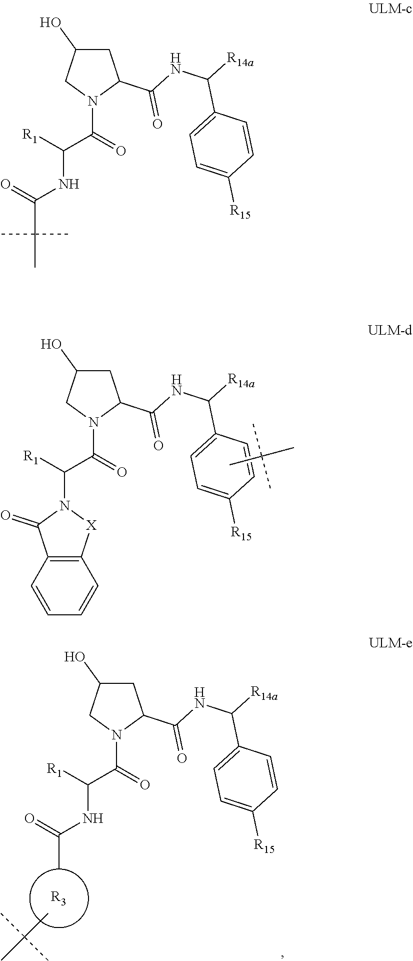

wherein:
R₁ of Formulas ULM-c, ULM-d, and ULM-e is H, ethyl, isopropyl, tert-butyl, sec-butyl, cyclopropyl, cyclobutyl, cyclopentyl, or cyclohexyl; optionally substituted alkyl, optionally substituted cycloalkyl, optionally substituted hydroxyalkyl, optionally substituted heteroaryl, or haloalkyl;
R₁₄ₐ of Formulas ULM-c, ULM-d, and ULM-e is H, haloalkyl, optionally substituted alkyl (e.g., C₁-C₆ alkyl), methyl, fluoromethyl, hydroxymethyl, ethyl, isopropyl, or cyclopropyl;
R₁₅ of Formulas ULM-c, ULM-d, and ULM-e is selected from the group consisting of H, halogen, CN, OH, NO₂, optionally substituted heteroaryl, optionally substituted aryl; optionally substituted alkyl, optionally substituted haloalkyl, optionally substituted haloalkoxy, optionally substituted cycloalkyl, or optionally substituted cycloheteroalkyl;
X of Formulas ULM-c, ULM-d, and ULM-e is C, CH₂, or C═O
R₃ of Formulas ULM-c, ULM-d, and ULM-e is absent or an optionally substituted 5 or 6 membered heteroaryl; and
the dashed line indicates the site of attachment of at least one PTM, another ULM (ULM′) or a chemical linker moiety coupling at least one PTM or a ULM′ or both to ULM.

In any aspect or embodiments described herein, ULM comprises a group according to the chemical structure:

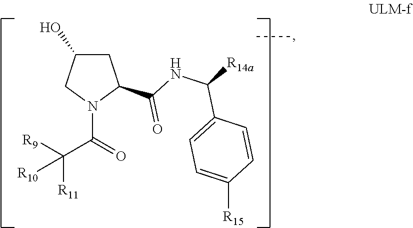

wherein:
R₁₄ₐ of Formula ULM-f is H, haloalkyl, optionally substituted alkyl (e.g., C₁-C₆ alkyl), methyl, fluoromethyl, hydroxymethyl, ethyl, isopropyl, or cyclopropyl;
R₉ of Formula ULM-f is H;
R₁₀ of Formula ULM-f is H, ethyl, isopropyl, tert-butyl, sec-butyl, cyclopropyl, cyclobutyl, cyclopentyl, or cyclohexyl;
R₁₁ of Formula ULM-f is

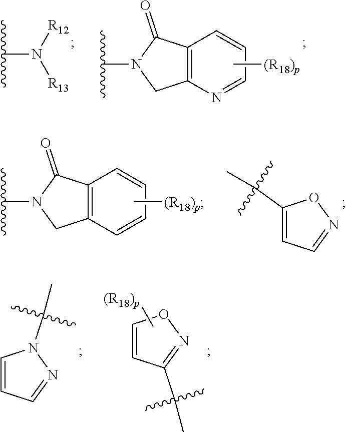

or optionally substituted heteroaryl;
p of Formula ULM-f is 0, 1, 2, 3, or 4;
each R₁₈ of Formula ULM-f is independently halo, optionally substituted alkoxy, cyano, optionally substituted alkyl, haloalkyl, haloalkoxy or a linker;
R₁₂ of Formula ULM-f is H, C═O;
R₁₃ of Formula ULM-f is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl,
R₁₅ of Formula ULM-f is selected from the group consisting of H, halogen, Cl, CN, OH, NO₂, optionally substituted haloalkyl, optionally substituted heteroaryl, optionally substituted aryl;

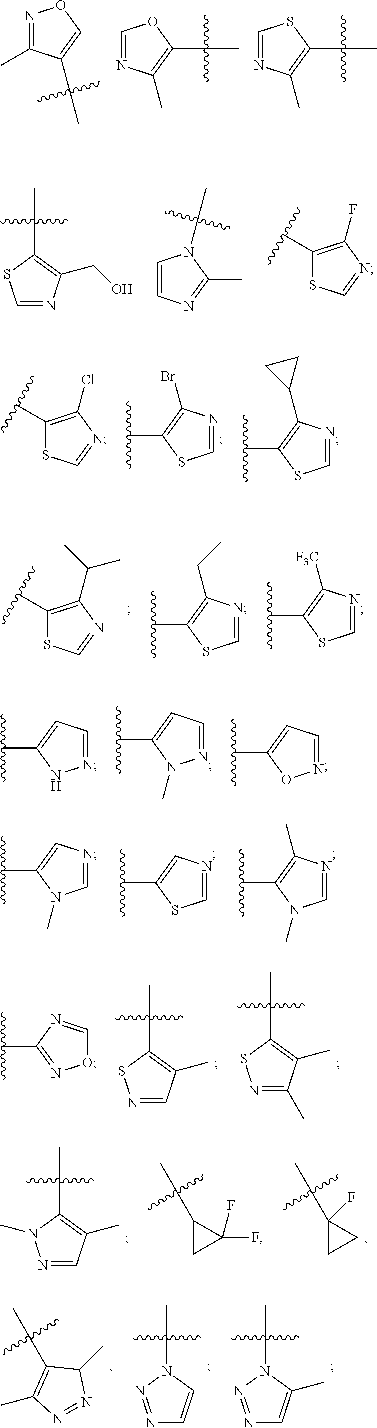

and
the dashed line of Formula ULM-f indicates the site of attachment of at least one PTM, another ULM (ULM′) or a chemical linker moiety coupling at least one PTM or a ULM′ or both to ULM.

In any aspect or embodiments described herein, the ULM is selected from the following structures:

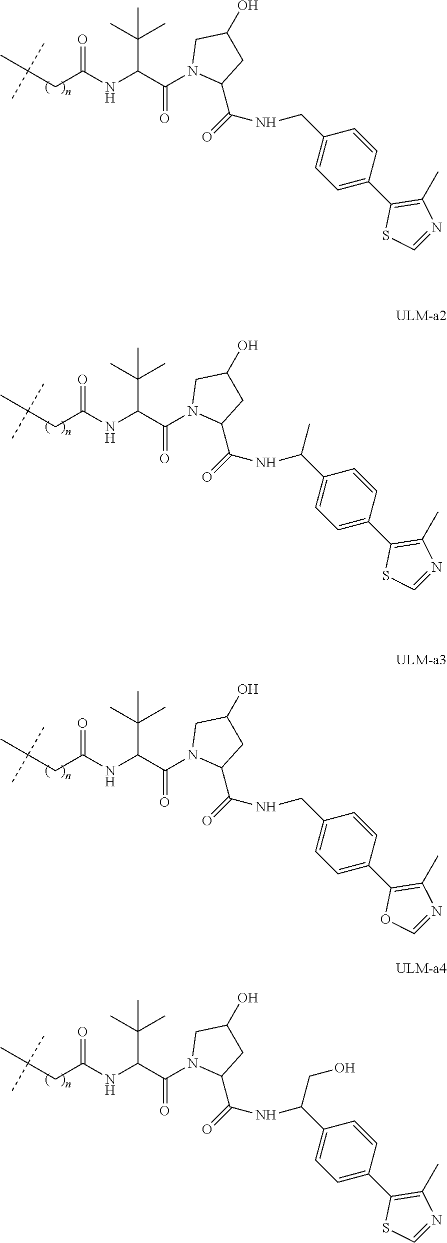

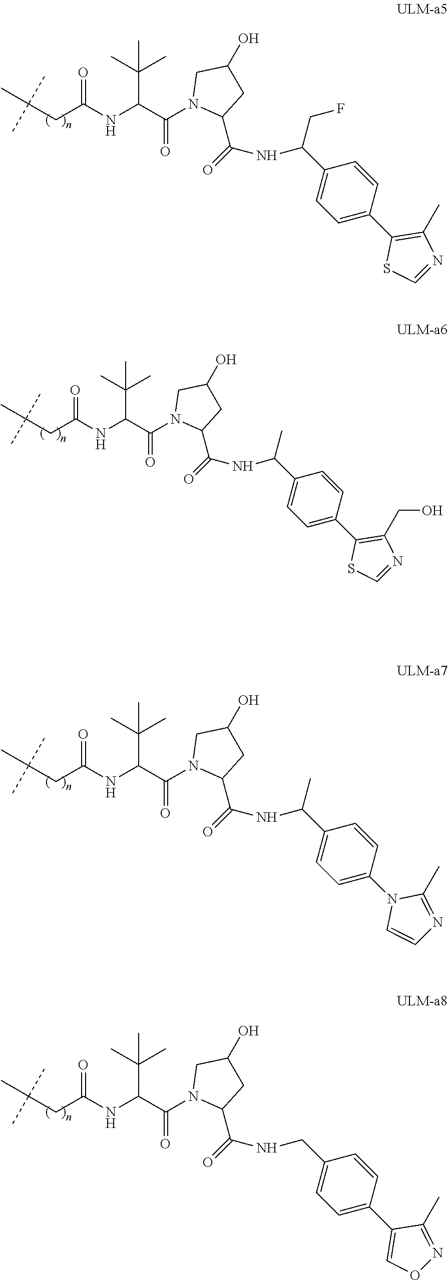

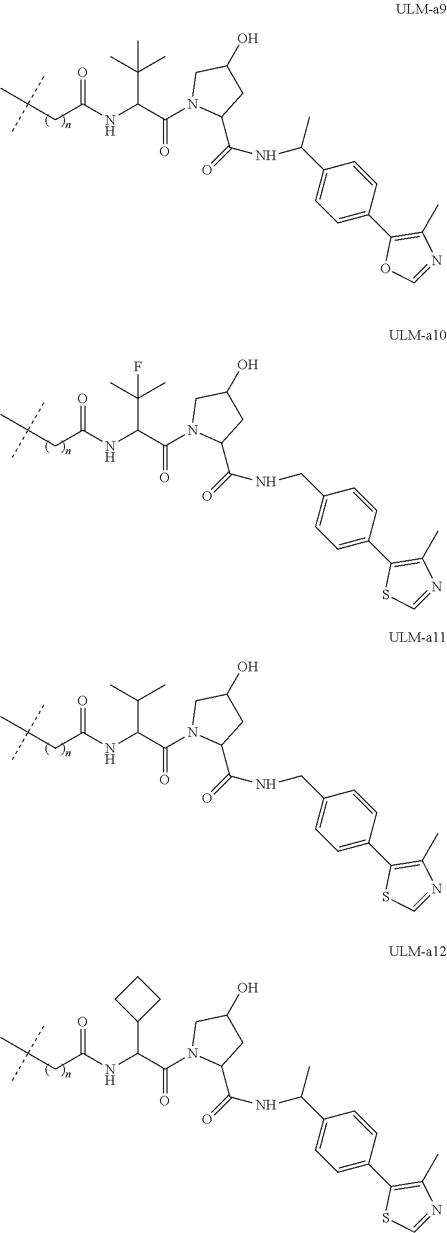

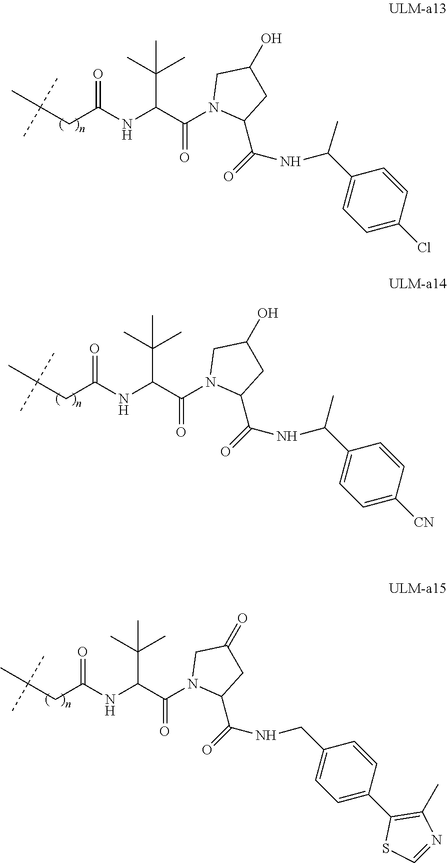

wherein n is 0 or 1.

In any aspect or embodiments described herein, the ULM is selected from the following structures:

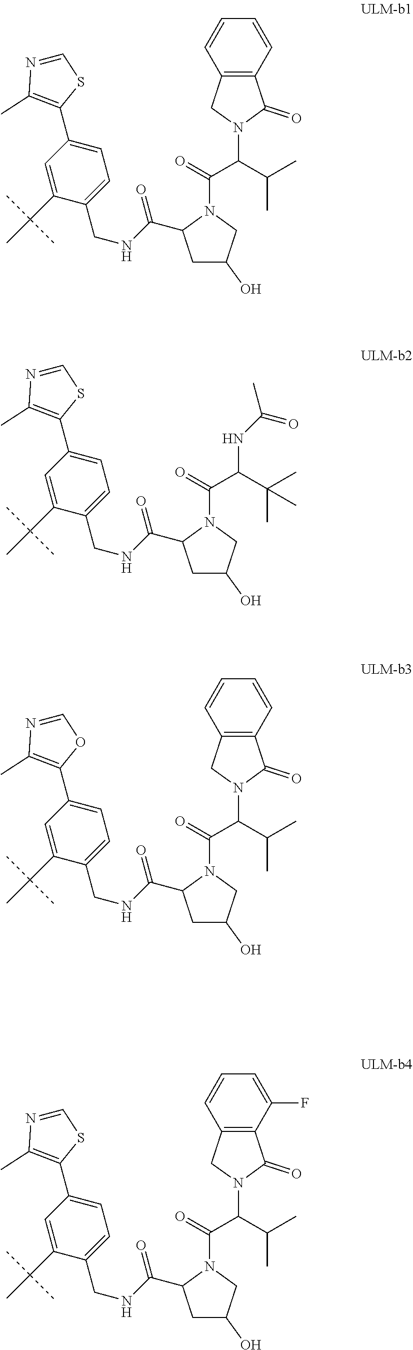

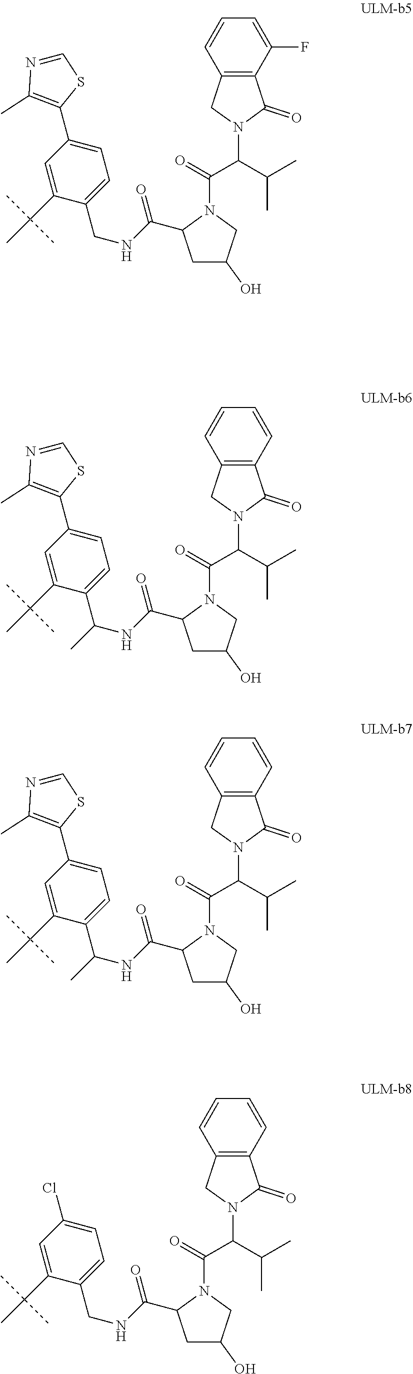

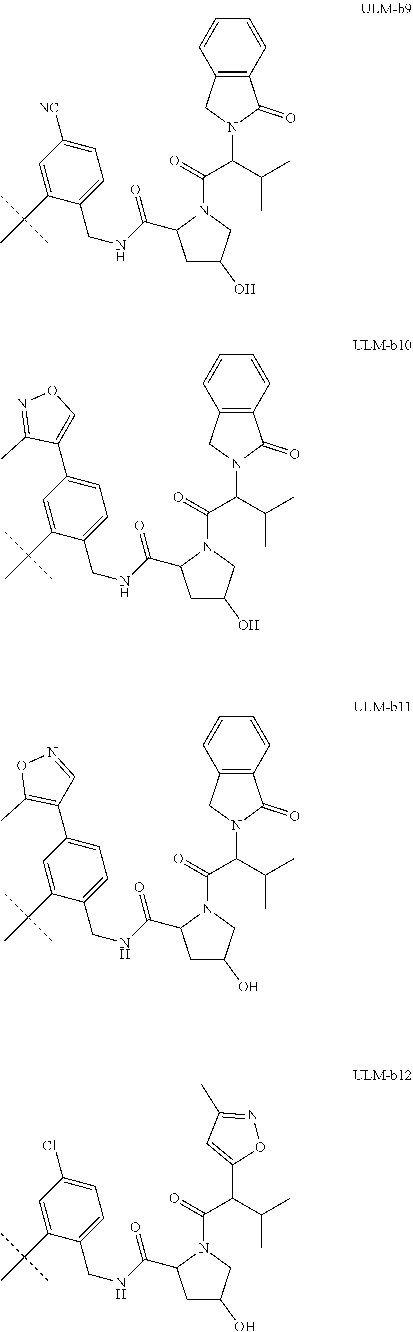

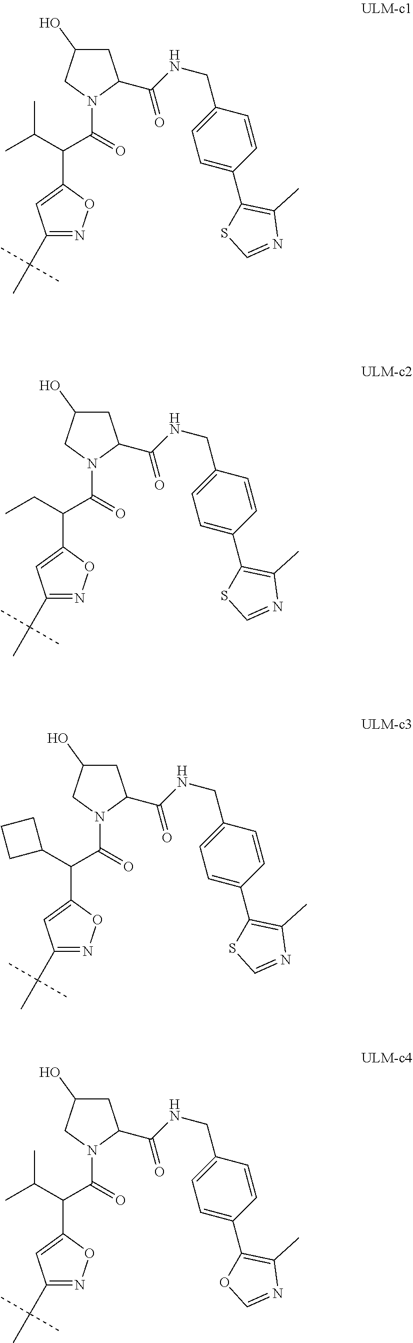

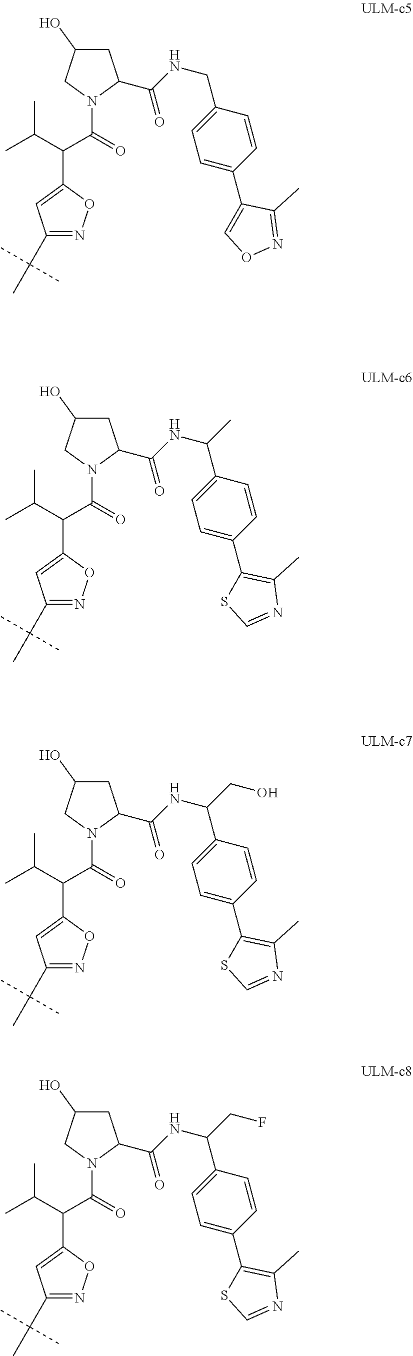

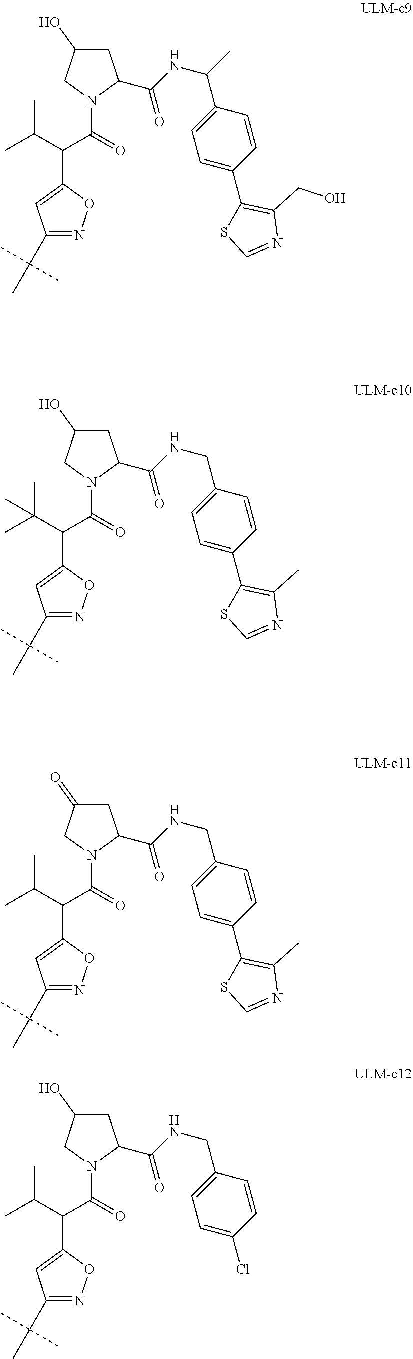

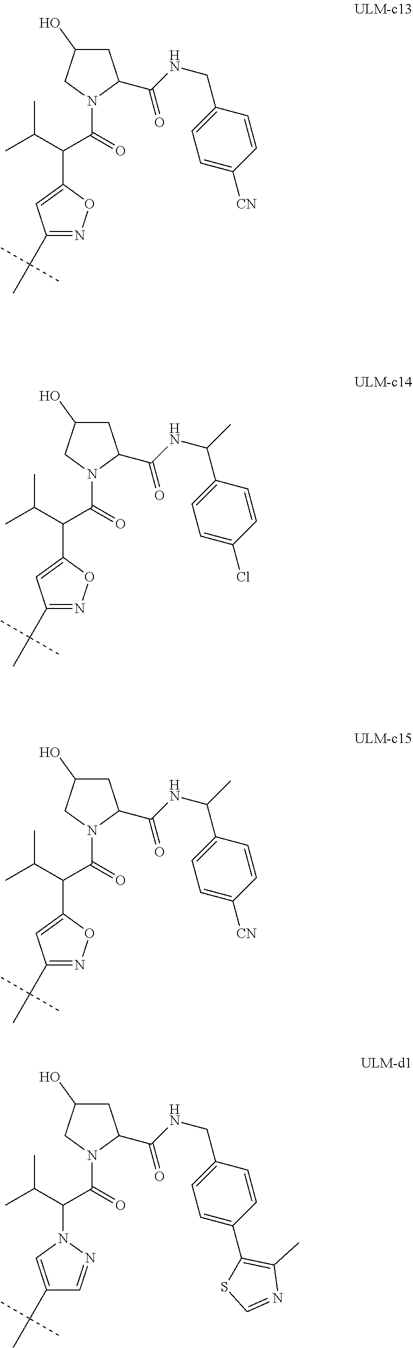

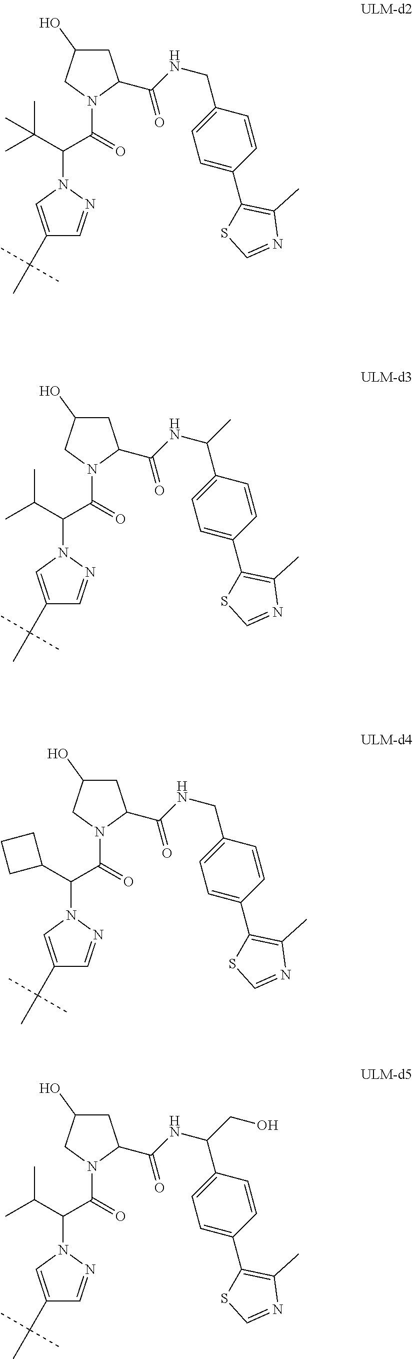

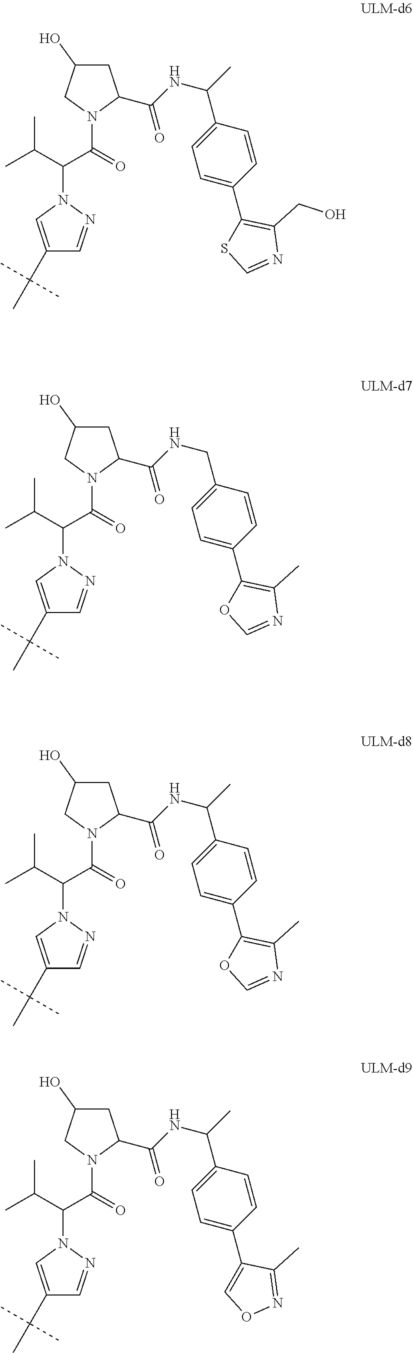

wherein, the phenyl ring in ULM-a1 through ULM-a15, ULM-b 1 through ULM-b 12, ULM-c1 through ULM-c15 and ULM-d1 through ULM-d9 is optionally substituted with fluorine, lower alkyl and alkoxy groups, and wherein the dashed line indicates the site of attachment of at least one PTM, another ULM (ULM′) or a chemical linker moiety coupling at least one PTM or a ULM′ or both to ULM-a.

In any aspect or embodiments described herein, the phenyl ring in ULM-a1 through ULM-a15, ULM-b 1 through ULM-b 12, ULM-c1 through ULM-c15 and ULM-d1 through ULM-d9 can be functionalized as the ester to make it a part of the prodrug.

In any aspect or embodiments described herein, the hydroxyl group on the pyrrolidine ring of ULM-a1 through ULM-a 15, ULM-b 1 through ULM-b 12, ULM-c1 through ULM-c15 and ULM-d1 through ULM-d9, respectively, comprises an ester-linked prodrug moiety.

In yet other embodiments, ULM is a Cereblon ligand of Formula (XXII) or a VHL ligand of Formula (XXIII):

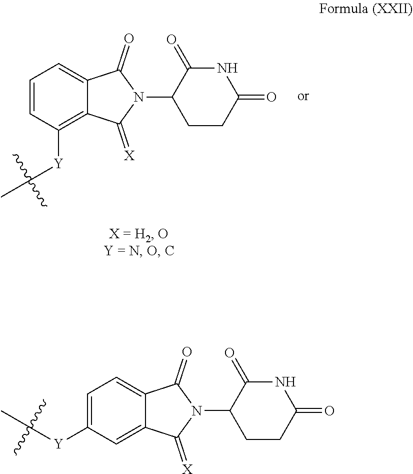

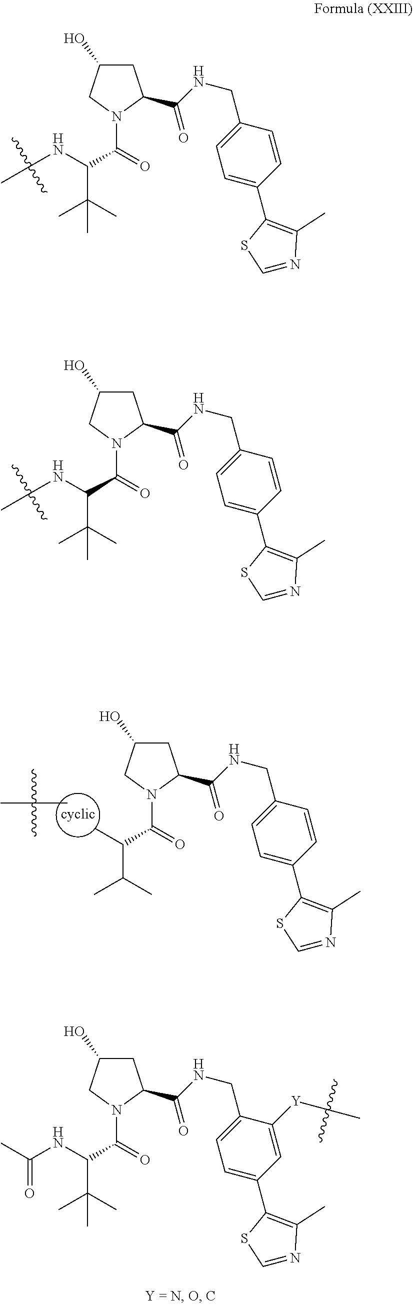

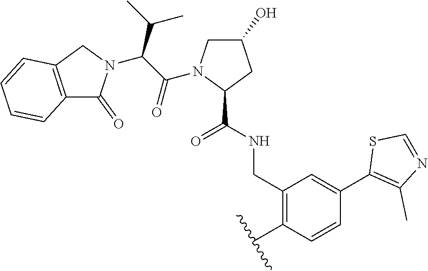 [SMILES: *c1ccc(-c2scnc2C)cc1CNC(=O)[C@@H]1C[C@@H](O)CN1C(=O)[C@H](C(C)C)N1Cc2ccccc2C1=O]

In various embodiments, the compounds described herein include a compound of Formula (XXIV):

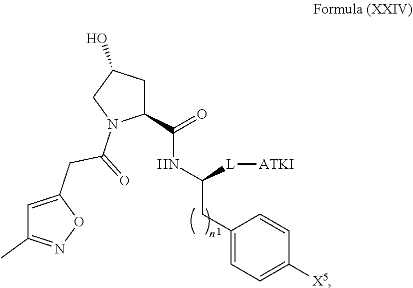

wherein n¹ is 0 or 1; X⁵ is H, F, Cl, C₁-C₃ alkyl or heterocycle.

In various embodiments, the compounds described herein include a compound of Formula (XXV):

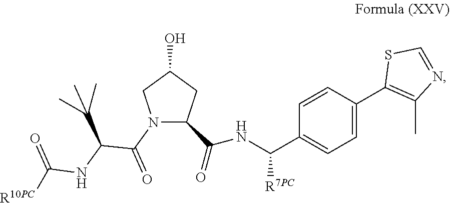

wherein either of R^{7PC} or R^{10PC} is an -L-ATKI group and the other R^{7PC} or R^{10PC} is H.

### Cereblon E3 Ubiquitin Ligase Binding Moieties

In any aspect or embodiment described herein, the CLM comprises a chemical structure selected from the group:

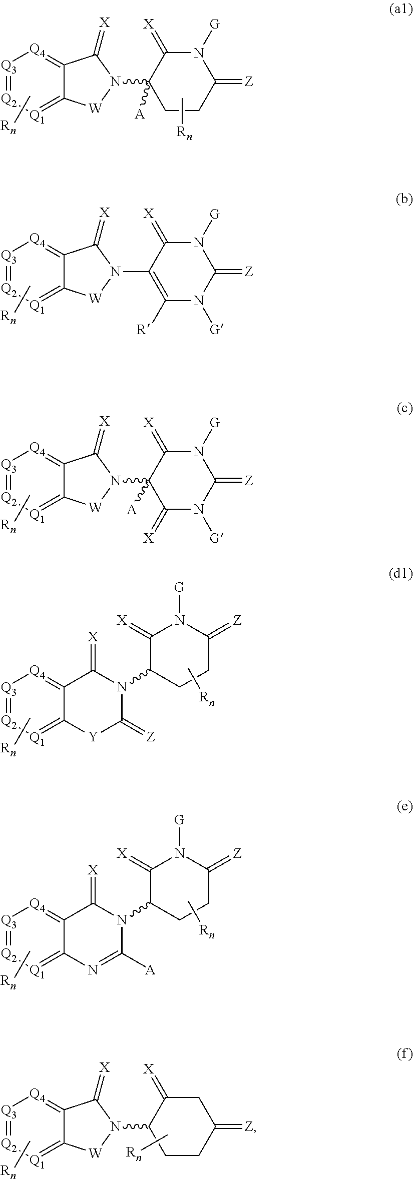

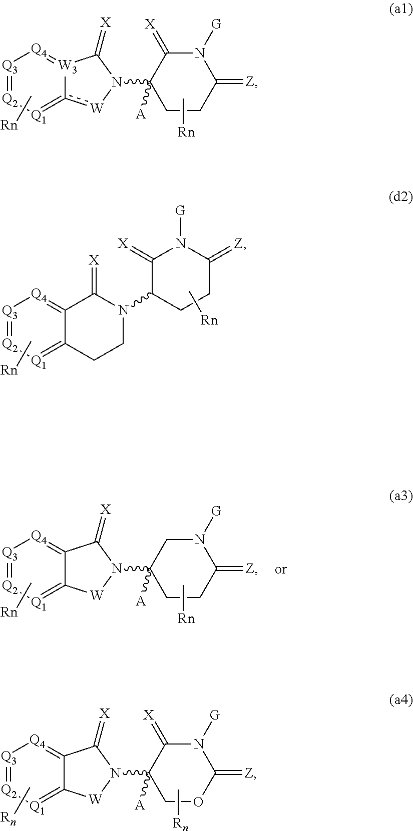

wherein:
W of Formulas (a) through (f) [e.g., (a1), (b), (c), (d1), (e), (f), (a2), (d2), (a3), and (a4)] is independently selected from the group CH₂, O, CHR, C═O, SO₂, NH, N, optionally substituted cyclopropyl group, optionally substituted cyclobutyl group, and N-alkyl;
W₃ is selected from C or N;
X of Formulas (a) through (f) is independently selected from the group absent, O, S and CH₂;
Y of Formulas (a) through (f) is independently selected from the group CH₂, —C═CR′, NH, N-alkyl, N-aryl, N-hetaryl, N-cycloalkyl, N-heterocyclyl, O, and S;
Z of Formulas (a) through (f) is independently selected from the group absent, O, S, and CH₂, except that both X and Z cannot be CH₂ or absent;
G and G′ of Formulas (a) through (f) are independently selected from the group H, optionally substituted linear or branched alkyl (e.g., optionally substituted with R′), OH, R′OCOOR, R′OCONRR″, CH₂-heterocyclyl optionally substituted with R′, and benzyl optionally substituted with R′;
Q1-Q4 of Formulas (a) through (f) each independently represent a carbon C or a nitrogen N substituted with a group independently selected from H, R, N or N-oxide;
A of Formulas (a) through (f) is independently selected from the group H, optionally substituted linear or branched alkyl, cycloalkyl, Cl and F;
R of Formulas (a) through (f) comprises, but is not limited to: H, —CONR′R″, —OR′, —NR′R″, —SR′, —SO₂R′, —SO₂NR′R″, —CR′R″—, —CR′NR′R″—, (—CR′O)_{n′}R″, optionally substituted-aryl (e.g., an optionally substituted C5-C7 aryl), optionally substituted alkyl-aryl (e.g., an alkyl-aryl comprising at least one of an optionally substituted C1-C6 alkyl, an optionally substituted C5-C7 aryl, or combinations thereof), optionally substituted heteroaryl (e.g., an optionally substituted C5-C7 heteroaryl), -optionally substituted linear or branched alkyl (e.g., a C1-C6 linear or branched alkyl optionally substituted with one or more halogen, cycloalkyl (e.g., a C3-C6 cycloalkyl), or aryl (e.g., C5-C7 aryl)), optionally substituted alkoxyl group (e.g., a methoxy, ethoxy, butoxy, propoxy, pentoxy, or hexoxy; wherein the alkoxyl may be substituted with one or more halogen, alkyl, haloalky, fluoroalkyl, cycloalkyl (e.g., a C3-C6 cycloalkyl), or aryl (e.g., C5-C7 aryl)), optionally substituted

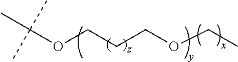

(e.g., optionally substituted with one or more halogen, alkyl, haloalky, fluoroalkyl, cycloalkyl (e.g., a C3-C6 cycloalkyl), or aryl (e.g., C5-C7 aryl)), optionally substituted

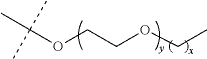

(e.g., optionally substituted with one or more halogen, alkyl, haloalky, fluoroalkyl, cycloalkyl (e.g., a C3-C6 cycloalkyl), or aryl (e.g., C5-C7 aryl)), optionally substitutedcycloalkyl (e.g., optionally substituted C3-C7 cycloalkyl), optionally substitutedheterocyclyl (e.g., optionally substituted C3-C7 heterocyclyl), —P(O)(OR′)R″, —P(O)R′R″, —OP(O)(OR′)R″, —OP(O)R′R″, —Cl, —F, —Br, —I, —CF₃, —CN, —NR′SO₂NR′R″, —NR′CONR′R″, —CONR′COR″, —NR′C(═N—CN)NR′ R″, —C(═N—CN)NR′R″, —NR′C(═N—CN)R″, —NR′C(═C—NO₂)NR′R″, —SO₂NR′COR″, —NO₂, —CO₂R′, —C(C═N—OR′)R″, —CR′═CR′R″, —CCR′, —S(C═O)(C═N—R′)R″, —SF₅ and —OCF₃, wherein at least one R (e.g., at least one of O, OH, N, NH, NH₂, C1-C6 alkyl, C1-C6 alkoxy, optionally substituted-cycloalkyl (e.g., optionally substituted C3-C7 cycloalkyl), optionally substituted-heterocyclyl (e.g., optionally substituted C3-C7 heterocyclyl), -alkyl-aryl (e.g., an -alkyl-aryl comprising at least one of C1-C6 alkyl, C4-C7 aryl, or a combination thereof), aryl (e.g., C5-C7 aryl), heteroaryl aryl (e.g., C5-C7 heteroaryl), amine, amide, or carboxy) is modified to be covalently joined to a PTM, a chemical linker group (L), a ULM, a CLM′ (e.g., CLM′ is an additional CLM that has the same or different structure as a first CLM), or a combination thereof;
each of x, y, and z are independently 0, 1, 2, 3, 4, 5, or 6;
each of n and n′ of Formulas (a) through (f) are independently an integer from 1 to 10 (e.g., 1-4, 1, 2, 3, 4, 5, 6, 7, 8, 9, or 10);
R′ and R″ of Formulas (a) through (f) are independently selected from a H, optionally substituted linear or branched alkyl, optionally substituted cycloalkyl, optionally substituted aryl, optionally substituted heteroaryl, optionally substituted heterocyclic, —C(═O)R, optionally substituted heterocyclyl;
and [symbol] represents a single bond or a double bond; and
[symbol] of Formulas (a) through (f) represents a bond that may be stereospecific ((R) or (S)) or non-stereospecific.

In any aspect or embodiment described herein, the CLM or ULM comprises a chemical structure selected from the group:

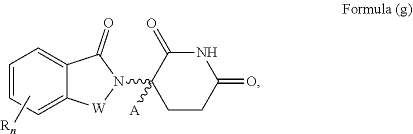

wherein:
W of Formula (g) is independently selected from the group CH₂, O, C═O, NH, and N-alkyl;
A of Formula (g) is selected from a H, methyl, or optionally substituted linear or branched alkyl;
R of Formula (g) is independently selected from a H, O, OH, N, NH, NH₂, methyl, optionally substituted linear or branched alkyl (e.g., optionally substituted linear or branched C1-C6 alkyl), optionally substituted C1-C6 alkoxy, optionally substituted-cycloalkyl (e.g., optionally substituted C3-C7 cycloalkyl), optionally substituted-heterocyclyl (e.g., optionally substituted C3-C7 heterocyclyl), optionally substituted-alkyl-aryl (e.g., an -alkyl-aryl comprising at least one of C1-C6 alkyl, C4-C7 aryl, or a combination thereof), optionally substituted aryl (e.g., C5-C7 aryl), amine, amide, or carboxy);
n of Formulas (g) represent an integer from 1 to 4 (e.g., 1, 2, 3, or 4), wherein at least one R (e.g., at least one of O, OH, N, NH, NH₂, C1-C6 alkyl, C1-C6 alkoxy, -alkyl-aryl (e.g., an -alkyl-aryl comprising at least one of C1-C6 alkyl, C4-C7 aryl, or a combination thereof), aryl (e.g., C5-C7 aryl), amine, amide, or carboxy) is modified to be covalently joined to a PTM, a chemical linker group (L), a ULM, CLM (or CLM′) or combination thereof; and
[symbol] of Formula (g) represents a bond that may be stereospecific ((R) or (S)) or non-stereospecific.

In any of the embodiments described herein, the W, X, Y, Z, G, G′, R, R′, R″, Q1-Q4, A, and Rn of Formulas (a) through (g) [e.g., (a1), (b), (c), (d1), (e), (f), (a2), (d2), (a3), (a4), and (g)] can independently be covalently coupled to a linker and/or a linker to which is attached one or more PTM, ULM, CLM or CLM′ groups.

In any of the aspects or embodiments described herein, the CLM comprises from 1 to 4 R groups independently selected functional groups or atoms, for example, O, OH, N, C1-C6 alkyl, C1-C6 alkoxy, optionally substituted-cycloalkyl (e.g., optionally substituted C3-C7 cycloalkyl), optionally substituted-heterocyclyl (e.g., optionally substituted C3-C7 heterocyclyl), -alkyl-aryl (e.g., an -alkyl-aryl comprising at least one of C1-C6 alkyl, C4-C7 aryl, or a combination thereof), aryl (e.g., C5-C7 aryl), amine, amide, or carboxy, and optionally, one of which is modified to be covalently joined to a PTM, a chemical linker group (L), a ULM, CLM (or CLM′) or combination thereof.

In some embodiments, the CLM is represented by the following structures with the dashed lines indicating linker attachment points:

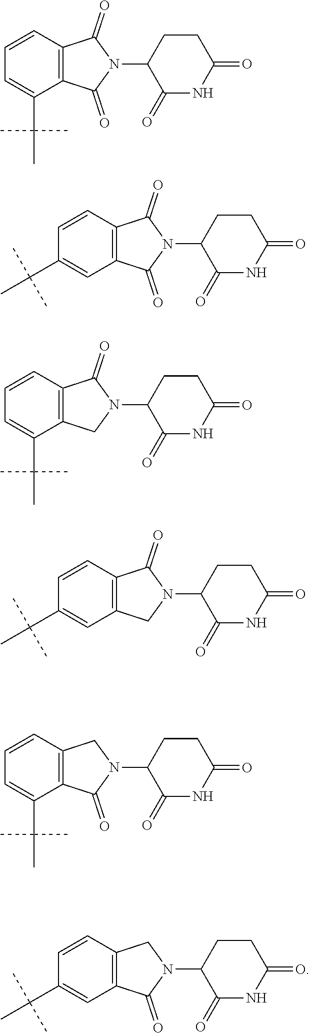

More specifically, non-limiting examples of CLMs include those shown below as well as those “hybrid” molecules that arise from the combination of 1 or more of the different features shown in the molecules below.

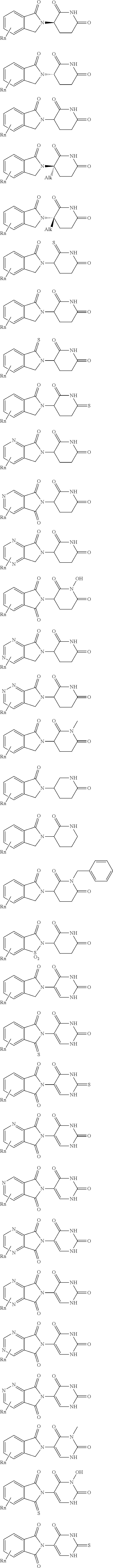

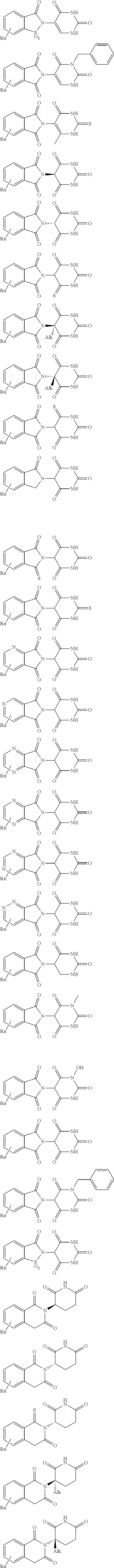

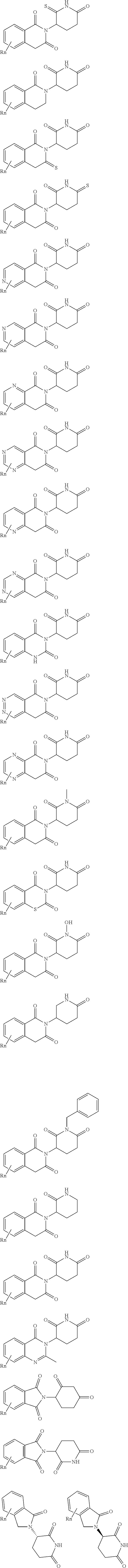

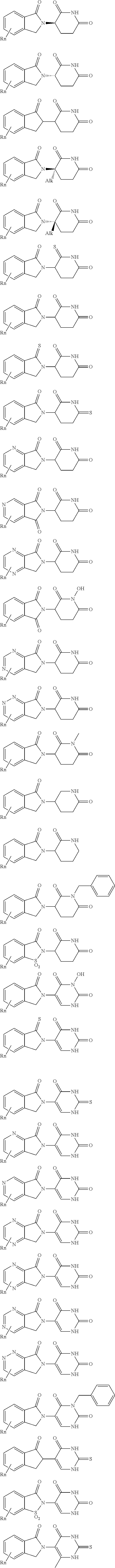

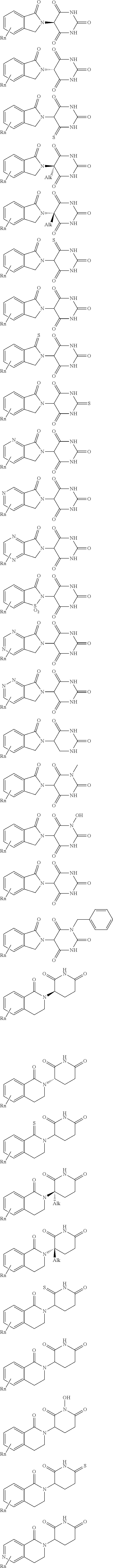

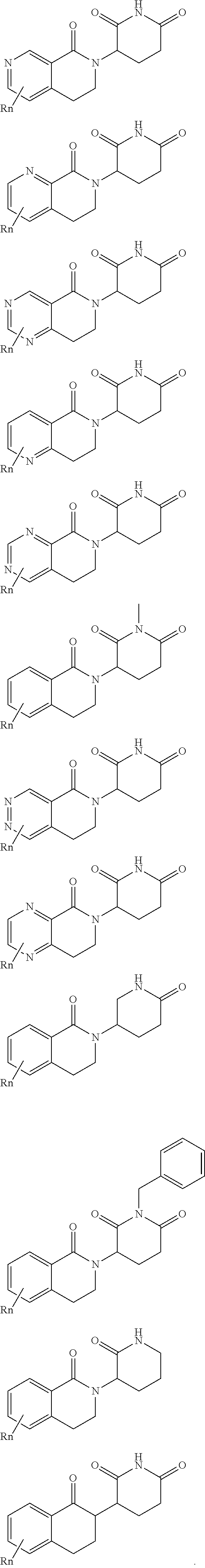

In various embodiments, the ULM is an MDM2 ligand of Formula (XXVI):

In Formula (XXVI),

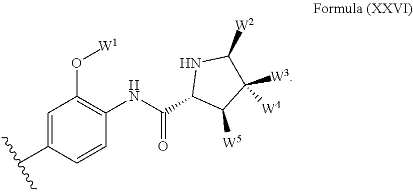

W¹ is independently H or optionally substituted C₁₋₆ alkyl,

W² is independently optionally substituted C₁₋₈ alkyl or optionally substituted C₁₋₈ alkoxy,

W³ is independently F, Cl, Br, I, OR, OC(═O)N(R)₂, CN, NO, NO₂, ONO₂, azido, CF₃, OCF₃, or R;

W⁴ and W⁵ are each independently an optionally substituted aryl or an optionally substituted heteroaryl. In various embodiments, in Formula (XXVI), W¹ is methyl, W² is CH₂C(CH₃)₃, W³ is CN, W⁴ is

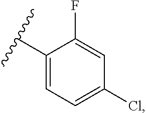 [SMILES: *c1ccc(Cl)cc1F]

### and W⁵ is

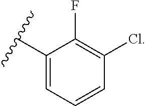 [SMILES: *c1cccc(Cl)c1F]

In various embodiments, the compound of Formula (I) is selected from the group consisting of:

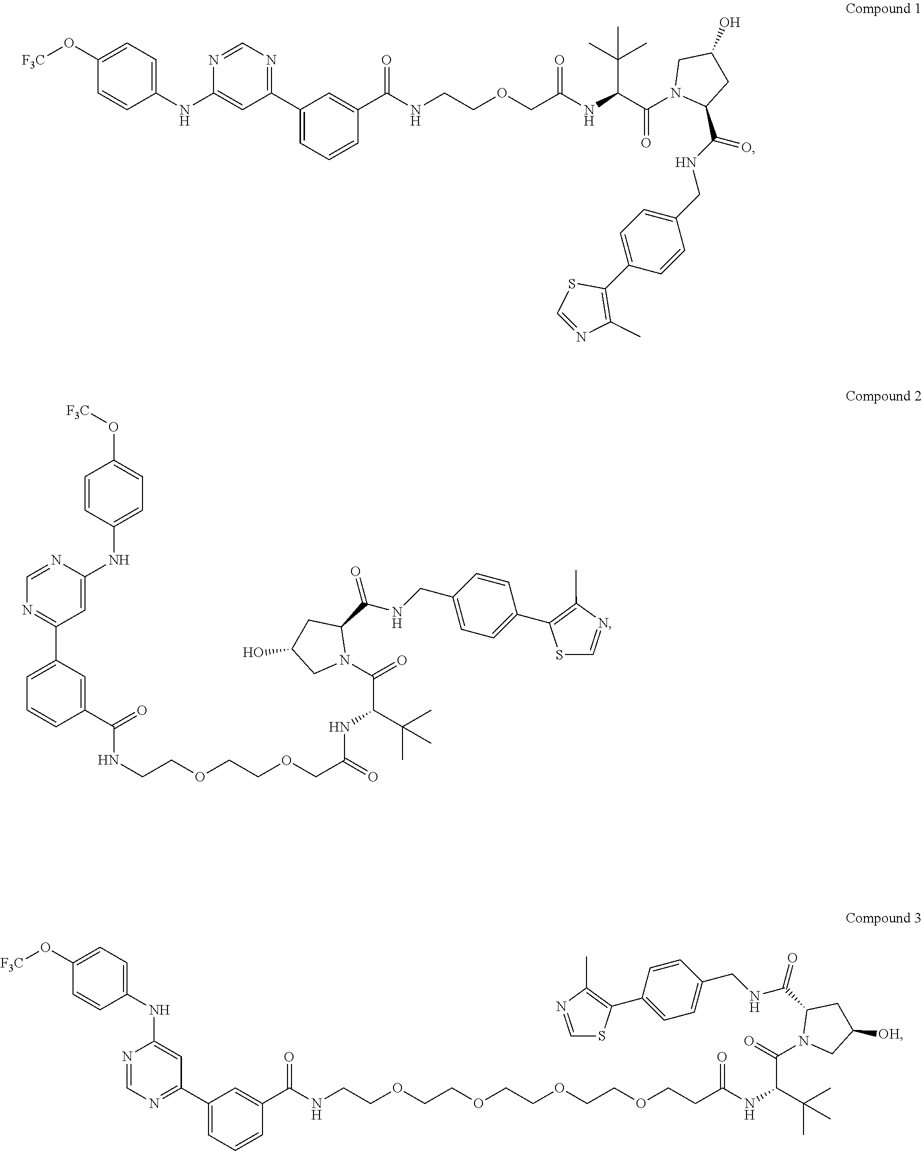 [SMILES: Cc1ncsc1-c1ccc(CNC(=O)[C@@H]2C[C@@H](O)CN2C(=O)[C@@H](NC(=O)COCCNC(=O)c2cccc(-c3cc(Nc4ccc(OC(F)(F)F)cc4)ncn3)c2)C(C)(C)C)cc1 ; Cc1ncsc1-c1ccc(CNC(=O)[C@@H]2C[C@@H](O)CN2C(=O)[C@@H](NC(=O)COCCOCCNC(=O)c2cccc(-c3cc(Nc4ccc(OC(F)(F)F)cc4)ncn3)c2)C(C)(C)C)cc1 ; Cc1ncsc1-c1ccc(CNC(=O)[C@@H]2C[C@@H](O)CN2C(=O)[C@@H](NC(=O)CCOCCOCCOCCOCCNC(=O)c2cccc(-c3cc(Nc4ccc(OC(F)(F)F)cc4)ncn3)c2)C(C)(C)C)cc1]

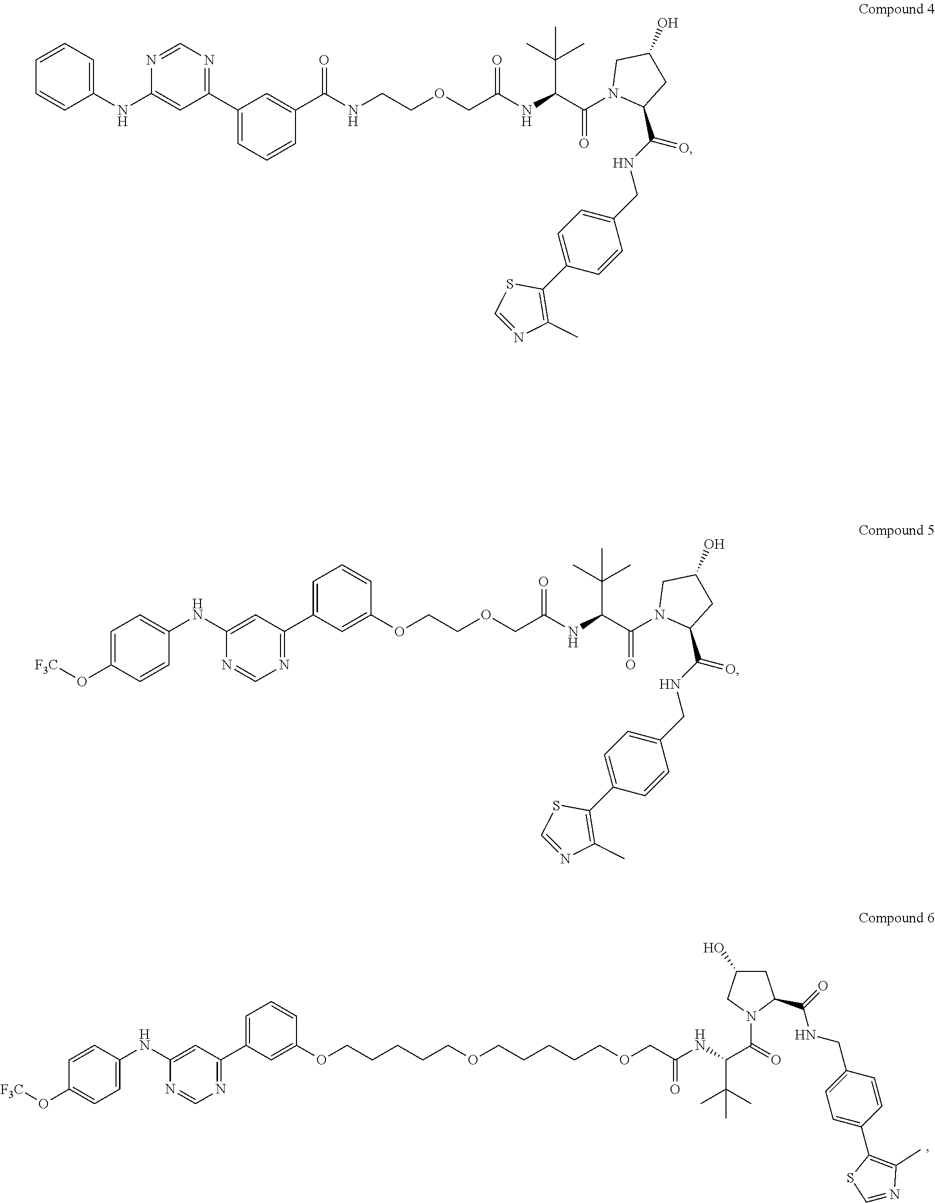 [SMILES: Cc1ncsc1-c1ccc(CNC(=O)[C@@H]2C[C@@H](O)CN2C(=O)[C@@H](NC(=O)COCCNC(=O)c2cccc(-c3cc(Nc4ccccc4)ncn3)c2)C(C)(C)C)cc1 ; Cc1ncsc1-c1ccc(CNC(=O)[C@@H]2C[C@@H](O)CN2C(=O)[C@@H](NC(=O)COCCOc2cccc(-c3cc(Nc4ccc(OC(F)(F)F)cc4)ncn3)c2)C(C)(C)C)cc1 ; Cc1ncsc1-c1ccc(CNC(=O)[C@@H]2C[C@@H](O)CN2C(=O)[C@@H](NC(=O)COCCCCCOCCCCCOc2cccc(-c3cc(Nc4ccc(OC(F)(F)F)cc4)ncn3)c2)C(C)(C)C)cc1]

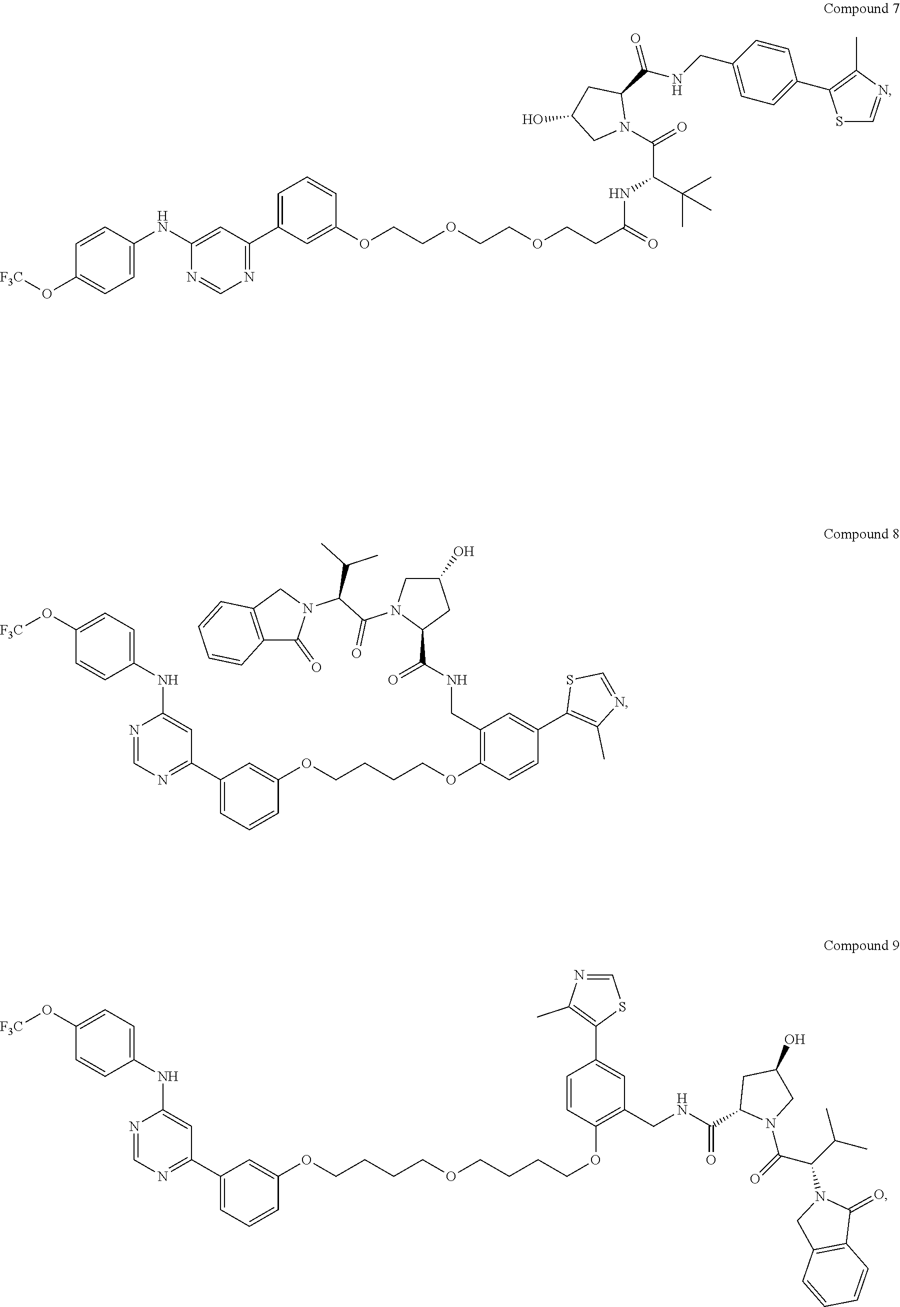 [SMILES: Cc1ncsc1-c1ccc(CNC(=O)[C@@H]2C[C@@H](O)CN2C(=O)[C@@H](NC(=O)CCOCCOCCOc2cccc(-c3cc(Nc4ccc(OC(F)(F)F)cc4)ncn3)c2)C(C)(C)C)cc1 ; Cc1ncsc1-c1ccc(OCCCCOc2cccc(-c3cc(Nc4ccc(OC(F)(F)F)cc4)ncn3)c2)c(CNC(=O)[C@@H]2C[C@@H](O)CN2C(=O)[C@H](C(C)C)N2Cc3ccccc3C2=O)c1 ; Cc1ncsc1-c1ccc(OCCCCOCCCCOc2cccc(-c3cc(Nc4ccc(OC(F)(F)F)cc4)ncn3)c2)c(CNC(=O)[C@@H]2C[C@@H](O)CN2C(=O)[C@H](C(C)C)N2Cc3ccccc3C2=O)c1]

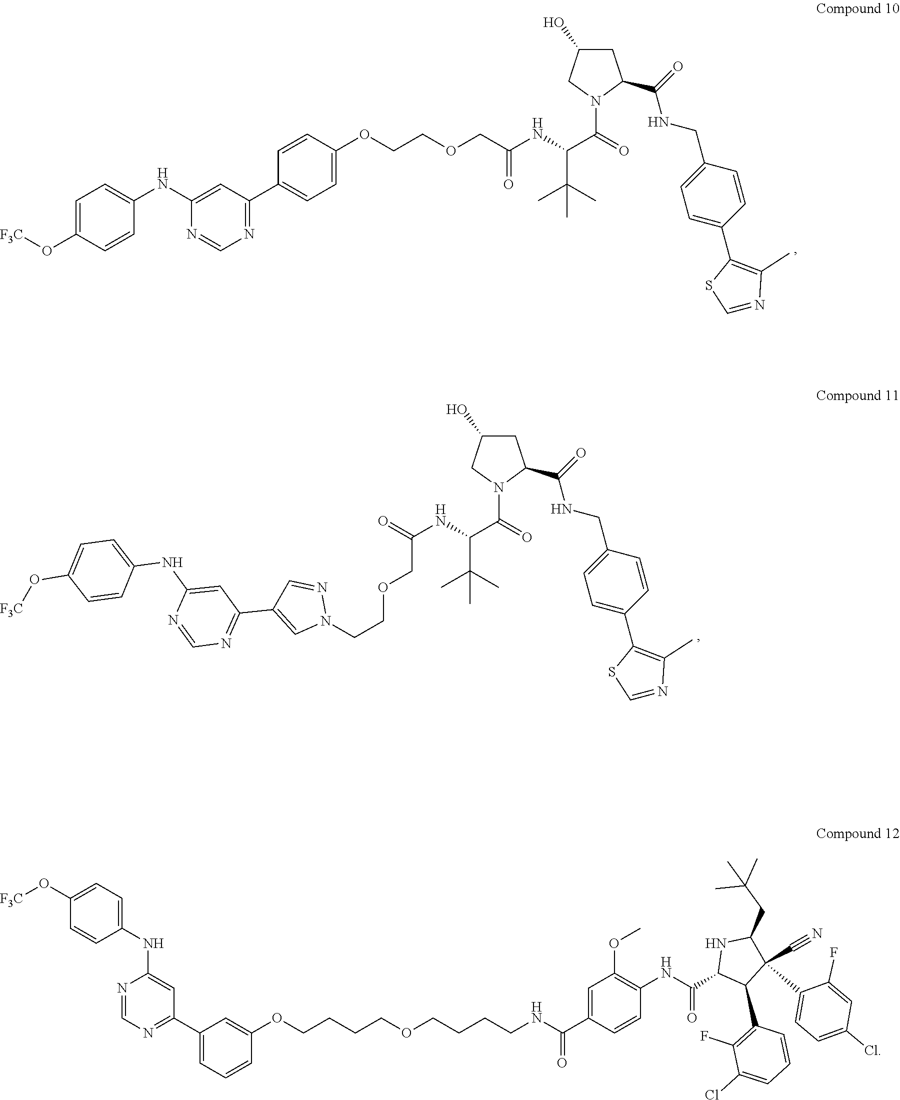 [SMILES: Cc1ncsc1-c1ccc(CNC(=O)[C@@H]2C[C@@H](O)CN2C(=O)[C@@H](NC(=O)COCCOc2ccc(-c3cc(Nc4ccc(OC(F)(F)F)cc4)ncn3)cc2)C(C)(C)C)cc1 ; Cc1ncsc1-c1ccc(CNC(=O)[C@@H]2C[C@@H](O)CN2C(=O)[C@@H](NC(=O)COCCn2cc(-c3cc(Nc4ccc(OC(F)(F)F)cc4)ncn3)cn2)C(C)(C)C)cc1 ; COc1cc(C(=O)NCCCCOCCCCOc2cccc(-c3cc(Nc4ccc(OC(F)(F)F)cc4)ncn3)c2)ccc1NC(=O)[C@@H]1N[C@@H](CC(C)(C)C)[C@](C#N)(c2ccc(Cl)cc2F)[C@H]1c1cccc(Cl)c1F]

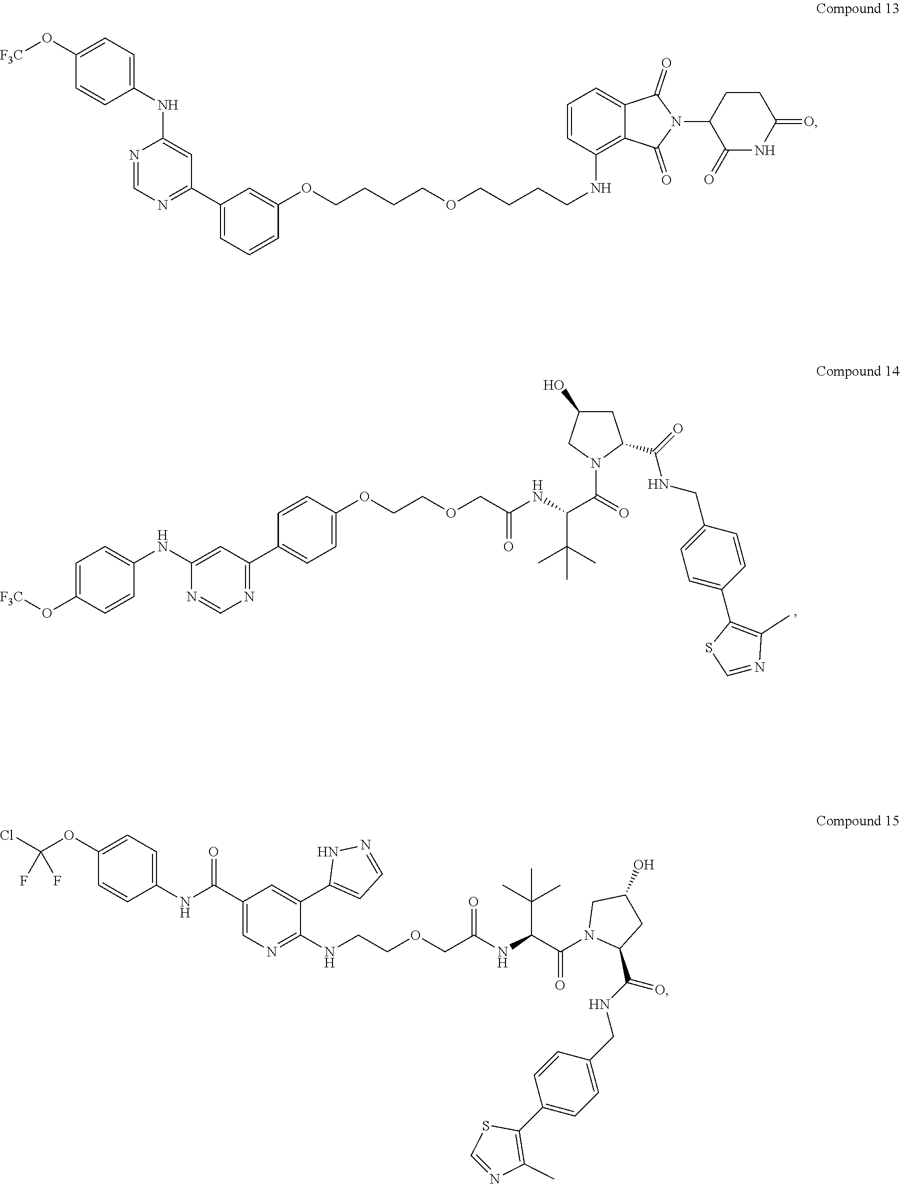 [SMILES: O=C1CCC(N2C(=O)c3cccc(NCCCCOCCCCOc4cccc(-c5cc(Nc6ccc(OC(F)(F)F)cc6)ncn5)c4)c3C2=O)C(=O)N1 ; Cc1ncsc1-c1ccc(CNC(=O)[C@H]2C[C@H](O)CN2C(=O)[C@@H](NC(=O)COCCOc2ccc(-c3cc(Nc4ccc(OC(F)(F)F)cc4)ncn3)cc2)C(C)(C)C)cc1 ; Cc1ncsc1-c1ccc(CNC(=O)[C@@H]2C[C@@H](O)CN2C(=O)[C@@H](NC(=O)COCCNc2ncc(C(=O)Nc3ccc(OC(F)(F)Cl)cc3)cc2-c2ccn[nH]2)C(C)(C)C)cc1]

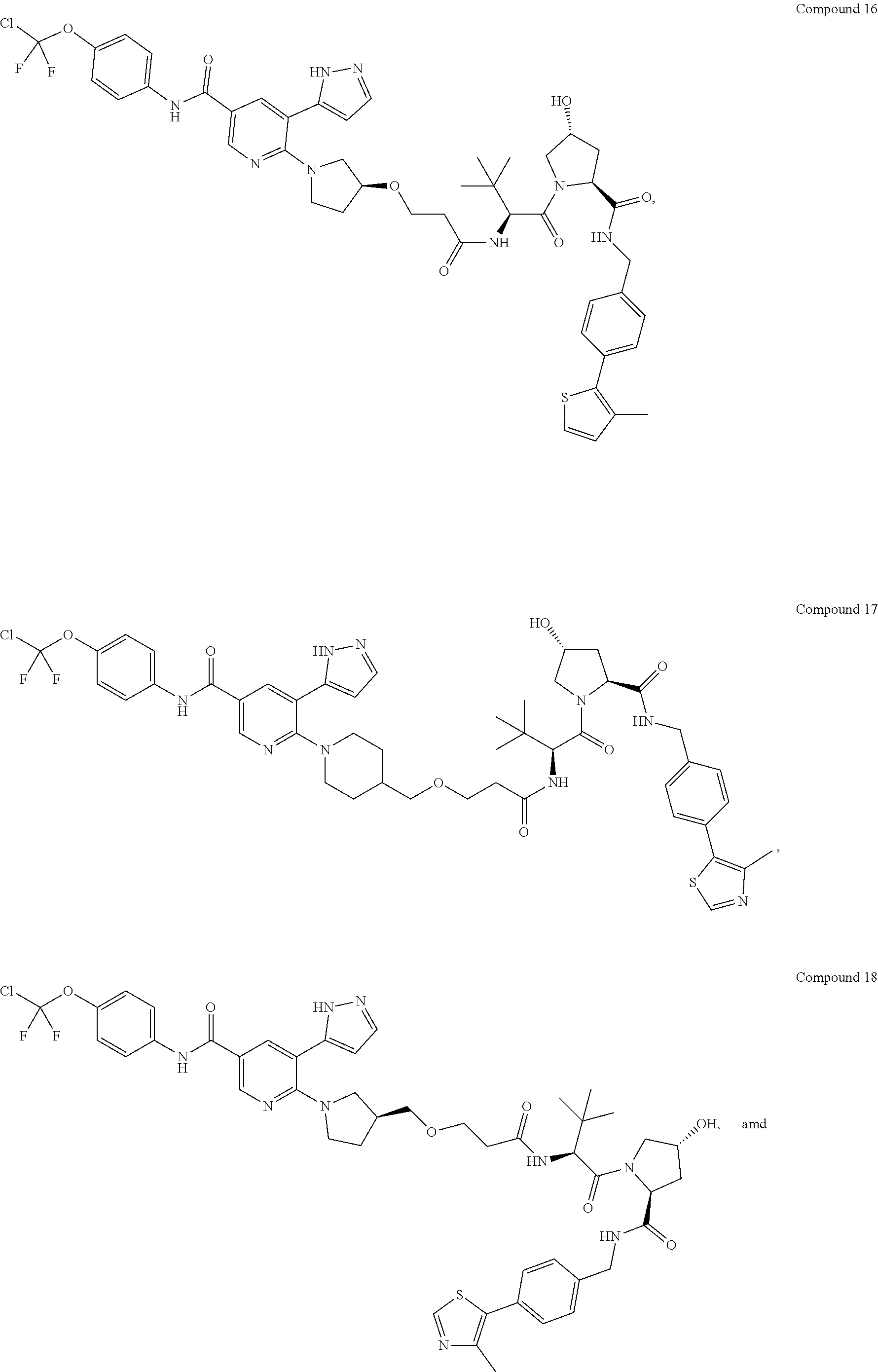

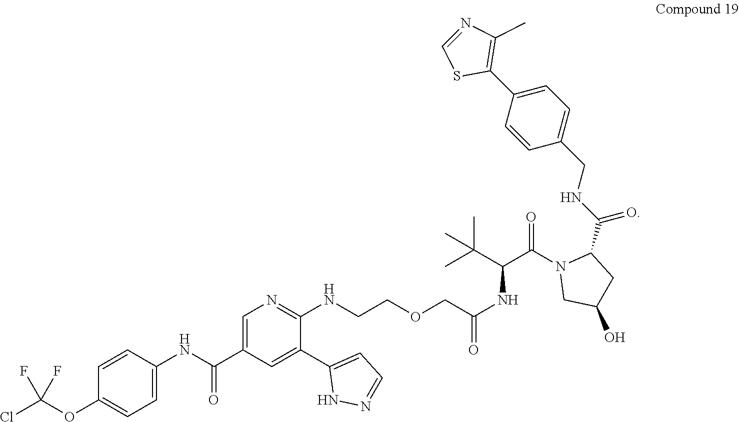 [SMILES: Cc1ncsc1-c1ccc(CNC(=O)[C@@H]2C[C@@H](O)CN2C(=O)[C@@H](NC(=O)COCCNc2ncc(C(=O)Nc3ccc(OC(F)(F)Cl)cc3)cc2-c2ccn[nH]2)C(C)(C)C)cc1]

### BCR-ABL1 Allosteric PROTAC Function in CML Model Systems

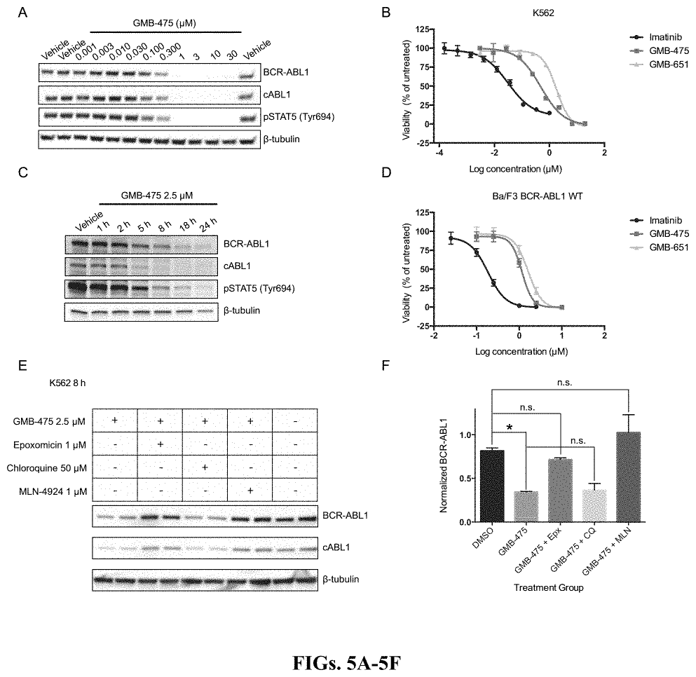

The function of Compound 10 was studied in CML model systems: human K562 cells and murine BCR-ABL1 transformed Ba/F3 cells. In various embodiments, compounds of Formula I such as Compound 10 induce the degradation of BCR-ABL1 and c-ABL1 in the context of both K562 (FIG. 5A) and Ba/F3 (FIG. 5C) cells with concomitant inhibition of downstream signaling via the STAT5 pathway, in a dose- and time-dependent fashion. In various embodiments, compounds of Formula (I) inhibit cell proliferation with an IC₅₀ of about 0.001 μM to about 100 μM, about 0.01 μM to about 90 μM, about 0.01 μM to about 90 μM, about 0.01 μM to about 90 μM, about 0.01 μM to about 80 μM, about 0.01 μM to about 70 μM, about 0.01 μM to about 60 μM, about 0.01 μM to about 50 μM, about 0.01 μM to about 40 μM, about 0.01 μM to about 30 μM, about 0.01 μM to about 20 μM, or about 0.01 μM to about 10 μM. In various embodiments, compounds of Formula (I) inhibit cell proliferation with an IC₅₀ of at least, greater than, or less than about 0.001 μM, 0.05 μM, 0.1 μM, 0.1 μM, 0.2 μM, 0.3 μM, 0.4 μM, 0.5 μM, 0.6 μM, 0.7 μM, 0.8 μM, 0.9 μM, 1 μM, 1.2 μM, 1.4 μM, 1.6 μM, 1.8 μM, 2 μM, 2.2 μM, 2.4 μM, 2.6 μM, 2.8 μM, 3 μM, 3.2 μM, 3.4 μM, 3.6 μM, 3.8 μM, 4 μM, 4. μM, 4.4 μM, 4.6 μM, 4.8 μM, 5 μM, 5.25 μM, 5.5 μM, 5.75 μM, 6 μM, 6.25 μM, 6.5 μM, 6.75 μM, 7 μM, 7.25 μM, 7.5 μM, 7.75 μM, 8 μM, 8.25 μM, 8.5 μM, 8.75 μM, 9 μM, 9.25 μM, 9. μM, 9.75 μM, 10 μM, 20 μM, 30 μM, 40 μM, 50 μM, 60 μM, 70 μM, 80 μM, 90 μM, or 10 μM.

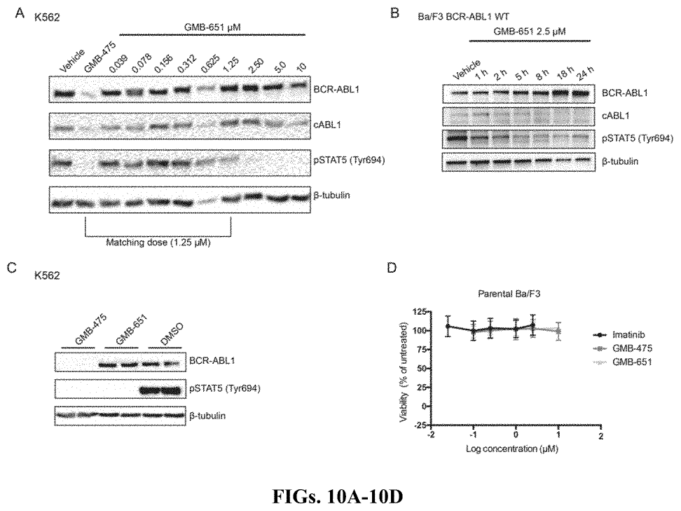

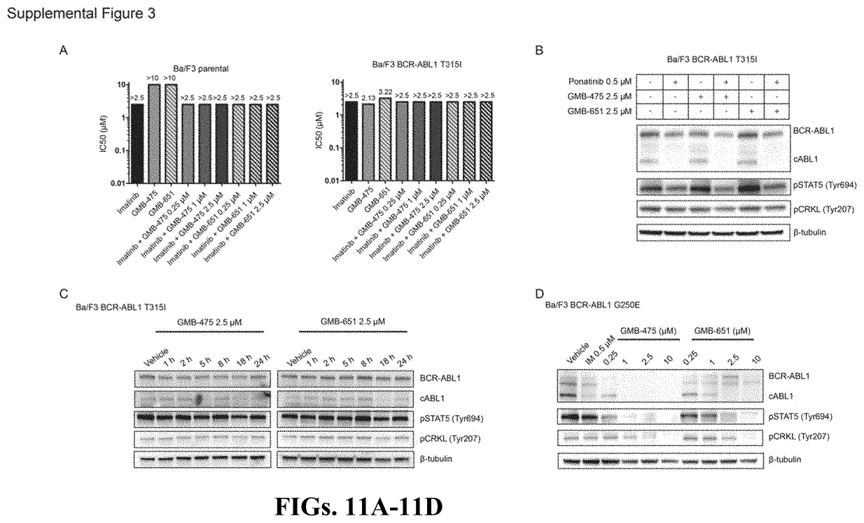

Compound 10 inhibits cell proliferation with an IC₅₀ of approximately 1 μM (FIG. 5B/D). In various embodiments, compounds of Formula (I) do not display toxicity against Ba/F3 cells up to about 10 μM, 15 μM, 20 μM, 25 μM, 30 μM, 35 μM, 40 μM, 45 μM, or 50 μM. For example, neither Compound 10 nor Compound 14 displayed toxicity against parental Ba/F3 cells up to 10 μM, emphasizing the selectivity of these compounds (FIG. 10D). In various embodiments, co-treatment of a subject with one or more pharmacological modulators and a compound of Formula (I), results in degradation of BCR-ABL1 by a ubiquitination and proteasome-dependent mechanism and is not lysosome-dependent (FIGS. 5E/5F). For example, co-treatment of K562 cells with the proteasome inhibitor epoxomicin and Compound 10 restored the levels of BCR-ABL1 and c-ABL1 compared to Compound 10 alone, whilst modulation of lysosomal pH with chloroquine had no effect. Additionally, inhibition of neddylation using MLN-4924 inhibited the degradation of BCR-ABL1 and ABL1, since VHL neddylation is required for its E3 ligase activity (FIGS. 5E/5F).

Combination Treatment with a TP-Competitive BCR-ABL1 TKIs

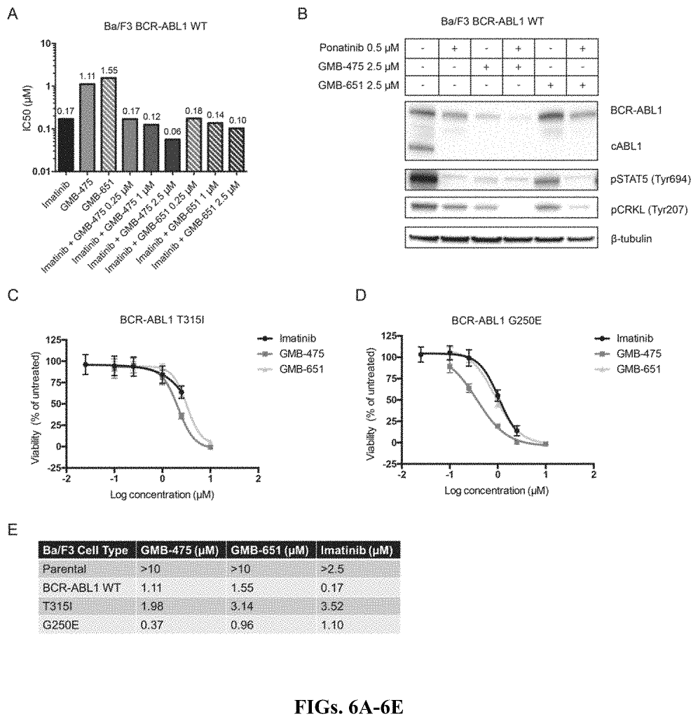

Compounds of Formula (I) and ATP-competitive inhibitors such as imatinib bind at orthogonal sites on protein kinases such as BCR-ABL1. Dose response titrations were performed with BCR-ABL1 transformed Ba/F3 cells for imatinib, Compound 10 and Compound 14 and IC₅₀ values were determined to be 0.17 μM, 1.11 μM, and 1.55 μM respectively (FIG. 6A). The IC₅₀ of imatinib in the presence of increasing concentrations of Compound 10 or Compound 14 (FIG. 6A) was also determined. Unexpectedly, co-treatment with 2.5 μM Compound 10 reduced the IC₅₀ of imatinib almost 3-fold, likely due to degradation reducing the BCR-ABL1 protein present, suggesting a lower dose of imatinib can entirely abrogate signaling. In various embodiments, co-administration of a compound of Formula (I) and at least one ATP-competitive tyrosine kinase inhibitor reduces the IC₅₀ of the ATP-competitive tyrosine kinase inhibitor by at least about 1.2, 1.4, 1.6, 1.8, 2.0, 2.2, 2.4, 2.6, 2.8, 3, 3.2, 3.4, 3.6, 3.8, 4, 4.2, 4.4, 4.6, 4.8, 5, 6, 7, 8, 9, 10, 15, 20, 25, 30, 35, 40, 45, 50, 55, 60, 65, 70, 75, 80, 85, 90, 95, or 100-fold in comparison to the ATP-competitive inhibitor alone. The co-treatment of the diastereomer Compound 14 slightly reduced the IC₅₀ value for imatinib, demonstrating that co-treatment with an active degrader is advantageous over co-treatment with the equivalent allosteric inhibitor.

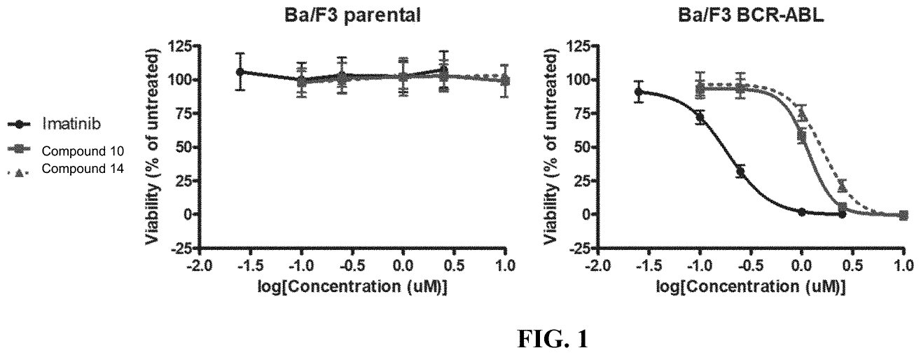

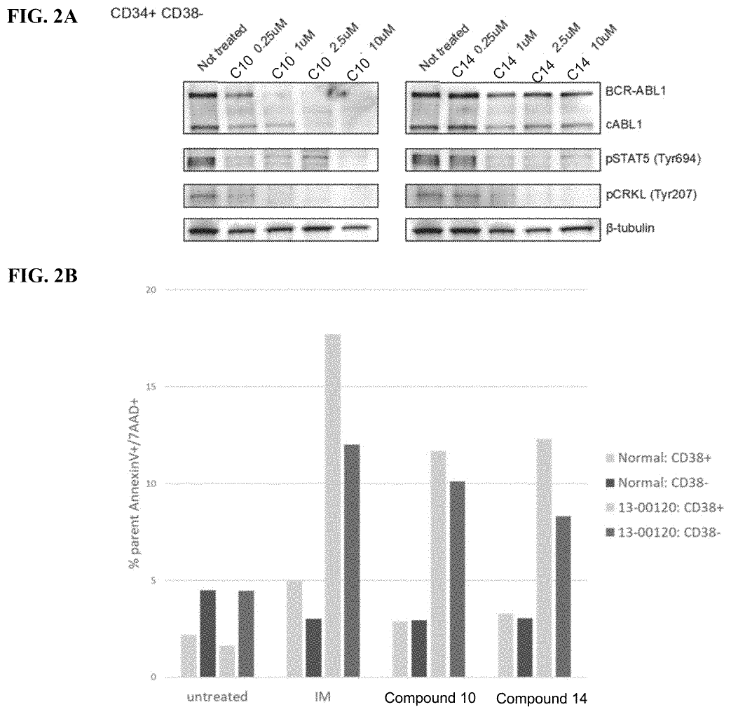

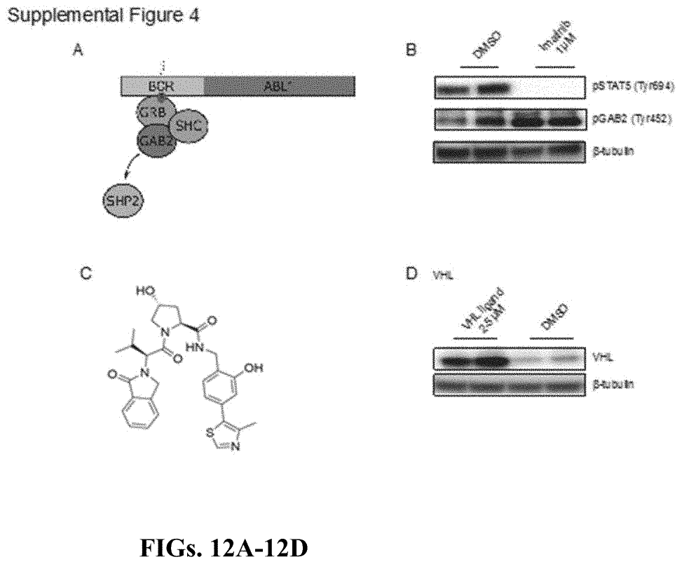

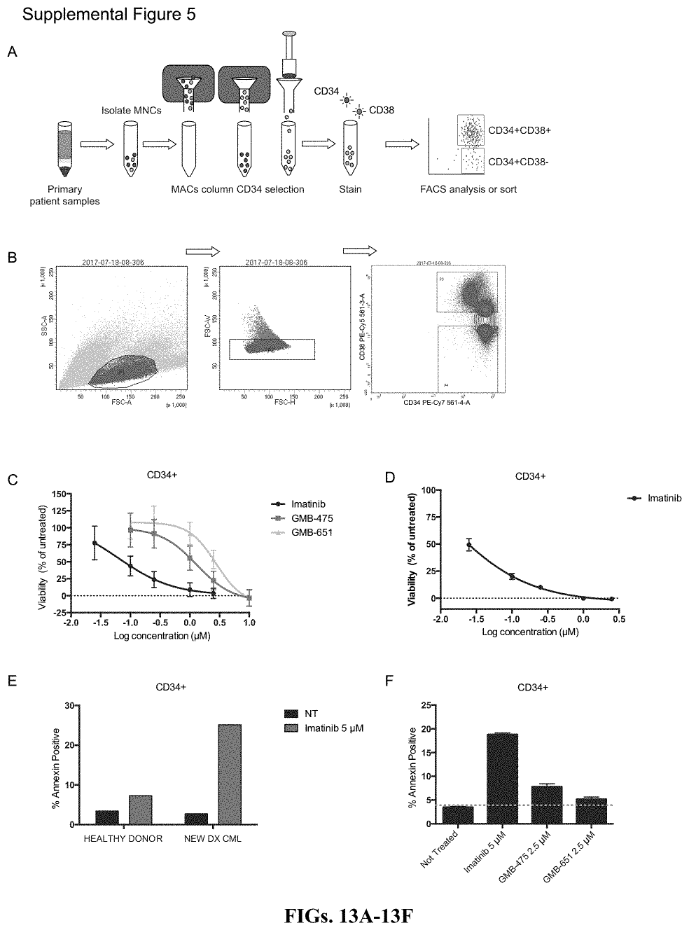

Co-treatment with ponatinib, a potent BCR-ABL1 inhibitor effective against imatinib-resistant kinase domain point mutations such as T315I, with compounds of Formula (I) was monitored by immunoblot blot (FIG. 6B). Dual treatment with ponatinib and a compound of Formula (I) was able to fully inhibit phosphorylation of CRKL in Ba/F3 BCR-ABL1 wild-type cells, yet had no significant additive effect in Ba/F3 BCR-ABL1 T315I cells (FIG. 11B). In various embodiments, the activity of compounds of Formula (I) is enhanced when binding to an inactive conformation of BCR-ABL1. Ponatinib stabilizes the inactive conformation of BCR-ABL 1 upon binding, which unexpectedly enhanced the ability of Compound 10 to induce degradation (FIG. 6B). Co-treatment of ponatinib and Compound 14 showed little additional effect beyond the level of inhibition of kinase activity of ponatinib alone (FIG. 11A-1C). Together, these data suggest that treatment with compounds of Formula (I) can be used in concert with traditional inhibitors to reduce the dose of inhibitor required and therefore potentially reduce side effects of ATP mimics. In various embodiments, co-administration of a compound of Formula (I) and at least one ATP-competitive tyrosine kinase inhibitor reduces the required dose of the ATP-competitive tyrosine kinase inhibitor, such as an FDA-approved dose of the ATP-competitive inhibitor, by at least about 1.2, 1.4, 1.6, 1.8, 2.0, 2.2, 2.4, 2.6, 2.8, 3, 3.2, 3.4, 3.6, 3.8, 4, 4.2, 4.4, 4.6, 4.8, 5, 6, 7, 8, 9, 10, 15, 20, 25, 30, 35, 40, 45, 50, 55, 60, 65, 70, 75, 80, 85, 90, 95, or 100-fold in comparison to the dose of the ATP-competitive inhibitor alone.

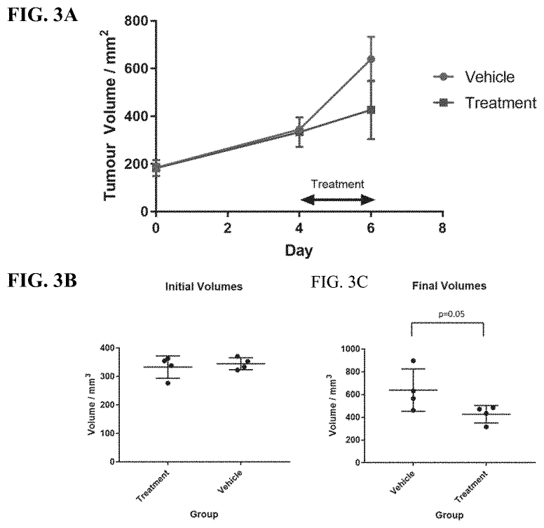

The Ba/F3 system was used to test the ability of Compound 10 to inhibit the proliferation of clinically relevant imatinib-resistant BCR-ABL1 point mutants (FIG. 6C-6E). As shown above, Compound 10 demonstrated a slight advantage over Compound 14 against Ba/F3 cells expressing wild-type BCR-ABL1 but neither were as potent as imatinib (FIG. 5E). In various embodiments, compounds of Formula (I) can treat any of the proliferative disorders (cancers) described herein in patients having a mutation in BCR-ABL1 that reduces the efficacy of ATP-competitive inhibitors with substantially no reduction in efficacy of compounds of Formula (I). For example, introduction of a T315I mutation significantly reduced the potency of imatinib but had little effect on the potency of Compound 10 (FIGS. 3C and 11C). In various embodiments, compounds of Formula (I) can treat any of the proliferative disorders (cancers) described herein in patients having a T315I mutation in BCR-ABL1 with substantially no reduction in efficacy of compounds of Formula (I).

Cells bearing a G250E mutation in BCR-ABL1 were particularly susceptible to Compound 10 displaying enhanced anti-proliferative activity (FIGS. 3D and 11D). In various embodiments, compounds of Formula (I) are more active against BCR-ABL1 kinases bearing a G250E mutation than against BCR-ABL1 kinases lacking a G250E mutation.

### Non-Kinase Roles of BCR-ABL1 Assessed by Protein Array Analysis in K562 Cells

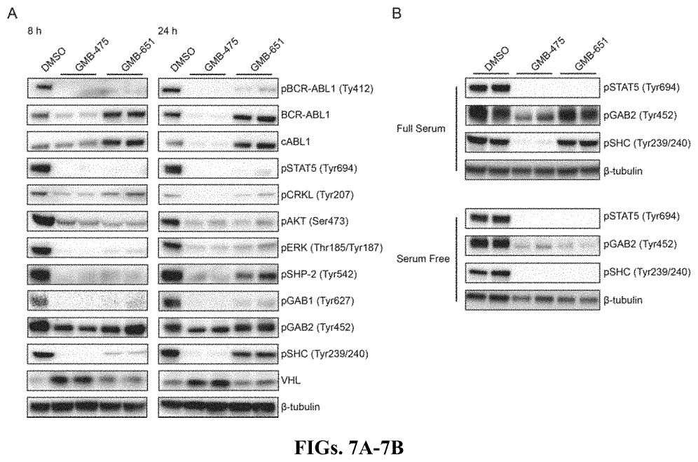

The non-kinase roles of BCR-ABL1 that can, without being bound by theory, contribute to the additional effect of the compounds of Formula (I), were studied using a functional proteomic approach to compare degradation to allosteric inhibition. Using reverse phase protein arrays (RPPA) changes in levels of proteins and post-translational modifications were analyzed in a rapid and efficient manner. K562 cells were treated with 5 μM of either Compound 10 or Compound 14 for 8 h to probe the acute changes in protein states which occur on the degradation/inhibition of BCR-ABL1. Treatment with Compound 10, but not Compound 14, showed a decrease in total ABL1 protein by RPPA (FIG. 10C). Immunoblot analysis was used to validate selected target proteins at 8 h and 24 h (FIG. 7A). Both Compound 10 and Compound 14 were able to inhibit the kinase activity of BCR-ABL1 as verified by both pBCR-ABL1 (Tyr412) and pSTAT-5 (FIG. 7A). Differences between degradation and inhibition alone became evident at 8 h and were further accentuated after 24 h of treatment. For example, downstream signaling of BCR-ABL1 via the pCRKL and pERK pathways was inhibited to a greater extent with the active PROTAC compared to the inhibitor only control (FIG. 7A), indicating the advantages of degradation on suppression of oncogenic signaling.

However, the most extreme differences observed were for pSHP-2, pGAB2, pSHC and VHL. The increase in VHL protein can be explained by the stabilizing effect the binding of a ligand imparts to the VHL protein itself, as exemplified by the increase in the intensity of VHL band in K562 cells treated with the VHL ligand alone (FIG. 12C). GAB2, SHC and SHP-2 are all in the canonical network of BCR-ABL1 and together contribute to the activation of the MAPK signaling cascade. Phosphorylation of Y177 of BCR-ABL1 yields a docking site for GRB2 which in turn recruits GAB2 and/or SHC. Once phosphorylated, GAB2 recruits and activates SHP-2. Inhibition of BCR-ABL1 had no effect on the phosphorylation state of GAB2 but degradation of BCR-ABL1 reduced the level of GAB2 phosphorylation (FIGS. 7 and 12B).

To further investigate this phenomenon, the experiment mentioned above was repeated in the presence and absence of serum. Under full serum (10% FBS) conditions, both Compound 10 and Compound 14 were able to inhibit the kinase activity of BCR-ABL1, as measured by loss of pSTAT5 signal, but only Compound 10 was able to reduce phosphorylation of GAB2 and SHC (FIG. 7B). Conversely, under serum-free conditions, both Compound 10 and Compound 14 were able to inhibit phosphorylation of GAB2 and SHC, as well as STAT5 (FIG. 7B). This suggests a scaffolding role for BCR-ABL1 in signaling via this pathway. Under serum-free conditions, only the constitutively active BCR-ABL1 kinase domain is able to (auto)-phosphorylate Y177, a key docking site, and thus both degrader (Compound 10) and inhibitor (Compound 14) are able to block signaling.

### Efficacy of Compounds of Formula (I) in Primary CML Patient Samples

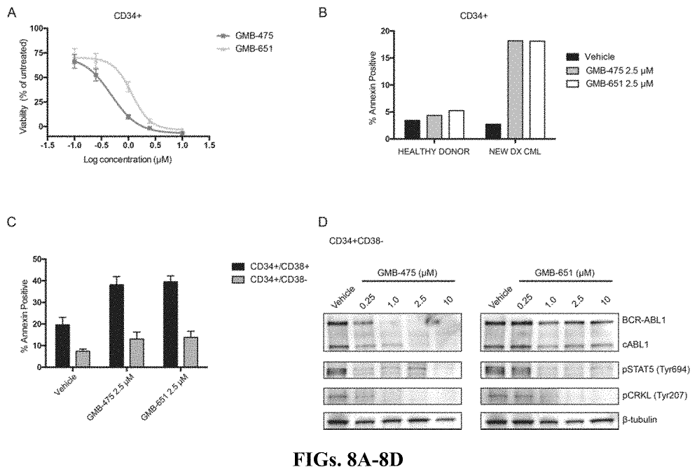

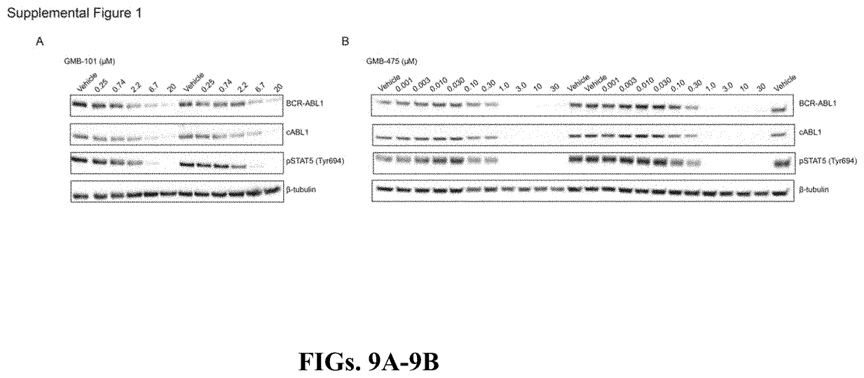

To further explore the scaffolding roles of BCR-ABL1 and the prosurvival effects in CML stem and progenitor cells in the context of targeted protein degradation compounds of Formula (I) were evaluated in experiments utilizing primary CML patient samples. Initial anti-proliferative activity was assessed for Compound 10 and Compound 14 in CD34+ cells from newly diagnosed CML patients (patient 1 and 2; FIGS. 8A/13C, respectively). Compounds of Formula (I), as well as imatinib as a positive control (FIG. 13D), inhibited in vitro proliferation and induced apoptosis in primary CML CD34+ patient cells but had no effect on healthy donor CD34+ cells (FIG. 8B/13E and FIG. 13F; patients 4 and 3, respectively). Notably, Compound 10 was greater than 2-fold more potent at inhibiting proliferation than Compound 14. The CD34+ cells from patient 1 were sorted into CD34+CD38+(progenitor cells) and CD34+CD38− (stem cells) and the cells were assayed for their ability to induce apoptosis in those populations (FIGS. 8C and 13B). Treatment of these cells with either Compound 10 or Compound 14 induced apoptosis in the progenitor cells and to a lesser extent in the stem cells but with no appreciable difference between the PROTAC and the diastereomer control, possibly due to the use of saturating doses. Finally, it was confirmed by immunoblot that Compound 10, but not Compound 14, was indeed able to induce degradation of both BCR-ABL1 and ABL1 in primary patient LSCs (FIG. 8D).

### Examination of Scaffold Hopping in Bifunctional Degradative Compounds

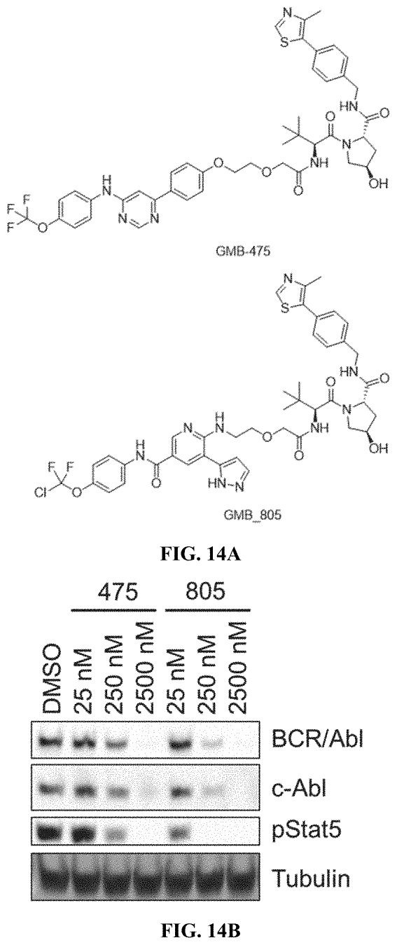

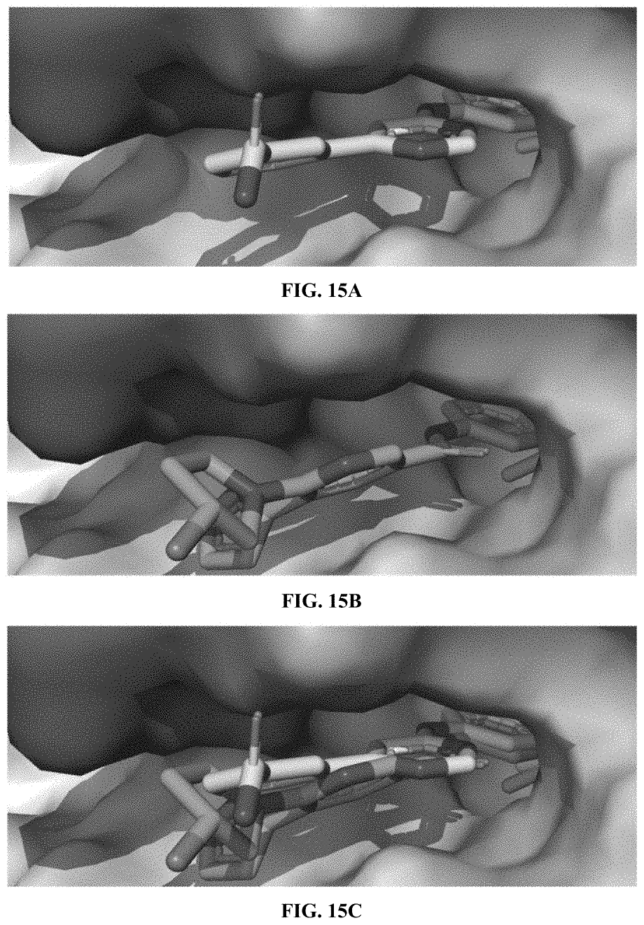

Herein is described the development of allosteric bifunctional degradative compound, including bifunctional compounds derived from the BCR-Abl inhibitor GNF-5, e.g. GMB-475 (Compound 10; FIG. 14A). A more potent allosteric BCR-Abl ligand than GNF-5 was developed, ABL001. The two allosteric BCR-Abl ligands were utilized to investigate whether scaffold hopping could enhance a bifunctional degradative compound's activity without the requirement for linker re-optimization. To this end, the GNF-5 derived portion of GMB-475 (Compound 10) was replaced with an Abl-001 derived recruiting element employing an identical linker length and composition, and with a very similar exit vector from the myristate binding pocket, where the allosteric recruiting elements bind (FIGS. 15A-15C). The resulting molecule, GMB-805 (Compound 19), demonstrated an enhanced ability to induce BCR-Abl degradation compared to GMB-475 (Compound 10) as shown in FIG. 14B.

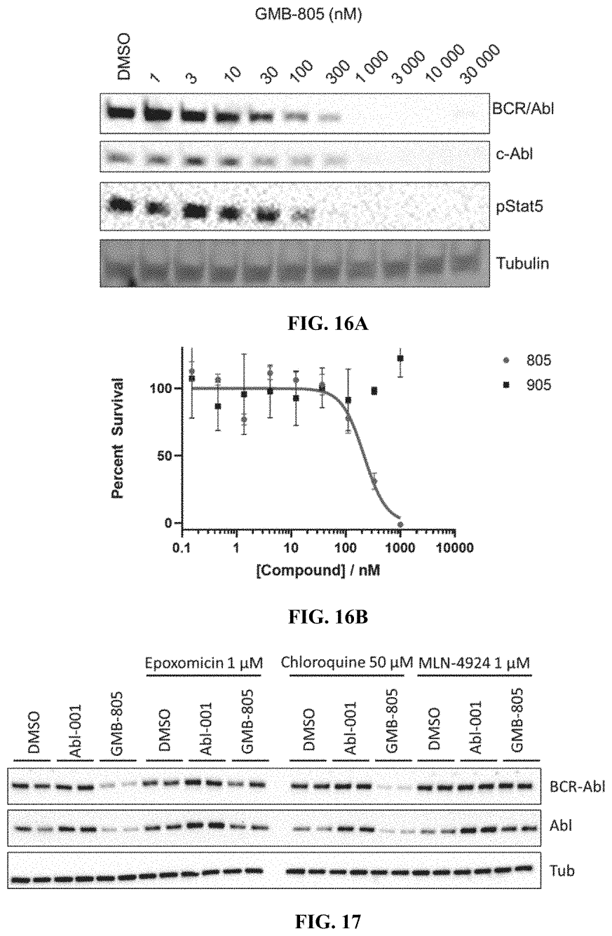

The bifunctional degradative compound GMB-805 (Compound 19) was fully characterised by performing an extended dose response (FIG. 16A), which enabled the calculation of a DC₅₀ value (the concentration at which half maximal degradation is observed) of 30 nM. Thus, demonstrating the validity of the scaffold hoping approach by enhancing the activity of GMB-805 (Compound 19) by over 10-fold compared to GMB-475 (Compound 10; DC₅₀ 340 nm) with an identical linker.

### GMB-805 (Compound 19) Functions Via Protein Degradation of the Target Protein

Additionally, it was confirmed via pharmacological co-treatment with modulators of various cellular protein degradation processes that GMB-805 (Compound 19) functioned via protein degradation of the target protein (FIG. 17). Firstly, it was demonstrate that GMB-805 (Compound 19) induces degradation via the proteasome by co-treatment with proteasome inhibitor epoxomicin, which restored protein concentration back to untreated levels. Co-treatment with chloroquine had no appreciable effect on the levels of either BCR-Abl or c-Abl suggesting that the lysosome is not important for this effect. Finally, co-treatment with MLN-4924 (NEDD8-Activating Enzyme inhibitor) was also able to rescue protein levels. The VHL-Cullin2-RING ligase complex requires neddylation for its activity, and the neddylation inhibitor MLN-4924 demonstrates that the degradation observed is Cullin-dependent. Interestingly, it was observed in this experiment that Abl-001 binding appears to stabilise c-Abl presumably via ligand induced stabilization.

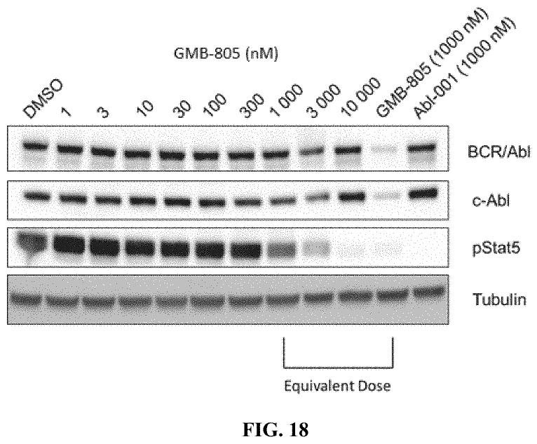

To further demonstrate the VHL dependency of this induced degradation, a control of GMB-805 (Compound 19) was prepared that is identical but for the inversion of the stereochemistry in the VHL ligand. This diastereomer control possess equivalent cell permeability to GMB-805 (Compound 19), but is unable to recruit VHL and as such is unable to induce degradation of c-Abl or BCR-Abl as shown in FIG. 18. The control compound retains the ability to inhibit BCR-Abl as demonstrated by the loss of Stat-5 phosphorylation, albeit at higher concentrations than the active bifunctional degradative compound, demonstrating an advantage in potency of degradation over inhibition. Crucially, GMB-805 (Compound 19) possesses potent antiproliferative activity against the BCR-Abl driven cell line K562 with an IC₅₀ of 169 nM while the control compound exhibits no antiproliferative activity up to 1 μM (FIG. 16B).

### In Vivo Activity of GMB-805 (Compound 19)

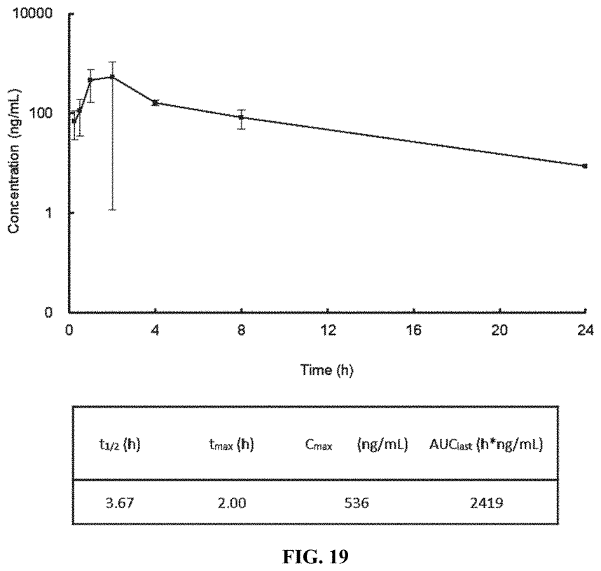

Given the potency of GMB-805 (Compound 19) in cellular model systems, the compounds activity in vivo was examined. Initial exploration of the pharmacokinetic properties revealed that GMB-805 (Compound 19) has an in vivo half-life of over 3½ hours when administered 10 mg/kg IP (FIG. 19) and Cₘₐₓ of well over above both the DC₅₀ and IC₅₀. Given these promising pharmacokinetic properties, an acute in vivo efficacy study with exemplary compound GMB-805 (Compound 19) was performed.

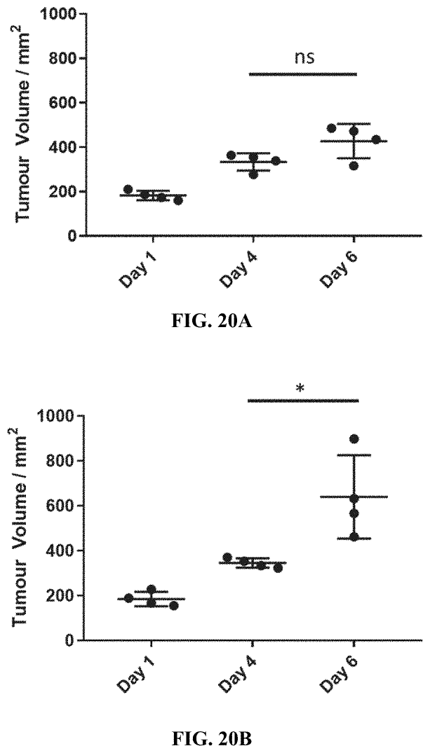

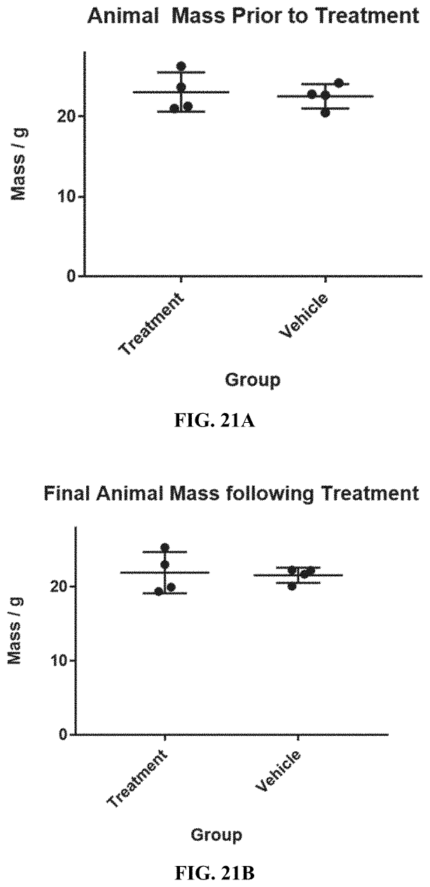

K562 cells were implanted subcutaneously into the flank of athymic mice and tumors were allowed to develop to approx. 200 mm³. Animals were then randomised into treatment or vehicle groups on day 1. On day 4-6, animals were either treated with exemplary compound GMB-805 (Compound 19; 200 mg/kg) or vehicle control by IP injection once every 24 hours. The volume of the tumors was monitored, and animals treated with GMB-805 (Compound 19) showed no significant increase in tumor volume (FIG. 20A), while vehicle treated animals' tumor volume increased significantly during the same time period (FIG. 20B). Despite the relatively high dose, no toxicity or weight loss was observed in the treated animals (FIGS. 21A and 21B).

GMB-805 (Compound 19) demonstrates a >10 fold increase in ability to induce degradation relative to GMB-475 (Compound 10) and possesses in vivo activity. The data herein demonstrates for the first time that scaffold hopping can enable the development of bifunctional compounds with significantly enhanced ability to induce degradation of an oncogenic protein without the need to repeat the time-consuming linker optimization. This will likely be an important finding as higher affinity ligands could be developed simultaneously to linker optimization to enable more rapid bifunctional degradative compound development. Furthermore, it enabled the discovery of a BCR-Abl bifunctional compounds with greater than 10-fold enhanced activity, improved pharmacokinetic properties and in vivo activity.

While the activity of the BCR-ABL1 tyrosine kinase is quintessential to the pathogenesis of CML and the justification for and basis of the successful implementation of molecularly targeted small-molecule therapies, clinical responses to ABL1 TKIs run a spectrum from deep and durable molecular remission in most patients to overt drug resistance and disease progression in others. These differences are attributable to several known (e.g. resistant BCR-ABL 1 kinase domain mutations, cellular drug transporter expression levels, drug intolerance) and not yet well characterized (e.g. primary, BCR-ABL1 kinase-independent resistance) mechanisms. Furthermore, clinical studies involving different approved ABL1 TKIs have identified a consensus association between rapid achievement of deep molecular response, such as a major molecular response or greater, and improved overall and progression-free survival. To that end, opportunities to improve outcomes further in CML will need to focus on strategies that more extensively deplete the resistant and/or persistent leukemic cells through combined targeting approaches.

Compounds that bind and target the BCR-ABL1 protein for degradation by employing ATP-competitive ligands as recruiting elements are frequently unable to induce complete degradation of BCR-ABL1 and likely suffered from issues of selectivity similar to those observed with other orthosteric kinase ligand-based degraders. Compounds of Formula (I) do not bind to the ATP-binding pocket of BCR-ABL1 as can be used as either mono- or combination-therapies as described herein to avoid the drug resistance frequently seen in clinical settings. In one example, Compound 10, which links a BCR-ABL1 allosteric site binding scaffold to the VHL ligand, achieved dramatic degradation of BCR-ABL1 protein in cell lines in a time- and concentration-dependent manner. Prior attempts to develop VHL-recruiting BCR-ABL1 PROTACs that employed active site recruiting elements were unsuccessful. Without being bound by theory, it is believed that favorable protein-protein interactions are crucial for successful PROTAC development. Without being bound by theory, the BCR-ABL1/PROTAC/CRBN trimer is functional at the ATP binding site while the BCR-ABL1/PROTAC/VHL trimer is functional only at the allosteric site.

Previous comparisons between PROTACs and inactive diastereomers have highlighted advantages of degradation versus inhibition alone as well as providing evidence for their use against hematological malignancies. In the present study, modestly greater inhibition of cell proliferation was observed for the degrader compared to the non-degrading control, suggesting that, consistent with previous studies using critical tyrosine-mutated and kinase dead BCR-ABL1 mutant constructs, much of the oncogenic signaling in native CML cells is critically dependent upon the tyrosine kinase activity of BCR-ABL1.

Utilizing an allosteric PROTAC with an orthosteric inhibitor can result in synergistic inhibitory effects as described herein. For example, when combined with imatinib, Compound 10 demonstrated greater inhibition of Ba/F3 BCR-ABL1 cells compared to the non-degrader control. Combining Compound 10 with low concentrations of the third-generation ATP-site ABL1 TKI ponatinib also showed increased degradation compared Compound 10 alone. Compound 10 also demonstrated varying degrees of retained sensitivity to imatinib-resistant BCR-ABL1 kinase domain mutants, suggesting that even partially limited target engagement is sufficient to induce degradation, which highlights the power of this occupancy driven pharmacology model.

While clinically approved ABL1 TKIs have highlighted the importance of the importance of downstream signaling activated by the tyrosine kinase activity of BCR-ABL1, selective BCR-ABL 1-targeted PROTACs are uniquely able to facilitate convenient interrogation of non-kinase-dependent scaffolding roles of this oncogene. Comparing Compound 10 and the inactive diastereomer, it was found that while both compounds inhibited BCR-ABL1 kinase activity, degradation of BCR-ABL1 uniquely resulted in decreased pSHC, pSHP2, and pGAB2 levels. Notably, this behavior phenocopies the mutation of a key autophosphorylation site (Y177) on the BCR portion of the fusion protein. Whilst Y177 is normally auto-phosphorylated by the kinase domain of BCR-ABL1, under serum-stimulated conditions another kinase (likely HCK38) appears to phosphorylate Y177 on BCR-ABL1, allowing it to continue to act as a scaffold.

Degradation of BCR-ABL1 prevents this scaffolding function, thus ameliorating signaling via GAB2, SHP-2 and SHC. This scaffolding role also partially explains the enhanced antiproliferative activity of Compound 10 compared to Compound 14 in both models systems (K562, Ba/F3) and primary patient samples. Destruction of the protein, it seems, rather than inhibition of its kinase domain, has a more potent and sustained inhibition of downstream signaling, at least in part, due to the loss of the Y177 docking domain.

It has been shown previously that while ABL1 TKIs such as imatinib effectively inhibit BCR-ABL1 kinase activity in CML stem and progenitor cells, the stem population is preferentially less susceptible to apoptosis induction. Compounds of Formula (I), such as Compound 10 effectively inhibited BCR-ABL1 kinase activity and degraded BCR-ABL1 protein in the context of isolated CML stem (CD34+CD38−) and progenitor (CD34+CD38+) cells. While significant induction of apoptosis to Compound 10 was observed in the progenitors, only a minor induction of apoptosis was observed in CML stem cells, adding to the growing body of evidence that CML stem cells are not dependent on BCR-ABL1 kinase activity for survival. Additionally, the lack of differential between Compound 10 and Compound 14 in this population suggests that they are not dependent on potential non-kinase scaffolding roles associated with the presence of the BCR-ABL1 protein either. While it has been postulated that the scaffolding roles of BCR-ABL1, including that of Y177, may be responsible for the survival of BCR-ABL1-positive LSCs, the results support a more limited dependence upon BCR-ABL1 in general in this population. Previous studies have also suggested the persistence of CML stem cells on treatment is likely attributable to several potential mechanisms, including quiescence, alternative survival signaling pathways, and protective signals from the bone marrow microenvironment niche.

### IAP E3 Ubiquitin Ligase Binding Moieties

### AVPI Tetrapeptide Fragments

In any of the compounds described herein, the ULM can comprise an alanine-valine-proline-isoleucine (AVPI) tetrapeptide fragment or an unnatural mimetic thereof. In certain embodiments, the ULM is selected from the group consisting of chemical structures represented by Formulas (IAP-I), (IAP-II), (IAP-III), (IAP-IV), and (IAP-V):

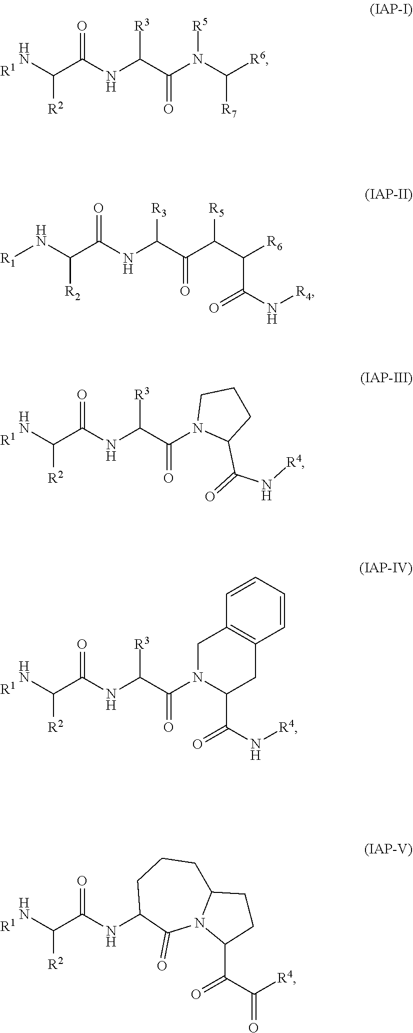

wherein:

each occurrence of R¹ in compounds of Formulas Formulas (AP-I), (IAP-IIV), and (IAP-V) is independently selected from the group consisting of H and alkyl;

each occurrence of R² in compounds of Formulas (IAP-I), (IAP-II), (AP-III), (AP-IV), and (IAP-V) is independently selected from the group consisting of H and alkyl;

each occurrence of R³ in compounds of Formulas (IAP-I), (IAP-II), (IAP-III), (IAP-IV), and (IAP-V) is independently selected from the group consisting of H, alkyl, cycloalkyl and heterocycloalkyl;

each occurrence of R⁵ and R⁶ in compounds of Formulas (IAP-I), (IAP-II), (IAP-III), (IAP-IV), and (IAP-V) are independently selected from the group consisting of H, alkyl, cycloalkyl, and heterocycloalkyl; or

R⁵ and R⁶ taken together independently in compounds of Formulas (IAP-I), (IAP-II), (IAP-III), (IAP-IV), and (IAP-V) form a pyrrolidine or a piperidine ring further optionally fused to 1-2 cycloalkyl, heterocycloalkyl, aryl, or heteroaryl rings, each of which is optionally fused to an additional cycloalkyl, heterocycloalkyl, aryl, or heteroaryl ring;

each occurrence of R³ and R⁵ in compounds of Formulas (IAP-I), (IAP-II), (IAP-III), (IAP-IV), and (IAP-V) are independently taken together can form a 5-8-membered ring and further optionally fused to 1-2 cycloalkyl, heterocycloalkyl, aryl, or heteroaryl rings;

each occurrence of R⁷ in compounds of Formulas (IAP-I), (IAP-II), (IAP-III), (IAP-IV), and (IAP-V) is independently selected from the group consisting of cycloalkyl, cycloalkylalkyl, heterocycloalkyl, aryl, aryl-C(O)—R⁴, arylalkyl, heteroaryl, heteroaryl-C(O)—R⁴, heteroaryl-R⁴, heteroaryl-naphthalene, heteroarylalkyl, or —C(O)NH—R⁴, each of which can be optionally substituted with 1-3 substituents selected from halogen, alkyl, haloalkyl, hydroxyl, alkoxy, cyano, (hetero)cycloalkyl, (hetero)aryl, —C(O)NH—R⁴, or —C(O)—R⁴; and

R⁴ for Formulas (IAP-I), (IAP-II), (IAP-III), (IAP-IV), and (IAP-V) is selected from alkyl, cycloalkyl, heterocycloalkyl, cycloalkylalkyl, heterocycloalkylalkyl, aryl, arylalkyl, heteroaryl, heteroarylalkyl, further optionally substituted.

In various embodiments, P1, P2, P3, and P4 in the compound of Formula (IAP-II) correspond to the A, V, P, and I residues, respectively, of the AVPI tetrapeptide fragment or an unnatural mimetic thereof. Similarly, each compound of Formulas (IAP-I) and (IAP-III) through (IAP-V) have portions corresponding to the A, V, P, and I residues of the AVPI tetrapeptide fragment or an unnatural mimetic thereof.

In various embodiments, the ULM moiety can have the structure of Formula (IAP-VI), as described in WO Pub. No. 2008/014236, or an unnatural mimetic thereof:

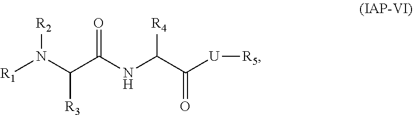

wherein:

each occurrence of R¹ in the compound of Formula (IAP-VI) is independently selected from the group consisting of H, C₁-C₄-alkyl, C₁-C₄-alkenyl, C₁-C₄-alkynyl, and C₃-C₁₀-cycloalkyl, each of which can be optionally substituted with 1-3 substituents selected from the group consisting of halogen, alkyl, haloalkyl, hydroxyl, alkoxy, cyano, heterocycloalkyl, and heteroaryl;

each occurrence of R₂ in the compound of Formula (IAP-VI) is independently selected from the group consisting of H, C₁-C₄-alkyl, C₁-C₄-alkenyl, C₁-C₄-alkynyl, and C₃-C₁₀-cycloalkyl, each of which can be optionally substituted with 1-3 substituents selected from the group consisting of halogen, alkyl, haloalkyl, hydroxyl, alkoxy, cyano, heterocycloalkyl, and heteroaryl;

each occurrence of R₃ in the compound of Formula (IAP-VI) is independently selected from the group consisting of H, —CF₃, —C₂H₅, C₁-C₄-alkyl, C₁-C₄-alkenyl, C₁-C₄-alkynyl, —CH₂—Z, and any R₂ and R₃ together form a heterocyclic ring, each of which can be optionally substituted with 1-3 substituents selected from the group consisting of halogen, alkyl, haloalkyl, hydroxyl, alkoxy, cyano, heterocycloalkyl, and heteroaryl;

each occurrence of Z in the compound of Formula (IAP-VI) is independently selected from the group consisting of H, —OH, F, Cl, —CH₃, —CF₃, —CH₂Cl, —CH₂F, and —CH₂OH; each occurrence of R₄ in the compound of Formula (IAP-VI) is independently selected from the group consisting of C₁-C₁₆ straight or branched alkyl, C₁-C₁₆-alkenyl, C₁-C₁₆-alkynyl, C₃-C₁₀-cycloalkyl, —(CH₂)₀₋₆—Z₁, —(CH₂)₀₋₆-aryl, and —(CH₂)₀₋₆-het, each of which can be optionally substituted with 1-3 substituents selected from the group consisting of halogen, alkyl, haloalkyl, hydroxyl, alkoxy, cyano, heterocycloalkyl, and heteroaryl;

each occurrence of R₅ in the compound of Formula (IAP-VI) is independently selected from the group consisting of H, C₁₋₁₀-alkyl, aryl, phenyl, C₃₋₇-cycloalkyl, —(CH₂)₁₋₆—C₃₋₇-cycloalkyl, —C₁₋₁₀-alkyl-aryl, —(CH₂)₀₋₆—C₃₋₇-cycloalkyl-(CH₂)₀₋₆-phenyl, —(CH₂)₀₋₄—CH[(CH₂)₁₋₄-phenyl]₂, indanyl, —C(═O)—C₁₋₁₀-alkyl, —C(═O)—(CH₂)₁₋₆—C₃₋₇-cycloalkyl, —C(═O)—(CH₂)₀₋₆-phenyl, —(CH₂)₀₋₆—C(═O)-phenyl, —(CH₂)₀₋₆-het, —C(═O)—(CH₂)₁₋₆-het, and a residue of an amino acid, each of which can be optionally substituted with 1-3 substituents selected from the group consisting of halogen, alkyl, haloalkyl, hydroxyl, alkoxy, cyano, heterocycloalkyl, and heteroaryl;

each occurrence of Z₁ in the compound of Formula (IAP-VI) is independently selected from the group consisting of —N(R₁₀)—C(═O)—C₁₋₁₀-alkyl, —N(R₁₀)—C(═O)—(CH₂)₀₋₆—C₃₋₇-cycloalkyl, —N(R₁₀)—C(═O)—(CH₂)₀₋₆-phenyl, —N(R₁₀)—C(═O)(CH₂)₁₋₆-het, —C(═O)—N(R₁₁)(R₁₂), —C(═O)—O—C₁₋₁₀-alkyl, —C(═O)—O—(CH₂)₁₋₆—C₃₋₇-cycloalkyl, —C(═O)—O—(CH₂)₀₋₆-phenyl, —C(═O)—O—(CH₂)₁₋₆-het, —O—C(═O)—C₁₋₁₀-alkyl, —O—C(═O)—(CH₂)₁₋₆—C₃₋₇-cycloalkyl, —O—C(═O)—(CH₂)₀₋₆-phenyl, and —O—C(═O)—(CH₂)₁₋₆-het, each of which can be optionally substituted with 1-3 substituents selected from the group consisting of halogen, alkyl, haloalkyl, hydroxyl, alkoxy, cyano, heterocycloalkyl, and heteroaryl;

each occurrence of het in the compound of Formula (IAP-VI) is independently selected from the group consisting of a 5-7 member heterocyclic ring containing 1-4 N, O, or S heteroatoms, and an 8-12 member fused ring system including at least one 5-7 member heterocyclic ring containing 1-3 N, O, or S heteroatoms, which heterocyclic ring or fused ring system is optionally substituted with 1-3 substituents selected from the group consisting of halogen, alkyl, haloalkyl, hydroxyl, alkoxy, cyano, heterocycloalkyl, and heteroaryl on a carbon or nitrogen atom in the heterocyclic ring or fused ring system;

each occurrence of R₁₀ in the compound of Formula (IAP-VI) is selected from the group consisting of H, —CH₃, —CF₃, —CH₂OH, and —CH₂Cl;

each occurrence of R₁₁ and R₁₂ in the compound of Formula (IAP-VI) is independently selected from the group consisting of H, C₁₋₄-alkyl, C₃₋₇-cycloalkyl, —(CH₂)₁₋₆—C₃₋₇-cycloakyl, (CH₂)₀₋₆-phenyl, each of which can be optionally substituted with 1-3 substituents selected from the group consisting of halogen, alkyl, haloalkyl, hydroxyl, alkoxy, cyano, heterocycloalkyl, and heteroaryl; or R₁₁ and R₁₂ together with the nitrogen form het, and each occurrence of U in the compound of Formula (IAP-VI) is independently of Formula (IAP-VII):

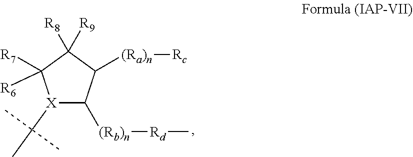

wherein:

each occurrence of n in the compound of Formula (IAP-VII) is independently selected from a whole number from 0 to 5;

each occurrence of X in the compound of Formula (IAP-VII) is independently selected from the group consisting of —CH and N;

each occurrence of Rₐ and R_{b} in the compound of Formula (IAP-VII) is independently selected from the group consisting of an O atom, a S atom, an N atom, and C₀₋₈-alkyl, wherein one or more of the carbon atoms in the C₀₋₈-alkyl is optionally replaced by a heteroatom selected from the group consisting of O, S, and N, and wherein each occurrence of C₀₋₈-alkyl is independently optionally substituted with 1-3 substituents selected from the group consisting of halogen, alkyl, haloalkyl, hydroxyl, alkoxy, cyano, heterocycloalkyl, and heteroaryl;

each occurrence of R_{d} in the compound of Formula (IAP-VII) is independently selected from the group consisting of Rₑ-Q-(R_{f})ₚ(R_{g})_{q}, and Ar₁-D-Ar₂;

each occurrence of Rₑ in the compound of Formula (IAP-VII) is independently selected from the group consisting of H and any R_{c} and R_{d} taken together form a cycloalkyl or het; with the proviso that if R_{c} and R_{d} form a cycloalkyl or het, R₅ is attached to the formed ring at a C or N atom;

each occurrence of p and q in the compound of Formula (IAP-VII) is independently 0 or 1;

each occurrence of Rₑ in the compound of Formula (IAP-VII) is selected from the group consisting of C₁₋₈-alkyl and alkylidene, each of which is optionally substituted with 1-3 substituents selected from the group consisting of halogen, alkyl, haloalkyl, hydroxyl, alkoxy, cyano, heterocycloalkyl, and heteroaryl;

each occurrence of Q is independently selected from the group consisting of N, O, S, S(═O), and S(═O)₂;

each occurrence of Ar₁ and Ar₂ in the compound of Formula (IAP-VII) is independently selected from the group consisting of aryl and het, each of which is optionally substituted with 1-3 substituents selected from the group consisting of halogen, alkyl, haloalkyl, hydroxyl, alkoxy, cyano, heterocycloalkyl, and heteroaryl;

each occurrence of R_{f} and R_{g} in the compound of Formula (IAP-VII) is independently selected from the group consisting of H, —C₁₋₁₀-alkyl, C₁₋₁₀-alkylaryl, —OH, —O—C₁₋₁₀-alkyl, —(CH₂)₀₋₆—C₃₋₇-cycloalkyl, —O—(CH₂)₀₋₆-aryl, phenyl, aryl, phenyl -phenyl, —(CH₂)₁₋₆-het, —O—(CH₂)₁₋₆-het, —OR₁₃, —C(═O)—R₁₃, —C(═O)—N(R₁₃)(R₁₄), —N(R₁₃)(R₁₄), —S—R₁₃, —S(═O)—R₁₃, —S(═O)₂—R₁₃, —S(═O)₂—NR₁₃R₁₄, —NR₁₃—S(═O)₂—R₁₄, —S—C₁₋₁₀-alkyl, aryl-C₁₋₄-alkyl, or het-C₁₋₄-alkyl, —SO₂—C₁₋₂-alkyl, —SO₂—C₁₋₂-alkylphenyl, —O—C₁₋₄-alkyl, and any R_{g} and R_{f} together form a ring selected from het or aryl, each of which is optionally substituted with 1-3 substituents selected from the group consisting of halogen, alkyl, haloalkyl, hydroxyl, alkoxy, cyano, heterocycloalkyl, and heteroaryl;

each occurrence of D in the compound of Formula (IAP-VII) is independently selected from the group consisting of —CO—, —C(═O)—C₁₋₇-alkylene, —C(═O)—C₁₋₇-arylene, —CF₂—, —O—, —S(═O)ᵣ where r is a whole number from 0-2, 1,3-dioxalane, C₁₋₇-alkyl-OH, and N(Rₕ), each of which is optionally substituted with one or more of halogen, OH, —O—C₁₋₆-alkyl, —S—C₁₋₆-alkyl, or —CF₃;

each occurrence of Rₕ in the compound of Formula (IAP-VII) is independently selected from the group consisting of H, unsubstituted or substituted C₁₋₇-alkyl, aryl, unsubstituted or substituted —O—(C₁₋₇-cycloalkyl), —C(═O)—C₁₋₁₀-alkyl, —C(═O)—C₀₋₁₀-alkyl-aryl, —C—O—C₀₋₁₀-alkyl, —C—O—C₀₋₁₀-alkyl-aryl, —SO₂—C₁₋₁₀-alkyl, and —SO₂—(C₀₋₁₀-alkylaryl); each occurrence of R₆, R₇, R₈, and R₉ in the compound of Formula (IAP-VII) is independently selected from the group consisting of H, —C₁₋₁₀-alkyl, —C₁₋₁₀-alkoxy, aryl-C₁₋₁₀-alkoxy, —OH, —O—C₁₋₁₀-alkyl, —(CH₂)₀₋₆—C₃₋₇-cycloalkyl, —O—(CH₂)₀₋₆-aryl, phenyl, —(CH₂)₁₋₆-het, —O—(CH₂)₁₋₆-het, —OR₁₃, —C(═O)—R₁₃, —C(═O)—N(R₁₃)(R₁₄), —N(R₁₃)(R₁₄), —S—R₁₃, —S(═O)—R₁₃, —S(═O)₂—R₁₃, —S(═O)₂—NR₁₃R₁₄, and —NR₁₃—S(═O)₂—R₁₄, each of which is optionally substituted with 1-3 substituents selected from the group consisting of halogen, alkyl, haloalkyl, hydroxyl, alkoxy, cyano, heterocycloalkyl, and heteroaryl; or any occurrence of R₆, R₇, R₈, and R₉ together optionally form a ring system;

each occurrence of R₁₃ and R₁₄ in the compound of Formula (IAP-VII) is independently selected from the group consisting of H, C₁₋₁₀-alkyl, —(CH₂)₀₋₆—C₃₋₇-cycloalkyl, —(CH₂)₀₋₆—(CH)₀₋₁-(aryl)₁₋₂, —C(═O)—C₁₋₁₀-alkyl, —C(═O)—(CH₂)₁₋₆—C₃₋₇-cycloalkyl, —C(═O)—O—(CH₂)₀₋₆-aryl, —C(═O)—(CH₂)₀₋₆—O-fluorenyl, —C(═O)—NH—(CH₂)₀₋₆-aryl, —C(═O)—(CH₂)₀₋₆-aryl, —C(═O)—(CH₂)₀₋₆-het, —C(═S)—C₁₋₁₀-alkyl, —C(═S)—(CH₂)₁₋₆—C₃₋₇-cycloalkyl, —C(═S)—O—(CH₂)₀₋₆-aryl, —C(═S)—(CH₂)₀₋₆—O-fluorenyl, —C(═S)—NH—(CH₂)₀₋₆-aryl, —C(═S)—(CH₂)₀₋₆-aryl, and —C(═S)—(CH₂)₁₋₆-het, each of which is optionally substituted with one or more substituents selected from the group consisting of C₁₋₁₀-alkyl, halogen, OH, —O—C₁₋₆-alkyl, —S—C₁₋₆-alkyl, —CF₃, halogen, hydroxyl, C₁₋₄-alkyl, C₁₋₄-alkoxy, nitro, —CN, —O—C(═O)—C₁₋₄-alkyl, and —C(═O)—O—C₁₋₄-aryl; or any R₁₃ and R₁₄ can join together with a nitrogen atom to form a het.

In various embodiments, the ULM can have the structure of Formula (IAP-VIII), as described in ACS Chem. Biol., 557-566, 4 (7) (2009), or an unnatural mimetic thereof:

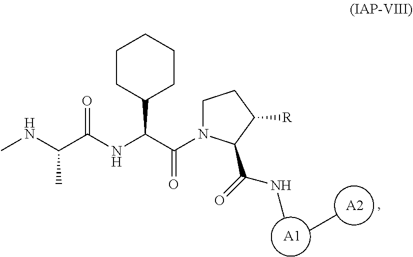

wherein each occurrence of of A1 and A2 in the compound of Formula (IAP-VIII) is independently selected from the group consisting of a monocyclic ring, a fused ring, an aryl, and a heteroaryl, each of which is optionally substituted with 1-3 substituents selected from the group consisting of halogen, alkyl, haloalkyl, hydroxyl, alkoxy, cyano, heterocycloalkyl, and heteroaryl; and each occurrence of R in the compound of Formula (IAP-VIII) is independently H or Me.

In a certain embodiment, the linker group L is attached to A1 of Formula (IAP-VIII). In another embodiment, the linker group L is attached to A2 of Formula (IAP-VIII).

In various embodiments, the ULM is selected from the group consisting of

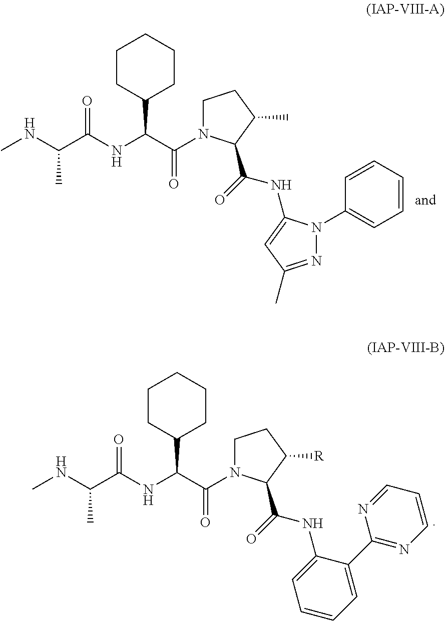

In various embodiments, the ULM can have the structure of Formula (IAP-IX), as described in Drug Discov. Today, 15 (5-6), 210-9 (2010), or an unnatural mimetic thereof:

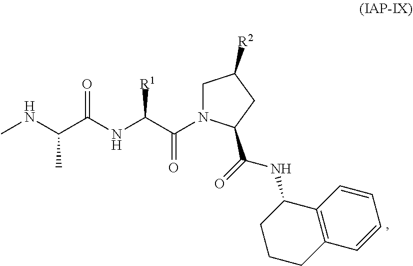

wherein each occurrence R¹ in the compound of Formula (IAP-IX) is independently selected from the group consisting of alkyl, cycloalkyl, heterocycloalkyl. In various embodiments, R¹ in the compound of Formula (IAP-IX) is independently selected from the group consisting of isopropyl, tert-butyl, cyclohexyl, and tetrahydropyranyl. In various embodiments, each occurrence of R² in the compound of Formula (IAP-IX) is selected from —OPh or H.

In various embodiments, the ULM can have the structure of Formula (X), as described in Drug Discov. Today, 15 (5-6), 210-9 (2010), or an unnatural mimetic thereof:

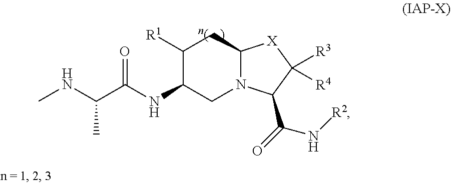

wherein:

each occurrence of R¹ in the compound of Formula (IAP-X) is independently selected from the group consisting of H, —CH₂OH, —CH₂CH₂OH, —CH₂NH₂, and —CH₂CH₂NH₂;

each occurrence of X in the compound of Formula (IAP-X) is independently selected from S and CH₂;

each occurrence of R² in the compound of Formula (IAP-X) is independently selected from the group consisting of:

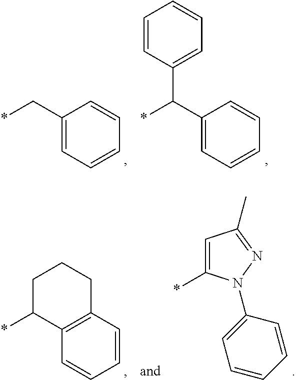

each occurrence of R³ and R⁴ in the compound of Formula (IAP-X) is independently selected from H and Me.

In various embodiments, the ULM can have the structure of Formula (IAP-XI), as described in Drug Discov. Today, 15 (5-6), 210-9 (2010), or an unnatural mimetic thereof:

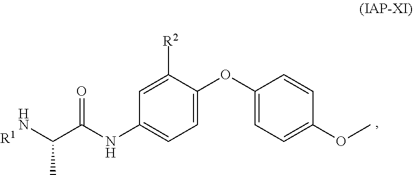

wherein each occurrence of R¹ in the compound of Formula (IAP-XI) is is independently selected from H and Me, and each occurrence of R² in the compound of Formula (IAP-XI) is independently selected from H and

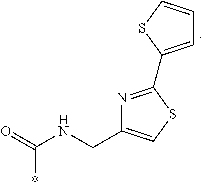 [SMILES: *C(=O)NCc1csc(-c2cccs2)n1]

In various embodiments, the ULM can have the structure of Formula (IAP-XII), as described in Drug Discov. Today, 15 (5-6), 210-9 (2010), or an unnatural mimetic thereof:

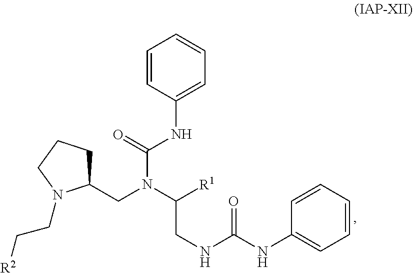

wherein:
each occurrence of R¹ in the compound of Formula (IAP-XII) is independently selected from the group consisting of:

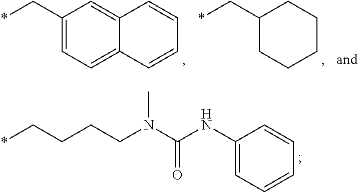

and each occurrence of R² in the compound of Formula (IAP-XII) is independently selected from the group consisting of:

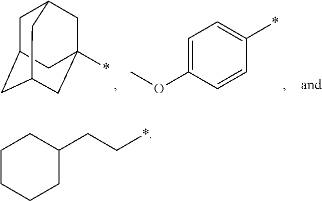

In various embodiments, the ULM moiety is selected from the group consisting of:

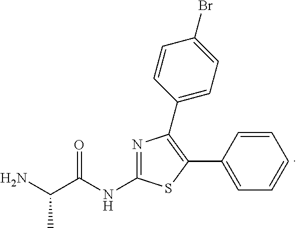 [SMILES: C[C@H](N)C(=O)Nc1nc(-c2ccc(Br)cc2)c(-c2ccccc2)s1]

In various embodiments, the ULM can have the structure of Formula (IAP-XIII) as described in Expert Opin. Ther. Pat., 20 (2), 251-67 (2010), or an unnatural mimetic thereof:

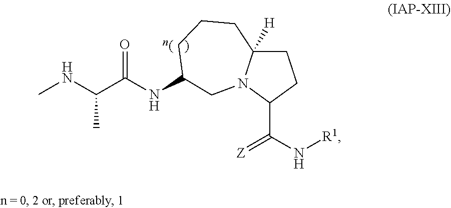

wherein:

at each occurrence, Z in the compound of Formula (IAP-XIII) is independently absent or O;

each occurrence of R¹ in the compound of Formula (IAP-XIII) is independently selected from the group consisting of:

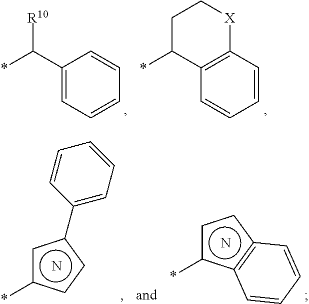

each occurrence of R¹⁰ in the compound of Formula (IAP-XIII) is selected from the group consisting of H, alkyl, and aryl;

each occurrence of X in the compound of Formula (IAP-XIII) is selected from CH₂ and O; and

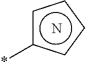

is a nitrogen-containing heteroaryl containing from 1-3 nitrogen atoms in the ring.

In various embodiments, the ULM can have the structure of Formula (IAP-XIV) as described in Expert Opin. Ther. Pat., 20 (2), 251-67 (2010), or an unnatural mimetic thereof:

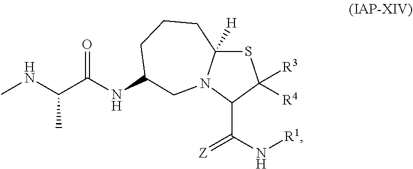

wherein:

at each occurrence, Z in the compound of Formula (IAP-XIV) is independently absent or 0;

each occurrence of R¹ in the compound of Formula (IAP-XIV) is independently selected from the group consisting of:

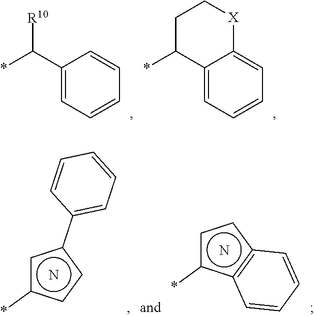

each occurrence of R³ and R⁴ in the compound of Formula (IAP-XIV) is independently selected from H and Me;

each occurrence of R¹⁰ in the compound of Formula (IAP-XIV) is selected from the group consisting of H, alkyl, and aryl;

each occurrence of X in the compound of Formula (IAP-XIV) is selected from the group consisting of CH₂ and O; and

each

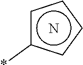

in

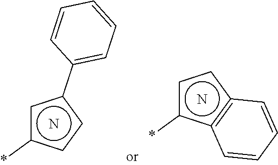

or is a nitrogen-containing heteroaryl containing from 1-3 nitrogen atoms in the ring.

In various embodiments, the ULM is selected from the group consisting of:

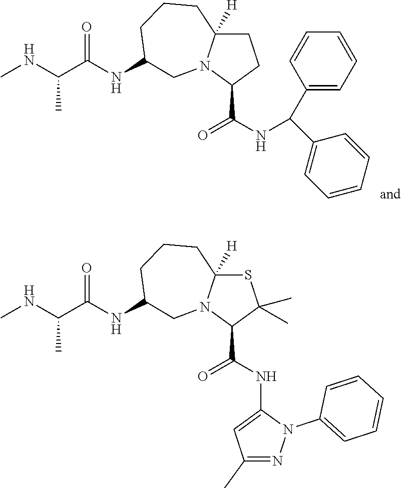

In various, the ULM can have the structure of Formula (IAP-XV), as described in WO Pub. No. 2008/128171, or an unnatural mimetic thereof:

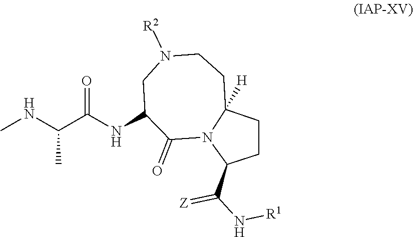

wherein:

at each occurrence Z in the compound of Formula (IAP-XV) is absent or O;

each occurrence of R¹ in the compound of Formula (IAP-XV) is independently selected from the group consisting of:

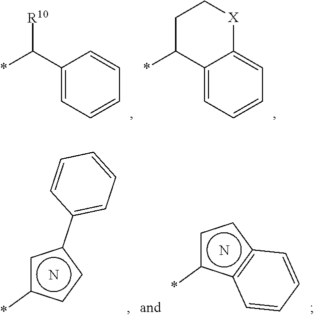

each occurrence of R² in the compound of Formula (IAP-XV) is independently selected from the group consisting of H, alkyl, and acyl;

each occurrence of R¹⁰ in the compound of Formula (IAP-XV) is selected from the group consisting of H, alkyl, and aryl;

each occurrence of X in the compound of Formula (IAP-XV) is selected from CH₂ and O; and

each

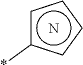

in

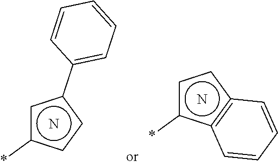

is a nitrogen-containing heteroaryl containing from 1-3 nitrogen atoms in the ring.

In a particular embodiment, the ULM has the structure:

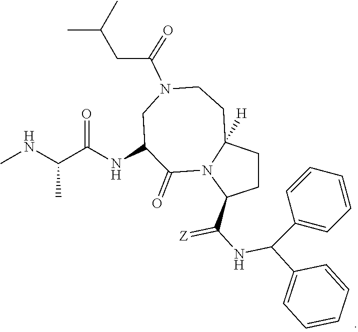

In various embodiments, the ULM can have the structure of Formula (IAP-XVI), as described in WO Pub. No. 2006/069063, or an unnatural mimetic thereof:

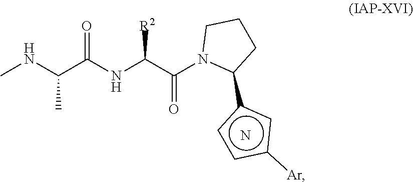

wherein:

each occurrence of R² in the compound of Formula (IAP-XVI) is independently selected from the group consisting of alkyl, cycloalkyl, heterocycloalkyl, isopropyl, tert-butyl, cyclohexyl, and tetrahydropyranyl. In various embodiments, R² in the compound of Formula (IAP-XVI) is independently selected from the group consisting of isopropyl, tert-butyl, and cyclohexyl.

each occurrence of

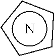

in the compound of Formula (IAP-XVI) is independently a 5- or 6-membered nitrogen-containing heteroaryl. In various embodiments,

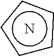

is a 5-membered nitrogen-containing heteroaryl. In various embodiments,

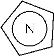

is thiazole. In various embodiments, each occurrence of Ar in the compound of Formula (IAP-XVI) is independently an aryl or a heteroaryl.

In various embodiments, the ULM can have the structure of Formula (IAP-XVII), as described in Bioorg. Med. Chem. Lett., 20(7), 2229-33 (2010), or an unnatural mimetic thereof:

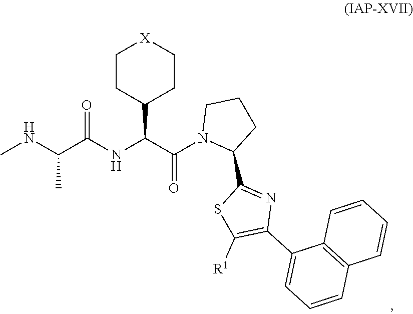

wherein each occurrence of R¹ in the compound of Formula (IAP-XVII) is independently selected from the group consisting of halogen, cyano, —C≡CH, —C≡CCH₃, —C≡CCH₂OCH₃, and —C≡CCH₂OH; and

each occurrence of X in the compound of Formula (IAP-XVII) is independently selected from the group consisting of O and CH₂.

In various embodiments, the ULM can have the structure of Formula (IAP-XVIII), as described in Bioorg. Med. Chem. Lett., 20(7), 2229-33 (2010), or an unnatural mimetic thereof:

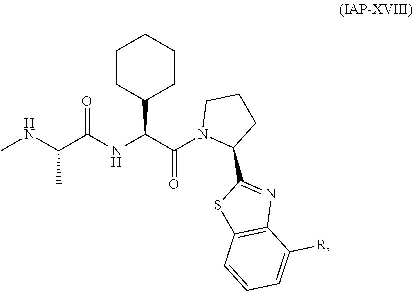

wherein each occurrence of R in the compound of Formula (IAP-XVIII) is independently selected from the group consisting of alkyl, aryl, heteroaryl, arylalkyl, heteroarylalkyl, and halogen (in variable substitution position).

In various embodiments, the ULM can have the structure of Formula (XIX) as described in Bioorg. Med. Chem. Lett., 20(7), 2229-33 (2010), or an unnatural mimetic thereof:

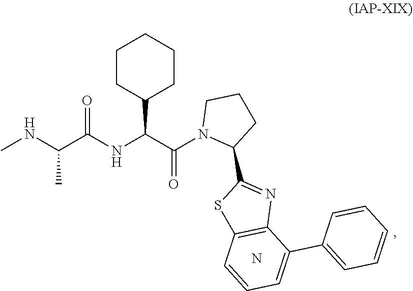

wherein

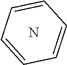

is a 6-member nitrogen heteroaryl.

In a certain embodiment, the ULM of the composition is selected from the group consisting of:

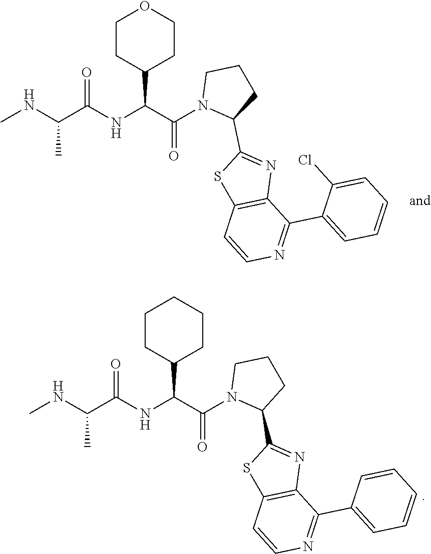 [SMILES: CN[C@@H](C)C(=O)N[C@H](C(=O)N1CCC[C@H]1c1nc2c(-c3ccccc3Cl)nccc2s1)C1CCOCC1 ; CN[C@@H](C)C(=O)N[C@H](C(=O)N1CCC[C@H]1c1nc2c(-c3ccccc3)nccc2s1)C1CCCCC1]

In certain embodiments, the ULM of the composition is selected from the group consisting of:

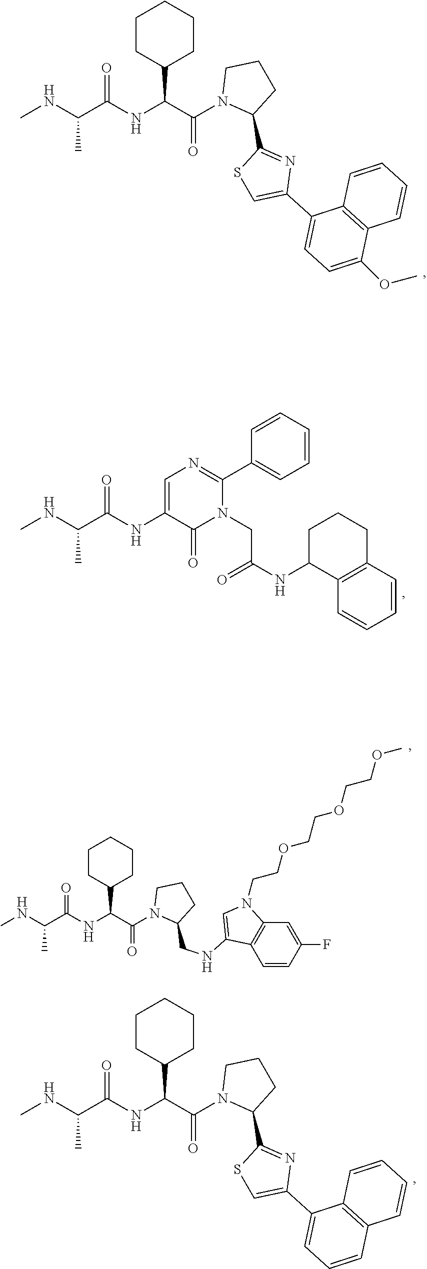

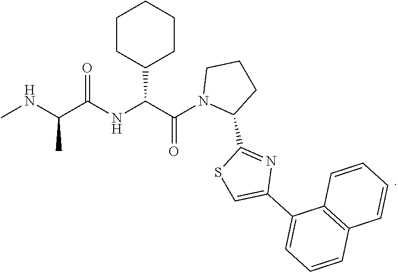 [SMILES: CN[C@H](C)C(=O)N[C@@H](C(=O)N1CCC[C@@H]1c1nc(-c2cccc3ccccc23)cs1)C1CCCCC1]

In various embodiments, the ULM can have the structure of Formula (IAP-XX), as described in WO Pub. No. 2007/101347, or an unnatural mimetic thereof:

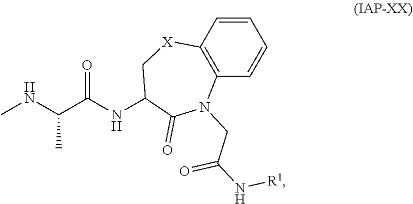

wherein each occurrence of X in the compound of Formula (IAP-XX) is independently selected from the group consisting of CH₂, O, NH, and S.

In certain embodiments, the ULM can have the structure of Formula (IAP-XXI), as described in U.S. Pat. Nos. 7,345,081 and 7,419,975, or an unnatural mimetic thereof:

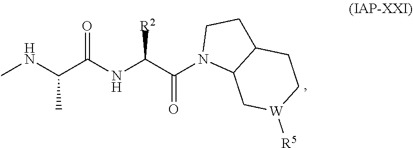

wherein:

each occurrence of R² in the compound of Formula (IAP-XXI) is independently selected from the group consisting of tert-butyl, iso-propyl, and cyclohexyl;

each occurrence of R⁵ in the compound of Formula (IAP-XXI) is independently selected from

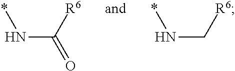

each occurrence of W in the compound of Formula (IAP-XXI) is independently selected from CH and N; and

each occurrence of R⁶ in the compound of Formula (IAP-XXI) is independently selected from the group consisting of a mono-cyclic fused aryl, a bicyclic fused aryl, and heteroaryl.

In certain embodiments, the ULM of the compound is selected from the group consisting of:

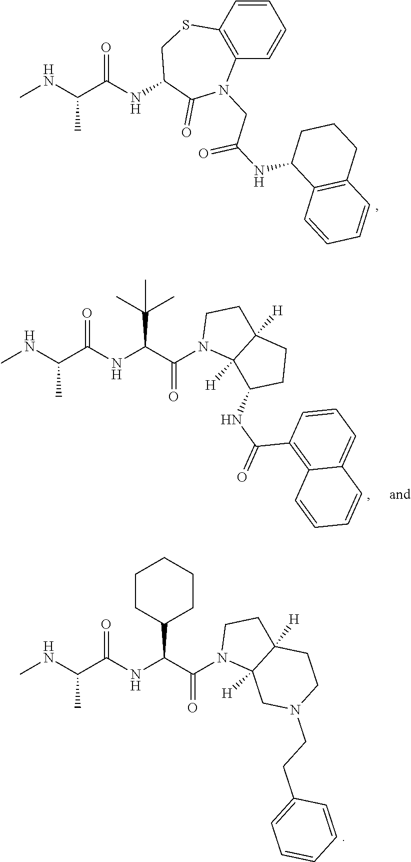 [SMILES: CN[C@@H](C)C(=O)N[C@@H]1CSc2ccccc2N(CC(=O)N[C@@H]2CCCc3ccccc32)C1=O ; CN[C@@H](C)C(=O)N[C@H](C(=O)N1CC[C@H]2CC[C@H](NC(=O)c3cccc4ccccc34)[C@H]21)C(C)(C)C ; CN[C@@H](C)C(=O)N[C@H](C(=O)N1CC[C@H]2CCN(CCc3ccccc3)C[C@H]21)C1CCCCC1]

In various embodiments, the ULM can have the structure of Formula (IAP-XXII), (IAP-XXIII), or (IAP-XXIV), as described in J. Med. Chem. 58(3), 1556-62 (2015), or an unnatural mimetic thereof, and the chemical linker to linker group L as shown:

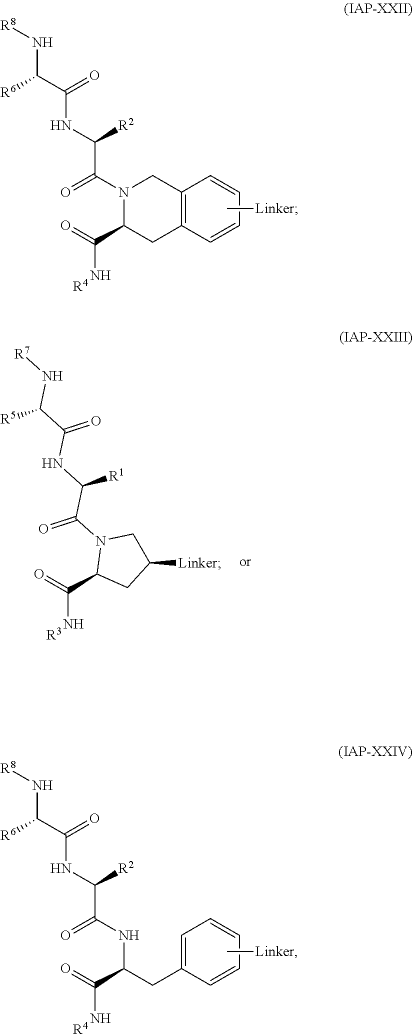

wherein:

each occurrence of R¹ and R² in the compounds of Formula (IAP-XXII), (IAP-XXIII) or (IAP-XXIV) is independently selected from the group consisting of alkyl, cycloalkyl, cycloalkylalkyl, heterocyclyl, arylalkyl, and aryl, each of which is optionally substituted;

or alternatively, each occurrence of R¹ and R² in the compounds of Formula (IAP-XXII), (IAP-XXIII) or (IAP-XXIV) is independently an optionally substituted thioalkyl, wherein the substituents attached to the S atom of the thioalkyl are selected from the group consisting of alkyl, branched alkyl, heterocyclyl, —(CH₂)ᵥCOR²⁰, —CH₂CHR²¹COR²², and —CH₂R²³, each of which is optionally substituted;

at each occurrence in the compounds of Formula (IAP-XXII), (IAP-XXIII) or (IAP-XXIV) v is independently an integer from 1-3;

each occurrence of R²⁰ and R²² in the compounds of Formula (IAP-XXII), (IAP-XXIII) or (IAP-XXIV) is independently selected from the group consisting of OH, NR²⁴R²⁵, and OR²⁶;

each occurrence of R²¹ in the compounds of Formula (IAP-XXII), (IAP-XXIII) or (IAP-XXIV) is independently the group NR²⁴R²⁵;

each occurrence of R²³ in the compounds of Formula (IAP-XXII), (IAP-XXIII) or (IAP-XXIV) is independently selected from the group consisting of aryl and heterocyclyl, each of which is optionally substituted by one or more of alkyl or halogen;

each occurrence of R²⁴ in the compounds of Formula (IAP-XXII), (IAP-XXIII) or (IAP-XXIV) is independently hydrogen or optionally substituted alkyl;

each occurrence of R²⁵ in the compounds of Formula (IAP-XXII), (IAP-XXIII) or (IAP-XXIV) is independently selected from the group consisting of hydrogen, alkyl, branched alkyl, arylalkyl, heterocyclyl, —CH₂(OCH₂CH₂O)ₘCH₃, and —[CH₂CH₂(CH₂)₆NH]CH₂CH₂(CH₂)NH₂, each of which is optionally substituted, wherein δ is a whole number from 0-2, x is an integer from 1-3, and m is a whole number from 0-2;

each occurrence of R²⁶ in the compounds of Formula (IAP-XXII), (IAP-XXIII) or (IAP-XXIV) is independently alkyl, optionally substituted by one or more of OH, halogen, or NH₂;

at each occurrence in the compounds of Formula (IAP-XXII), (IAP-XXIII) or (IAP-XXIV) m is independently an integer from 1-8;

each occurrence of R³ and R⁴ in the compounds of Formula (IAP-XXII), (IAP-XXIII) or (IAP-XXIV) is independently selected from the group consisting of alkyl, cycloalkyl, aryl, arylalkyl, arylalkoxy, heteroaryl, heterocyclyl, heteroarylalkyl, and heterocycloalkyl, each of which is optionally substituted by one or more of alkyl, halogen, or OH;

each occurrence of R⁵, R⁶, R⁷ and R⁸ in the compounds of Formula (IAP-XXII), (IAP-XXIII) or (IAP-XXIV) is independently selected from the group consisting of hydrogen, alkyl, and cycloalkyl, each of which is optionally substituted.

In any aspect or embodiment described herein, R²⁵ in the compounds of Formula (IAP-XXII), (IAP-XXIII) or (IAP-XXIV) is spermine or spermidine.

In various embodiments, the ULM has the structure according to Formulas (IAP-XXII) through (IAP-XXIV), wherein

each occurrence of R⁷ and R⁸ in the compounds of Formulas (IAP-XXII) through (IAP-XXIV) is independently selected from H or Me;

each occurrence of R⁵ and R⁶ in the compounds of Formulas (IAP-XXII) through (IAP-XXIV) is independently selected from the group consisting of

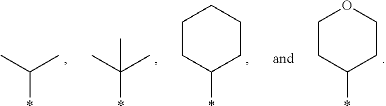

each occurrence of R³ and R⁴ in the compounds Formulas (IAP-XXII) through (IAP-XXIV) is independently selected from the group consisting of:

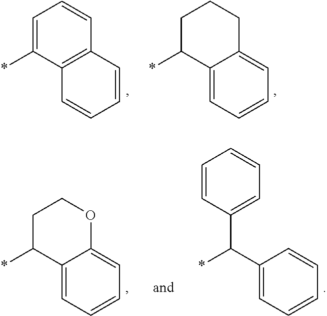

In various embodiments, the ULM can have the structure of Formula (IAP-XXV), (IAP-XXVI), (IAP-XXVII), or (IAP-XXVIII), as described in WO Pub. No. 2014/055461 and Bioorg. Med. Chem. Lett. 24(21), 5022-9 (2014), or an unnatural mimetic thereof, and the chemical linker to linker group L as shown:

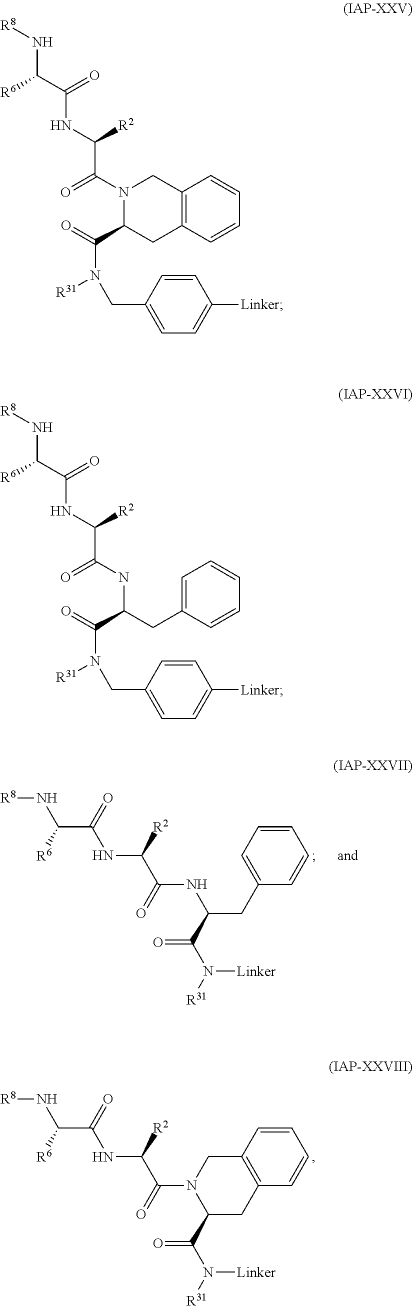

wherein:

each occurrence of R² in the compounds of Formula (IAP-XXV) through (IAPXXVIII) independently selected from the group consisting of alkyl, cycloalkyl, cycloalkylalkyl, heterocyclyl, arylalkyl, and aryl, each of which is optionally substituted;

or alternatively;

each occurrence R¹ and R² in the compounds of Formula (IAP-XXV) through (IAPXXVIII) are independently selected from H, an optionally substituted thioalkyl —CR⁶⁰R⁶¹SR⁷⁰ wherein R⁶⁰ and R⁶¹ are selected from H or methyl, and R⁷⁰ is an optionally substituted alkyl, optionally substituted branched alkyl, optionally substituted heterocyclyl, —(CH₂)ᵥCOR²⁰, —CH₂CHR²¹COR²² or —CH₂R²³;

wherein:

at each occurrence in the compounds of Formula (IAP-XXV) through (IAPXXVIII), v is independently an integer from 1-3;

each occurrence of R²⁰ and R²² in the compounds of Formula (IAP-XXV) through (IAPXXVIII) is independently selected from the group consisting of OH, NR²⁴R²⁵, and OR²⁶;

each occurrence of R²¹ in the compounds of Formula (IAP-XXV) through (IAPXXVIII) is independently the group NR²⁴R²⁵;

each occurrence of R²³ in the compounds of Formula (IAP-XXV) through (IAPXXVIII) is independently selected from the group consisting of aryl and heterocyclyl, each of which is optionally substituted by one or more of alkyl or halogen;

each occurrence of R²⁴ in the compounds of Formula (IAP-XXV) through (IAPXXVIII)) is independently hydrogen or optionally substituted alkyl;

each occurrence of R²⁵ in the compounds of Formula (IAP-XXV) through (IAPXXVIII) is independently selected from the group consisting of hydrogen, alkyl, branched alkyl, arylalkyl, heterocyclyl, —CH₂(OCH₂CH₂O)ₘCH₃, and —[CH₂CH₂(CH₂)NH]_{Ψ}CH₂CH₂(CH₂)ω̅NH₂, each of which is optionally substituted, wherein δ is a whole number from 0-2, xy is an integer from 1-3, and m is a whole number from 0-2;

each occurrence of R²⁶ in the compounds of Formula (IAP-XXV) through (IAPXXVIII) is independently alkyl, optionally substituted by one or more of OH, halogen, or NH₂;

at each occurrence in the compounds of Formula (IAP-XXV) through (IAPXXVIII) m is independently an integer from 1-8;

each occurrence of R⁶ and R⁸ in the compounds of Formula (IAP-XXV) through (IAPXXVIII) is independently selected from the group consisting of hydrogen, optionally substituted alkyl, and optionally substituted cycloalkyl; and

each occurrence of R³¹ in the compounds of Formulas (IAP-XXV) through (IAPXXVIII) is independently selected from the group consisting of alkyl, aryl, arylalkyl, heteroaryl or heteroarylalkyl, each of which is optionally substituted. In various embodiments, R³¹ in the compounds of Formulas (IAP-XXV) through (IAPXXVIII) is independently selected from the group consisting of

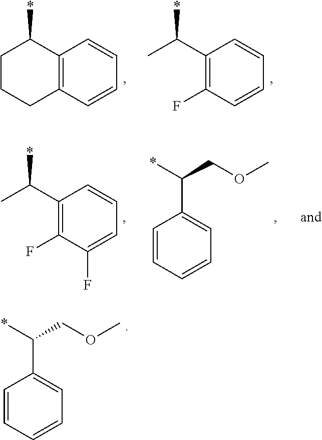

In any aspect or embodiment described herein, R²⁵ in the compounds of Formula (IAP-XXV) through (IAPXXVIII) is spermine or spermidine.

In various embodiments, the ULM can have the structure of Formula (IAP-XXXIX) or (IAP-XL), as described in WO Pub. No. 2013/071039, or an unnatural mimetic thereof:

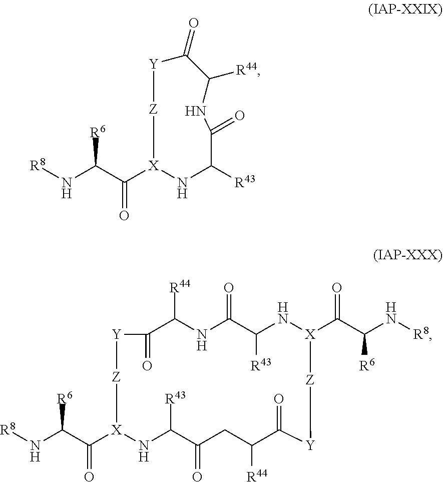

wherein:

each occurrence of R⁴³ and R⁴⁴ of Formulas (IAP-XXIX) and (IAP-XXX) is independently selected from hydrogen, alkyl, aryl, arylalkyl, heteroaryl, heteroarylalkyl, cycloalkyl, cycloalkylalkyl further optionally substituted, and

each occurrence of R⁶ and R⁸ of Formulas (IAP-XXIX) and (IAP-XXX) is independently selected from hydrogen, optionally substituted alkyl or optionally substituted cycloalkyl.

each occurrence of X of Formulas (IAP-XXIX) and (IAP-XXX) is independently selected from:

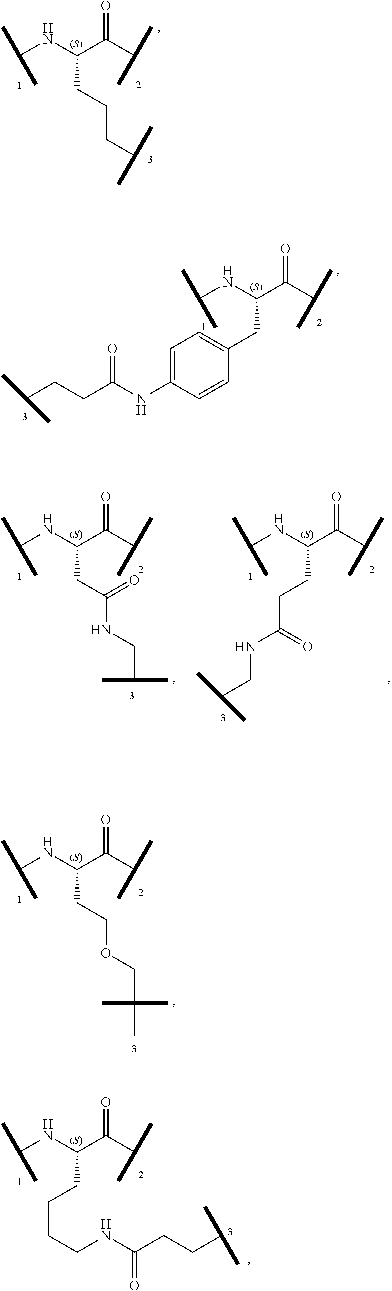

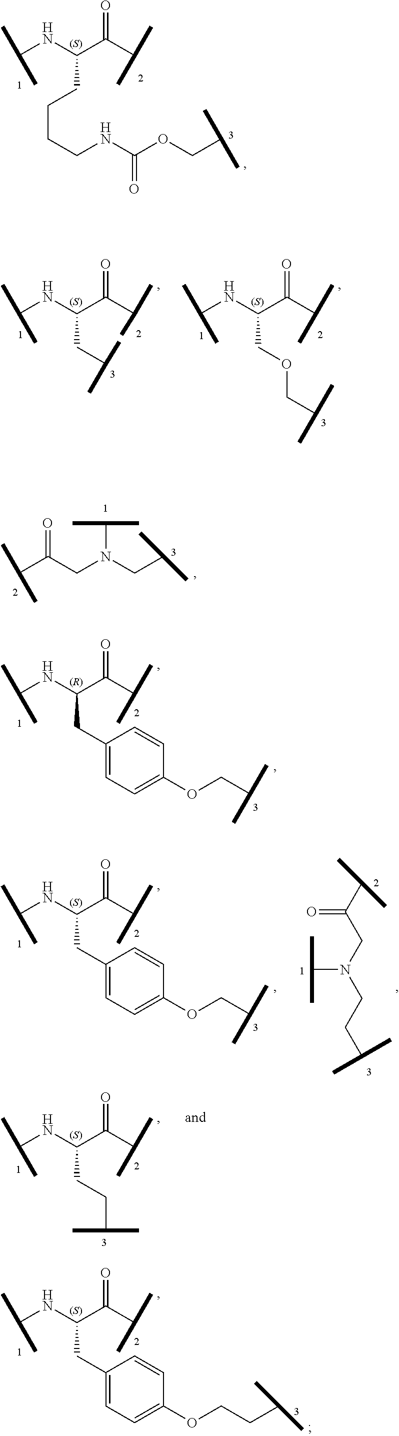

each occurrence of Z of Formulas (IAP-XXIX) and (IAP-XXX) is independently selected from

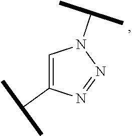

wherein each

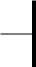

represents a point of attachment to the compound; and each Y is independently selected from:

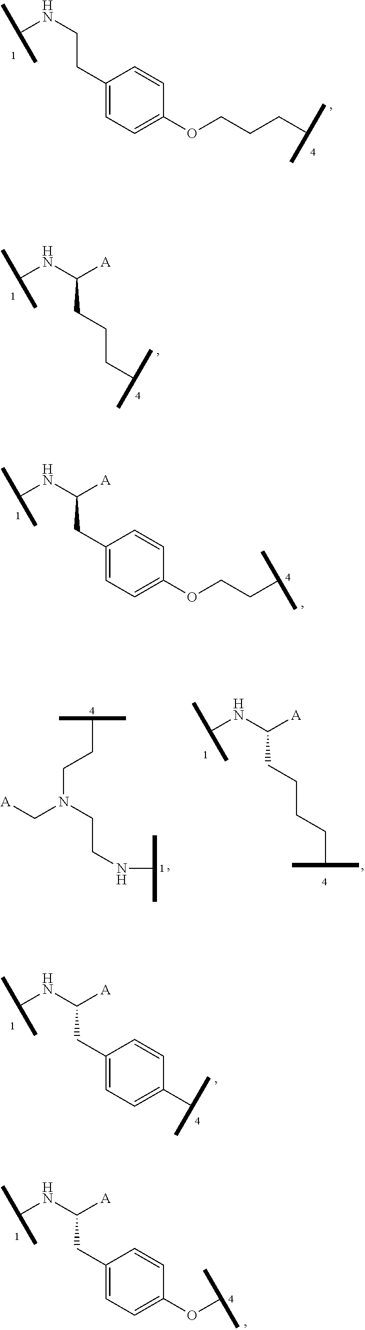

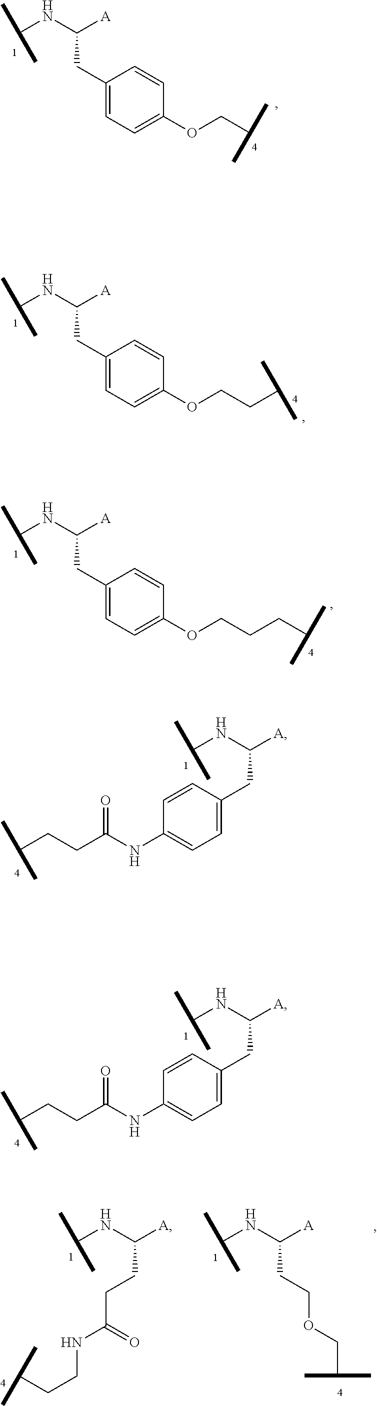

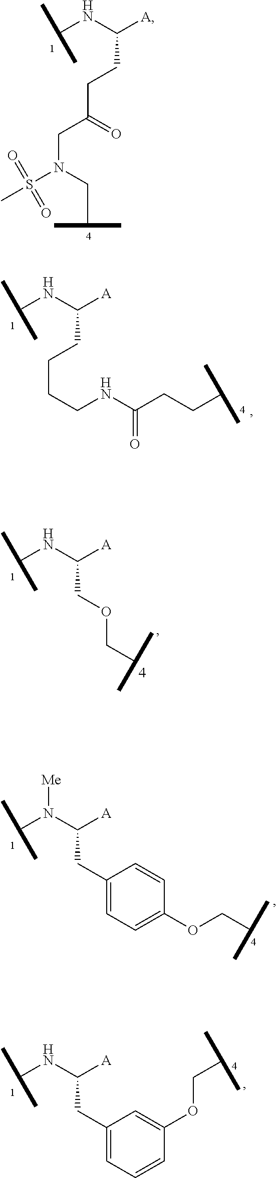

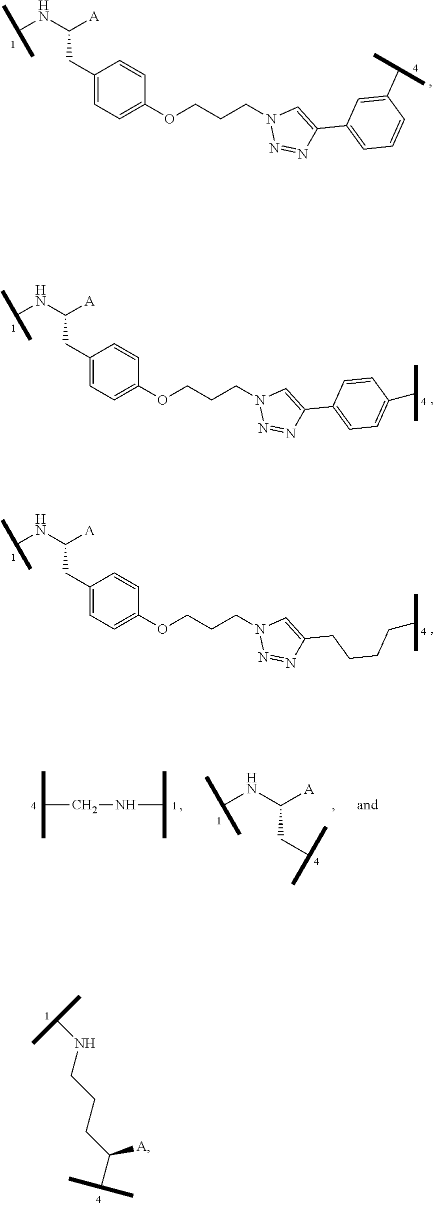

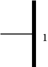

represents a point of attachment to a —C(═O) portion of the compound;

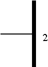

represents a point of attachment to an amino portion of the compound;

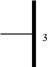

represents a first point of attachment to Z;

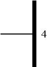

represents a second point of attachment to Z; and

each occurrence of A of Formulas (IAP-XXIX) and (IAP-XXX) is independently selected from —C(═O)R³ or

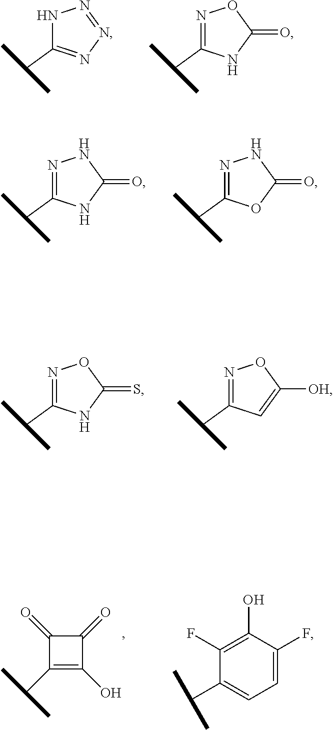

or a tautomeric form of any of the foregoing, wherein:

each occurrence of R³ of —C(═O)R³ of Formulas (IAP-XXIX) and (IAP-XXX) is selected from OH, NHCN, NHSO₂R¹⁰, NHOR¹¹ or N(R¹²)(R¹³);

each occurrence of R¹⁰ and R″ of NHSO₂R¹⁰ and NHOR¹¹ of Formulas (IAP-XXIX) and (IAP-XXX) is independently selected from —C₁-C₄ alkyl, cycloalkyl, aryl, heteroaryl, or heterocycloalkyl, any of which are optionally substituted, and hydrogen;

each occurrence of R¹² and R¹³ of N(R¹²)(R¹³) of Formulas (IAP-XXIX) and (IAP-XXX) is independently selected from hydrogen, —C₁-C₄ alkyl, —(C₁-C₄ alkylene)-NH—(C₁-C₄ alkyl), benzyl, —(C₁-C₄ alkylene)-C(═O)OH, —(C₁-C₄ alkylene)-C(═O)CH₃, —CH(benzyl)-COOH, —C₁-C₄ alkoxy, and

—(C₁-C₄ alkylene)-O—(C₁-C₄ hydroxyalkyl); or R¹² and R¹³ of N(R¹²)(R¹³) are taken together with the nitrogen atom to which they are commonly bound to form a saturated heterocyclyl optionally comprising one additional heteroatom selected from N, O and S, and wherein the saturated heterocycle is optionally substituted with methyl.

In various embodiments, the ULM can have the structure of Formula (IAP-XLI) as described in WO Pub. No. 2013/071039, or an unnatural mimetic thereof:

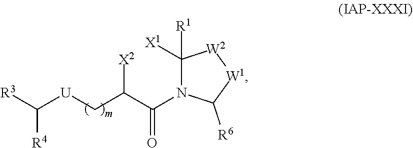

wherein:

each occurrence of W¹ of Formula (IAP-XXXI) is independently selected from O, S, N—R^{A}, or C(R^{8a})(R^{8b});

each occurrence of W² of Formula (IAP-XXXI) is independently selected from O, S, N—R^{A}, or C(R^{8c})(R^{8d}); provided that W¹ and W² are not both O, or both S;

each occurrence of R¹ of Formula (IAP-XXXI) is independently selected from H, C₁-C₆alkyl, C₃-C₆cycloalkyl, —C₁-C₆alkyl-(substituted or unsubstituted C₃-C₆cycloalkyl), substituted or unsubstituted aryl, substituted or unsubstituted heteroaryl, —C₁-C₆alkyl-(substituted or unsubstituted aryl), or —C₁-C₆alkyl-(substituted or unsubstituted heteroaryl);

when each occurrence of X¹ of Formula (IAP-XXXI) is independently selected from O, N—R^{A}, S, S(═O), or S(═O)₂, then X² is C(R^{2a}R^{2b});

or:

each occurrence of X¹ of Formula (IAP-XXXI) is independently selected from CR^{2c}R^{2d} and X² is CR^{2a}R^{2b}, and R^{2c} and R^{2a} together form a bond;

or:

each occurrence of X¹ and X² of Formula (IAP-XXXI) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring;

or:

each occurrence of X¹ of Formula (IAP-XXXI) is independently selected from CH₂ and X² is C(═O), C═C(R^{C})₂, or C═NR^{C}; where each R^{c} is independently selected from H, —CN, —OH, alkoxy, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);

each occurrence of R^{A} of N—R^{A} of Formula (IAP-XXXI) is independently selected from H, C₁-C₆alkyl, —C(═O)C₁-C₂alkyl, substituted or unsubstituted aryl, or substituted or unsubstituted heteroaryl;

each occurrence of R^{2a}, R^{2b}, R^{2c}, R^{2d} of CR^{2c}R^{2d} and CR^{2a}R^{2b} of Formula (IAP-XXXI) is independently selected from H, substituted or unsubstituted C₁-C₆alkyl, substituted or unsubstituted C₁-C₆heteroalkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl) and —C(═O)R^{B};

each occurrence of R^{B} of —C(═O)R^{B} of Formula (IAP-XXXI) is independently selected from substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl), or —NR^{D}R^{E};

each occurrence of R^{D} and R^{E} of NR^{D}R^{E} of Formula (IAP-XXXI) is independently selected from H, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);

each occurrence of m of Formula (IAP-XXXI) is independently selected from 0, 1 or 2;

each occurrence of —U— of Formula (IAP-XXXI) is independently selected from —NHC(═O)—, —C(═O)NH—, —NHS(═O)₂—, —S(═O)₂NH—, —NHC(═O)NH—, —NH(C═O)O—, —O(C═O)NH—, or —NHS(═O)₂NH—;

each occurrence of R³ of Formula (IAP-XXXI) is independently selected from C₁-C₃alkyl, or C₁-C₃fluoroalkyl;

each occurrence of R⁴ of Formula (IAP-XXXI) is independently selected from —NHR⁵, —N(R⁵)₂, —N⁺(R⁵)₃ or —OR⁵;

each occurrence of each R⁵ of —NHR⁵, —N(R⁵)₂, —N(R⁵)₃⁺ and —OR⁵ of Formula (IAP-XXXI) is independently selected from H, C₁-C₃alkyl, C₁-C₃haloalkyl, C₁-C₃heteroalkyl and —C₁-C₃alkyl-(C₃-C₅cycloalkyl);

or:

each occurrence of R³ and R⁵ of Formula (IAP-XXXI) together with the atoms to which they are attached form a substituted or unsubstituted 5-7 membered ring;

or:

at each occurrence R³ of Formula (IAP-XXXI) is bonded to a nitrogen atom of U to form a substituted or unsubstituted 5-7 membered ring;

each occurrence of R⁶ of Formula (IAP-XXXI) is independently selected from —NHC(═O)R⁷, —C(═O)NHR⁷, —NHS(═O)₂R⁷, —S(═O)₂NHR⁷, —NHC(═O)NHR⁷, —NHS(═O)₂NHR^{7′}—(C₁-C₃alkyl)-NHC(═O)R⁷, —(C₁-C₃alkyl)-C(═O)NHR⁷, —(C₁-C₃alkyl)-NHS(═O)₂R⁷, —(C₁-C₃alkyl)-S(═O)₂NHR⁷, —(C₁-C₃alkyl)-NHC(═O)NHR, —(C₁-C₃alkyl)-NHS(═O)₂NHR⁷, substituted or unsubstituted C₂-C₁₀heterocycloalkyl, or substituted or unsubstituted heteroaryl;

each occurrence of R⁷ of —NHC(═O)R⁷, —C(═O)NHR⁷, —NHS(═O)₂R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)₂R⁷, —(C₁-C₃alkyl)-S(═O)₂NHR⁷, —(C₁-C₃alkyl)-NHC(═O)NHR⁷, —(C₁-C₃alkyl)-NHS(═O)₂NHR⁷ of Formula (IAP-XLI) is independently selected from C₁-C₆alkyl, C₁-C₆haloalkyl, C₁-C₆heteroalkyl, a substituted or unsubstituted C₃-C₁₀cycloalkyl, a substituted or unsubstituted C₂-C₁₀heterocycloalkyl, a substituted or unsubstituted aryl, a substituted or unsubstituted heteroaryl, —C₁-C₆alkyl-(substituted or unsubstituted C₃-C₁₀cycloalkyl), —C₁-C₆alkyl-(substituted or unsubstituted C₂-C₁₀heterocycloalkyl, —C₁-C₆alkyl-(substituted or unsubstituted aryl), —C₁-C₆alkyl-(substituted or unsubstituted heteroaryl), —(CH₂)ₚ—CH(substituted or unsubstituted aryl)₂, —(CH₂)ₚ—CH(substituted or unsubstituted heteroaryl)₂, —(CH₂)ₚ—CH(substituted or unsubstituted aryl)(substituted or unsubstituted heteroaryl), -(substituted or unsubstituted aryl)-(substituted or unsubstituted aryl), -(substituted or unsubstituted aryl)-(substituted or unsubstituted heteroaryl), -(substituted or unsubstituted heteroaryl)-(substituted or unsubstituted aryl), or -(substituted or unsubstituted heteroaryl)-(substituted or unsubstituted heteroaryl);

each occurrence of p of R⁷ of Formula (IAP-XXXI) is independently selected from 0, 1 or 2;

each occurrence of R^{8a}, R^{8b}, R^{8c}, and R^{8d} of C(R^{8a})(R^{8b}) and C(R^{8c})(R^{8d}) of Formula (IAP-XXXI) is independently selected from H, C₁-C₆alkyl, C₁-C₆fluoroalkyl, C₁-C₆ alkoxy, C₁-C₆heteroalkyl, and substituted or unsubstituted aryl;

or:

each occurrence of R^{8a} and R^{8d} of Formula (IAP-XXXI) are as defined above, and R^{8b} and R^{8c} together form a bond;

or:

each occurrence of R^{8a} and R^{8d} of Formula (IAP-XXXI) are as defined above, and R^{8b} and R^{8c} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;

or:

each occurrence of R^{8c} and R^{8d} of Formula (IAP-XXXI) are as defined above, and R^{8a} and R^{8b} together with the atoms to which they are attached form a substituted or unsubstituted saturated, or partially saturated 3-7 membered spirocycle or heterospirocycle comprising 1-3 heteroatoms selected from S, O and N;

or:

each occurrence of R^{8a} and R^{8b} of Formula (IAP-XXXI) are as defined above, and R^{8c} and R^{8d} together with the atoms to which they are attached form a substituted or unsubstituted saturated, or partially saturated 3-7 membered spirocycle or heterospirocycle comprising 1-3 heteroatoms selected from S, O and N;

where each substituted alkyl, heteroalkyl, fused ring, spirocycle, heterospirocycle, cycloalkyl, heterocycloalkyl, aryl or heteroaryl is substituted with 1-3 R⁹; and

each occurrence of R⁹ of R^{8a}, R^{8b}, R^{8c} and R^{8d} of Formula (XXXI) is independently selected from halogen, —OH, —SH, (C═O), CN, C₁-C₄alkyl, C₁-C₄fluoroalkyl, C₁-C₄ alkoxy, C₁-C₄ fluoroalkoxy, —NH₂, —NH(C₁-C₄alkyl), —NH(C₁-C₄alkyl)₂, —C(═O)OH, —C(═O)NH₂, —C(═O)C₁-C₃alkyl, —S(═O)₂CH₃, —NH(C₁-C₄alkyl)-OH, —NH(C₁-C₄alkyl)-O—(C₁-C₄alkyl), —O(C₁-C₄alkyl)-NH2, O(C₁-C₄alkyl)-NH—(C₁-C₄alkyl), and —O(C₁-C₄alkyl)-N—(C₁-C₄alkyl)₂, or two R⁹ together with the atoms to which they are attached form a methylene dioxy or ethylene dioxy ring substituted or unsubstituted with halogen, —OH, or C₁-C₃alkyl.

In various embodiments, the ULM can have the structure of Formula (IAP-XXXII), as described in WO Pub. No. 2013/071039, or an unnatural mimetic thereof:

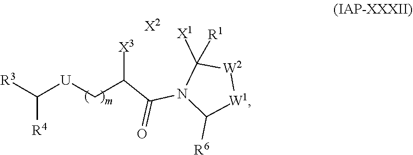

wherein:

each occurrence of W¹ in Formula (IAP-XXXII) is independently O, S, N—R^{A}, or C(R^{8a})(R^{8b});

each occurrence of W² in Formula (IAP-XXXII) is independently O, S, N—R^{A}, or C(R^{8c})(R^{8d}); provided that W¹ and W² are not both O, or both S;

each occurrence of R¹ in Formula (IAP-XXXII) is independently selected from H, C₁-C₆alkyl, C₃-C₆cycloalkyl, —C₁-C₆alkyl-(substituted or unsubstituted C₃-C₆cycloalkyl), substituted or unsubstituted aryl, substituted or unsubstituted heteroaryl, —C₁-C₆alkyl-(substituted or unsubstituted aryl), or —C₁-C₆alkyl-(substituted or unsubstituted heteroaryl); when X¹ of Formula (IAP-XXXII) is N—R^{A}, then X² is C═O, or CR^{2c}R^{2d}, and X³ is CR^{2a}R^{2b};

or:

when X¹ of Formula (IAP-XXXII) is selected from S, S(═O), or S(═O)₂, then X² is CR^{2c}R^{2d}, and X³ is CR^{2a}R^{2b};

or:

when X¹ of Formula (IAP-XXXII) is O, then X² is CR^{2c}R^{2d} Or N—R^{A} and X³ is CR^{2a}R^{2b};

or:

when X¹ of Formula (IAP-XXXII) is CH₃, then X² is independently selected from O, N—R^{A}, S, S(═O), or S(═O)₂, and X³ is CR^{2a}R^{2b};

when X¹ of Formula (IAP-XXXII) is CR^{2e}R^{2f} then X₂ is CR^{2c}R^{2d}, and R^{2c} and R^{2c} together form a bond, and X³ of Formula (VXXII) is CR^{2a}R^{2b};

or:

when X¹ and X³ of Formula (IAP-XXXII) are both CH₂ and X² of Formula (IAP-XLII) is C═O, C═C(R^{C})₂, or C═NR^{C}; where each R^{c} is independently selected from H, —CN, —OH, alkoxy, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);

or:

X¹ and X² of Formula (IAP-XXXII) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X³ is CR^{2a}R^{2b};

or:

X² and X³ of Formula (IAP-XXXII) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X¹ of Formula (IAP-XLII) is CR^{2e}R^{2f};

Each occurrence of R^{A} of N—R^{A} of Formula (IAP-XXXII) is independently selected from H, C₁-C₆alkyl, —C(═O)C₁-C₂alkyl, substituted or unsubstituted aryl, or substituted or unsubstituted heteroaryl;

at each occurrence R^{2a}, R^{2b}, R^{2c}, R^{2d}, R^{2e}, and R^{2f} of CR²CR^{2d}, CR^{2a}R^{2b} and CR^{2e}R^{2f} of Formula (IAP-XXXII) are independently selected from H, substituted or unsubstituted C₁-C₆alkyl, substituted or unsubstituted C₁-C₆heteroalkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl) and —C(═O)R^{B};

at each occurrence R^{B} of —C(═O)R^{B} of Formula (IAP-XXXII) is independently selected from substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl), or —NR^{D}R^{E};

at each occurrence R^{D} and R^{E} of NR^{D}R^{E} are independently selected from H, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);

at each occurrence m of Formula (IAP-XXXII) is independently selected from 0, 1 or 2;

at each occurrence —U— of Formula (IAP-XXXII) is independently selected from —NHC(═O)—, —C(═O)NH—, —NHS(═O)₂—, —S(═O)₂NH—, —NHC(═O)NH—, —NH(C═O)O—, —O(C═O)NH—, or —NHS(═O)₂NH—;

at each occurrence R³ of Formula (IAP-XXXII) is independently selected from C₁-C₃alkyl, or C₁-C₃fluoroalkyl;

at each occurrence R⁴ of Formula (IAP-XXXII) is independently selected from —NHR⁵, —N(R⁵)₂, —N(R⁵)₃⁺ or —OR⁵;

at each occurrence each R⁵ of —NHR⁵, —N(R⁵)₂, —N(R⁵)₃⁺ and —OR⁵ is independently selected from H, C₁-C₃alkyl, C₁-C₃haloalkyl, C₁-C₃heteroalkyl and —C₁-C₃alkyl-(C₃-C₅cycloalkyl);

or:

at each occurrence R³ and R⁵ of Formula (IAP-XXXII) together with the atoms to which they are attached form a substituted or unsubstituted 5-7 membered ring;

or:

at each occurrence R³ of Formula (IAP-XXXII) is bonded to a nitrogen atom of U to form a substituted or unsubstituted 5-7 membered ring;

at each occurrence R⁶ of Formula (IAP-XXXII) is independently selected from —NHC(═O)R⁷, —C(═O)NHR⁷, —NHS(═O)₂R⁷, —S(═O)₂NHR⁷, —NHC(═O)NHR⁷, —NHS(═O)₂NHR^{7′}—(C₁-C₃alkyl)-NHC(═O)R⁷, —(C₁-C₃alkyl)-C(═O)NHR⁷, —(C₁-C₃alkyl)-NHS(═O)₂R⁷, —(C₁-C₃alkyl)-S(═O)₂NHR⁷, —(C₁-C₃alkyl)-NHC(═O)NHR⁷, —(C₁-C₃alkyl)-NHS(═O)₂NHR⁷, substituted or unsubstituted C₂-C₁₀heterocycloalkyl, or substituted or unsubstituted heteroaryl;

at each occurrence R⁷ of —NHC(═O)R⁷, —C(═O)NHR⁷, —NHS(═O)₂R⁷, —S(═O)₂NHR⁷, NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)₂R⁷, —(C₁-C₃alkyl)-S(═O)₂NHR⁷, —(C₁-C₃alkyl)-NHC(═O)NHR⁷, —(C₁-C₃alkyl)-NHS(═O)₂NHR⁷ is independently selected from C₁-C₆alkyl, C₁-C₆haloalkyl, C₁-C₆heteroalkyl, a substituted or unsubstituted C₃-C₁₀cycloalkyl, a substituted or unsubstituted C₂-C₁₀heterocycloalkyl, a substituted or unsubstituted aryl, a substituted or unsubstituted heteroaryl, —C₁-C₆alkyl-(substituted or unsubstituted C₃-C₁₀cycloalkyl), —C₁-C₆alkyl-(substituted or unsubstituted C₂-C₁₀heterocycloalkyl, —C₁-C₆alkyl-(substituted or unsubstituted aryl), —C₁-C₆alkyl-(substituted or unsubstituted heteroaryl), —(CH₂)ₚ—CH(substituted or unsubstituted aryl)₂, —(CH₂)ₚ—CH(substituted or unsubstituted heteroaryl)₂, —(CH₂)ₚ—CH(substituted or unsubstituted aryl)(substituted or unsubstituted heteroaryl), -(substituted or unsubstituted aryl)-(substituted or unsubstituted aryl), -(substituted or unsubstituted aryl)-(substituted or unsubstituted heteroaryl), -(substituted or unsubstituted heteroaryl)-(substituted or unsubstituted aryl), or -(substituted or unsubstituted heteroaryl)-(substituted or unsubstituted heteroaryl);

at each occurrence p of R⁷ is independently selected from 0, 1 or 2;

at each occurrence R^{8a}, R^{8b}, R^{8c}, and R^{8d} of C(R^{8a})(R^{8b}) and C(R^{8c})(R^{8d}) of Formula (IAP-XXXII) are independently selected from H, C₁-C₆alkyl, C₁-C₆fluoroalkyl, C₁-C₆ alkoxy, C₁-C₆heteroalkyl, and substituted or unsubstituted aryl;

or:

at each occurrence R^{8a} and R^{8d} of Formula (IAP-XXXII) are as defined above, and R^{8b} and R^{8c} together form a bond;

or:

at each occurrence R^{8a} and R^{8d} of Formula (IAP-XXXII) are as defined above, and R^{8b} and R^{8c} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;

or:

at each occurrence R^{8c} and R^{8d} of Formula (IAP-XXXII) are as defined above, and R^{8a} and R^{8b} together with the atoms to which they are attached form a substituted or unsubstituted saturated, or partially saturated 3-7 membered spirocycle or heterospirocycle comprising 1-3 heteroatoms selected from S, O and N;

or:

at each occurrence R^{8a} and R^{8b} of Formula (IAP-XXXII) are as defined above, and R^{8c} and R^{8d} together with the atoms to which they are attached form a substituted or unsubstituted saturated, or partially saturated 3-7 membered spirocycle or heterospirocycle comprising 1-3 heteroatoms selected from S, O and N;

where each substituted alkyl, heteroalkyl, fused ring, spirocycle, heterospirocycle, cycloalkyl, heterocycloalkyl, aryl or heteroaryl is substituted with 1-3 R⁹; and

at each occurrence R⁹ of R^{8a}, R^{8b}, R^{8c} and R^{8d} is independently selected from halogen, —OH, —SH, C(═O), CN, C₁-C₄alkyl, C₁-C₄fluoroalkyl, C₁-C₄ alkoxy, C₁-C₄ fluoroalkoxy, —NH₂, —NH(C₁-C₄alkyl), —NH(C₁-C₄alkyl)₂, —C(═O)OH, —C(═O)NH₂, —C(═O)C₁-C₃alkyl, —S(═O)₂CH₃, —NH(C₁-C₄alkyl)-OH, —NH(C₁-C₄alkyl)-O—(C₁-C₄alkyl), —O(C₁-C₄alkyl)-NH₂, —O(C₁-C₄alkyl)-NH—(C₁-C₄alkyl), and —O(C₁-C₄alkyl)-N—(C₁-C₄alkyl)₂, or two R⁹ taken together with the atoms to which they are attached form a methylene dioxy or ethylene dioxy ring substituted or unsubstituted with halogen, —OH, or C₁-C₃alkyl.

In various embodiments, the ULM can have the structure of Formula (IAP-XLIII), as described in WO Pub. No. 2013/071039, or an unnatural mimetic thereof:

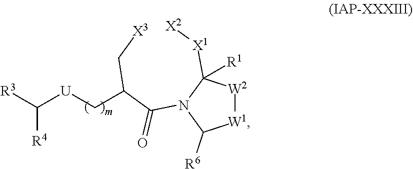

wherein:

at each occurrence W¹ of Formula (IAP-XXXIII) is independently selected from O, S, N—R^{A}, or C(R^{8a})(R^{8b});

at each occurrence W² of Formula (IAP-XXXIII) is independently selected from O, S, N—R^{A}, or C(R^{8c})(R^{8d}); provided that W¹ and W² are not both O, or both S;

at each occurrence R¹ of Formula (IAP-XXXIII) is independently selected from H, C₁-C₆alkyl, C₃-C₆cycloalkyl, —C₁-C₆alkyl-(substituted or unsubstituted C₃-C₆cycloalkyl), substituted or unsubstituted aryl, substituted or unsubstituted heteroaryl, —C₁-C₆alkyl-(substituted or unsubstituted aryl), or —C₁-C₆alkyl-(substituted or unsubstituted heteroaryl);

when X¹ of Formula (IAP-XXXIII) is independently selected from N—R^{A}, S, S(═O), or S(═O)₂, then X² of Formula (IAP-XXXIII) is CR^{2c}R^{2d}, and X³ of Formula (IAP-XXXIII) is CR^{2a}R^{2b};

or:

when X¹ of Formula (IAP-XXXIII) is O, then X² of Formula (IAP-XXXIII) is independently selected from O, N—R^{A}, S, S(═O), or S(═O)₂, and X³ of Formula (IAP-XXXIII) is CR^{2a}R^{2b};

or:

when X¹ of Formula (IAP-XLIII) is CR^{2e}R^{2f}, then X² of Formula (IAP-XXXIII) is CR²CR^{2d}, and R^{2e} and R^{2c} together form a bond, and X³ of Formula (IAP-XXXIII) is CR^{2a}R^{2b};

or:

at each occurrence X¹ and X² of Formula (IAP-XXXIII) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X³ of Formula (IAP-XLIII) is CR^{2a}R^{2b};

or:

at each occurrence X² and X³ of Formula (IAP-XXXIII) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X¹ of Formula (IAP-XXXIII) is CR^{2e}R^{2f};

at each occurrence R^{A} of N—R^{A} of Formula (IAP-XXXIII) is independently H, C₁-C₆alkyl, —C(═O)C₁-C₂alkyl, substituted or unsubstituted aryl, or substituted or unsubstituted heteroaryl;

at each occurrence R^{2a}, R^{2b}, R^{2c}, R^{2d}, R^{2e}, and R^{2f} of CR²CR^{2d}, CR^{2a}R^{2b} and CR^{2e}R^{2f} of Formula (IAP-XXXIII) are independently selected from H, substituted or unsubstituted C₁-C₆alkyl, substituted or unsubstituted C₁-C₆heteroalkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl) and —C(═O)R^{B};

at each occurrence R^{B} of —C(═O)R^{B} of Formula (IAP-XXXIII) is substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl), or —NR^{D}R^{E};

at each occurrence R^{D} and R^{E} of NR^{D}R^{E} of Formula (IAP-XXXIII) are independently selected from H, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);

at each occurrence m of Formula (IAP-XXXIII) is independently 0, 1 or 2;

at each occurrence —U— of Formula (IAP-XXXIII) is independently —NHC(═O)—, —C(═O)NH—, —NHS(═O)₂—, —S(═O)₂NH—, —NHC(═O)NH—, —NH(C═O)O—, —O(C═O)NH—, or —NHS(═O)₂NH—;

at each occurrence R³ of Formula (IAP-XXXIII) is independently C₁-C₃alkyl, or C₁-C₃fluoroalkyl;

at each occurrence R⁴ of Formula (IAP-XXXIII) is independently —NHR⁵, —N(R⁵)₂, —N⁺(R⁵)₃ or —OR⁵;

at each occurrence R⁵ of —NHR⁵, —N(R⁵)₂, —N⁺(R⁵)₃ and —OR⁵ is independently selected from H, C₁-C₃alkyl, C₁-C₃haloalkyl, C₁-C₃heteroalkyl and —C₁-C₃alkyl-(C₃-C₅cycloalkyl);

or:

at each occurrence R³ and R⁵ of Formula (IAP-XXXIII) together with the atoms to which they are attached form a substituted or unsubstituted 5-7 membered ring;

or:

at each occurrence R³ of Formula (IAP-XXXIII) is bonded to a nitrogen atom of U to form a substituted or unsubstituted 5-7 membered ring;

at each occurrence R⁶ of Formula (IAP-XXXIII) is independently selected from —NHC(═O)R⁷, —C(═O)NHR⁷, —NHS(═O)₂R⁷, —S(═O)₂NHR⁷, —NHC(═O)NHR⁷, —NHS(═O)₂NHR⁷, —(C₁-C₃alkyl)-NHC(═O)R⁷, —(C₁-C₃alkyl)-C(═O)NHR, —(C₁-C₃alkyl)-NHS(═O)₂R⁷, —(C₁-C₃alkyl)-S(═O)₂NHR⁷, —(C₁-C₃alkyl)-NHC(═O)NHR⁷, —(C₁-C₃alkyl)-NHS(═O)₂NHR⁷, substituted or unsubstituted C₂-C₁₀heterocycloalkyl, or substituted or unsubstituted heteroaryl;

at each occurrence R⁷ of —NHC(═O)R⁷, —C(═O)NHR⁷, —NHS(═O)₂R⁷, —S(═O)₂NHR⁷, —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)₂R⁷, —(C₁-C₃alkyl)-S(═O)₂NHR⁷, —(C₁-C₃alkyl)-NHC(═O)NHR⁷, —(C₁-C₃alkyl)-NHS(═O)₂NHR⁷ is independently selected from C₁-C₆alkyl, C₁-C₆haloalkyl, C₁-C₆heteroalkyl, a substituted or unsubstituted C₃-C₁₀cycloalkyl, a substituted or unsubstituted C₂-C₁₀heterocycloalkyl, a substituted or unsubstituted aryl, a substituted or unsubstituted heteroaryl, —C₁-C₆alkyl-(substituted or unsubstituted C₃-C₁₀cycloalkyl), —C₁-C₆alkyl-(substituted or unsubstituted C₂-C₁₀heterocycloalkyl, —C₁-C₆alkyl-(substituted or unsubstituted aryl), —C₁-C₆alkyl-(substituted or unsubstituted heteroaryl), —(CH₂)ₚ—CH(substituted or unsubstituted aryl)₂, —(CH₂)ₚ—CH(substituted or unsubstituted heteroaryl)₂, —(CH₂)ₚ—CH(substituted or unsubstituted aryl)(substituted or unsubstituted heteroaryl), -(substituted or unsubstituted aryl)-(substituted or unsubstituted aryl), -(substituted or unsubstituted aryl)-(substituted or unsubstituted heteroaryl), -(substituted or unsubstituted heteroaryl)-(substituted or unsubstituted aryl), or -(substituted or unsubstituted heteroaryl)-(substituted or unsubstituted heteroaryl);

at each occurrence p of R⁷ of Formula (IAP-XXXIII) is independently 0, 1 or 2;

at each occurrence R^{8a}, R^{8b}, R^{8c}, and R^{8d} of C(R^{8a})(R^{8b}) and C(R^{8c})(R^{8d}) of Formula (IAP-XLIII) are independently selected from H, C₁-C₆alkyl, C₁-C₆fluoroalkyl, C₁-C₆ alkoxy, C₁-C₆heteroalkyl, and substituted or unsubstituted aryl;

or:

at each occurrence R^{8a} and R^{8d} of Formula (IAP-XXXIII) are as defined above, and R^{8b} and R^{8c} together form a bond;

or:

at each occurrence R^{8a} and R^{8d} of Formula (IAP-XXXIII) are as defined above, and R^{8b} and R^{8c} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;

or:

at each occurrence R^{8c} and R^{8d} of Formula (IAP-XXXIII) are as defined above, and R^{8a} and R^{8b} together with the atoms to which they are attached form a substituted or unsubstituted saturated, or partially saturated 3-7 membered spirocycle or heterospirocycle comprising 1-3 heteroatoms selected from S, O and N;

or:

at each occurrence R^{8a} and R^{8b} of Formula (IAP-XXXIII) are as defined above, and R^{8c} and R^{8d} together with the atoms to which they are attached form a substituted or unsubstituted saturated, or partially saturated 3-7 membered spirocycle or heterospirocycle comprising 1-3 heteroatoms selected from S, O and N; where each substituted alkyl, heteroalkyl, fused ring, spirocycle, heterospirocycle, cycloalkyl, heterocycloalkyl, aryl or heteroaryl is substituted with 1-3 R⁹; and

at each occurrence R⁹ of R^{8a}, R^{8b}, R^{8c} and R^{8d} of Formula (IAP-XXXIII) is independently selected from halogen, —OH, —SH, C(═O), CN, C₁-C₄alkyl, C₁-C₄fluoroalkyl, C₁-C₄ alkoxy, C₁-C₄ fluoroalkoxy, —NH₂, —NH(C₁-C₄alkyl), —NH(C₁-C₄alkyl)₂, —C(═O)OH, —C(═O)NH₂, —C(═O)C₁-C₃alkyl, —S(═O)₂CH₃, —NH(C₁-C₄alkyl)-OH, —NH(C₁-C₄alkyl)-O—(C₁-C₄alkyl), —O(C₁-C₄alkyl)-NH₂, —O(C₁-C₄alkyl)-NH—(C₁-C₄alkyl), and —O(C₁-C₄alkyl)-N—(C₁-C₄alkyl)₂, or two R⁹ together with the atoms to which they are attached form a methylene dioxy or ethylene dioxy ring substituted or unsubstituted with halogen, —OH, or C₁-C₃alkyl.

In various embodiments, the ULM can have the structure of Formula (IAP-XLIV), as described in WO Pub. No. 2013/071039, or an unnatural mimetic thereof:

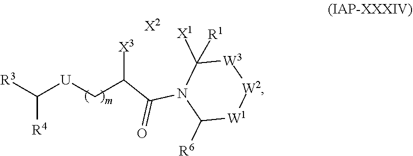

wherein:

at each occurrence W¹ of Formula (IAP-XXXIV) is independently selected from O, S, N—R^{A}, or C(R^{8a})(R^{8b});

at each occurrence W² of Formula (IAP-XXXIV) is independently selected from O, S, N—R^{A}, or C(R^{8c})(R^{8d}); provided that W¹ and W² are not both O, or both S;

at each occurrence W³ of Formula (IAP-XXXIV) is independently selected from O, S, N—R^{A}, or C(R^{8e})(R^{8f}), providing that the ring comprising W¹, W², and W³ does not comprise two adjacent oxygen atoms or sulfur atoms;

at each occurrence R¹ of Formula (IAP-XXXIV) is independently selected from H, C₁-C₆alkyl, C₃-C₆cycloalkyl, —C₁-C₆alkyl-(substituted or unsubstituted C₃-C₆cycloalkyl), substituted or unsubstituted aryl, substituted or unsubstituted heteroaryl, —C₁-C₆alkyl-(substituted or unsubstituted aryl), or —C₁-C₆alkyl-(substituted or unsubstituted heteroaryl);

when X¹ of Formula (IAP-XXXIV) is O, then X² of Formula (IAP-XXXXIVLIV) is independently selected from CR^{2C}R^{2d} and N—R^{A}, and X³ of Formula (IAP-XXXIV) is CR^{2a}R^{2b};

or:

when X¹ of Formula (IAP-XXXIV) is CH₂, then X² of Formula (IAP-XXXIV) is independently selected from O, N—R^{A}, S, S(═O), or S(═O)₂, and X³ of Formula (IAP-XXXIV) is CR^{2a}R^{2b};

or:

when X¹ of Formula (IAP-XXXIV) is CR^{2e}R^{2f}, then X² of Formula (IAP-XXXIV) is CR²CR^{2d}, and R^{2e} and R^{2c} together form a bond, and X³ of Formula (IAP-XXXIV) is CR^{2a}R^{2b};

or:

when X¹ and X³ of Formula (IAP-XXXIV) are both CH₂, then X² of Formula (IAP-XXXXIVLII) is C═O, C═C(R^{C})₂, or C═NR^{C}; where each R^{c} is independently selected from H, —CN, —OH, alkoxy, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);

or:

at each occurrence X¹ and X² of Formula (IAP-XXXIV) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X³ of Formula (IAP-XXXIV) is CR^{2a}R^{2b};

or:

at each occurrence X² and X³ of Formula (IAP-XXXIV) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X¹ of Formula (IAP-XXXIV) is CR^{2e}R^{2f};

at each occurrence R^{A} of N—R^{A} of Formula (IAP-XXXIV) is independently selected from H, C₁-C₆alkyl, —C(═O)C₁-C₂alkyl, substituted or unsubstituted aryl, or substituted or unsubstituted heteroaryl;

at each occurrence R^{2a}, R^{2b}, R^{2c}, R^{2d}, R^{2e}, and R^{2f} of CR²CR^{2d}, CR^{2a}R^{2b} and CR^{2e}R^{2f} of Formula (IAP-XXXIV) are independently selected from H, substituted or unsubstituted C₁-C₆alkyl, substituted or unsubstituted C₁-C₆heteroalkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl) and —C(═O)R^{B};

at each occurrence R^{B} of —C(═O)R^{B} of Formula (IAP-XXXIV) is independently selected from substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl), or —NR^{D}R^{E};

at each occurrence R^{D} and R^{E} of NR^{D}R^{E} of Formula (IAP-XXXIV) are independently selected from H, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl-(substituted or unsubstituted heteroaryl);

at each occurrence m of Formula (IAP-XXXIV) is independently selected from 0, 1 or 2;

at each occurrence —U— of Formula (IAP-XXXIV) is independently selected from —NHC(═O)—, —C(═O)NH—, —NHS(═O)₂—, —S(═O)₂NH—, —NHC(═O)NH—, —NH(C═O)O—, —O(C═O)NH—, or —NHS(═O)₂NH—;

at each occurrence R³ of Formula (IAP-XXXIV) is independently selected from C₁-C₃alkyl, or C₁-C₃fluoroalkyl;

at each occurrence R⁴ of Formula (IAP-XXXIV) is independently selected from —NHR⁵, —N(R⁵)₂, —N⁺(R⁵)₃ or —OR⁵;

at each occurrence R⁵ of —NHR⁵, —N(R⁵)₂, —N⁺(R⁵)₃ and —OR⁵ of Formula (IAP-XXXIV) is independently selected from H, C₁-C₃alkyl, C₁-C₃haloalkyl, C₁-C₃heteroalkyl and —C₁-C₃alkyl-(C₃-C₅cycloalkyl);

or:

at each occurrence R³ and R⁵ of Formula (IAP-XXXIV) together with the atoms to which they are attached form a substituted or unsubstituted 5-7 membered ring;

or:

at each occurrence R³ of Formula (IAP-XXXIV) is bonded to a nitrogen atom of U to form a substituted or unsubstituted 5-7 membered ring;

at each occurrence R⁶ of Formula (IAP-XXXIV) is independently selected from —NHC(═O)R⁷, —C(═O)NHR⁷, —NHS(═O)₂R⁷, —S(═O)₂NHR⁷, —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)₂R⁷, —(C₁-C₃alkyl)-S(═O)₂NHR⁷, —(C₁-C₃alkyl)-NHC(═O)NHR, —(C₁-C₃alkyl)-NHS(═O)₂NHR⁷, substituted or unsubstituted C₂-C₁₀heterocycloalkyl, or substituted or unsubstituted heteroaryl;

at each occurrence R⁷ of —NHC(═O)R⁷, —C(═O)NHR⁷, —NHS(═O)₂R⁷, —S(═O)₂NHR⁷, NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)₂R⁷, —(C₁-C₃alkyl)-S(═O)₂NHR⁷, —(C₁-C₃alkyl)-NHC(═O)NHR⁷, —(C₁-C₃alkyl)-NHS(═O)₂NHR⁷ is independently selected from C₁-C₆alkyl, C₁-C₆haloalkyl, C₁-C₆heteroalkyl, a substituted or unsubstituted C₃-C₁₀cycloalkyl, a substituted or unsubstituted C₂-C₁₀heterocycloalkyl, a substituted or unsubstituted aryl, a substituted or unsubstituted heteroaryl, —C₁-C₆alkyl-(substituted or unsubstituted C₃-C₁₀cycloalkyl), —C₁-C₆alkyl-(substituted or unsubstituted C₂-C₁₀heterocycloalkyl, —C₁-C₆alkyl-(substituted or unsubstituted aryl), —C₁-C₆alkyl-(substituted or unsubstituted heteroaryl), —(CH₂)ₚ—CH(substituted or unsubstituted aryl)₂, —(CH₂)ₚ—CH(substituted or unsubstituted heteroaryl)₂, —(CH₂)ₚ—CH(substituted or unsubstituted aryl)(substituted or unsubstituted heteroaryl), -(substituted or unsubstituted aryl)-(substituted or unsubstituted aryl), -(substituted or unsubstituted aryl)-(substituted or unsubstituted heteroaryl), -(substituted or unsubstituted heteroaryl)-(substituted or unsubstituted aryl), or -(substituted or unsubstituted heteroaryl)-(substituted or unsubstituted heteroaryl);

at each occurrence p of R⁷ is independently selected from 0, 1 or 2;

at each occurrence R^{8a}, R^{8b}, R^{8c}, R^{8d}, R^{8e}, and R^{8f} of C(R^{8a})(R^{8b}), C(R^{8c})(R^{8d}) and C(R^{8e})(R^{8f}) of Formula (IAP-XXXIV) are independently selected from H, C₁-C₆alkyl, C₁-C₆fluoroalkyl, C₁-C₆ alkoxy, C₁-C₆heteroalkyl, and substituted or unsubstituted aryl;

or:

at each occurrence R^{8a}, R^{8d}, R^{8e}, and R^{8f} of C(R^{8a})(R^{8b}), C(R^{8e})(R^{8d}) and C(R^{8e})(R^{8f}) of Formula (IAP-XXXIV) are as defined above, and R^{8b} and R^{8C} together form a bond;

or:

at each occurrence R^{8a}, R^{8b}, R^{8d}, and R^{8f} of C(R^{8a})(R^{8b}), C(R^{8c})(R^{8d}) and C(R^{8e})(R^{8f}) of Formula (IAP-XXXIV) are as defined above, and R^{8c} and R^{8e} together form a bond;

or:

at each occurrence R^{8a}, R^{8d}, R^{8e}, and R^{8f} of C(R^{8a})(R^{8b}), C(R^{8c})(R^{8d}) and C(R^{8e})(R^{8f}) of Formula (IAP-XXXIV) are as defined above, and R^{8b} and R^{8c} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;

or:

at each occurrence R^{8a}, R^{8b}, R^{8d}, and R^{8f} of C(R^{8a})(R^{8b}), C(R^{8c})(R^{8d}) and C(R^{8e})(R^{8f}) of Formula (IAP-XXXIV) are as defined above, and R^{8c} and R^{8e} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;

or:

at each occurrence R^{8c}, R^{8d}, R^{8e}, and R^{8f} of C(R^{8c})(R^{8d}) and C(R^{8e})(R^{8f}) of Formula (IAP-XXXIV) are as defined above, and R^{8a} and R^{8b} together with the atoms to which they are attached form a substituted or unsubstituted saturated, or partially saturated 3-7 membered spirocycle or heterospirocycle comprising 1-3 heteroatoms selected from S, O and N;

or:

at each occurrence R^{8a}, R^{8b}, R^{8c}, and R^{8f} of C(R^{8a})(R^{8b}) and C(R^{8e})(R^{8f}) of Formula (IAP-XXXIV) are as defined above, and R^{8c} and R^{8d} together with the atoms to which they are attached form a substituted or unsubstituted saturated, or partially saturated 3-7 membered spirocycle or heterospirocycle comprising 1-3 heteroatoms selected from S, O and N;

or:

at each occurrence R^{8a}, R^{8b}, R^{8c}, and R^{8d} of C(R^{8a})(R^{8b}) and C(R^{8c})(R^{8d}) of Formula (IAP-XXXIV) are as defined above, and R^{8e} and R^{8f} together with the atoms to which they are attached form a substituted or unsubstituted saturated, or partially saturated 3-7 membered spirocycle or heterospirocycle comprising 1-3 heteroatoms selected from S, O and N;

or:

where each substituted alkyl, heteroalkyl, fused ring, spirocycle, heterospirocycle, cycloalkyl, heterocycloalkyl, aryl or heteroaryl is substituted with 1-3 R⁹; and

at each occurrence R⁹ of R^{8a}, R^{8b}, R^{8c}, R^{8d}, R^{8e}, and R^{8f} is independently selected from halogen, —OH, —SH, C(═O), CN, C₁-C₄alkyl, C₁-C₄fluoroalkyl, C₁-C₄ alkoxy, C₁-C₄ fluoroalkoxy, —NH₂, —NH(C₁-C₄alkyl), —NH(C₁-C₄alkyl)₂, —C(═O)OH, —C(═O)NH₂, —C(═O)C₁-C₃alkyl, —S(═O)₂CH₃, —NH(C₁-C₄alkyl)-OH, —NH(C₁-C₄alkyl)-O—(C₁-C₄alkyl), —O(C₁-C₄alkyl)-NH₂, —O(C₁-C₄alkyl)-NH—(C₁-C₄alkyl), and —O(C₁-C₄alkyl)-N—(C₁-C₄alkyl)₂, or two R⁹ taken together with the atoms to which they are attached form a methylene dioxy or ethylene dioxy ring substituted or unsubstituted with halogen, —OH, or C₁-C₃alkyl.

In various embodiments, the ULM can have the structure of Formula (IAP-XXXV), (IAP-XXXVI) or (IAP-XXXVII), as described in ACS Chem. Biol., 8(4), 725-32 (2013), or an unnatural mimetic thereof:

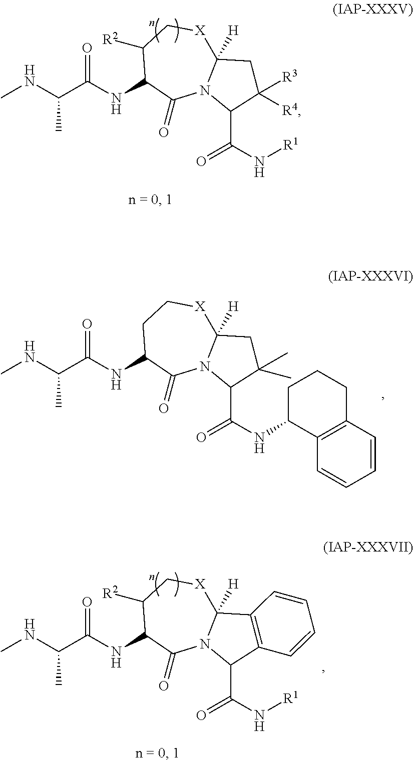

wherein:

at each occurrence of R² of Formula (IAP-XXXV) and (IAP-XXXVII) are independently selected from H or ME;

at each occurrence R³ and R⁴ of Formula (IAP-XXXV) are independently selected from H or Me;

at each occurrence X of Formulas (XXXV) through (XXXVII) is independently selected from O or S; and

at each occurrence R¹ of Formulas (XXXV) and (XXXVII) is independently selected from:

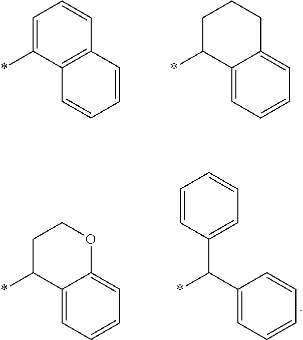 [SMILES: *c1cccc2ccccc12 ; *C1CCCc2ccccc21 ; *C1CCOc2ccccc21 ; *C(c1ccccc1)c1ccccc1]

In a particular embodiment, the ULM has a structure according to Formula (IAP-XXXVIII):

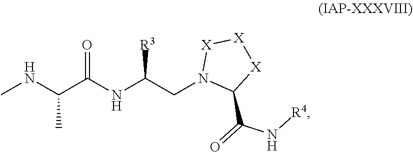

wherein R³ and R⁴ of Formula (IAP-XXXVIII) are independently selected from H or Me;

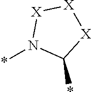

is a 5-member heterocycle independently selected from:

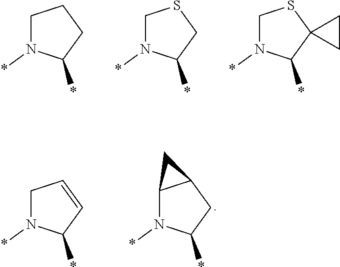 [SMILES: *[C@@H]1CCCN1* ; *[C@@H]1CSCN1* ; *[C@H]1N(*)CSC12CC2 ; *[C@@H]1C=CCN1* ; *[C@@H]1C[C@@H]2C[C@@H]2N1*]

In a particular embodiment, the

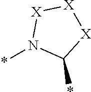

### of Formula (IAP-XXXVIII) is

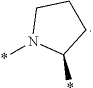 [SMILES: *[C@@H]1CCCN1*]

In a particular embodiment, the ULM has a structure and attached to a linker group L as shown below:

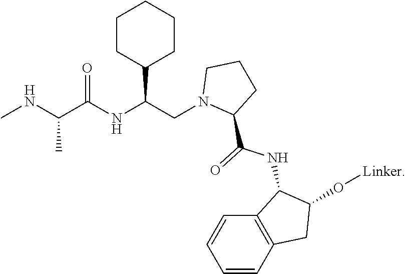

In various embodiments, the ULM can have the structure of Formula (IAP-XXXIX) or (IAP-XL), as described in Bioorg. Med. Chem. Lett., 22(4), 1960-4 (2012), or an unnatural mimetic thereof:

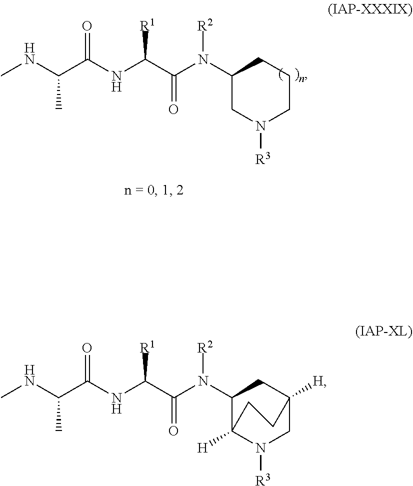

wherein:

at each occurrence R¹ of Formulas (IAP-XXXIX) and (IAP-XL) is independently selected from:

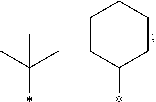

at each occurrence R² of Formulas (IAP-XXXIX) and (IAP-XL) is independently selected from H or Me;

at each occurrence R³ of Formulas (IAP-XXXIX) and (IAP-XL) is independently selected from:

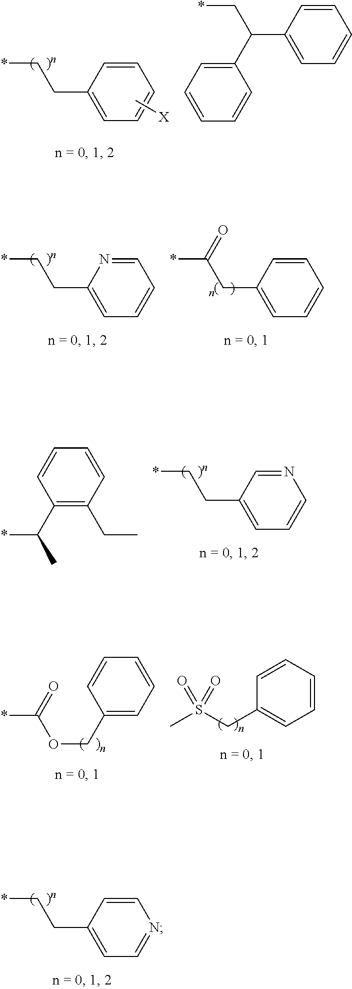

at each occurrence X of of Formulas (IAP-XXXIX) and (IAP-XL) is independently selected from H, halogen, methyl, methoxy, hydroxy, nitro or trifluoromethyl.

In various embodiments, the ULM can have the structure shown in Formula (IAP-XLI) or (IAP-XLII), where the the linker is as described herein, or an unnatural mimetic thereof:

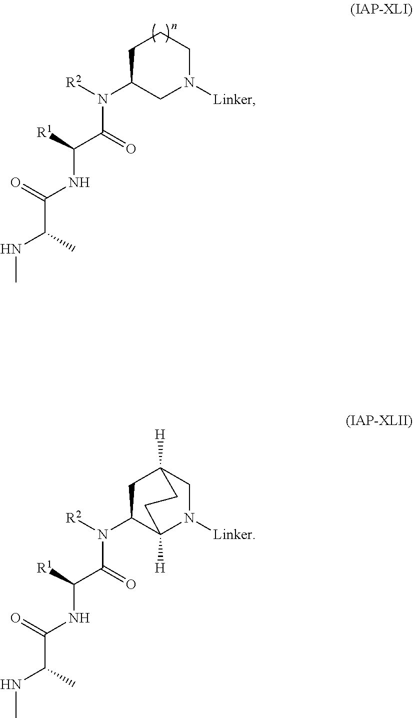

In various embodiments, the ULM can have the structure of Formula (IAP-XLIII), as described in J. Med. Chem., 52(6), 1723-30 (2009), or an unnatural mimetic thereof:

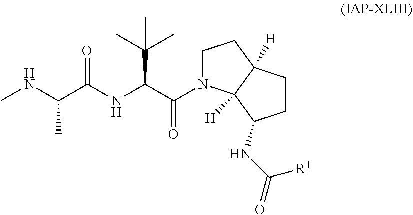

wherein:

at each occurrence R¹ of Formula (IAP-XLIII) is independently selected from:

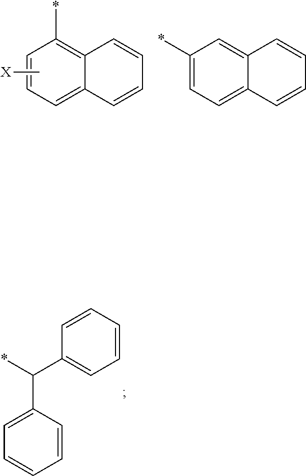

at each occurrence X of

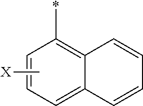

of Formula (IAP-XLIII) is independently selected from H, fluoro, methyl or methoxy.

In a particular embodiment, the ULM is represented by the following structure:

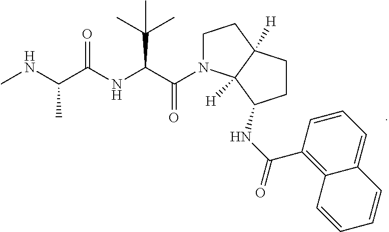 [SMILES: CN[C@@H](C)C(=O)N[C@H](C(=O)N1CC[C@H]2CC[C@H](NC(=O)c3cccc4ccccc34)[C@H]21)C(C)(C)C]

In a particular embodiment, the ULM, which has the chemical link between the ULM and linker group L as shown below. is selected from the group consisting of:

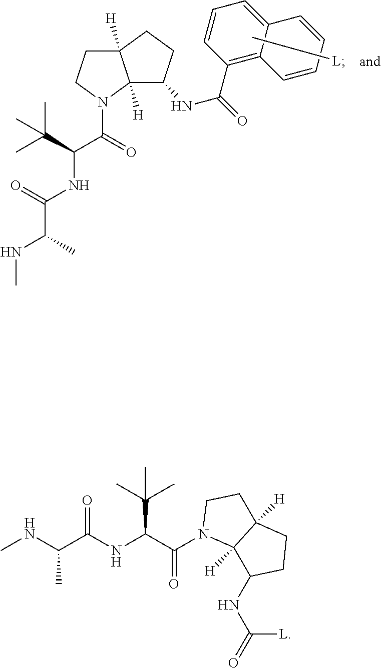

In various embodiments, the ULM is selected from the group consisting of, or an unnatural mimetic thereof:

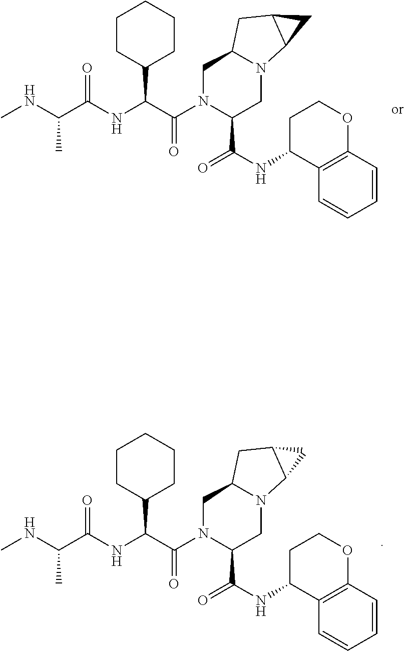 [SMILES: CN[C@@H](C)C(=O)N[C@H](C(=O)N1C[C@H]2C[C@H]3C[C@H]3N2C[C@H]1C(=O)N[C@@H]1CCOc2ccccc21)C1CCCCC1 ; CN[C@@H](C)C(=O)N[C@H](C(=O)N1C[C@H]2C[C@@H]3C[C@@H]3N2C[C@H]1C(=O)N[C@@H]1CCOc2ccccc21)C1CCCCC1]

In a particular embodiment, the ULM, in which the chemical link between the ULM and linker group L is shown below, is independently selected from the group consisting of:

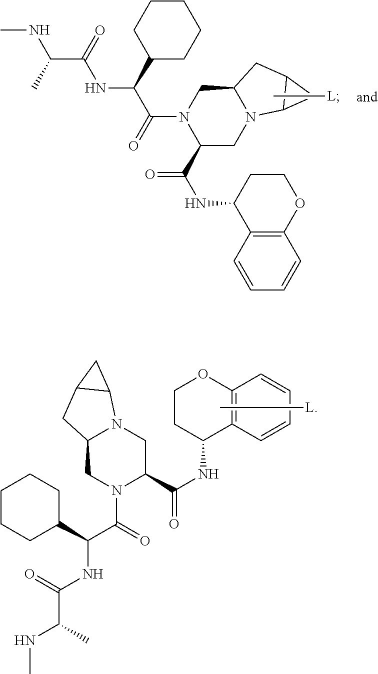

In various embodiments, the ULM can have the structure of Formula (IAP-XLIV), as described in Bioorg. Med. Chem., 21(18): 5725-37 (2013), or an unnatural mimetic thereof:

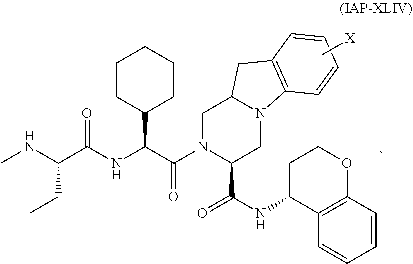

wherein at each occurrence X of Formula (IAP-XLIV) is one or two substituents independently selected from H, halogen or cyano.

In various embodiments, the ULM can have the structure of and be chemically linked to the linker group L as shown in Formula (IAP-XLV) or (IAPXLVI), or an unnatural mimetic thereof:

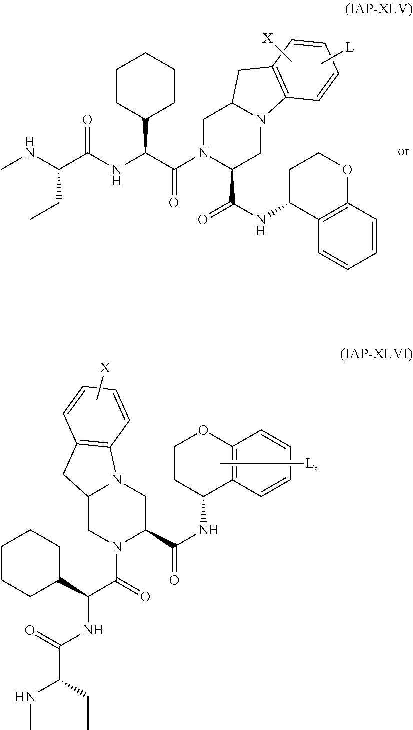

wherein X of Formulas (IAP-XLV) and (IAP-XLVI) is one or two substituents independently selected from H, halogen or cyano, and L of Formulas (IAP-XLV) and (IAP-XLVI) is a linker group as described herein.

In various embodiments, the ULM can have the structure of Formula (IAP-XLVII) as described in Bioorg. Med. Chem., 23(14): 4253-7 (2013), or an unnatural mimetic thereof:

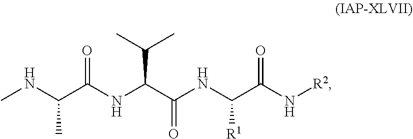

wherein:

at each occurrence

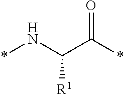

of Formula (IAP-XLVII) is a natural or unnatural amino acid; and

at each occurrence R² of Formula (IAP-XLVII) is independently selected from:

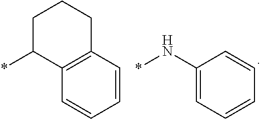

In various embodiments, the ULM can have the structure of and be chemically linked to the linker group L as shown in Formula (IAP-XLVIII) or (IAP-XLIX), or an unnatural mimetic thereof:

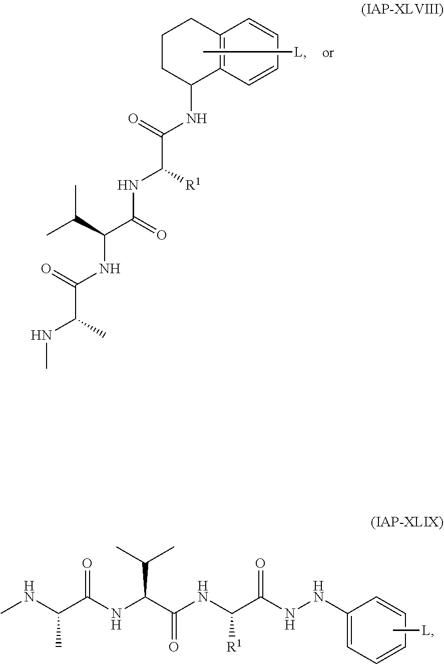

at each occurrence

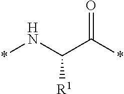

of Formula (IAP-XLVIII) or (IAP-XLIX) is a linker group as described herein.

In various embodiments, the ULM can have the structure selected from the group consisting of:

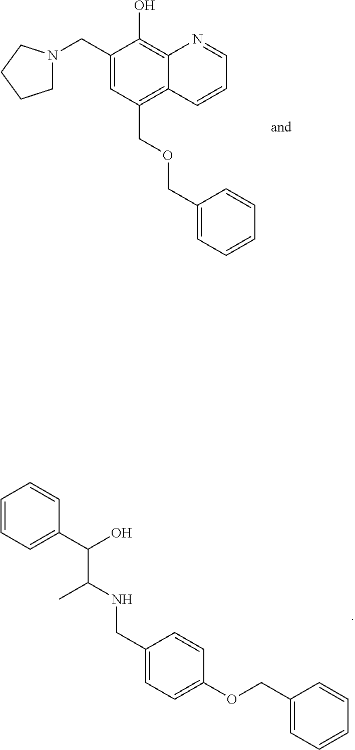 [SMILES: Oc1c(CN2CCCC2)cc(COCc2ccccc2)c2cccnc12 ; CC(NCc1ccc(OCc2ccccc2)cc1)C(O)c1ccccc1]

In various embodiments, the ULM has a structure according to Formula (IAP-L), as described in Bioorg. Med. Chem. Lett., 24(7): 1820-4 (2014), or an unnatural mimetic thereof:

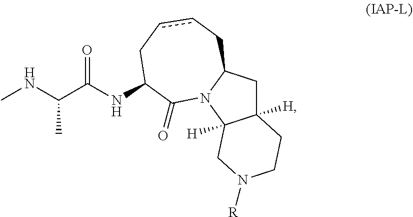

wherein at each occurrence R of Formula (IAP-L) is independently selected from the group consisting of:

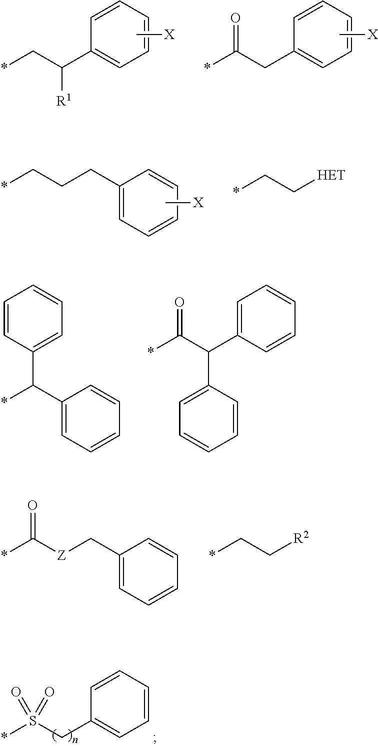

at each occurrence R¹ of

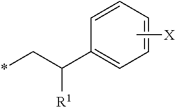

of Formula (IAP-L) is independently selected from H or Me;

at each occurrence R² of

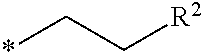

of Formula (IAP-L) is independently selected from alkyl or cycloalkyl;

at each occurrence X of

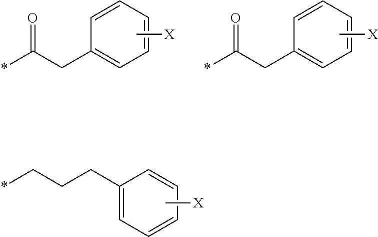

of Formula (IAP-L) is 1-2 substitutents independently selected from halogen, hydroxy, methoxy, nitro and trifluoromethyl

at each occurrence Z of

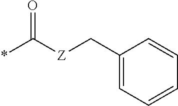

### of Formula (IAP-L) is O or NH;

at each occurrence HET of

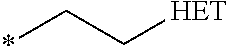

is mono- or fused bicyclic heteroaryl; and

at each occurrence --- of Formula (IAP-L) is an optional double bond.

In a particular embodiment, the ULM has a structure selected from the group consisting of:

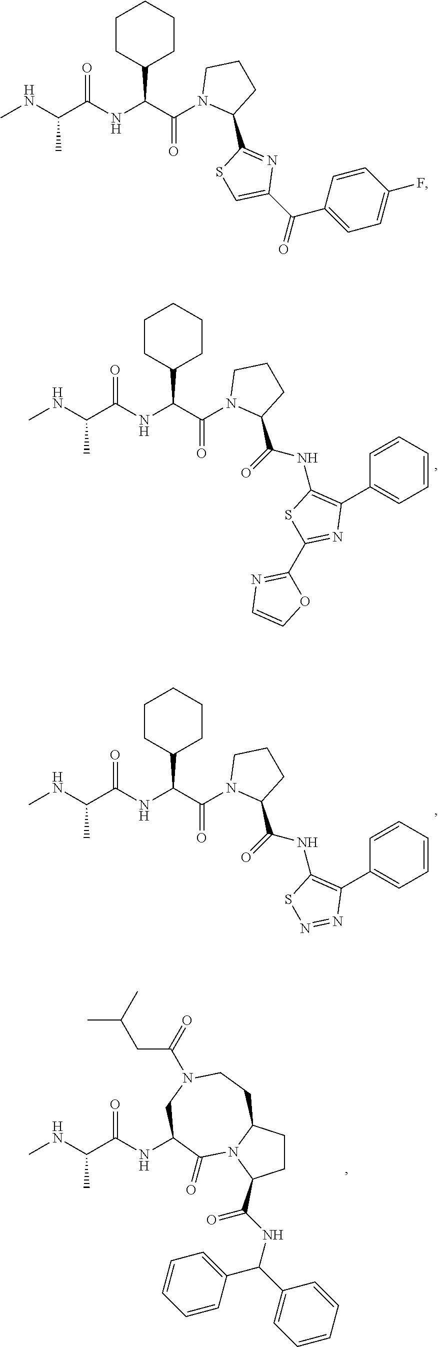 [SMILES: CN[C@@H](C)C(=O)N[C@H](C(=O)N1CCC[C@H]1c1nc(C(=O)c2ccc(F)cc2)cs1)C1CCCCC1 ; CN[C@@H](C)C(=O)N[C@H](C(=O)N1CCC[C@H]1C(=O)Nc1sc(-c2ncco2)nc1-c1ccccc1)C1CCCCC1 ; CN[C@@H](C)C(=O)N[C@H](C(=O)N1CCC[C@H]1C(=O)Nc1snnc1-c1ccccc1)C1CCCCC1 ; CN[C@@H](C)C(=O)N[C@H]1CN(C(=O)CC(C)C)CC[C@H]2CC[C@@H](C(=O)NC(c3ccccc3)c3ccccc3)N2C1=O]

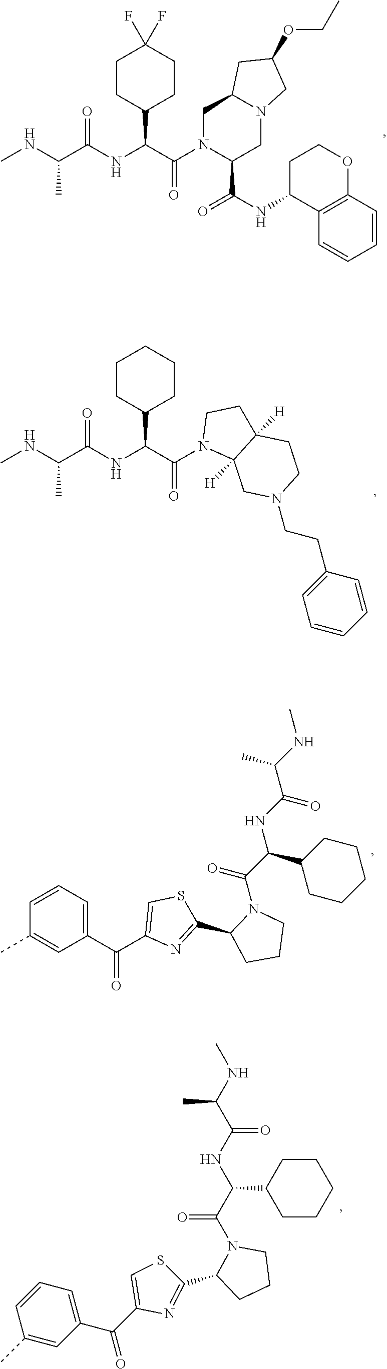

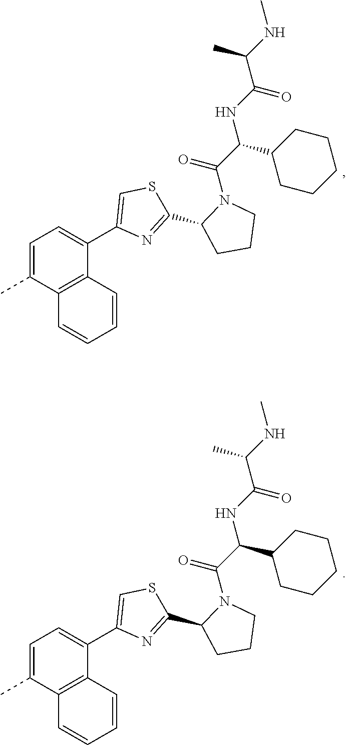

### Mouse Double Minute 2 Homolog E3 Ubiquitin Ligase Binding Moieties

In certain embodiments, the ULM of the compound includes chemical moieties such as substituted imidazolines, substituted spiro-indolinones, substituted pyrrolidines, substituted piperidinones, substituted morpholinones, substituted pyrrolopyrimidines, substituted imidazolopyridines, substituted thiazoloimidazoline, substituted pyrrolopyrrolidinones, and substituted isoquinolinones.

In additional embodiments, the ULM comprises the core structures mentioned above with adjacent bis-aryl substitutions positioned in cis- or trans-configurations.

In still additional embodiments, the ULM includes part of the structural features as in compounds RG7112, RG7388, SAR405838, AMG-232, AM-7209, DS-5272, MK-8242, and NVP-CGM-097, and analogs or derivatives thereof.

In certain embodiments, ULM is a compound of Formula (A-1), or thiazoloimidazoline represented as Formula (A-2), or spiro indolinone represented as Formula (A-3), or pyrollidine represented as Formula (A-4), or piperidinone/morphlinone represented as Formula (A-5), or isoquinolinone represented as Formula (A-6), or pyrollopyrimi dine/imidazolopyridine represented as Formula (A-7), or pyrrolopyrrolidinone/imidazolopyrrolidinone represented as Formula (A-8).

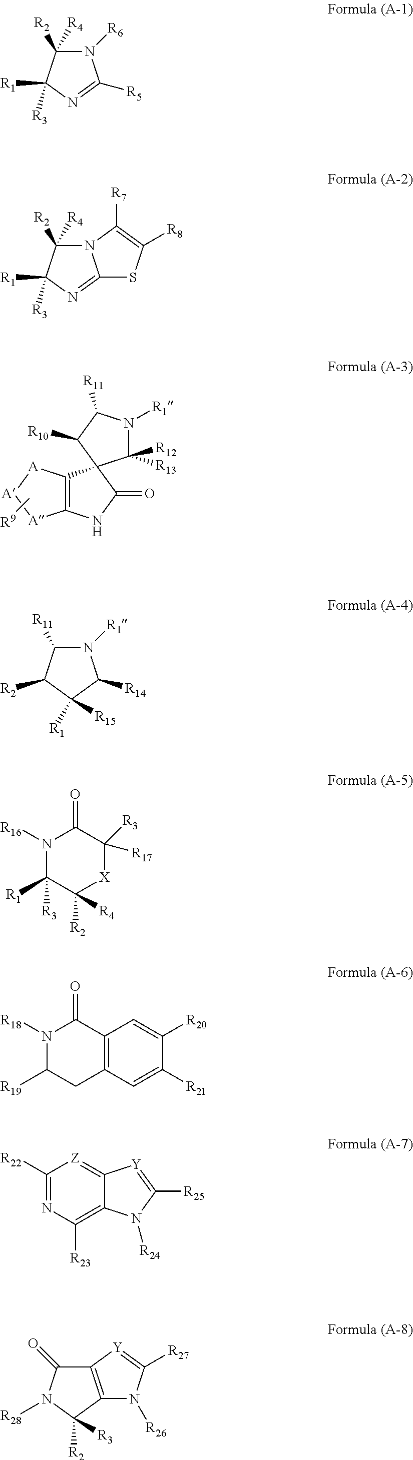

wherein in Formula (A-1) through Formula (A-8),

at each occurrence X of Formula (A-1) through Formula (A-8) is independently selected from the group consisting of carbon, oxygen, sulfur, sulfoxide, sulfone, and N—R^{a};

at each occurrence R^{a} of Formula (A-1) through Formula (A-8) is independently H or an alkyl group with carbon number 1 to 6;

at each occurrence Y and Z of Formula (A-1) through Formula (A-8) are independently carbon or nitrogen;

at each occurrence A, A′ and A″ of Formula (A-1) through Formula (A-8) are independently selected from C, N, O or S, can also be one or two atoms forming a fused bicyclic ring, or a 6,5- and 5,5-fused aromatic bicyclic group;

at each occurrence R¹, R² of Formula (A-1) through Formula (A-8) are independently selected from the group consisting of an aryl or heteroaryl group, a heteroaryl group having one or two heteroatoms independently selected from sulfur or nitrogen, wherein the aryl or heteroaryl group can be mono-cyclic or bi-cyclic, or unsubstituted or substituted with one to three substituents independently selected from the group consisting of: halogen, —CN, C₁₋₆ alkyl group, C₃₋₆ cycloalkyl, —OH, alkoxy with 1 to 6 carbons, fluorine substituted alkoxy with 1 to 6 carbons, sulfoxide with 1 to 6 carbons, sulfone with 1 to 6 carbons, ketone with 2 to 6 carbons, amides with 2 to 6 carbons, and dialkyl amine with 2 to 6 carbons;

at each occurrence R³, R⁴ of Formula (A-1) through Formula (A-8) are independently selected from the group consisting of H, methyl and C₁₋₆ alkyl;

at each occurrence R⁵ of Formula (A-1) through Formula (A-8) is independently selected from the group consisting of an aryl or heteroaryl group, a heteroaryl group having one or two heteroatoms independently selected from sulfur or nitrogen, wherein the aryl or heteroaryl group can be mono-cyclic or bi-cyclic, or unsubstituted or substituted with one to three substituents independently selected from the group consisting of: halogen, —CN, C₁₋₆ alkyl group, C₃₋₆ cycloalkyl, —OH, alkoxy with 1 to 6 carbons, fluorine substituted alkoxy with 1 to 6 carbons, sulfoxide with 1 to 6 carbons, sulfone with 1 to 6 carbons, ketone with 2 to 6 carbons, amides with 2 to 6 carbons, dialkyl amine with 2 to 6 carbons, alkyl ether (C₂-6), alkyl ketone (C₃₋₆), morpholinyl, alkyl ester (C₃₋₆), alkyl cyanide (C₃₋₆);

at each occurrence R⁶ of Formula (A-1) through Formula (A-8) is independently H or —C(═O)R^{b}, wherein

at each occurrence R^{b} of Formula (A-1) through Formula (A-8) is independently selected from the group consisting of alkyl, cycloalkyl, mono-, di- or tri-substituted aryl or heteroaryl, 4-morpholinyl, 1-(3-oxopiperazinyl), 1-piperidinyl, 4-N—R^{C}-morpholinyl, 4-R^{c}-1-piperidinyl, and 3-R^{c}-1-piperidinyl, wherein

at each occurrence R^{c} of Formula (A-1) through Formula (A-8) is independently selected from the group consisting of alkyl, fluorine substituted alkyl, cyano alkyl, hydroxyl-substituted alkyl, cycloalkyl, alkoxyalkyl, amide alkyl, alkyl sulfone, alkyl sulfoxide, alkyl amide, aryl, heteroaryl, mono-, bis- and tri-substituted aryl or heteroaryl, CH₂CH₂R^{d}, and CH₂CH₂CH₂R^{d}, wherein

at each occurrence R^{d} of Formula (A-1) through Formula (A-8) is independently selected from the group consisting of alkoxy, alkyl sulfone, alkyl sulfoxide, N-substituted carboxamide, —NHC(═O)-alkyl, —NH—SO₂-alkyl, aryl, substituted aryl, heteroaryl, substituted heteroaryl;

at each occurrence R⁷ of Formula (A-1) through Formula (A-8) is independently selected from the group consisting of H, C₁₋₆ alkyl, cyclic alkyl, fluorine substituted alkyl, cyano substituted alkyl, 5- or 6-membered hetero aryl or aryl, substituted 5- or 6-membered hetero aryl or aryl;

at each occurrence R⁸ of Formula (A-1) through Formula (A-8) is independently selected from the group consisting of —R^{e}—C(═O)—R^{f}, —R^{e}-alkoxy, —R^{e}-aryl, —R^{e}-heteroaryl, and —R^{e}—C(═O)—R^{f}—C(═O)—R^{g}, wherein:

at each occurrence R^{e} of Formula (A-1) through Formula (A-8) is an alkylene with 1 to 6 carbons, or a bond;

at each occurrence R^{f} of Formula (A-1) through Formula (A-8) is a substituted 4- to 7-membered heterocycle;

at each occurrence R^{g} of Formula (A-1) through Formula (A-8) is independently selected from the group consisting of aryl, hetero aryl, substituted aryl or heteroaryl, and 4- to 7-membered heterocycle;

at each occurrence R₉ of Formula (A-1) through Formula (A-8) is independently selected from the group consisting of a mono-, bis- or tri-substituent on the fused bicyclic aromatic ring in Formula (A-3), wherein the substitutents are independently selected from the group consisting of halogen, alkene, alkyne, alkyl, unsubstituted or substituted with C₁ or F;

at each occurrence R₁₀ of Formula (A-1) through Formula (A-8) is independently selected from the group consisting of an aryl or heteroaryl group, wherein the heteroaryl group can contain one or two heteroatoms as sulfur or nitrogen, aryl or heteroaryl group can be mono-cyclic or bi-cyclic, the aryl or heteroaryl group can be unsubstituted or substituted with one to three substituents, including a halogen, F, Cl, —CN, alkene, alkyne, C₁₋₆ alkyl group, C₁₋₆cycloalkyl, —OH, alkoxy with 1 to 6 carbons, fluorine substituted alkoxy with 1 to 6 carbons, sulfoxide with 1 to 6 carbons, sulfone with 1 to 6 carbons, ketone with 2 to 6 carbons;

at each occurrence R₁₁ of Formula (A-1) through Formula (A-8) is —C(═O)—N(R^{h})(Rⁱ), wherein R^{h} and Rⁱ are selected from groups consisting of: H; optionally substituted linear or branched C₁ to C₆ alkyl; alkoxy substituted alkyl; mono- and di-hydroxy substituted alkyl (e.g., a C₃ to C₆), sulfone substituted alkyl; optionally substituted aryl; optionally substituted heteraryl; mono-, bis- or tri-substituted aryl or heteroaryl; phenyl-4-carboxylic acid; substituted phenyl-4-carboxylic acid, alkyl carboxylic acid; optionally substituted heteroaryl carboxylic acid; alkyl carboxylic acid; fluorine substituted alkyl carboxylic acid; optionally substituted cycloalky, 3-hydroxycyclobutane, 4-hydroxycyclohehexane, aryl substituted cycloalkyl; heteroaryl substituted cycloalkyl; or Rh and Ri taken together form a ring;

at each occurrence R₁₂ and R₁₃ of Formula (A-1) through Formula (A-8) are independently selected from H, lower alkyl (C₁₋₆), lower alkenyl (C₂-6), lower alkynyl (C₂-6), cycloalkyl (4, 5 and 6-membered ring), substituted cycloalkyl, cycloalkenyl, substituted cycloalkenyl, 5- and 6-membered aryl and heteroaryl, R₁₂ and R₁₃ can be connected to form a 5- and 6-membered ring with or without substitution on the ring;

at each occurrence R₁₄ of Formula (A-1) through Formula (A-8) is selected from the group consisting of alkyl, substituted alkyl, alkenyl, substituted alkenyl, aryl, substituted aryl, heteroaryl, substituted heteroaryl, heterocycle, substituted heterocycle, cycloalkyl, substituted cycloalkyl, cycloalkenyl and substituted cycloalkenyl;

at each occurrence R₁₅ of Formula (A-1) through Formula (A-8) is CN;

at each occurrence R₁₆ of Formula (A-1) through Formula (A-8) is selected from the group consisting of C₁₋₆ alkyl, C₁₋₆ cycloalkyl, C₂₋₆ alkenyl, C₁₋₆ alkyl or C₃₋₆ cycloalkyl with one or multiple hydrogens replaced by fluorine, alkyl or cycloalkyl with one CH₂ replaced by S(═O), —S, or —S(═O)₂, alkyl or cycloalkyl with terminal CH₃ replaced by S(═O)₂N(alkyl)(alkyl), —C(═O)N(alkyl)(alkyl), —N(alkyl)S(═O)₂(alkyl), —C(═O)₂(alkyl), —O(alkyl), C1-6 alkyl or alkyl-cycloalkyl with hydron replaced by hydroxyl group, a 3 to 7 membered cycloalkyl or heterocycloalkyl, optionally containing a —(C═O)— group, or a 5 to 6 membered aryl or heteroaryl group, which heterocycloalkyl or heteroaryl group can contain from one to three heteroatoms independently selected from O, N or S, and the cycloalkyl, heterocycloalkyl, aryl or heteroaryl group can be unsubstituted or substituted with from one to three substituents independently selected from halogen, C₁₋₆ alkyl groups, hydroxylated C₁₋₆ alkyl, C₁₋₆ alkyl containing thioether, ether, sulfone, sulfoxide, fluorine substituted ether or cyano group;

at each occurrence R₁₇ of Formula (A-1) through Formula (A-8) is independently selected from the group consisting of (CH₂)ₙC(═O)NR^{k}R^{l}, wherein R^{k} and R^{l} are independently selected from H, C₁₋₆ alkyl, hydroxylated C₁₋₆ alkyl, C₁₋₆ alkoxy alkyl, C₁₋₆ alkyl with one or multiple hydrogens replaced by fluorine, C₁₋₆ alkyl with one carbon replaced by S(═O), S(═O)(O), C₁₋₆ alkoxyalkyl with one or multiple hydrogens replaced by fluorine, C₁₋₆ alkyl with hydrogen replaced by a cyano group, 5 and 6 membered aryl or heteroaryl, aklyl aryl with alkyl group containing 1-6 carbons, and alkyl heteroaryl with alkyl group containing 1-6 carbons, wherein the aryl or heteroaryl group can be further substituted;

at each occurrence R₁₈ of Formula (A-1) through Formula (A-8) is independently selected from the group consisting of substituted aryl, heteroaryl, alkyl, cycloalkyl, the substitution is preferably —N(C₁₋₄ alkyl)(cycloalkyl), —N(C₁₋₄ alkyl)alkyl-cycloalkyl, and —N(C₁₋₄ alkyl)[(alkyl)-(heterocycle-substituted)-cycloalkyl];

at each occurrence R₁₉ of Formula (A-1) through Formula (A-8) is independently selected from the group consisting of aryl, heteroaryl, bicyclic heteroaryl, and these aryl or heteroaryl groups can be substituted with halogen, C₁₋₆ alkyl, C₁₋₆ cycloalkyl, CF₃, F, CN, alkyne, alkyl sulfone, the halogen substitution can be mon- bis- or tri-substituted;

at each occurrence R₂₀ and R₂₁ of Formula (A-1) through Formula (A-8) are independently selected from C₁₋₆ alkyl, C₁₋₆ cycloalkyl, C₁₋₆ alkoxy, hydroxylated C₁₋₆ alkoxy, and fluorine substituted C₁₋₆ alkoxy, wherein R₂₀ and R₂₁ can further be connected to form a 5, 6 and 7-membered cyclic or heterocyclic ring, which can further be substituted;

at each occurrence R₂₂ of Formula (A-1) through Formula (A-8) is independently selected from the group consisting of H, C₁₋₆ alkyl, C₁₋₆ cycloalkyl, carboxylic acid, carboxylic acid ester, amide, reverse amide, sulfonamide, reverse sulfonamide, N-acyl urea, nitrogen-containing 5-membered heterocycle, the 5-membered heterocycles can be further substituted with C₁₋₆ alkyl, alkoxy, fluorine-substituted alkyl, CN, and alkylsulfone;

at each occurrence R₂₃ of Formula (A-1) through Formula (A-8) is independently selected from aryl, heteroaryl, —O-aryl, —O-heteroaryl, —O-alkyl, —O-alkyl-cycloalkyl, —NH-alkyl, —NH— alkyl-cycloalkyl, —N(H)-aryl, —N(H)-heteroaryl, —N(alkyl)-aryl, —N(alkyl)-heteroaryl, the aryl or heteroaryl groups can be substituted with halogen, C₁₋₆ alkyl, hydroxylated C₁₋₆ alkyl, cycloalkyl, fluorine-substituted C₁₋₆ alkyl, CN, alkoxy, alkyl sulfone, amide and sulfonamide;

at each occurrence R₂₄ of Formula (A-1) through Formula (A-8) is selected from the group consisting of —CH₂—(C₁₋₆ alkyl), —CH₂-cycloalkyl, —CH₂-aryl, CH₂-heteroaryl, where alkyl, cycloalkyl, aryl and heteroaryl can be substituted with halogen, alkoxy, hydroxylated alkyl, cyano-substituted alkyl, cycloalkyl and substituted cycloalkyl;

at each occurrence R₂₅ of Formula (A-1) through Formula (A-8) is independently selected from the group consisting of C₁₋₆ alkyl, C₁₋₆ alkyl-cycloalkyl, alkoxy-substituted alkyl, hydroxylated alkyl, aryl, heteroaryl, substituted aryl or heteroaryl, 5, 6, and 7-membered nitrogen-containing saturated heterocycles, 5,6-fused and 6,6-fused nitrogen-containing saturated heterocycles and these saturated heterocycles can be substituted with C₁₋₆ alkyl, fluorine-substituted C₁₋₆ alkyl, alkoxy, aryl and heteroaryl group;

at each occurrence R₂₆ of Formula (A-1) through Formula (A-8) is independently selected from the group consisting of C₁₋₆ alkyl, C₃₋₆ cycloalkyl, the alkyl or cycloalkyl can be substituted with —OH, alkoxy, fluorine-substituted alkoxy, fluorine-substituted alkyl, —NH₂, —NH-alkyl, NH—C(═O)alkyl, —NH—S(═O)₂-alkyl, and —S(═O)₂-alkyl;

at each occurrence R₂₇ of Formula (A-1) through Formula (A-8) is independently selected from the group consisting of aryl, heteroaryl, bicyclic heteroaryl, wherein the aryl or heteroaryl groups can be substituted with C₁₋₆ alkyl, alkoxy, NH₂, NH-alkyl, halogen, or —CN, and the substitution can be independently mono-, bis- and tri-substitution;

at each occurrence R₂₈ of Formula (A-1) through Formula (A-8) is independently selected from the group consisting of aryl, 5 and 6-membered heteroaryl, bicyclic heteroaryl, cycloalkyl, saturated heterocycle such as piperidine, piperidinone, tetrahydropyran, N-acyl-piperidine, wherein the cycloalkyl, saturated heterocycle, aryl or heteroaryl can be further substituted with —OH, alkoxy, mono-, bis- or tri-substitution including halogen, —CN, alkyl sulfone, and fluorine substituted alkyl groups; and

at each occurrence R_{1″} of Formula (A-1) through Formula (A-8) is independently selected from the group consisting of alkyl, aryl substituted alkyl, alkoxy substituted alkyl, cycloalkyl, aryl-substituted cycloalkyl, and alkoxy substituted cycloalkyl.

In certain embodiments, the heterocycles in R_{f} and R_{g} of Formula (A-1) through Formula (A-8) are independently substituted pyrrolidine, substituted piperidine, substituted piperizine.

In various embodiments, the ULMs of Formula A-1 through A-8, can be used to prepare PROTACs as described herein to target a particular protein for degradation, where L is a linker group, and ATKI is a ligand binding to a target protein.

In certain embodiments, the compounds include a molecule with a structure selected from the group consisting of:

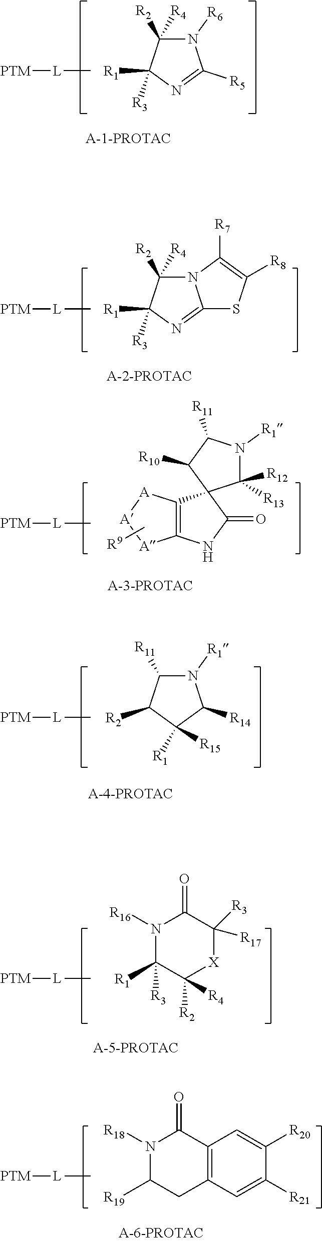

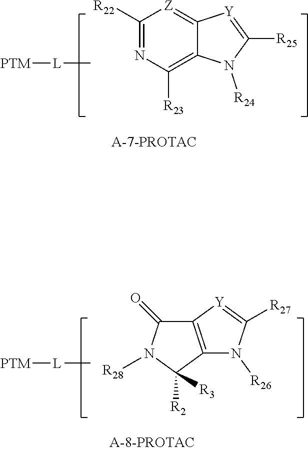

wherein at each occurrence X, R^{a}, Y, Z, A, A′, A″, R₁, R₂, R₃, R₄, R₅, R₆, R^{b}, R^{c}, R^{d}, R₇, R^{e}, R^{f}, R^{g}, R₉, R₁₀, R₁₁, R₁₂, R₁₃, R₁₄, R₁₅, R₁₆, R₁₇, R^{k}, R^{l}, R₁₈, R₁₉, R₂₀, R₂₁, R₂₂, R₂₃, R₂₄, R₂₅, R₂₆, R₂₇, R₂₈, and R_{1″} are independently as defined herein with regard to Formulas (A-1) through (A-8).

In certain embodiments, the compound includes molecules with the structure: ATKI-L-ULM, wherein ATKI is a protein target binding moiety coupled to an ULM by L, wherein L is a bond (i.e., absent) or a chemical linker. In some embodiments, the PTM in the structures of A-1-1, A-1-2, A-1-3, and A-1-4 is an ATKI as described herein. In certain embodiments, the ULM has a structure selected from the group consisting of A-1-1, A-1-2, A-1-3, and A-1-4:

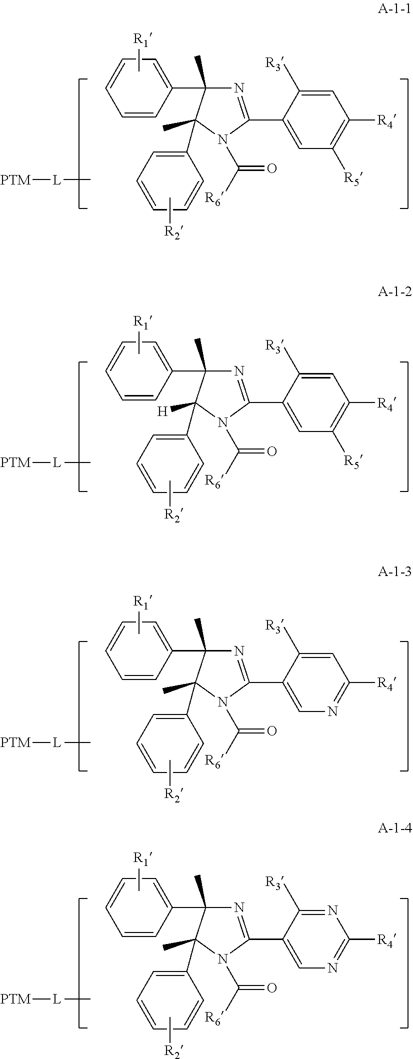

wherein:

at each occurrence R₁, and R_{2′} of Formulas A-1-1 through A-1-4 are independently selected from the group consisting of F, Cl, Br, I, ethynyl, CN, CF₃ and NO₂;

at each occurrence R_{3′} of Formulas A-1-1 through A-1-4 is independently selected from the group consisting of —OCH₃, —OCH₂CH₃, —OCH₂CH₂F, —OCH₂CH₂OCH₃, and —OCH(CH₃)₂;

at each occurrence R_{4′} of Formulas A-1-1 through A-1-4 is independently selected from the group consisting of H, halogen, —CH₃, —CF₃, —OCH₃, —C(CH₃)₃, —CH(CH₃)₂, -cyclopropyl, —CN, —C(CH₃)₂₀H, —C(CH₃)₂OCH₂CH₃, —C(CH₃)₂CH₂OH, —C(CH₃)₂CH₂OCH₂CH₃, —C(CH₃)₂CH₂OCH₂CH₂OH, —C(CH₃)₂CH₂OCH₂CH₃, —C(CH₃)₂CN, —C(CH₃)₂C(═O)CH₃, —C(CH₃)₂C(═O)NHCH₃, —C(CH₃)₂C(═O)N(CH₃)₂, —SCH₃, —SCH₂CH₃, —S(═O)₂CH₃, —S(O₂)CH₂CH₃, —NHC(CH₃)₃, —N(CH₃)₂, pyrrolidinyl, and 4-morpholinyl;

at each occurrence R_{5′} of Formulas A-1-1 through A-1-4 is independently selected from the group consisting of halogen, -cyclopropyl, —S(═O)₂CH₃, —S(═O)₂CH₂CH₃, 1-pyrrolidinyl, —NH₂, —N(CH₃)₂, and —NHC(CH₃)₃; and

at each occurrence R₆, of Formulas A-1-1 through A-1-4 is independently selected from the group consisting of H,

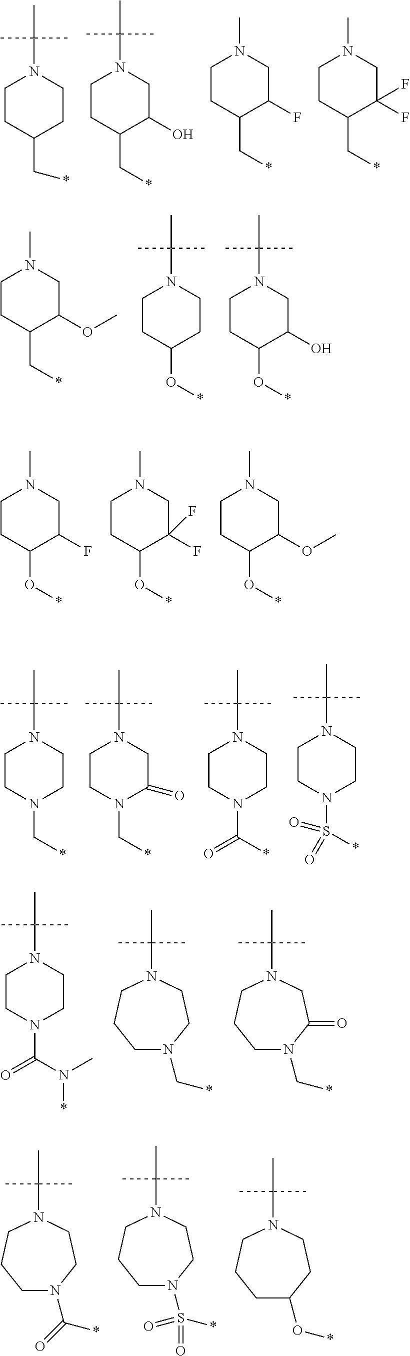

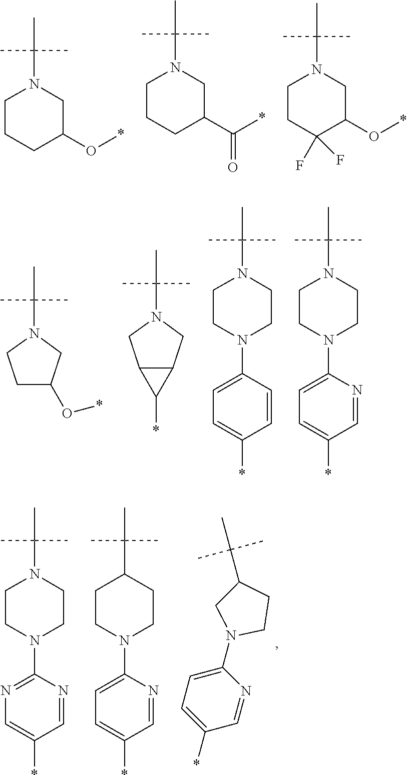

wherein “*” indicates the point of attachment of the linker.

In various embodiments, R_{4′} can also serve as the linker attachment position at any open valance in a terminal atom of any of the R_{4′} groups of Formulas A-1-1 through A-1-4.

In certain embodiments, the linker connection position of Formulas A-1-1 through A-1-4 is at least one of R_{4′} or R_{6′} Or both.

In certain embodiments, the linker of Formula A-4-1 through A-4-6 is attached to at least one of R₁, R_{2′}, R_{3′}, R_{4′}, R_{5′}, R_{6′}, r a combination thereof.

In certain embodiments, the description provides bifunctional or chimeric molecules with the structure: ATKI-L-ULM, wherein ATKI is a protein target binding moiety coupled to an ULM by L, wherein L is a bond or a chemical linker. In certain embodiments, the ULM has a structure selected from the group consisting of A-4-1, A-4-2, A-4-3, A-4-4, A-4-5, and A-4-6:

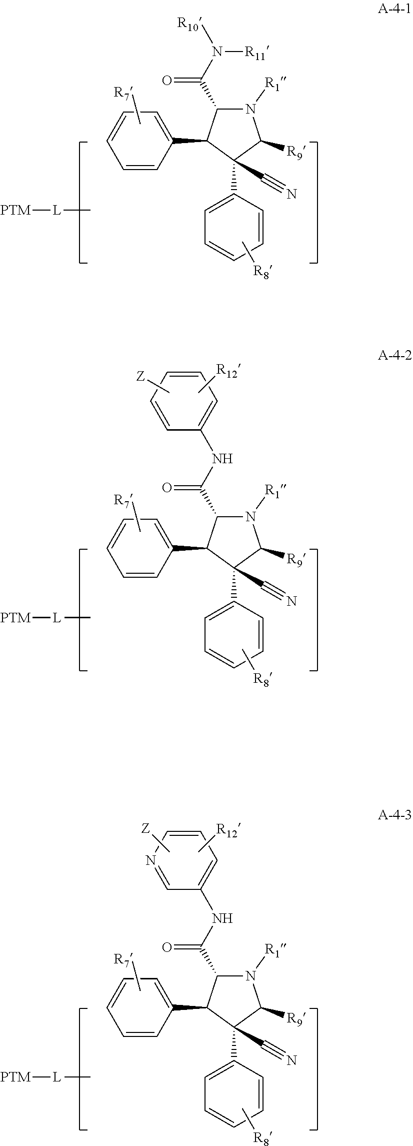

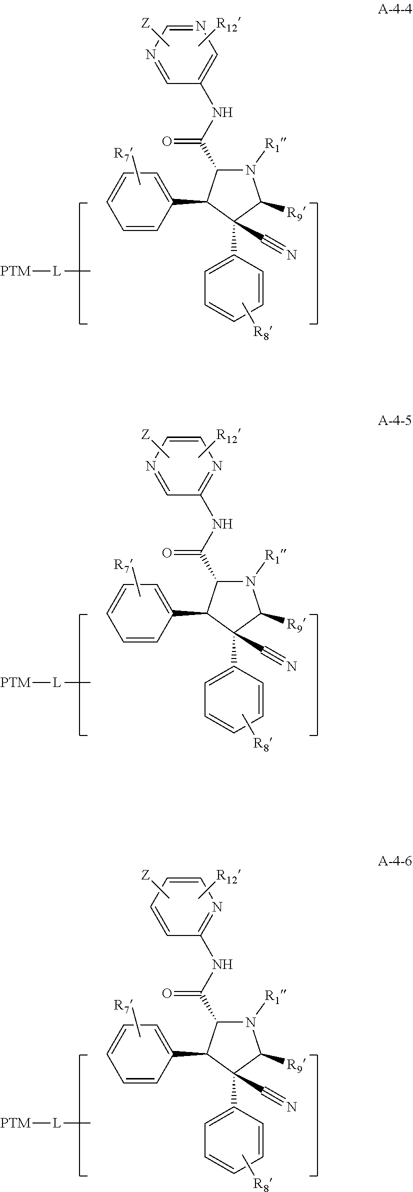

wherein:

R_{7′} of Formula A-4-1 through A-4-6 is one or more (e.g., 1, 2, 3, or 4) independently selected halogen;

R_{g′} of Formula A-4-1 through A-4-6 is one or more groups (e.g., 1, 2, 3, or 4 groups) independently selected from the group consisting of H, —F, —Cl, —Br, —I, —CN, —NO₂, ethylnyl, cyclopropyl, methyl, ethyl, isopropyl, vinyl, methoxy, ethoxy, isopropoxy, —OH, other C₁_₆ alkyl, other C₁₋₆ alkenyl, and C₁₋₆ alkynyl, mono-, di- or tri-substituted;

at each occurrence R_{9′} of Formula A-4-1 through A-4-6 is independently selected from the group consisting of alkyl, substituted alkyl, alkenyl, substituted alkenyl, alkynyl, substituted alkynyl, aryl, substituted aryl, hetero aryl, substituted heteroaryl, cycloalkyl, substituted cycloalkyl, alkenyl, and substituted cycloalkenyl;

at each occurrence Z of Formula A-4-1 through A-4-6 is independently selected from the group consisting of H, —OCH₃, —OCH₂CH₃, and halogen;

at each occurrence R_{10′} and R_{11′} of Formula A-4-1 through A-4-6 are each independently selected from the group consisting of H, (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH)ₙ—CONR′R″, (CH₂)ₙ—OR′, (CH₂)ₙ—SR′, (CH₂)ₙ—SOR′, (CH₂)ₙ—CH(OH)—R′, (CH₂)ₙ—COR′, (CH₂)ₙ—SO₂R′, (CH₂)ₙ—SONR′R″, (CH₂)ₙ—SO₂NR′R″, (CH₂CH₂O)ₘ—(CH₂)ₙ—R′, (CH₂CH₂O)ₘ—(CH₂)ₙ—OH, (CH₂CH₂O)ₘ—(CH₂)ₙ—OR′, (CH₂CH₂O)ₘ—(CH₂)ₙ—NR′R″, (CH₂CH₂O)ₘ—(CH₂)ₙ—NR′COR″, (CH₂CH₂O)ₘ(CH₂)ₙ—NR′SO₂R″, (CH₂CH₂O)ₘ(CH₂)ₙ—COOH, (CH₂CH₂O)ₘ(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ SONR′R″, (CH₂CH₂O)ₘ—(CH₂)ₙ—SO₂NR′R″, (CH₂)ₚ—(CH₂CH₂O)ₘ—(CH₂)ₙR′, (CH₂)ₚ—(CH₂CH₂O)ₘ—(CH₂)ₙ OH, (CH₂)ₚ—(CH₂CH₂O)ₘ—(CH₂)ₙ—OR′, (CH₂)ₚ—(CH₂CH₂O)ₘ—(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂CH₂O)ₘ—(CH₂)ₙ—COOR′, (CH₂)ₚ—(CH₂CH₂O)ₘ—(CH₂)ₙ—CONR′R″, (CH₂)ₚ—(CH₂CH₂O)ₘ—(CH₂)ₙ—SO₂R′, (CH₂)ₚ—(CH₂CH₂O)ₘ—(CH₂)ₙ—COR′, (CH₂)ₚ—(CH₂CH₂O)ₘ—(CH₂)ₙ—SONR′R″, (CH₂)ₚ—(CH₂CH₂O)ₘ—(CH₂)ₙ—SO₂NR′R″, Aryl-(CH₂)ₙ—COOH, and heteroaryl-alkyl-CO-alkyl-NR′R″ₘ, wherein the alkyl may be substituted with OR′, and heteroaryl-(CH₂)ₙ-heterocycle wherein the heterocycle may optionally be substituted with alkyl, hydroxyl, COOR′ and COR′; wherein R′ and R″ are selected from H, alkyl, alkyl substituted with halogen, hydroxyl, NH2, NH(alkyl), N(alkyl)₂, oxo, carboxy, cycloalkyl and heteroaryl; m, n, and p are independently 0 to 6;

at each occurrence R_{12′} of Formula A-4-1 through A-4-6 is independently selected from the group consisting of —O-(alkyl), —O-(alkyl)-alkoxy, —C(═O)-(alkyl), —C(═O)-alkyl-alkoxy, —C(═O)—NH-(alkyl), —C(═O)—N-(alkyl)₂, —S(═O)-(alkyl), S(═O)₂-(alkyl), —C(═O)-(cyclic amine), and —O-aryl-(alkyl), —O-aryl-(alkoxy);

at each occurrence R_{1″} of Formula A-4-1 through A-4-6 is independently selected from the group consisting of alkyl, aryl substitituted alkyl, aloxy substituted alkyl, cycloalkyl, ary-substituted cycloalkyl, and alkoxy substituted cycloalkyl.

In various embodiments, the alkyl or alkoxy groups in Formula A-4-1 through A-4-6 can be a lower alkyl or lower alkoxy, respectively.

In certain embodiments, the linker connection position of Formula A-4-1 through A-4-6 is at least one of Z, R_{8′}, R_{9′}, R_{10′}, R_{11″}, R_{12″}, or R_{1″}.

Suitable MDM2 binding moieties include, but are not limited to, the following:

1. The HDM2/MDM2 inhibitors identified in SCIENCE vol: 303, page: 844-848 (2004) and Bioorg. Med. Chem. Lett. 18 (2008) 5904-5908, including (or additionally) the compounds nutlin-3, nutlin-2, and nutlin-1 (derivatized) as described below, as well as all derivatives and analogs thereof:

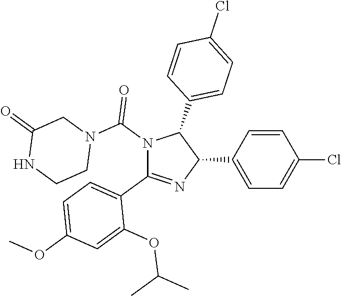 [SMILES: COc1ccc(C2=N[C@@H](c3ccc(Cl)cc3)[C@@H](c3ccc(Cl)cc3)N2C(=O)N2CCNC(=O)C2)c(OC(C)C)c1]

(derivatized where a linker group L or a -(L-ULM) group is attached, for example, at the methoxy group or as a hydroxyl group);

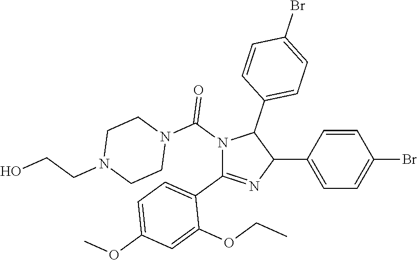 [SMILES: CCOc1cc(OC)ccc1C1=NC(c2ccc(Br)cc2)C(c2ccc(Br)cc2)N1C(=O)N1CCN(CCO)CC1]

(derivatized where a linker group L or a -(L-ULM) group is attached, for example, at the methoxy group or hydroxyl group);

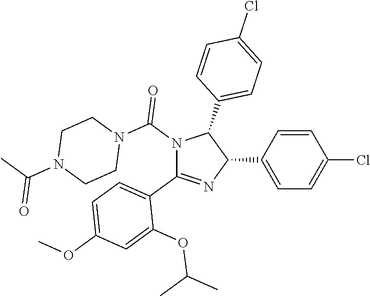 [SMILES: COc1ccc(C2=N[C@@H](c3ccc(Cl)cc3)[C@@H](c3ccc(Cl)cc3)N2C(=O)N2CCN(C(C)=O)CC2)c(OC(C)C)c1]

(derivatized where a linker group L or a -(L-ULM) group is attached, for example, via the methoxy group or as a hydroxyl group).

2. Trans-4-Iodo-4′-Boranyl-Chalcone

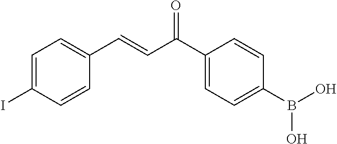 [SMILES: O=C(/C=C/c1ccc(I)cc1)c1ccc(B(O)O)cc1]

(derivatized where a linker group L or a a linker group L or a-(L-ULM) group is attached, for example, via a hydroxy group).

Preparation of Compounds of the Invention

Compounds of Formulas (I)-(XXIV) or otherwise described herein can be prepared by the general schemes described herein, using the synthetic method known by those skilled in the art. The following examples illustrate non-limiting embodiments of the invention.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compounds of the invention may exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compounds of the invention are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

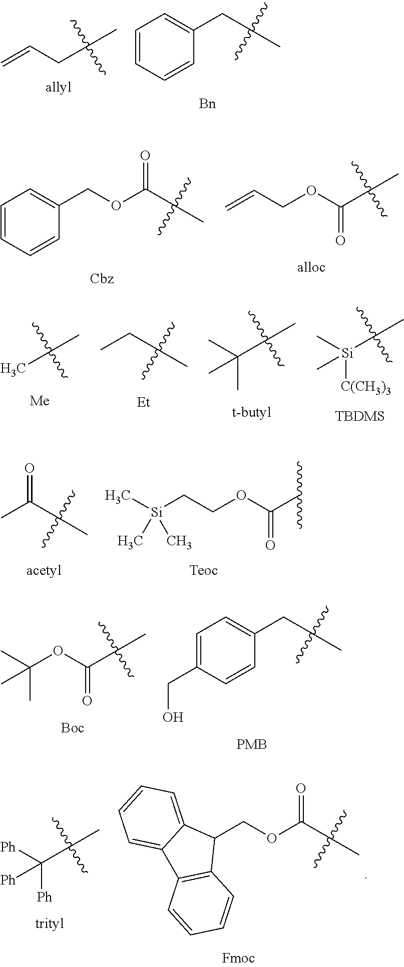

Other protecting groups, plus a detailed description of techniques applicable to the creation of protecting groups and their removal are described in Greene & Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

### Compositions

The invention includes a pharmaceutical composition comprising at least one compound of the invention and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

### Methods of Treatment

The invention includes a method of treating or preventing a disease associated with and/or caused by overexpression and/or uncontrolled activation of a tyrosine kinase in a subject in need thereof. The invention further includes a method of treating or preventing a cancer associated with and/or caused by an oncogenic tyrosine kinase in a subject in need thereof. In certain embodiments, the disease comprises a cancer. In other embodiments, the tyrosine kinase is c-ABL and/or BCR-ABL. In yet other embodiments, the cancer is chronic myelogenous leukemia (CML).

Examples of cancers that can be treated or prevented by the present invention include but are not limited to: squamous cell cancer, lung cancer including small cell lung cancer, non-small cell lung cancer, vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, and head and neck cancer. In certain embodiments, the cancer is at least one selected from the group consisting of ALL, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, Philadelphia chromosome positive CML, lymphoma, leukemia, multiple myeloma myeloproliferative diseases, large B cell lymphoma, and B cell Lymphoma.

The methods of the invention comprise administering to the subject a therapeutically effective amount of at least one compound of the invention, which is optionally formulated in a pharmaceutical composition. In various embodiments, a therapeutically effective amount of at least one compound of the invention present in a pharmaceutical composition is the only therapeutically active compound in a pharmaceutical composition. In certain embodiments, the method further comprises administering to the subject an additional therapeutic agent that treats or prevents cancer.

In certain embodiments, administering the compound of the invention to the subject allows for administering a lower dose of the additional therapeutic agent as compared to the dose of the additional therapeutic agent alone that is required to achieve similar results in treating or preventing a cancer in the subject. For example, in certain embodiments, the compound of the invention enhances the anti-cancer activity of the additional therapeutic compound, thereby allowing for a lower dose of the additional therapeutic compound to provide the same effect.

In certain embodiments, the compound of the invention and the therapeutic agent are co-administered to the subject. In other embodiments, the compound of the invention and the therapeutic agent are coformulated and co-administered to the subject.

In certain embodiments, the subject is a mammal. In other embodiments, the mammal is a human.

### Combination Therapies

The compounds useful within the methods of the invention may be used in combination with one or more additional therapeutic agents useful for treating a cancer. These additional therapeutic agents may comprise compounds that are commercially available or synthetically accessible to those skilled in the art. These additional therapeutic agents are known to treat, prevent, or reduce the symptoms, of a cancer.

In non-limiting examples, the compounds useful within the invention may be used in combination with one or more of the following therapeutic agents: Erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), pemetrexed (ALIMTA®, Eli Lilly), trastuzumab (HERCEPTIN®, Genentech), temozolomide (4-methyl-5-oxo-2,3,4,6,8-pentazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, rapamycin, oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®), ALK TKI inhibitors, antibodies such as avastin and cetuximab that target VEGFR and EGFR respectively, other RTK TKIs for PDGFR or RET, immunotherapies such as ipiliumimab and nivolumab, and radiation therapy.

In various embodiments, the compounds described herein, which are allosteric inhibitors of certain tyrosine kinases, can be used with an inhibitor that binds to the ATP binding site (catalytic site) of a tyrosine kinase. Without being bound by theory, it is believed that a combination of an allosteric inhibitor of a tyrosine kinase and an inhibitor that binds to the ATP site of the same tyrosine kinase can result in synergistic inhibition of the tyrosine kinase and/or reduce the resistance of patients to catalytic site inhibitor therapy. Resistance to catalytic site inhibitors (e.g., imatinib) can result from chronic use of such inhibitors to treat the cancers described herein.

In various embodiments, the compounds described herein can be used in combination with Ruxolitinib, Tofacitinib, Lapatinib, Vandetanib, Sorafenib, Sunitinib, Axitinib, Nintedanib, Regorafenib, Pazopanib, Lenvatinib, Crizotinib, Ceritinib, Cabozantinib, DWF, Afatinib, Ibrutinib, B43, KU004, Foretinib, KRCA-0008, PF-06439015, PF-06463922, Canertinib, GSA-10, GW2974, GW583340, WZ4002, CP-380736, D2667, Mubritinib, PD153035, PD168393, Pelitinib, PF-06459988, PF-06672131, PF-6422899, PKI-166, Reveromycin A, Tyrphostin 1, Tyrphostin 23, Tyrphostin 51, Tyrphostin AG 528, Tyrphostin AG 658, Tyrphostin AG 825, Tyrphostin AG 835, Tyrphostin AG 1478, Tyrphostin RG 13022, Tyrphostin RG 14620, B178, GSK1838705A, PD-161570, PD 173074, SU-5402, Roslin 2, Picropodophyllotoxin, PQ401, I-OMe-Tyrphostin AG 538, GNF 5837, GW441756, Tyrphostin AG 879, DMPQ, JNJ-10198409, PLX647, Trapidil, Tyrphostin A9, Tyrphostin AG 370, Lestaurtinib, DMH4, Geldanamycin, Genistein, GW2580, Herbimycin A, Lavendustin C, Midostaurin, NVP-BHG712, PD158780, PD-166866, PF-06273340, PP2, RPI, SU 11274, SU5614, Symadex, Tyrphostin AG 34, Tyrphostin AG 974, Tyrphostin AG 1007, UNC2881, Honokiol, SU1498, SKLB1002, CP-547632, JK-P3, KRN633, SC-1, ST638, SU 5416, Sulochrin, Tyrphostin SU 1498, S8567, rociletinib, Dacomitinib, Tivantinib, Neratinib, Masitinib, Vatalanib, Icotinib, XL-184, OSI-930, AB 1010, Quizartinib, AZD9291, Tandutinib, HM61713, Brigantinib, Vemurafenib (PLX-4032), Semaxanib, AZD2171, Crenolanib, Damnacanthal, Fostamatinib, Motesanib, Radotinib, OSI-027, Linsitinib, BIX02189, PF-431396, PND-1186, PF-03814735, PF-431396, sirolimus, temsirolimus, everolimus, deforolimus, zotarolimus, BEZ235, INK128, Omipalisib, AZD8055, MHY1485, PI-103, KU-0063794, ETP-46464, GDC-0349, XL388, WYE-354, WYE-132, GSK1059615, WAY-600, PF-04691502, WYE-687, PP121, BGT226, AZD2014, PP242, CH5132799, P529, GDC-0980, GDC-0994, XMD8-92, Ulixertinib, FR180204, SCH772984, Trametinib, PD184352, PD98059, Selumetinib, PD325901, UO 0126, Pimasertinib, TAK-733, AZD8330, Binimetinib, PD318088, SL-327, Refametinib, GDC-0623, Cobimetinib, BI-847325, Adaphostin, GNF 2, PPY A, AIM-100, ASP 3026, LFM A13, PF 06465469, (−)-Terreic acid, AG-490, BIBU 1361, BIBX 1382, BMS 599626, CGP 52411, GW 583340, HDS 029, HKI 357, JNJ 28871063, WHI-P 154, PF 431396, PF 573228, FIIN 1, PD 166285, SUN 11602, SR 140333, TCS 359, BMS 536924, NVP ADW 742, PQ 401, BMS 509744, CP 690550, NSC 33994, WHI-P 154, KB SRC 4, DDRI-IN-1, PF 04217903, PHA 665752, SU 16f, A 419259, AZM 475271, PP 1, PP 2, 1-Naphthyl PP1, Src II, ANA 12, PD 90780, Ki 8751, Ki 20227, ZM 306416, ZM 323881, AEE 788, GTP 14564, PD 180970, R 1530, SU 6668, and Toceranib, and combinations thereof.

In certain embodiments, the compounds of the present invention are used in combination with radiation therapy. In other embodiments, the combination of administration of the compounds of the present invention and application of radiation therapy is more effective in treating or preventing cancer than application of radiation therapy by itself. In yet other embodiments, the combination of administration of the compounds of the present invention and application of radiation therapy allows for use of lower amount of radiation therapy in treating the subject.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a cancer. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a cancer in the patient. An effective amount of the therapeutic compound necessary to achieve a therapeutic effect may vary according to factors such as the state of the disease or disorder in the patient; the age, sex, and weight of the patient; and the ability of the therapeutic compound to treat a cancer in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a cancer in a patient.

In certain embodiments, the compositions of the invention are formulated using one or more pharmaceutically acceptable excipients or carriers. In certain embodiments, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the invention are administered to the patient in dosages that range from one to five times per day or more. In other embodiments, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a cancer in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY—P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends on the age, sex and weight of the patient, the current medical condition of the patient and the progression of a cancer in the patient being treated. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

### EXAMPLES

Various embodiments of the present invention can be better understood by reference to the following Examples which are offered by way of illustration. The present invention is not limited to the Examples given herein.

### Methods and Materials

1. Biology

### Cell Lines and Materials

K562 cells were obtained from ATCC and were grown at 37° C., 5% CO₂ in Iscove's Modified Dulbecco's Media (IMDM) supplemented with 10% FBS, 100 U/mL penicillin and 100 μg/mL streptomycin. Phospho-STAT5 Y694 (#4322) and phospho-CrkL Y207 (#3181) antibodies were obtained from Cell Signaling Technologies. c-ABL (24-11) antibody was obtained from Santa Cruz Biotechnologies. α-Tubulin antibody (T9026) was purchased from Sigma-Aldrich.

Ba/F3 murine cell lines, either parental or with stable expression of BCR-ABL1 via pSRc vector backbone, were grown in R10 media consisting of RPMI (Invitrogen) supplemented with 10% FBS (Atlanta Biologicals), L-glutamine, penicillin/streptomycin (Invitrogen) and amphotericin B (HyClone). In Ba/F3 parental cells, WEHI-3B-conditioned medium (15%) was included as a source of IL-3. K562 cell lines were purchased from ATCC and cultured in IMDM (Gibco) supplemented with 10% FBS and penicillin/streptomycin (Invitrogen).

Patient Samples

Mononuclear cells (MNCs) were isolated from either peripheral blood, bone marrow or leukapheresis samples by Ficoll gradient. MNCs were then treated with ammonium-chloride-potassium (ACK) lysis buffer to remove any residual red blood cells. CD34+ cells were isolated via magnetic bead isolation (MACS, Miltenyi Biotec #130-046-703) and stored in liquid nitrogen storage in 90% FBS plus 10% DMSO for long term storage. For cell proliferation assays, samples were thawed and cultured in R10. For immunoblot assays, samples were thawed and cultured in IMDM with 40 μg/mL low-density lipoprotein (LDL, Stem Cell), 20% FBS, and 100 μM beta-mercaptaethanol (Sigma).

Flow Cytometry

Samples sorted for CD38 were stained with CD34 PE-Cy™7 (BD Biosciences #348801) and BV421 Mouse Anti-Human CD38 (BD Biosciences #562444), washed in sterile phosphate buffered saline (PBS) with 10% BSA, and sorted on a BD FACS Aria instrument. Sorted samples were recovered overnight in culture in IMDM with 40 μg/mL low-density lipoprotein (LDL, Stem Cell), 20% FBS, and 100 μM beta-mercaptaethanol (Sigma).

Western Blotting

K562 cells (1-1.5×10⁶) were treated for 24 hours with the indicated compounds solubilized in DMSO. The cells were collected at 300 g for 3 min. The cells were then lysed in lysis buffer (25 mM Tris, 1% Triton, 0.25% deoxycholic acid) with Roche protease inhibitor complete cocktail and phosphatase inhibitors (10 mM sodium fluoride, 10 mM sodium pyrophosphate, 1 mM sodium orthovanadate and 20 mM P3-glycerophosphate). The total protein concentrations were determined by Pierce BCA Protein Assay and 30-50 μg of protein was loaded onto 10% Tris-Glycine gels. After standard gel electrophoresis, the separated proteins were transferred to nitrocellulose by wet transfer. The immunoblots were then processed by standard procedures and incubated with the respective antibodies. Band intensities were quantified by Bio-Rad's Image Lab software.

Immunoblot Analysis

Samples were lysed in 1× Cell Signaling lysis buffer (#9803S) supplemented with PMSF and complete mini tablets (Sigma #11836153001). Lysates were quantified and separated on 4-15% Tris-glycine polyacrylamide gels. Gels were transferred and then blocked in TBS-Tween buffer with 5% BSA. The following primary antibodies were used: tABL, BD #554148 anti-mouse 1:400; c-ABL1, SantaCruz #24-11 anti-mouse 1:1000; B-tubulin, Millipore #05-66-MI anti-mouse 1:5000; pSTAT5, # CS9351S anti-rabbit 1:1000 pSTAT5, # CS4322 anti-rabbit 1:1000; pBCR-Abl, # CS2865 anti-rabbit 1:1000; pCRKL, # CS491 anti-rabbit 1:1000; pAKT, # CS4060 anti-rabbit 1:1000; pERK, # CS4695 anti-rabbit 1:1000; pSHP-2, Abcam #62322 anti-rabbit 1:1000; pGAB1, # CS3233 anti-rabbit 1:1000; pGAB2, # CS3882 anti-rabbit 1:1000; pSHC, # CS2434 anti-rabbit 1:1000; VHL, # CS68547 anti-rabbit 1:1000; pCRKL, # CS3181S anti-rabbit 1:1000. Following incubation with appropriate HRP-conjugated secondary antibodies, membranes were imaged on a BioRad ChemiDoc using BioRad Clarity Western ECL substrate and Thermo Super Signal West Femto Maximum Sensitivity substrate.

Cell Viability Assay

Cell lines and patient samples were exposed to dose ranges of single or combination agents and incubated for 3 days at 37° C., 5% CO₂ and subjected to a CellTiter 96 AQueous One solution cell proliferation assay (Promega). IC₅₀ values calculated and analyzed using Prism 6 software (GraphPad).

Apoptosis Analysis

Patient samples were incubated ranging from 48 to 96 h, stained and analyzed according to the Guava Nexin Reagent analysis kit (Milipore #4500-0450) or ApoScreen Annexin V-FITC (Southern Biotech #10040-02) for flow cytometry analysis.

Reverse Phase Protein Arrays (RPPA)

K562 cells were treated with DMSO, Compound 10 (5 μM) or Compound 14 (5 μM) for 8 h in duplicate, washed twice with PBS and lysed in RPPA lysis buffer (1% Triton X-100, 50 mM HEPES, pH 7.4, 150 mM NaCl, 1.5 mM MgCl₂, 1 mM EGTA, 100 mM NaF, 10 mM Na pyrophosphate, 1 mM Na₃VO₄, 10% glycerol, containing freshly added protease and phosphatase inhibitors from Roche Applied Science). RPPA was performed in the MDACC CCSG core as described at http://www.mdanderson.org/education-and-research/resources-for-professionals/scientific-resources/core-facilities-and-services/functional-proteomics-rppa-core/index.html.

Bioassays

Ba/F3 cells expressing wild-type BCR-ABL, were distributed in 384-well plates in complete medium (RPMI 1640+10% FBS, pen/strep, L-glutamine, and fungizone) at 1000 cells/well; Ba/F3 parental cells in complete medium supplemented with 15% WEHI-3B-conditioned media (as a source of IL-3) were also tested.

Imatinib, Compound 10, and Compound 14 were plated using an HP D300 Drug Printer with each condition in quadruplicate. Each of imatinib, Compound 10, and Compound 14 were plated with the following concentrations:

Imatinib: 0, 0.025, 0.1, 0.25, 1, 2. μM.

Compound 10 and Compound 14 (each): 0, 0.1, 0.25, 1, 2.5, 1 μM.

Plates were incubated at 37° C. for 72 h and analyzed by standard MTS-based colorimetric assay. Non-linear regression curve-fit analysis and IC₅₀ calculations were performed using Graphpad Prism software.

MNCs isolated from specimens via ficoll, then selected for CD34+ by MACs column. Frozen viables were thawed and cultured in IMDM+40 ug/mL LDL+20% FBS+100 uM BME overnight. Sorted using FACS Aria III, stained with CD34 (BD348801) and CD38 (BD555461).

Serum starved for 3 h then treated with increasing concentrations of Compound 1 and Compound 14 overnight. Cells were lysed in Cell Signaling lysis buffer supplemented with PMSF and Complete Mini protease inhibitor tablets. Protein concentration assessed via BCA assay, ran on 4-15% tris-glycine polyacrylamide gel at 180V for 1 h, transferred overnight at 25V, blocked with 5% BSA in TBST for 1 h and incubated at 4 degree in following primary antibodies.

tABL: BD 554148 anti-mouse; 1:400

pCRKL: CS3181S anti-rabbit; 1:1000

B-tubulin: Millipore 05-66—MI anti-mouse; 1:5000

pSTAT5: CS9351S anti-rabbit; 1:1000

Imaged on BioRad ChemiDoc using BioRad Clarity Western ECL substrate and Thermo Super Signal West Femto Maximum Sensitivity substrate.

K562 cells were implanted subcutaneously in the flank of athymic mice and tumors allowed to develop to approximately 300 mm3. Mice were treated with Compound 15 or the relevant control every 24 hours (200 mg/kg, IP) for 3 days and tumor volumes measured (FIG. 3). Select biological data for compounds of Formula (I) is shown in Table 1.

TABLE 1
Biological data for compounds of Formula (I)

| | | |
|---|---|---|
| | DC₅₀ or IC₅₀(μM) | Dₘₐₓ (%) |
| Compound Name | [cell line] | [cell line] |

| | | | | |
|---|---|---|---|---|
| Compound 1 | 1 μM | [K562] | | |
| Compound 10 | 515 nM | [K562] | 95% | [K562] |
| Compound 14 | 30 nM | [K562] | >95% | [K562] |

Immunoblot Analysis for FIGS. 14B, 16A, 17, and 18

All cell lines were confirmed mycoplasma negative prior to experimental use.

K562 cells were treated with the indicated concentrations of compound for 24 hours, washed with PBS and then harvested in lysis buffer (50 mM Tris (pH 7.4), 150 mM NaCl, 1 mM EDTA, 0.5% NP-40, phosphatase and protease inhibitors). Following centrifugation at 13,000×g for 15 minutes at 4° C. to pellet insoluble materials, the protein concentrations of the supernatants were quantitated by BCA assay (Thermo Fisher Scientific). Protein samples were resolved by SDS-PAGE, transferred to nitrocellulose, blocked with 5% non-fat milk and probed with the marked antibodies. Immunoblots were developed using enhanced chemiluminescence and visualized using a Bio-Rad Chemi-Doc MP Imaging System with Image Lab v.5.2.1 software (Bio-Rad Laboratories).

Pharmacokinetics

The pharmacokinetic properties were determined at Pharmaron, China. The exemplary compound was formulated (in 5% EtOH & 5% Solutol HS15 in D5W(ESD-2)) and administered by intraperitoneal (I.P.) injection at a dosing level of 10 mg/kg. Plasma samples were collected at 0.25 hours, 0.5 hours, 1 hour, 2 hours, 4 hours, 8 hours, and 24 hours following dosing.

Acetonitrile was added to precipitate protein and the samples were vortexed for 30 second, centrifuged (4000 rpm, 15 minutes), diluted with water and the compound concentration determined by LC/MS/MS according to a standard curve.

In Vivo Xenograft Model

All in vivo experiments were conducted in accordance with institutional guidelines and were approved by Yale Institutional Animal Care & Use Committee. Mice were housed in pathogen-free animal facilities and with access to food and water ad libitum. K562 cells in Matrigel (Corning Life Sciences) were injected subcutaneously into the flank of athymic mice (Charles River) and allowed to proliferate until tumors reached −200 mm³ at which point mice were randomized into treatment groups and treated I.P. with vehicle or exemplary compound once a day at 200 mg/kg for 3 days. Tumors were measured via caliper and volumes calculated following equation:

V=0.5×L×W²,

where V=volume, L=Length and W=Width

2. Chemistry

### General Methods

All reactions were carried out under an atmosphere of dry nitrogen or argon. Glassware was oven-dried prior to use. Unless otherwise indicated, common reagents or materials were obtained from commercial source and used without further purification. N,N-Diisopropylethylamine (DIPEA) was obtained anhydrous by distillation over potassium hydroxide. Tetrahydrofuran (THF), Dichloromethane (CH₂Cl₂), and dimethylforamide (DMF) was dried by a PureSolv™ solvent drying system. Flash column chromatography was performed using silica gel 60 (230-400 mesh). Analytical thin layer chromatography (TLC) was carried out on Merck silica gel plates with QF-254 indicator and visualized by UV or KMnO₄.

A synthetic scheme for synthesizing ATKI moieties derived from GNF-2 is shown Scheme 1:

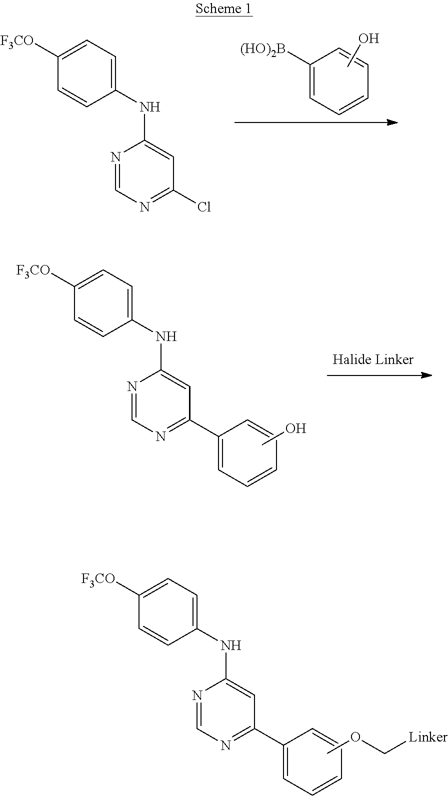

A synthetic scheme for synthesizing ATKI moieties derived from asciminib and covalently bonded to L with ether linkages is shown in Scheme 2:

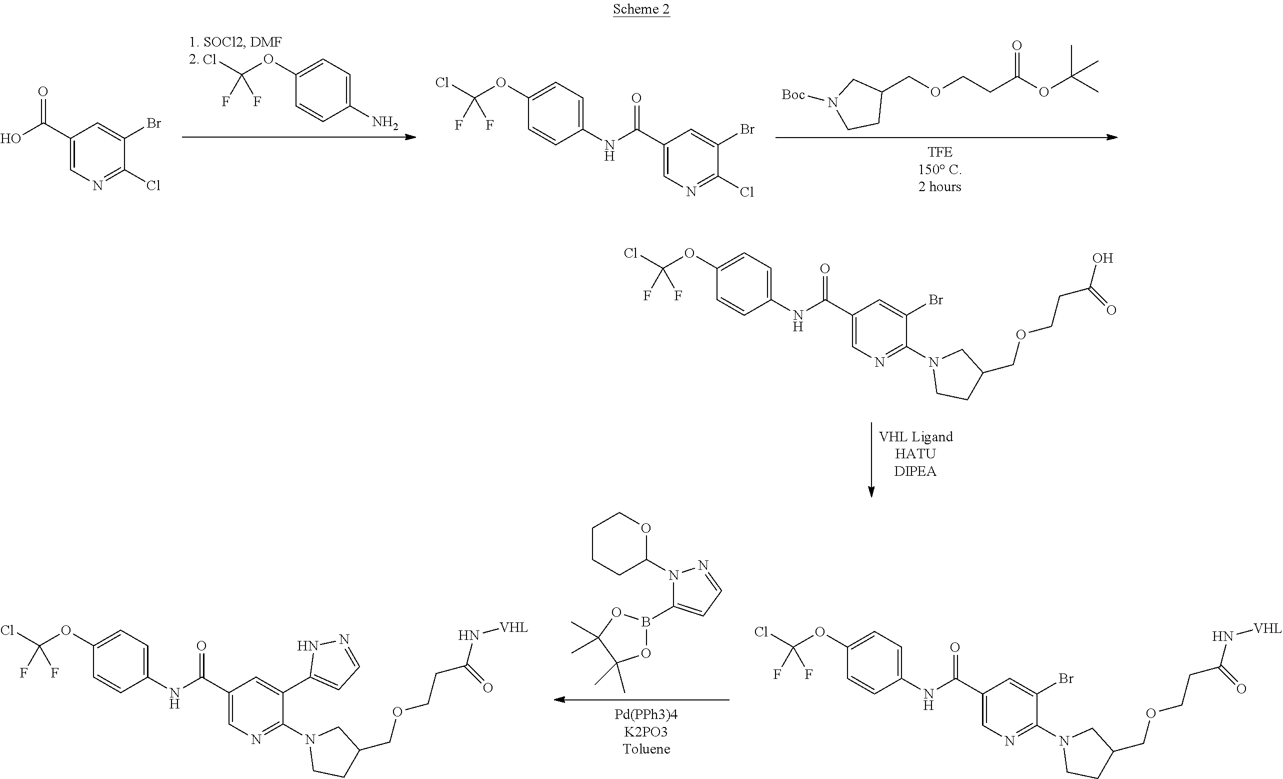

A synthetic scheme for the complete synthesis of an ATKI-containing PROTAC is shown in Scheme 3:

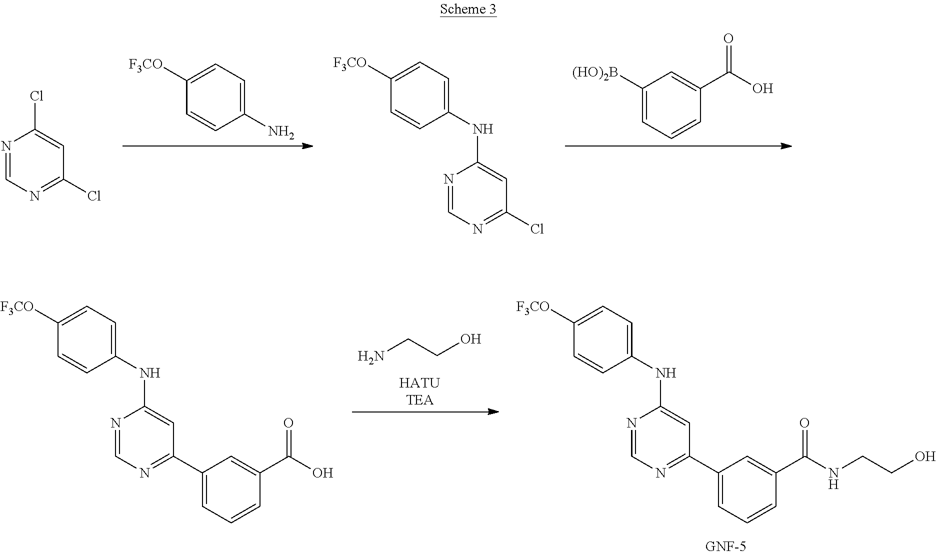

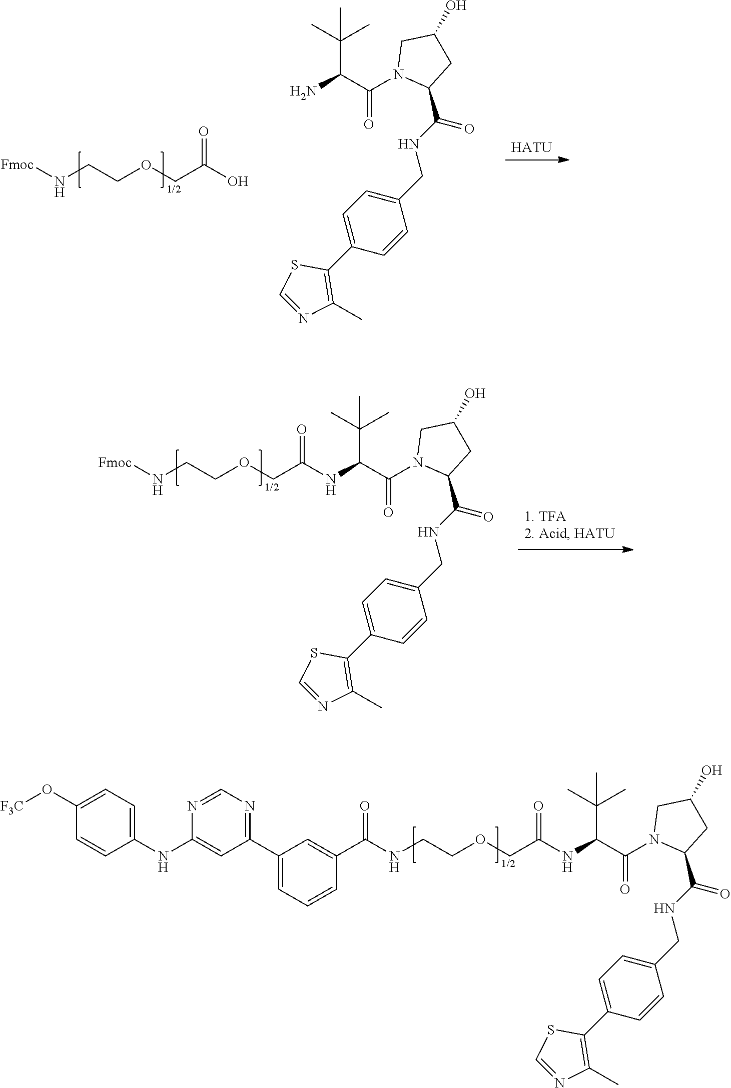

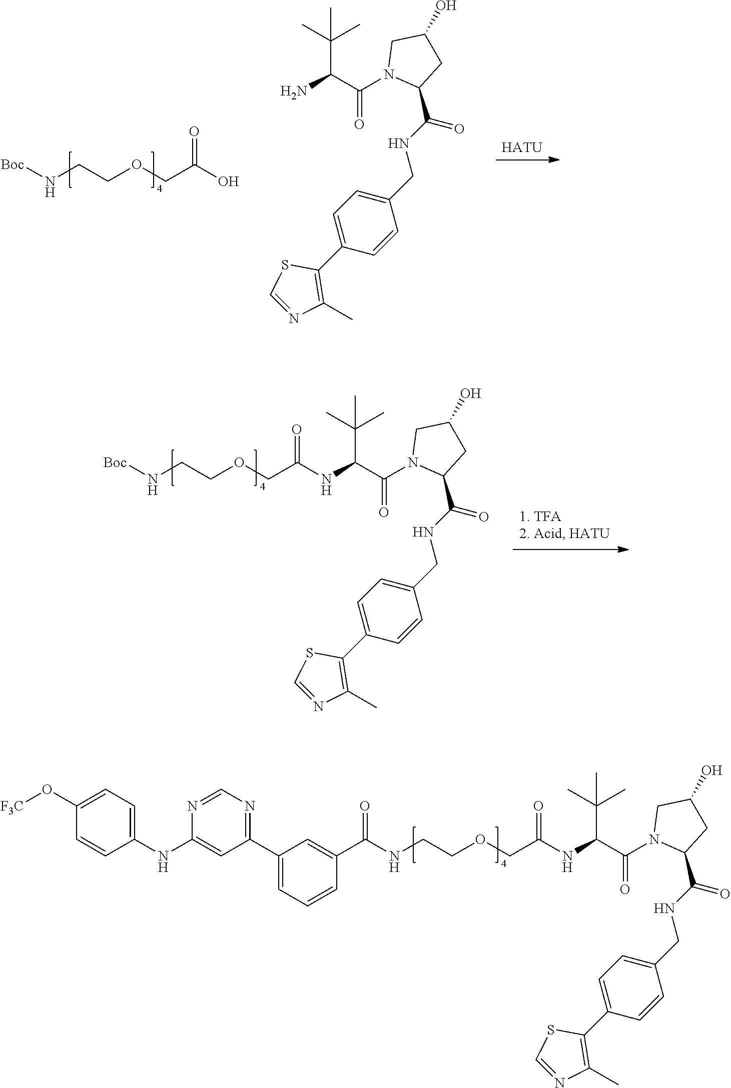

### 6-chloro-N-(4-(trifluoromethoxy)phenyl)pyrimidin-4-amine

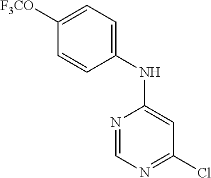 [SMILES: FC(F)(F)Oc1ccc(Nc2cc(Cl)ncn2)cc1]

Following a literature procedure, 4,6-dichloropyrimidine (1 g, 6.71 mmol) and 4-(trifluoromethoxy)aniline (1189 mg, 6.71 mmol) suspended in ethanol (30 ml) and triethylamine (0.93 ml, 6.71 mmol) added. Reaction heated to reflux overnight, allowed to cool to r.t. and concentrated in vacuo. Residue purified by column chromatography eluting with 0-6% methanol/DCM to yield the title compound as an off white solid (1.04 g, 54%).

Characterization data matched the literature report in X. Deng, et al., Journal of Medicinal Chemistry, 2010, 53, 6934-6946.

### 3-(6-((4-(trifluoromethoxy)phenyl)amino)pyrimidin-4-yl)benzoic acid

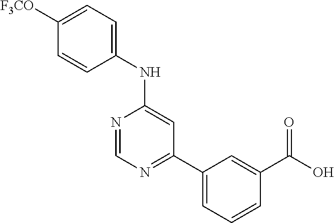 [SMILES: O=C(O)c1cccc(-c2cc(Nc3ccc(OC(F)(F)F)cc3)ncn2)c1]

Following a literature procedure, 6-chloro-N-(4-(trifluoromethoxy)phenyl)pyrimidin-4-amine (300 mg, 1.04 mmol), 3-boronobenzoic acid (172 mg, 1.04 mmol), tetrakis(triphenylphosphine)palladium(0) (120 mg, 10 mol %) and sodium carbonate (439 mg, 4.14 mmol) were heated to reflux in a 1:1 mixtures of acetonitrile/water (20 ml) overnight. The resulting suspension was filtered hot, cooled to r.t., adjusted to pH 4 with conc. HCl. The resulting precipitate was collected by filtration, washed with water and dried in vacuo (232 mg, 60%)

Characterization data matched the literature report in X. Deng, et al., Journal of Medicinal Chemistry, 2010, 53, 6934-6946.

### (9H-fluoren-9-yl)methyl (2-(2-(((S)-1-((2S,4R)-4-hydroxy-2-((4-(4-methylthiazol-5-yl)benzyl) carbamoyl)pyrrolidin-1-yl)-3,3-dimethyl-1-oxobutan-2-yl)amino)-2-oxoethoxy)ethyl)carbamate

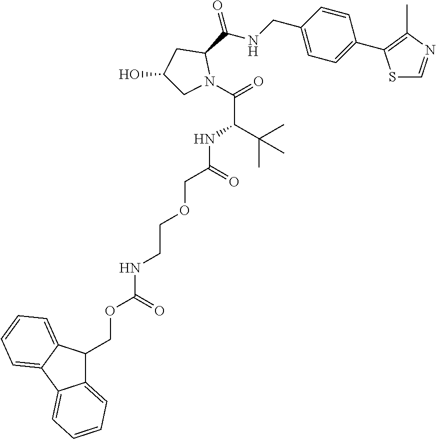 [SMILES: Cc1ncsc1-c1ccc(CNC(=O)[C@@H]2C[C@@H](O)CN2C(=O)[C@@H](NC(=O)COCCNC(=O)OCC2c3ccccc3-c3ccccc32)C(C)(C)C)cc1]

2-[2-(9H-Fluoren-9-ylmethoxycarbonylamino)ethoxy]acetic acid (87 mg, 0.255 mmol) was dissolved in DMF (20 ml) and treated with HATU (97 mg, 0.255 mmol) and triethylamine (97 μl, 0.697 mmol) followed by (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (100 mg, 0.232 mmol). The reaction was stirred at r.t. overnight, diluted with water (20 ml) and extracted with ethyl acetate (2×15 ml). The organic layers were combined, washed with 2 M HCL (10 ml) and 10% LiCl (aq) (2×10 ml), dried over MgSO₄ and concentrated in vacuo. The residue was purified by column chromatography eluting with 5% MeOH/DCM to yield the title compound (34 mg, 20%). HRMS: calc. [M+H]⁺ for C₄₁H₄₇N₅O₇S=754.3269; found=754.3395 [M+H]⁺.

### (9H-fluoren-9-yl)methyl (2-(2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)ethyl)carbamate

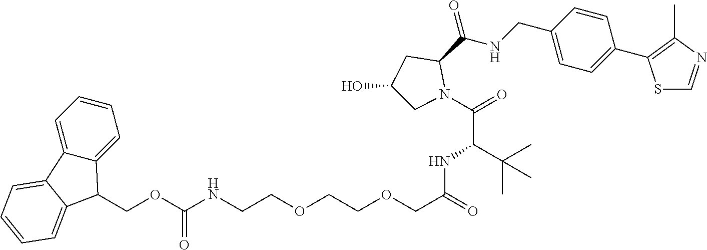 [SMILES: Cc1ncsc1-c1ccc(CNC(=O)[C@@H]2C[C@@H](O)CN2C(=O)[C@@H](NC(=O)COCCOCCNC(=O)OCC2c3ccccc3-c3ccccc32)C(C)(C)C)cc1]

2-[2-[2-(9H-Fluoren-9-ylmethoxycarbonylamino)ethoxy]ethoxy]acetic acid (98 mg, 0.255 mmol) was dissolved in DMF (20 ml) and treated with HATU (97 mg, 0.255 mmol) and triethylamine (97 μl, 0.697 mmol) followed by (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (100 mg, 0.232 mmol). The reaction was stirred at r.t. overnight, diluted with water (20 ml) and extracted with ethyl acetate (2×15 ml). The organic layers were combined, washed with 2 M HCL (10 ml) and 10% LiCl (aq) (2×10 ml), dried over MgSO₄ and concentrated in vacuo. The residue was purified by column chromatography eluting with 5% MeOH/DCM to yield the title compound (142 mg, 76%). HRMS: calc. [M+H]⁺ for C₄₃H₅₁N₅O₈S=798.3531; found=798.3671 [M+H]⁺.

### tert-butyl ((S)-17-((2S,4R)-4-hydroxy-2-((4-(4-methylthiazol-5-yl)benzyl)carbamoyl)pyrrolidine-1-carbonyl)-18,18-dimethyl-15-oxo-3,6,9,12-tetraoxa-16-azanonadecyl)carbamate

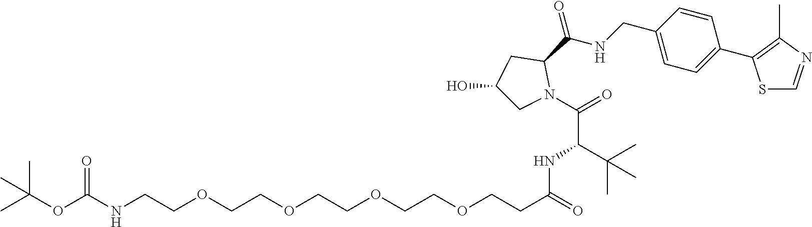 [SMILES: Cc1ncsc1-c1ccc(CNC(=O)[C@@H]2C[C@@H](O)CN2C(=O)[C@@H](NC(=O)CCOCCOCCOCCOCCNC(=O)OC(C)(C)C)C(C)(C)C)cc1]

3-[2-[2-[2-[2-(tert-Butoxycarbonylamino)ethoxy]ethoxy]ethoxy]ethoxy]propanoic acid (94 mg, 0.255 mmol) was dissolved in DMF (20 ml) and treated with HATU (97 mg, 0.255 mmol) and triethylamine (97 μl, 0.697 mmol) followed by (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (100 mg, 0.232 mmol). The reaction was stirred at r.t. overnight, diluted with water (20 ml) and extracted with ethyl acetate (2×15 ml). The organic layers were combined, washed with 2 M HCL (10 ml) and 10% LiCl (aq) (2×10 ml), dried over MgSO₄ and concentrated in vacuo. The residue was purified by column chromatography eluting with 5% MeOH/DCM to yield the title compound (166 mg, 92%). HRMS: calc. [M+H]⁺ for C₃₈H₅₉N₅O₁₀S=778.4055; found=778.4243 [M+H]⁺.

### (2S,4R)-1-((S)-3,3-dimethyl-2-(2-(2-(3-(6-((4-(trifluoromethoxy)phenyl)amino) pyrimidin-4-yl)benzamido)ethoxy)acetamido)butanoyl)-4-hydroxy-N-(4-(4-methylthiazol-5-yl)benzyl)pyrrolidine-2-carboxamide

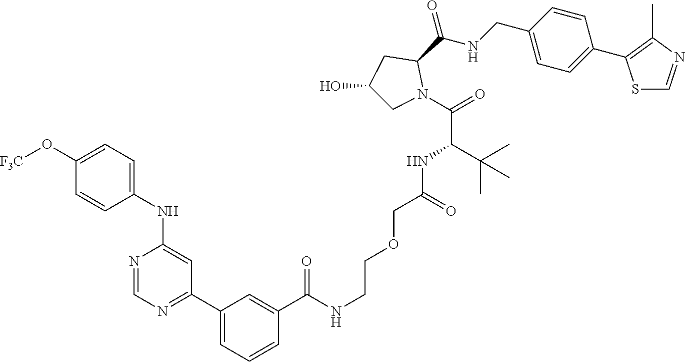 [SMILES: Cc1ncsc1-c1ccc(CNC(=O)[C@@H]2C[C@@H](O)CN2C(=O)[C@@H](NC(=O)COCCNC(=O)c2cccc(-c3cc(Nc4ccc(OC(F)(F)F)cc4)ncn3)c2)C(C)(C)C)cc1]

(9H-Fluoren-9-yl)methyl (2-(2-(((S)-1-((2S,4R)-4-hydroxy-2-((4-(4-methylthiazol-5-yl)benzyl) carbamoyl)pyrrolidin-1-yl)-3,3-dimethyl-1-oxobutan-2-yl)amino)-2-oxoethoxy)ethyl)carbamate (32 mg, 0.042 mmol) dissolved in DMF (5 ml) and treated with TBAF (27 mg, 0.085 mmol) and reaction mixture stirred for 5 minutes. After which time a solution of 3-[6-[4-(trifluoromethoxy)anilino]pyrimidin-4-yl]benzoic acid (24 mg, 0.064 mmol), HATU (33 mg, 0.085 mmol) and triethylamine (30 μl, 0.212 mmol) in DMF (5 ml) was added and the reaction mixture was stirred overnight at r.t. The reaction mixture diluted with water (20 ml) and extracted with ethyl acetate (2×20 ml). Organics washed with brine and 10% lithium chloride, then extracted with 2M HCl (2×20 ml). Acidic extract adjusted to pH 4 with NaOH (aq) and extracted with ethyl acetate (2×10 ml). Organics combined, dried over MgSO₄ and concentrated in vacuo. Purified by column chromatography eluting with 5-10% MeOH/DCM to give a colorless solid (12 mg, 32%). HRMS: calc. [M+H]⁺ for C₄₄H₄₇F₃N₈O₇S=889.3313; found=889.3423 [M+H]⁺.

### (2S,4R)-1-((S)-3,3-dimethyl-2-(2-(2-(3-(6-(phenylamino)pyrimidin-4-yl)benzamido)ethoxy) acetamido)butanoyl)-4-hydroxy-N-(4-(4-methylthiazol-5-yl)benzyl)pyrrolidine-2-carboxamide

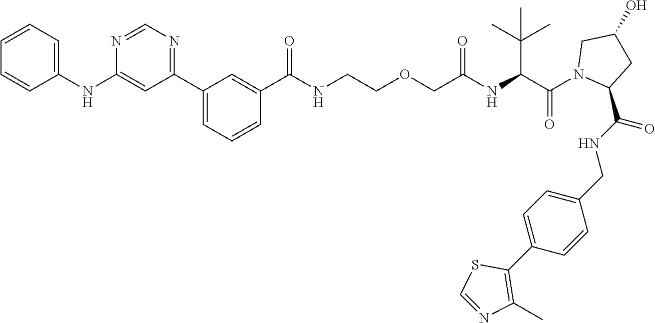 [SMILES: Cc1ncsc1-c1ccc(CNC(=O)[C@@H]2C[C@@H](O)CN2C(=O)[C@@H](NC(=O)COCCNC(=O)c2cccc(-c3cc(Nc4ccccc4)ncn3)c2)C(C)(C)C)cc1]

Prepared in the same manner as (2S,4R)-1-((S)-3,3-dimethyl-2-(2-(2-(3-(6-((4-(trifluoromethoxy)phenyl)amino)pyrimidin-4-yl)benzamido)ethoxy)acetamido)butanoyl)-4-hydroxy-N-(4-(4-methylthiazol-5-yl)benzyl)pyrrolidine-2-carboxamide. HRMS: calc. [M+H]⁺ for C₄₃H₄₈N₈O₆S=805.3490; found=805.3968 [M+H]⁺.

### (2S,4R)-1-((S)-12-(tert-butyl)-1,10-dioxo-1-(3-(6-((4-(trifluoromethoxy)phenyl)amino)pyrimidin-4-yl)phenyl)-5,8-dioxa-2,11-diazatridecan-13-oyl)-4-hydroxy-N-(4-(4-methylthiazol-5-yl)benzyl)pyrrolidine-2-carboxamide

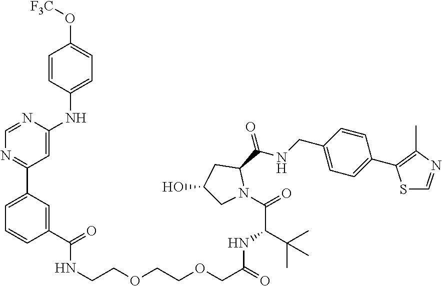 [SMILES: Cc1ncsc1-c1ccc(CNC(=O)[C@@H]2C[C@@H](O)CN2C(=O)[C@@H](NC(=O)COCCOCCNC(=O)c2cccc(-c3cc(Nc4ccc(OC(F)(F)F)cc4)ncn3)c2)C(C)(C)C)cc1]

(9H-Fluoren-9-yl)methyl (2-(2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy) ethoxy)ethyl)carbamate (64 mg, 0.080 mmol) dissolved in DMF (5 ml) and treated with TBAF (27 mg, 0.085 mmol) and reaction mixture stirred for 5 minutes. After which time a solution of 3-[6-[4-(trifluoromethoxy)anilino]pyrimidin-4-yl]benzoic acid (45 mg, 0.120 mmol), HATU (61 mg, 0.160 mmol) and triethylamine (56 μl, 0.40 mmol) in DMF (5 ml) was added and the reaction mixture was stirred overnight at r.t. The reaction mixture diluted with water (20 ml) and extracted with ethyl acetate (2×20 ml). Organics washed with brine and 10% lithium chloride, then extracted with 2M HCl (2×20 ml). Acidic extract adjusted to pH 4 with NaOH (aq) and extracted with ethyl acetate (2×10 ml). Organics combined, dried over MgSO₄ and concentrated in vacuo. Purified by column chromatography eluting with 5-10% MeOH/DCM to give a colorless solid (18 mg, 24%). HRMS: calc. [M+H]⁺ for C₄₆H₅₁F₃N₈O₈S=933.3575; found=933.3671 [M+H]⁺.

### (2S,4R)-1-((S)-1-amino-17-(tert-butyl)-15-oxo-3,6,9,12-tetraoxa-16-azaoctadecan-18-oyl)-4-hydroxy-N-(4-(4-methylthiazol-5-yl)benzyl)pyrrolidine-2-carboxamide

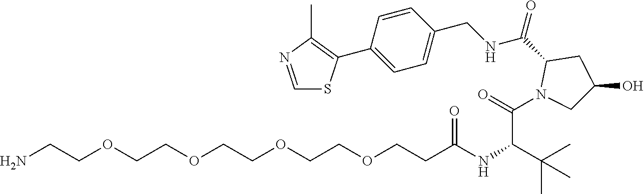 [SMILES: Cc1ncsc1-c1ccc(CNC(=O)[C@@H]2C[C@@H](O)CN2C(=O)[C@@H](NC(=O)CCOCCOCCOCCOCCN)C(C)(C)C)cc1]

Tert-butyl ((S)-17-((2S,4R)-4-hydroxy-2-((4-(4-methylthiazol-5-yl)benzyl) carbamoyl)pyrrolidine-1-carbonyl)-18,18-dimethyl-15-oxo-3,6,9,12-tetraoxa-16-azanonadecyl)carbamate (20 mg, 0.026 mmol) dissolved in 20% TFA/DCM and stirred overnight at r.t. Concentrated in vacuo and used immediately.

### (2S,4R)-1-((S)-19-(tert-butyl)-1,17-dioxo-1-(3-(6-((4-(trifluoromethoxy)phenyl)amino)pyrimidin-4-yl)phenyl)-5,8,11,14-tetraoxa-2,18-diazaicosan-20-oyl)-4-hydroxy-N-(4-(4-methylthiazol-5-yl)benzyl)pyrrolidine-2-carboxamide

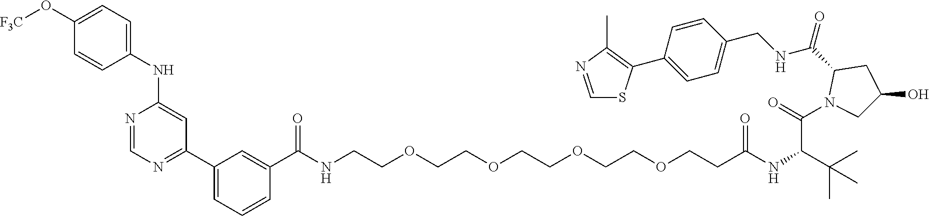 [SMILES: Cc1ncsc1-c1ccc(CNC(=O)[C@@H]2C[C@@H](O)CN2C(=O)[C@@H](NC(=O)CCOCCOCCOCCOCCNC(=O)c2cccc(-c3cc(Nc4ccc(OC(F)(F)F)cc4)ncn3)c2)C(C)(C)C)cc1]

(2S,4R)-1-((S)-1-Amino-17-(tert-butyl)-15-oxo-3,6,9,12-tetraoxa-16-azaoctadecan-18-oyl)-4-hydroxy-N-(4-(4-methylthiazol-5-yl)benzyl)pyrrolidine-2-carboxamide (17 mg, 0.026 mmol) dissolved in DMF (5 ml) and treated with 3-[6-[4-(trifluoromethoxy)anilino]pyrimidin-4-yl]benzoic acid (10 mg, 0.026 mmol), HATU (12 mg, 0.032 mmol) and triethylamine (15 μl, 0.10 mmol) and the reaction mixture was stirred overnight at r.t. The reaction mixture diluted with water (20 ml) and extracted with ethyl acetate (2×20 ml). Organics washed with brine and 10% lithium chloride, then extracted with 2M HCl (2×20 ml). Acidic extract adjusted to pH 4 with NaOH (aq) and extracted with ethyl acetate (2×10 ml). Organics combined, dried over MgSO₄ and concentrated in vacuo. Purified by column chromatography eluting with 5-10% MeOH/DCM to give a colorless solid (7 mg, 27%). HRMS: calc. [M+H]⁺ for C₅₁H₆₁F₃N₈O₈S=1035.4256; found=1035.4401 [M+H]⁺.

### 3-(6-((4-(trifluoromethoxy)phenyl)amino)pyrimidin-4-yl)phenol

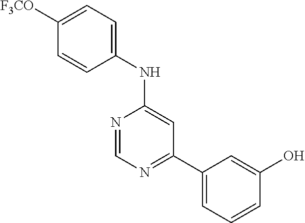 [SMILES: Oc1cccc(-c2cc(Nc3ccc(OC(F)(F)F)cc3)ncn2)c1]

Following an adapted literature procedure,¹ 6-chloro-N-(4-(trifluoromethoxy)phenyl) pyrimidin-4-amine (500 mg, 1.73 mmol), (3-hydroxyphenyl)boronic acid (238 mg, 1.73 mmol), tetrakis(triphenylphosphine)palladium(0) (199 mg, 10 mol %) and sodium carbonate (732 mg, 6.91 mmol) were heated to reflux in a 1:1 mixtures of acetonitrile/water (20 ml) overnight. The resulting suspension was filtered hot, cooled to r.t., adjusted to pH 4 with conc. HCl. The resulting precipitate was collected by filtration, washed with water and dried in vacuo (472 mg, 79%) ¹H NMR (400 MHz, DMSO-d₆) δ 9.83 (s, 1H), 9.65 (s, 1H), 8.68 (d, J=1.0 Hz, 1H), 7.80 (d, J=9.1 Hz, 2H), 7.47-7.36 (m, 2H), 7.31 (dd, J=8.3, 5.8 Hz, 3H), 7.16 (d, J=1.2 Hz, 1H), 6.87 (dd, J=7.8, 2.2 Hz, 1H). HRMS: calc. [M+H]⁺ for C₁₇H₁₂F₃N₃O₂=348.0882; found=348.0921 [M+H]⁺.

### tert-butyl 2-(2-(3-(6-((4-(trifluoromethoxy)phenyl)amino)pyrimidin-4-yl)phenoxy)ethoxy)acetate

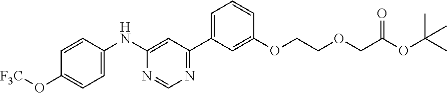 [SMILES: CC(C)(C)OC(=O)COCCOc1cccc(-c2cc(Nc3ccc(OC(F)(F)F)cc3)ncn2)c1]

3-[6-[4-(Trifluoromethoxy)anilino]pyrimidin-4-yl]phenol (50 mg, 0.144 mmol) dissolved in DMF (10 ml) and treat with tert-butyl 2-(2-iodoethoxy)acetate (45 mg, 0.158 mmol) and cesium carbonate (141 mg, 0.432 mmol) and the reaction mixture stirred at 65° C. overnight. The reaction was diluted with water (10 ml) and extracted with ethyl acetate (3×10 ml). The combined organics were washed sequentially with brine and 10% LiCl (aq.), dried over MgSO4 and concentrated in vacuo. The residue was purified by column chromatography eluting with 0-3% methanol/DCM to yield the title compound (13 mg, 18%). ¹H NMR (400 MHz, Chloroform-d) δ 8.76 (d, J=1.2 Hz, 1H), 7.60-7.45 (m, 4H), 7.35 (t, J=8.0 Hz, 1H), 7.24 (dd, J=9.3, 4.4 Hz, 3H), 7.08-6.96 (m, 2H), 4.29-4.21 (m, 2H), 4.10 (s, 2H), 4.01-3.87 (m, 2H), 1.47 (s, 9H). HRMS: calc. [M+H]⁺ for C₂₅H₂₆F₃N₃O₅=506.1897; found=506.2046 [M+H]⁺.

### 2-(2-(3-(6-((4-(trifluoromethoxy)phenyl)amino)pyrimidin-4-yl)phenoxy)ethoxy)acetic acid

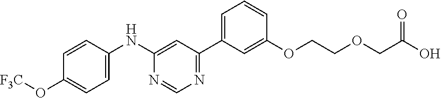 [SMILES: O=C(O)COCCOc1cccc(-c2cc(Nc3ccc(OC(F)(F)F)cc3)ncn2)c1]

Tert-butyl 2-(2-(3-(6-((4-(trifluoromethoxy)phenyl)amino)pyrimidin-4-yl)phenoxy)ethoxy)acetate (13 mg, 0.025 mmol) dissolved 20% TFA in DCM (10 ml) and stirred for 6 hours at r.t. Reaction concentrated in vacuo and used immediately in next step.

### (2S,4R)-1-((S)-3,3-dimethyl-2-(2-(2-(3-(6-((4-(trifluoromethoxy)phenyl)amino) pyrimidin-4-yl)phenoxy)ethoxy)acetamido)butanoyl)-4-hydroxy-N-(4-(4-methylthiazol-5-yl)benzyl)pyrrolidine-2-carboxamide

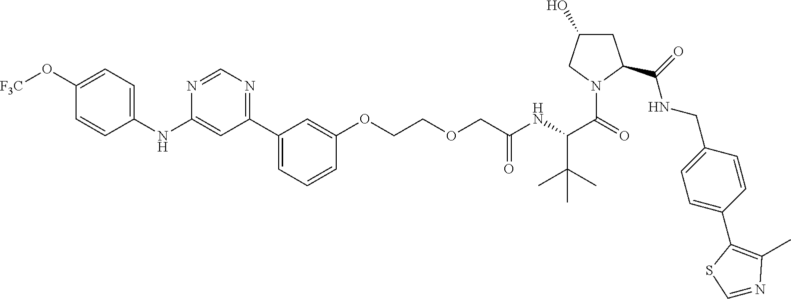 [SMILES: Cc1ncsc1-c1ccc(CNC(=O)[C@@H]2C[C@@H](O)CN2C(=O)[C@@H](NC(=O)COCCOc2cccc(-c3cc(Nc4ccc(OC(F)(F)F)cc4)ncn3)c2)C(C)(C)C)cc1]

2-(2-(3-(6-((4-(Trifluoromethoxy)phenyl)amino)pyrimidin-4-yl)phenoxy)ethoxy)acetic acid (11 mg, 0.025 mmol) was dissolved in DMF (10 ml) and treated with (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (11 mg, 0.025 mmol), HATU (11.5 mg, 0.030 mmol) and triethylamine (21 μl, 0.150 mmol). The reaction mixture was stirred overnight at r.t., diluted with ethyl acetate (30 ml) and washed with water (2×10 ml) and brine (10 ml). The organic layer was dried over MgSO₄ and concentrated in vacuo. The residue was purified by column chromatography eluting with 0-10% methanol/DCM. ¹H NMR (400 MHz, Chloroform-d) δ 8.71 (s, 1H), 8.63 (s, 1H), 8.24 (s, 1H), 7.68 (t, J=2.1 Hz, 1H), 7.66-7.57 (m, 4H), 7.50 (d, J=8.9 Hz, 1H), 7.37-7.27 (m, 2H), 7.25 (s, 1H), 7.22-7.08 (m, 7H), 7.02-6.88 (m, 2H), 4.71-4.56 (m, 2H), 4.50 (s, 1H), 4.40-4.19 (m, 2H), 4.13 (t, J=3.7 Hz, 2H), 4.07 (d, J=3.7 Hz, 2H), 4.05-3.98 (m, 1H), 3.95-3.80 (m, 2H), 3.71 (dd, J=11.3, 3.6 Hz, 1H), 2.41 (s, 3H), 0.98 (s, 9H). HRMS: calc. [M+H]⁺ for C₄₃H₄₆F₃N₇O₇S=862.3204; found=862.3690 [M+H]⁺.

### (2S,4R)-1-((S)-3,3-dimethyl-2-(3-(2-(2-(3-(6-((4-(trifluoromethoxy)phenyl)amino) pyrimidin-4-yl)phenoxy)ethoxy)ethoxy)propanamido)butanoyl)-4-hydroxy-N-(4-(4-methylthiazol-5-yl)benzyl)pyrrolidine-2-carboxamide

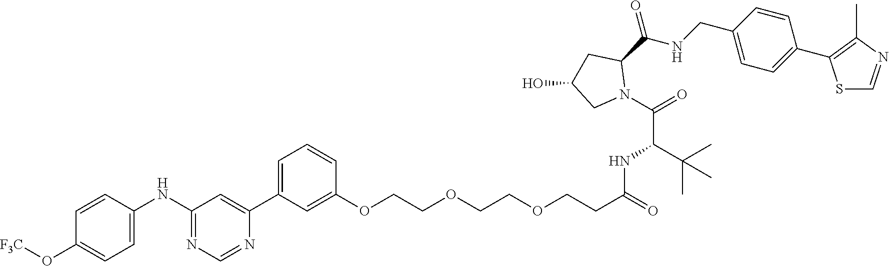 [SMILES: Cc1ncsc1-c1ccc(CNC(=O)[C@@H]2C[C@@H](O)CN2C(=O)[C@@H](NC(=O)CCOCCOCCOc2cccc(-c3cc(Nc4ccc(OC(F)(F)F)cc4)ncn3)c2)C(C)(C)C)cc1]

Prepared in the same manner as (2S,4R)-1-((S)-3,3-dimethyl-2-(2-(2-(3-(6-((4-(trifluoromethoxy)phenyl)amino)pyrimidin-4-yl)phenoxy)ethoxy)acetamido)butanoyl)-4-hydroxy-N-(4-(4-methylthiazol-5-yl)benzyl)pyrrolidine-2-carboxamide but employing tert-butyl 3-[2-[2-(p-tolylsulfonyloxy)ethoxy]ethoxy]propanoate in the place of tert-butyl 2-(2-iodoethoxy)acetate. HRMS: calc. [M+H]⁺ for C₄₆H₅₃F₃N₇O₈S=920.3623; found=920.3975 [M+H]⁺.

### (2S,4R)-1-((S)-3,3-dimethyl-2-(2-((5-((5-(3-(6-((4-(trifluoromethoxy)phenyl)amino) pyrimidin-4-yl)phenoxy)pentyl)oxy)pentyl)oxy)acetamido)butanoyl)-4-hydroxy-N-(4-(4-methylthiazol-5-yl)benzyl)pyrrolidine-2-carboxamide

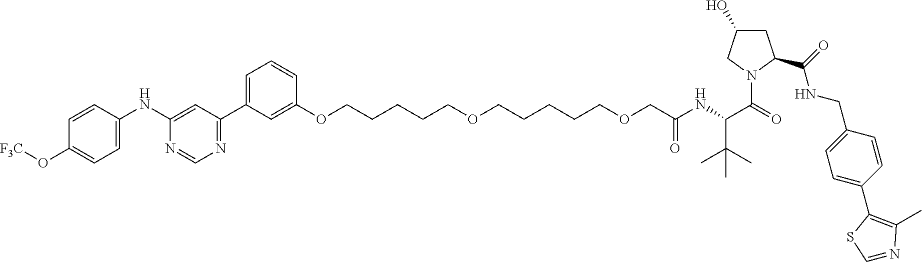 [SMILES: Cc1ncsc1-c1ccc(CNC(=O)[C@@H]2C[C@@H](O)CN2C(=O)[C@@H](NC(=O)COCCCCCOCCCCCOc2cccc(-c3cc(Nc4ccc(OC(F)(F)F)cc4)ncn3)c2)C(C)(C)C)cc1]

Prepared in the same manner as (2S,4R)-1-((S)-3,3-dimethyl-2-(2-(2-(3-(6-((4-(trifluoromethoxy)phenyl)amino)pyrimidin-4-yl)phenoxy)ethoxy)acetamido)butanoyl)-4-hydroxy-N-(4-(4-methylthiazol-5-yl)benzyl)pyrrolidine-2-carboxamide but employing tert-butyl 2-[5-(5-iodopentoxy)pentoxy]acetate in the place of tert-butyl 2-(2-iodoethoxy)acetate. ¹H NMR (400 MHz, Chloroform-d) δ 8.76 (d, J=1.2 Hz, 1H), 8.67 (s, 1H), 7.55-7.45 (m, 5H), 7.43-7.29 (m, 8H), 7.25 (s, 6H), 7.17 (d, J=8.6 Hz, 1H), 7.02-6.95 (m, 2H), 4.72 (t, J=7.9 Hz, 1H), 4.58-4.49 (m, 2H), 4.46 (d, J=8.6 Hz, 1H), 4.32 (dd, J=14.9, 5.3 Hz, 1H), 4.11-3.99 (m, 3H), 3.88 (q, J=15.5 Hz, 2H), 3.59 (dd, J=11.4, 3.7 Hz, 1H), 3.51-3.34 (m, 7H), 2.50 (s, 4H), 1.80 (q, J=7.0 Hz, 2H), 1.71-1.46 (m, 13H), 1.40 (td, J=8.6, 4.6 Hz, 2H), 0.93 (s, 9H). HRMS: calc. [M+H]⁺ for C₅₁H₆₂F₃N₇O₈S=990.4405; found=990.5181 [M+H]⁺.

### 6-(3-(4-bromobutoxy)phenyl)-N-(4-(trifluoromethoxy)phenyl)pyrimidin-4-amine

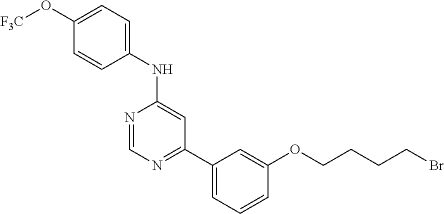 [SMILES: FC(F)(F)Oc1ccc(Nc2cc(-c3cccc(OCCCCBr)c3)ncn2)cc1]

3-[6-[4-(Trifluoromethoxy)anilino]pyrimidin-4-yl]phenol (80 mg, 0.230 mmol) was suspended in 1,4-dioxane (4 ml) and treated with 1,4-dibromobutane (138 μl, 1.15 mmol) and caesium carbonate (113 mg, 0.346 mmol). The reaction mixture was heated to 120° C. under microwave conditions for 2 hours, cooled to r.t., filtered and concentrated in vacuo. The residue was purified by column chromatography eluting with 0-50% ethyl acetate/hexane to yield the title compound (64 mg, 58%). ¹H NMR (400 MHz, Chloroform-d) δ 8.76 (d, J=1.2 Hz, 1H), 7.54 (dd, J=2.6, 1.6 Hz, 1H), 7.46 (dd, J=9.2, 2.6 Hz, 3H), 7.33 (d, J=7.7 Hz, 2H), 7.25 (dd, J=7.1, 2.1 Hz, 2H), 7.02 (d, J=1.2 Hz, 1H), 6.98 (ddd, J=8.2, 2.6, 0.9 Hz, 1H), 4.05 (t, J=6.0 Hz, 2H), 3.48 (t, J=6.6 Hz, 2H), 2.11-2.01 (m, 2H), 1.99-1.88 (m, 2H). LC-MS (ESI) m/z: 482,484

### (2S,4R)-4-hydroxy-1-((S)-3-methyl-2-(1-oxoisoindolin-2-yl)butanoyl)-N-(5-(4-methylthiazol-5-yl)-2-(4-(3-(6-((4-(trifluoromethoxy)phenyl)amino) pyrimidin-4-yl) phenoxy)butoxy) benzyl)pyrrolidine-2-carboxamide

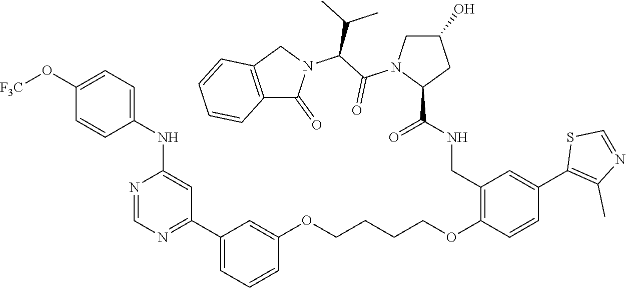 [SMILES: Cc1ncsc1-c1ccc(OCCCCOc2cccc(-c3cc(Nc4ccc(OC(F)(F)F)cc4)ncn3)c2)c(CNC(=O)[C@@H]2C[C@@H](O)CN2C(=O)[C@H](C(C)C)N2Cc3ccccc3C2=O)c1]

6-(3-(4-Bromobutoxy)phenyl)-N-(4-(trifluoromethoxy)phenyl)pyrimidin-4-amine (32 mg, 0.066 mmol), (2S,4R)-4-(tert-butoxy)-N-(2-hydroxy-5-(4-methylthiazol-5-yl)benzyl)-1-((S)-3-methyl-2-(1-oxoisoindolin-2-yl)butanoyl)pyrrolidine-2-carboxamide (44 mg, 0.073 mmol) and cesium carbonate (32 mg, 0.099 mmol) suspended in 1,4-dioxane and heated to 120° C. for 4 hours under microwave conditions. The reaction mixture was filtered and concentrated in vacuo. The residue was re-dissolved in 20% TFA/DCM and stirred at r.t. overnight. The reaction mixture was concentrated in vacuo and purified by preparative TLC eluting with 5% methanol/DCM. ¹H NMR (400 MHz, Chloroform-d) δ 8.72 (d, J=1.1 Hz, 1H), 8.66 (s, 1H), 7.71 (d, J=7.5 Hz, 1H), 7.65-7.56 (m, 2H), 7.55-7.42 (m, 3H), 7.40-7.30 (m, 3H), 7.30-7.19 (m, 7H), 6.98-6.92 (m, 3H), 6.87 (d, J=1.6 Hz, 1H), 4.74-4.38 (m, 7H), 4.31 (d, J=17.6 Hz, 1H), 4.13 (dt, J=14.6, 5.6 Hz, 4H), 3.63 (dd, J=11.4, 3.5 Hz, 1H), 2.51 (s, 3H), 2.38-2.25 (m, 1H), 2.05 (dq, J=17.4, 10.4, 8.3 Hz, 5H), 0.83 (dd, J=12.3, 6.6 Hz, 7H). HRMS: calc. [M+H]⁺ for C₅₀H₅₀F₃N₇O₇S=950.3517; found=950.5543 [M+H]⁺.

### 6-(3-(4-(4-iodobutoxy)butoxy)phenyl)-N-(4-(trifluoromethoxy)phenyl)pyrimidin-4-amine

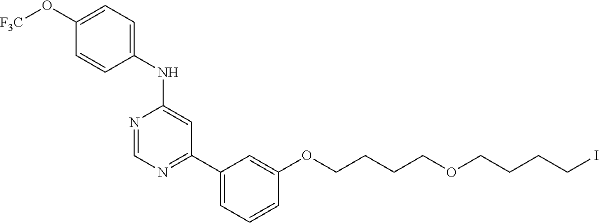 [SMILES: FC(F)(F)Oc1ccc(Nc2cc(-c3cccc(OCCCCOCCCCI)c3)ncn2)cc1]

3-[6-[4-(Trifluoromethoxy)anilino]pyrimidin-4-yl]phenol (140 mg, 0.403 mmol) was suspended in 1,4-dioxane (4 ml) and treated with 1-iodo-4-(4-iodobutoxy)butane (770 μl, 2.02 mmol) and caesium carbonate (144 mg, 0.443 mmol). The reaction mixture was heated to 120° C. under microwave conditions for 2 hours, cooled to r.t., filtered and concentrated in vacuo. The residue was purified by column chromatography eluting with 0-50% ethyl acetate/hexane to yield the title compound (83 mg, 34%). ¹H NMR (400 MHz, Chloroform-d) δ 8.75 (d, J=1.1 Hz, 1H), 7.53 (d, J=2.1 Hz, 1H), 7.48-7.41 (m, 5H), 7.34 (t, J=7.9 Hz, 1H), 7.28-7.20 (m, 3H), 7.02 (d, J=1.2 Hz, 1H), 6.98 (dd, J=8.1, 2.6 Hz, 1H), 4.03 (t, J=6.3 Hz, 3H), 3.44 (dt, J=12.4, 6.3 Hz, 5H), 3.19 (t, J=7.0 Hz, 3H), 1.94-1.80 (m, 5H), 1.80-1.59 (m, 6H). HRMS: calc. [M+H]⁺ for C₂₅H₂₇F₃IN₃O₃=602.1122; found=602.0941 [M+H]⁺.

### (2S,4R)-4-hydroxy-1-((S)-3-methyl-2-(1-oxoisoindolin-2-yl)butanoyl)-N-(5-(4-methylthiazol-5-yl)-2-(4-(4-(3-(6-((4-(trifluoromethoxy)phenyl)amino)pyrimidin-4-yl)phenoxy)butoxy)butoxy)benzyl)pyrrolidine-2-carboxamide

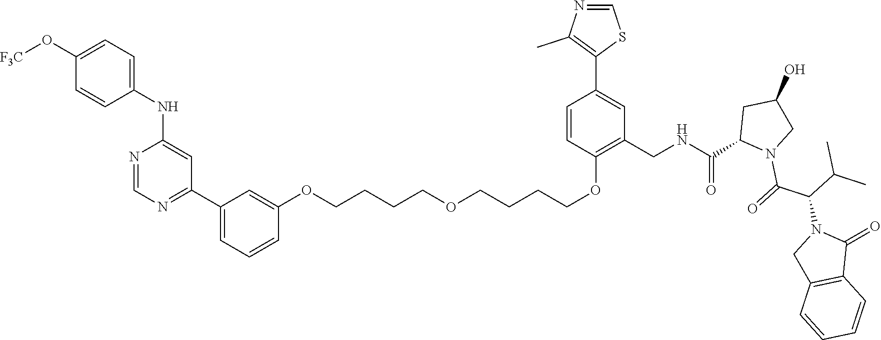 [SMILES: Cc1ncsc1-c1ccc(OCCCCOCCCCOc2cccc(-c3cc(Nc4ccc(OC(F)(F)F)cc4)ncn3)c2)c(CNC(=O)[C@@H]2C[C@@H](O)CN2C(=O)[C@H](C(C)C)N2Cc3ccccc3C2=O)c1]

6-(3-(4-(4-Iodobutoxy)butoxy)phenyl)-N-(4-(trifluoromethoxy)phenyl)pyrimidin-4-amine (20 mg, 0.033 mmol), (2S,4R)-4-(tert-butoxy)-N-(2-hydroxy-5-(4-methylthiazol-5-yl)benzyl)-1-((S)-3-methyl-2-(1-oxoisoindolin-2-yl)butanoyl)pyrrolidine-2-carboxamide (20 mg, 0.036 mmol) and cesium carbonate (13 mg, 0.039 mmol) suspended in acetonitrile and heated to 80° C. overnight. The reaction mixture was concentrated in vacuo and purified by preparative TLC eluting with 5% methanol/DCM. ¹H NMR (400 MHz, Chloroform-d) δ 8.74 (s, 1H), 8.65 (s, 1H), 7.72 (d, J=7.5 Hz, 1H), 7.60-7.53 (m, 2H), 7.52-7.43 (m, 3H), 7.40-7.26 (m, 4H), 7.23-7.17 (m, 3H), 7.04-6.99 (m, 1H), 6.94 (dd, J=8.0, 2.5 Hz, 1H), 6.89 (dd, J=7.6, 1.6 Hz, 1H), 6.83 (d, J=1.6 Hz, 1H), 4.78-4.64 (m, 2H), 4.60 (t, J=7.9 Hz, 1H), 4.53-4.29 (m, 6H), 4.04-3.89 (m, 4H), 3.67 (dd, J=11.3, 3.5 Hz, 1H), 3.48 (dt, J=12.4, 6.2 Hz, 5H), 2.49 (s, 3H), 2.38 (ddt, J=32.8, 13.1, 5.4 Hz, 2H), 2.10-2.01 (m, 1H), 1.96-1.62 (m, 14H), 0.86 (dd, J=23.8, 6.5 Hz, 6H). HRMS: calc. [M+H]⁺ for C₅₄H₅₈F₃N₇O₈S=1022.4092; found=1022.4176 [M+H]⁺.

### 6-(3-(4-(4-azidobutoxy)butoxy)phenyl)-N-(4-(trifluoromethoxy)phenyl)pyrimidin-4-amine

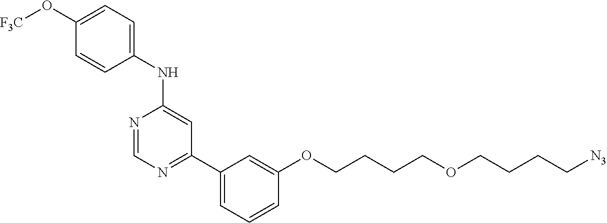 [SMILES: [N-]=[N+]=NCCCCOCCCCOc1cccc(-c2cc(Nc3ccc(OC(F)(F)F)cc3)ncn2)c1]

6-(3-(4-(4-Iodobutoxy)butoxy)phenyl)-N-(4-(trifluoromethoxy)phenyl)pyrimidin-4-amine (60 mg, 0.010 mmol) was dissolved in DMF (10 ml) and treated with sodium azide (20 mg, 0.300 mmol). The reaction mixture was stirred overnight at r.t., diluted with ethyl acetate (20 ml) and washed with water (10 ml), brine (10 ml) and 10% LiCl (aq.) (10 ml), dried over MgSO₄ and concentrated in vacuo to yield the title compound (50 mg, 97%). ¹H NMR (400 MHz, Chloroform-d) δ 8.80-8.70 (m, 1H), 7.72-7.62 (m, 1H), 7.55-7.42 (m, 4H), 7.32 (t, J=7.9 Hz, 1H), 7.23 (d, J=8.2 Hz, 3H), 7.05 (s, 1H), 6.98 (dd, J=8.0, 2.5 Hz, 1H), 4.03 (t, J=6.3 Hz, 2H), 3.44 (dt, J=11.8, 6.0 Hz, 4H), 3.28 (t, J=6.4 Hz, 2H), 1.85 (dq, J=11.4, 6.4 Hz, 2H), 1.80-1.54 (m, 6H). LC-MS (ESI) m/z: 517.

### 6-(3-(4-(4-aminobutoxy)butoxy)phenyl)-N-(4-(trifluoromethoxy)phenyl)pyrimidin-4-amine

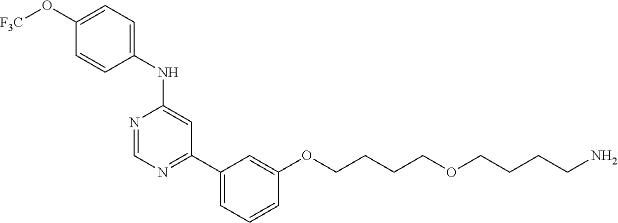 [SMILES: NCCCCOCCCCOc1cccc(-c2cc(Nc3ccc(OC(F)(F)F)cc3)ncn2)c1]

6-(3-(4-(4-Azidobutoxy)butoxy)phenyl)-N-(4-(trifluoromethoxy)phenyl)pyrimidin-4-amine (50 mg, 0.097 mmol) was dissolved in THE and placed under an N₂ atmosphere. Pd/C (10%) was added and the atmosphere replaced with H2. The reaction mixture was stirred overnight, filtered through Celite with THE and concentrated in vacuo to yield the title compound which was used immediately in the next step. LC-MS (ESI) m/z: 491.

### (2R,3S,4R,5S)-3-(3-chloro-2-fluorophenyl)-4-(4-chloro-2-fluorophenyl)-4-cyano-N-(2-methoxy-4-((4-(4-(3-(6-((4-(trifluoromethoxy)phenyl)amino)pyrimidin-4-yl)phenoxy)butoxy)butyl)carbamoyl)phenyl)-5-neopentylpyrrolidine-2-carboxamide

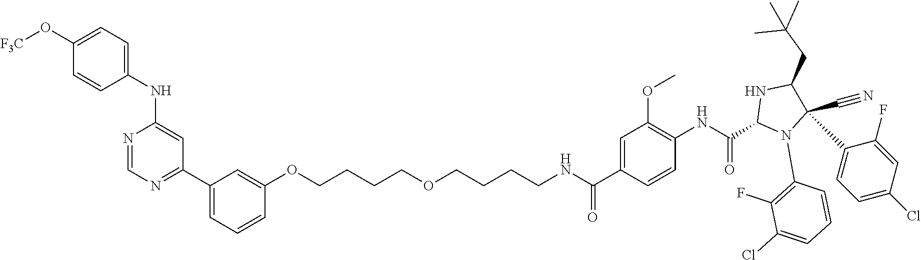 [SMILES: COc1cc(C(=O)NCCCCOCCCCOc2cccc(-c3cc(Nc4ccc(OC(F)(F)F)cc4)ncn3)c2)ccc1NC(=O)[C@@H]1N[C@@H](CC(C)(C)C)[C@](C#N)(c2ccc(Cl)cc2F)N1c1cccc(Cl)c1F]

6-(3-(4-(4-Azidobutoxy)butoxy)phenyl)-N-(4-(trifluoromethoxy)phenyl)pyrimidin-4-amine (23 mg, 0.048 mmol) was dissolved in DMA (2 ml) and treated with 4-[[(2R,3S,4R,5S)-3-(3-chloro-2-fluoro-phenyl)-4-(4-chloro-2-fluoro-phenyl)-4-cyano-5-(2,2-dimethylpropyl)pyrrolidine-2-carbonyl]amino]-3-methoxy-benzoic acid (33 mg, 0.053 mmol), HATU (22 mg, 0.057 mmol) and triethylamine (34 μl, 0.240 mmol). The reaction mixture was stirred overnight at r.t., diluted with ethyl acetate (10 ml) and washed with water (10 ml) and brine (10 ml), dried over MgSO4 and concentrated in vacuo. The residue was purified by preparative TLC eluting with 10% methanol/DCM. HRMS: calc. [M+H]⁺ for C₅₆H₅₆C₁₂F₃N₇O₆=1088.3662; found=1088.3964 [M+H]⁺.

### 2-(2,6-dioxopiperidin-3-yl)-4-((4-(4-(3-(6-((4-(trifluoromethoxy)phenyl)amino) pyrimidin-4-yl)phenoxy)butoxy)butyl)amino) isoindoline-1,3-dione

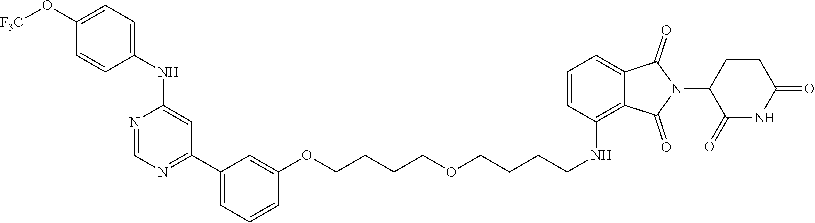 [SMILES: O=C1CCC(N2C(=O)c3cccc(NCCCCOCCCCOc4cccc(-c5cc(Nc6ccc(OC(F)(F)F)cc6)ncn5)c4)c3C2=O)C(=O)N1]

6-(3-(4-(4-Azidobutoxy)butoxy)phenyl)-N-(4-(trifluoromethoxy)phenyl)pyrimidin-4-amine (23 mg, 0.048 mmol) was dissolved in DMA (2 ml) and treated with 2-(2,6-dioxo-3-piperidyl)-4-fluoro-isoindoline-1,3-dione (15 mg, 0.053 mmol) and triethylamine (34 μl, 0.240 mmol). The reaction mixture was heated to 140° C. for 30 minutes under microwave conditions. After cooling, the reaction mixture was diluted with ethyl acetate (10 ml) and washed with water (10 ml) and brine (10 ml), dried over MgSO4 and concentrated in vacuo. The residue was purified by column chromatography eluting with 0-10% methanol/DCM. ¹H NMR (400 MHz, Chloroform-d) δ 8.75 (s, 1H), 8.37 (s, 1H), 7.62-7.51 (m, 1H), 7.51-7.40 (m, 4H), 7.35 (d, J=8.0 Hz, 1H), 7.23-7.21 (m, 1H), 7.07-6.96 (m, 3H), 6.85 (d, J=8.5 Hz, 1H), 6.21 (t, J=5.7 Hz, 1H), 4.89 (dd, J=12.1, 5.3 Hz, 1H), 4.06 (t, J=6.3 Hz, 2H), 3.47 (dt, J=10.0, 5.9 Hz, 5H), 3.26 (q, J=6.3 Hz, 2H), 2.91-2.63 (m, 4H), 2.11 (dt, J=7.5, 4.6 Hz, 1H), 1.92-1.61 (m, 19H). HRMS: calc. [M+H]⁺ for C₃₈H₃₇F₃N₆O₇=747.2749; found=747.1461 [M+H]⁺.

### 6-(1H-pyrazol-4-yl)-N-(4-(trifluoromethoxy)phenyl)pyrimidin-4-amine

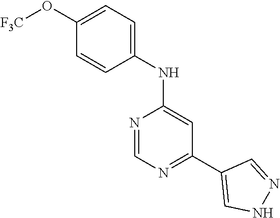 [SMILES: FC(F)(F)Oc1ccc(Nc2cc(-c3cn[nH]c3)ncn2)cc1]

Following a literature procedure, 6-chloro-N-(4-(trifluoromethoxy)phenyl)pyrimidin-4-amine (300 mg, 1.04 mmol), (1H-pyrazol-4-yl)boronic acid (116 mg, 1.04 mmol), tetrakis(triphenylphosphine)palladium(0) (120 mg, 10 mol %) and sodium carbonate (439 mg, 4.14 mmol) were heated to reflux in a 1:1 mixtures of acetonitrile/water (20 ml) overnight. The resulting suspension was filtered hot, cooled to r.t., adjusted to pH 4 with conc. HCl. The resulting precipitate was collected by filtration, washed with water and dried in vacuo (213 mg, 64%).

Characterization data matched the literature report in X. Deng, et al., Journal of Medicinal Chemistry, 2010, 53, 6934-6946.

### tert-butyl 2-(2-(4-(6-((4-(trifluoromethoxy)phenyl)amino)pyrimidin-4-yl)-1H-pyrazol-1-yl)ethoxy)acetate

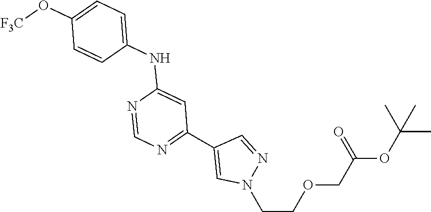 [SMILES: CC(C)(C)OC(=O)COCCn1cc(-c2cc(Nc3ccc(OC(F)(F)F)cc3)ncn2)cn1]

6-(1H-Pyrazol-4-yl)-N-(4-(trifluoromethoxy)phenyl)pyrimidin-4-amine (50 mg, 0.156 mmol) was suspended in 1,4-dioxane (4 ml) and treated with tert-butyl 2-(2-iodoethoxy)acetate (45 mg, 0.156 mmol) and cesium carbonate (61 mg, 0.187 mmol). The reaction mixture was heated to 120° C. under microwave conditions for 4 hours, cooled to r.t., filtered and concentrated in vacuo. The residue was purified by column chromatography eluting with 0-50% ethyl acetate/hexane to yield the title compound (38 mg, 51%). ¹H NMR (400 MHz, Acetone-d₆) δ 8.81 (s, 1H), 8.58 (d, J=1.1 Hz, 1H), 8.27-8.11 (m, 1H), 7.94 (d, J=0.7 Hz, 1H), 7.91-7.82 (m, 2H), 7.36-7.24 (m, 3H), 6.98 (d, J=1.2 Hz, 1H), 4.37 (t, J=5.3 Hz, 2H), 3.97 (s, 2H), 3.95 (t, J=5.3 Hz, 2H), 1.42 (s, 12H). LC-MS (ESI) m/z: 480.

### 2-(2-(4-(6-((4-(trifluoromethoxy)phenyl)amino)pyrimidin-4-yl)-1H-pyrazol-1-yl)ethoxy)acetic acid

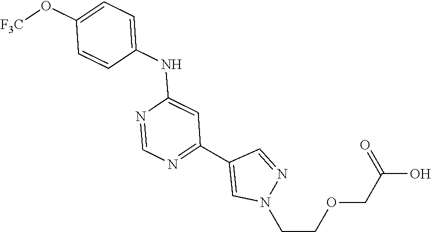 [SMILES: O=C(O)COCCn1cc(-c2cc(Nc3ccc(OC(F)(F)F)cc3)ncn2)cn1]

Tert-butyl 2-(2-(4-(6-((4-(trifluoromethoxy)phenyl)amino)pyrimidin-4-yl)-1H-pyrazol-1-yl)ethoxy)acetate (38 mg, 0.079 mmol) dissolved 20% TFA in DCM (10 ml) and stirred for 2 hours at r.t. Reaction concentrated in vacuo and used immediately in next step.

### (2S,4R)-1-((S)-3,3-dimethyl-2-(2-(2-(4-(6-((4-(trifluoromethoxy)phenyl)amino) pyrimidin-4-yl)-1H-pyrazol-1-yl)ethoxy)acetamido)butanoyl)-4-hydroxy-N-(4-(4-methylthiazol-5-yl)benzyl)pyrrolidine-2-carboxamide

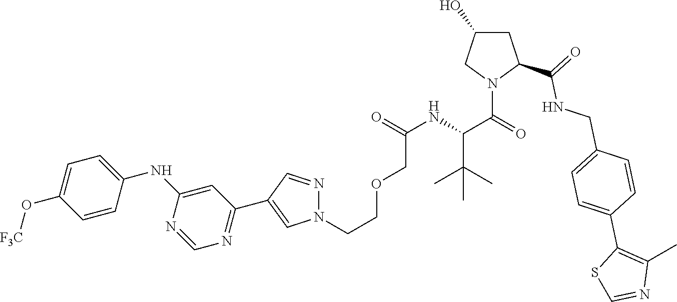 [SMILES: Cc1ncsc1-c1ccc(CNC(=O)[C@@H]2C[C@@H](O)CN2C(=O)[C@@H](NC(=O)COCCn2cc(-c3cc(Nc4ccc(OC(F)(F)F)cc4)ncn3)cn2)C(C)(C)C)cc1]

2-(2-(4-(6-((4-(Trifluoromethoxy)phenyl)amino)pyrimidin-4-yl)-1H-pyrazol-1-yl)ethoxy)acetic acid (33 mg, 0.079 mmol) was dissolved in DMF (10 ml) and treated with (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (34 mg, 0.079 mmol), HATU (36 mg, 0.095 mmol) and triethylamine (55 μl, 0.395 mmol). The reaction mixture was stirred overnight at r.t., diluted with ethyl acetate (30 ml) and washed with water (2×10 ml) and brine (10 ml). The organic layer was dried over MgSO₄ and concentrated in vacuo. The residue was purified by column chromatography eluting with 0-10% methanol/DCM. ¹H NMR (400 MHz, Acetone-d6) δ 9.01 (s, 1H), 8.80 (s, 1H), 8.58 (s, 1H), 8.51 (s, 1H), 8.17 (t, J=6.2 Hz, 1H), 8.04 (s, 1H), 7.98-7.90 (m, 2H), 7.41-7.33 (m, 3H), 7.29 (d, J=8.1 Hz, 2H), 7.23 (d, J=8.7 Hz, 2H), 7.09-6.99 (m, 1H), 4.73 (dd, J=9.7, 7.4 Hz, 1H), 4.67-4.53 (m, 4H), 4.49-4.37 (m, 4H), 3.96-3.78 (m, 5H), 2.37 (s, 3H), 2.04 (p, J=2.2 Hz, 4H), 0.93 (s, 9H). HRMS: calc. [M+H]⁺ for C₄₀H₄₄F₃N₉O₆S=836.3160; found=836.2014 [M+H]⁺.

### 4-(6-((4-(trifluoromethoxy)phenyl)amino)pyrimidin-4-yl)phenol

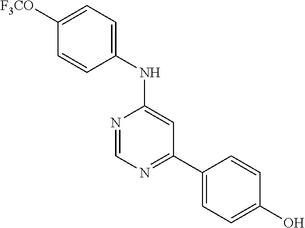 [SMILES: Oc1ccc(-c2cc(Nc3ccc(OC(F)(F)F)cc3)ncn2)cc1]

Following an adapted literature procedure (Journal of Medicinal Chemistry, 2010, 53, 6934-6946), 6-chloro-N-(4-(trifluoromethoxy)phenyl)pyrimidin-4-amine (300 mg, 1.04 mmol), (4-hydroxyphenyl)boronic acid (143 mg, 1.04 mmol), tetrakis(triphenylphosphine)palladium(0) (120 mg, 10 mol %) and sodium carbonate (439 mg, 4.14 mmol) were heated to reflux in a 1:1 mixtures of acetonitrile/water (20 ml) overnight. The resulting suspension was filtered hot, cooled to r.t., adjusted to pH 4 with conc. HCl. The resulting precipitate was collected by filtration, washed with water and dried in vacuo (345 mg, 95%). ¹H NMR (400 MHz, DMSO-d₆) δ 10.42 (s, 1H), 10.18 (s, 1H), 8.71 (s, 1H), 7.82 (dd, J=17.2, 8.8 Hz, 4H), 7.35 (d, J=8.6 Hz, 2H), 7.17 (s, 1H), 6.91 (d, J=8.5 Hz, 2H). LC-MS (ESI) m/z: 348

### tert-butyl 2-(2-(4-(6-((4-(trifluoromethoxy)phenyl)amino)pyrimidin-4-yl)phenoxy)ethoxy)acetate

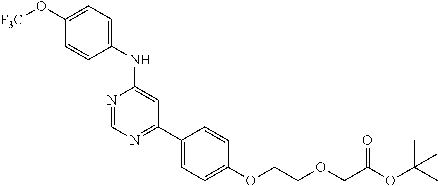 [SMILES: CC(C)(C)OC(=O)COCCOc1ccc(-c2cc(Nc3ccc(OC(F)(F)F)cc3)ncn2)cc1]

4-(6-((4-(Trifluoromethoxy)phenyl)amino)pyrimidin-4-yl)phenol (100 mg, 0.288 mmol) was suspended in 1,4-dioxane (4 ml) and treated with tert-butyl 2-(2-iodoethoxy)acetate (82 mg, 0.288 mmol) and cesium carbonate (113 mg, 0.346 mmol). The reaction mixture was heated to 120° C. under microwave conditions for 4 hours, cooled to r.t., filtered and concentrated in vacuo. The residue was purified by column chromatography eluting with 0-30% ethyl acetate/hexane to yield the title compound (60 mg, 42%). ¹H NMR (400 MHz, Chloroform-d) δ 8.72 (d, J=1.1 Hz, 1H), 7.90 (d, J=8.9 Hz, 2H), 7.45 (d, J=9.0 Hz, 2H), 7.27-7.20 (m, 4H), 6.97 (dd, J=5.0, 3.8 Hz, 3H), 4.23-4.16 (m, 2H), 4.08 (s, 2H), 3.96-3.85 (m, 2H), 1.47 (s, 8H). LC-MS (ESI) m/z: 506.

### 2-(2-(4-(6-((4-(trifluoromethoxy)phenyl)amino)pyrimidin-4-yl)phenoxy)ethoxy) acetic acid

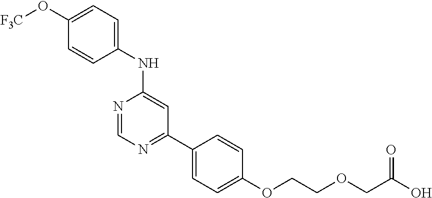 [SMILES: O=C(O)COCCOc1ccc(-c2cc(Nc3ccc(OC(F)(F)F)cc3)ncn2)cc1]

Tert-butyl 2-(2-(4-(6-((4-(trifluoromethoxy)phenyl)amino)pyrimidin-4-yl)phenoxy)ethoxy)acetate (40 mg, 0.079 mmol) dissolved 20% TFA in DCM (10 ml) and stirred for 2 hours at r.t. Reaction concentrated in vacuo and used immediately in next step.

### (2S,4R)-1-((S)-3,3-dimethyl-2-(2-(2-(4-(6-((4-(trifluoromethoxy)phenyl)amino) pyrimidin-4-yl)phenoxy)ethoxy)acetamido)butanoyl)-4-hydroxy-N-(4-(4-methylthiazol-5-yl)benzyl)pyrrolidine-2-carboxamide

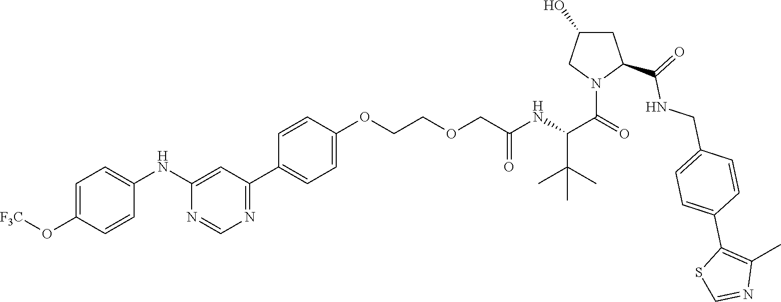 [SMILES: Cc1ncsc1-c1ccc(CNC(=O)[C@@H]2C[C@@H](O)CN2C(=O)[C@@H](NC(=O)COCCOc2ccc(-c3cc(Nc4ccc(OC(F)(F)F)cc4)ncn3)cc2)C(C)(C)C)cc1]

2-[2-[4-[6-[4-(Trifluoromethoxy)anilino]pyrimidin-4-yl]phenoxy]ethoxy]acetic acid (35 mg, 0.079 mmol) was dissolved in DMF (10 ml) and treated with (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (34 mg, 0.079 mmol), HATU (36 mg, 0.095 mmol) and triethylamine (55 μl, 0.395 mmol). The reaction mixture was stirred overnight at r.t., diluted with ethyl acetate (30 ml) and washed with water (2×10 ml) and brine (10 ml). The organic layer was dried over MgSO₄ and concentrated in vacuo. The residue was purified by column chromatography eluting with 0-10% methanol/DCM. ¹H NMR (400 MHz, Chloroform-d) δ 8.74-8.55 (m, 2H), 7.84 (d, J=8.8 Hz, 2H), 7.55 (d, J=9.0 Hz, 2H), 7.38-7.18 (m, 9H), 6.97 (d, J=8.8 Hz, 2H), 6.84 (d, J=1.2 Hz, 1H), 4.65-4.44 (m, 4H), 4.18 (t, J=4.3 Hz, 2H), 4.05 (d, J=4.8 Hz, 2H), 3.88 (d, J=4.2 Hz, 1H), 3.68-3.55 (m, 1H), 2.46 (s, 3H), 0.93 (s, 9H). HRMS: calc. [M+H]⁺ for C₄₃H₄₆F₃N₇O₇S=862.3165; found=863.2714 [M+H]⁺.

### (2R,4S)-1-((S)-3,3-dimethyl-2-(2-(2-(4-(6-((4-(trifluoromethoxy)phenyl)amino) pyrimidin-4-yl)phenoxy)ethoxy)acetamido)butanoyl)-4-hydroxy-N-(4-(4-methylthiazol-5-yl)benzyl)pyrrolidine-2-carboxamide

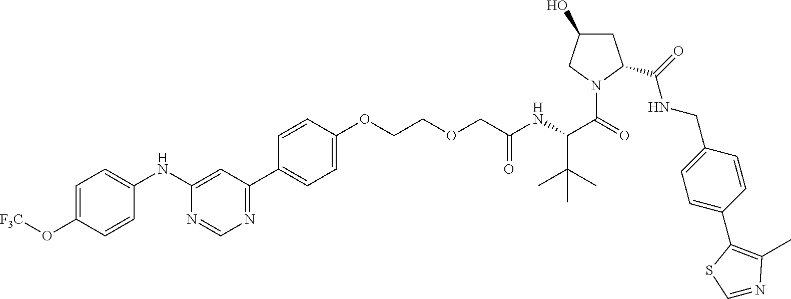 [SMILES: Cc1ncsc1-c1ccc(CNC(=O)[C@H]2C[C@H](O)CN2C(=O)[C@@H](NC(=O)COCCOc2ccc(-c3cc(Nc4ccc(OC(F)(F)F)cc4)ncn3)cc2)C(C)(C)C)cc1]

2-[2-[4-[6-[4-(Trifluoromethoxy)anilino]pyrimidin-4-yl]phenoxy]ethoxy]acetic acid (16 mg, 0.036 mmol) was dissolved in DMF (10 ml) and treated with (2R,4S)-1-[(2S)-2-amino-3,3-dimethyl-butanoyl]-4-hydroxy-N-[[4-(4-methylthiazol-5-yl)phenyl]methyl]pyrrolidine-2-carboxamide (15.5 mg, 0.036 mmol), HATU (17 mg, 0.043 mmol) and triethylamine (25 μl, 0.180 mmol). The reaction mixture was stirred overnight at r.t., diluted with ethyl acetate (30 ml) and washed with water (2×10 ml) and brine (10 ml). The organic layer was dried over MgSO₄ and concentrated in vacuo. The residue was purified by column chromatography eluting with 0-10% methanol/DCM. ¹H NMR (400 MHz, Chloroform-d) δ 8.72 (d, J=1.1 Hz, 1H), 8.62 (s, 1H), 7.92-7.85 (m, 2H), 7.50 (td, J=6.3, 5.7, 2.2 Hz, 3H), 7.34-7.29 (m, 3H), 7.24-7.20 (m, 1H), 7.17 (d, J=6.9 Hz, 1H), 6.96 (s, 1H), 6.93 (d, J=1.4 Hz, 2H), 4.74 (dd, J=8.6, 4.4 Hz, 1H), 4.64-4.49 (m, 2H), 4.31 (d, J=7.0 Hz, 1H), 4.21 (dd, J=15.5, 5.0 Hz, 1H), 4.12-4.05 (m, 3H), 3.91 (d, J=15.7 Hz, 1H), 3.71 (dd, J=5.9, 3.2 Hz, 2H), 3.63 (dd, J=10.6, 4.9 Hz, 1H), 3.51 (d, J=15.7 Hz, 1H), 2.47 (s, 3H), 1.05 (s, 9H). HRMS: calc. [M+H]⁺ for C₄₃H₄₆F₃N₇O₇S=862.3165; found=862.3298 [M+H]⁺.

### 5-bromo-6-chloro-N-(4-(chlorodifluoromethoxy)phenyl)nicotinamide

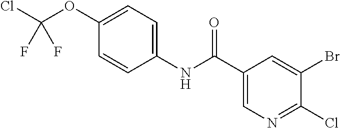 [SMILES: O=C(Nc1ccc(OC(F)(F)Cl)cc1)c1cnc(Cl)c(Br)c1]

Following a literature procedure, 5-bromo-6-chloronicotinic acid (300 mg, 1.27 mmol) was suspended in toluene and treated with DMF (30 μl, 30 mol %) followed by thionyl chloride (278 μl, 3.81 mmol). The reaction mixture was then heated to 80° C. for 1 hour with stirring before being concentrated in vacuo and resuspended in anhydrous THF. The acid chloride solution was treated with triethylamine (442 μl, 3.17 mmol) followed by a solution of 4-(chlorodifluoromethoxy)aniline (258 mg, 1.33 mmol) in THF. The reaction mixture was stirred at r.t. for 1 hour, concentrated in vacuo and purified by column chromatography eluting with 0-50% ethyl acetate/hexane to yield the title compound (395 mg, 76%). Characterization data matched the literature report in A. A. Wylie, et al., Nature, 2017, 543, 733.

### 2-(2-((3-bromo-5-((4-(chlorodifluoromethoxy)phenyl)carbamoyl)pyridin-2-yl)amino)ethoxy)acetic acid

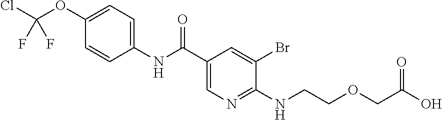 [SMILES: O=C(O)COCCNc1ncc(C(=O)Nc2ccc(OC(F)(F)Cl)cc2)cc1Br]

5-Bromo-6-chloro-N-(4-(chlorodifluoromethoxy)phenyl)nicotinamide (50 mg, 0.121 mmol) and 2-(2-aminoethoxy)acetic acid (17 mg, 0.146 mmol) were suspended in isopropanol and treated with triethylamine (37 μl, 0.267 mmol) and heated to 140° C. under microwave conditions for 6 hours. The reaction mixture was concentrated in vacuo and purified by column chromatography eluting with 0-15% methanol/DCM. ¹H NMR (400 MHz, Methanol-d4) δ 8.61 (d, J=2.1 Hz, 1H), 8.22 (d, J=2.1 Hz, 1H), 7.74 (d, J=9.1 Hz, 2H), 7.22 (d, J=8.7 Hz, 2H), 4.02 (s, 2H), 3.18 (q, J=7.3 Hz, 1H), 1.29 (t, J=7.3 Hz, 3H). LC-MS (ESI) m/z: 494, 496.

### 5-bromo-N-(4-(chlorodifluoromethoxy)phenyl)-6-((2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethyl)amino)nicotinamide

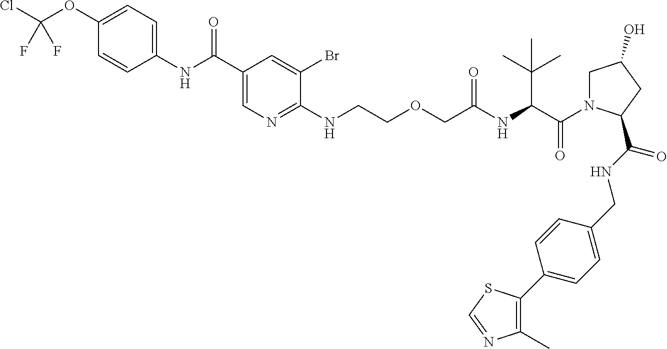 [SMILES: Cc1ncsc1-c1ccc(CNC(=O)[C@@H]2C[C@@H](O)CN2C(=O)[C@@H](NC(=O)COCCNc2ncc(C(=O)Nc3ccc(OC(F)(F)Cl)cc3)cc2Br)C(C)(C)C)cc1]

2-[2-[[3-Bromo-5-[[4-[chloro(difluoro)methoxy]phenyl]carbamoyl]-2-pyridyl]amino]ethoxy]acetic acid (35 mg, 0.072 mmol) dissolved in DMF (10 ml) and treated sequentially with HATU (27 mg, 0.072 mmol), triethylamine (30 μl, 0.216 mmol) and (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (31 mg, 0.072 mmol). The reaction mixture was stirred overnight at r.t., diluted with ethyl acetate (20 ml), washed with water (10 ml) and brine (10 ml), dried over MgSO₄ and concentrated in vacuo. Purified by column chromatography eluting with 0-10% methanol/DCM to yield the title compound (18 mg, 28%). LC-MS (ESI) m/z: 906,908. ¹H NMR (400 MHz, DMSO-d₆) δ 10.17 (s, 1H), 8.94 (s, 1H), 8.63 (d, J=2.1 Hz, 1H), 8.55 (t, J=6.0 Hz, 1H), 8.27 (d, J=2.1 Hz, 1H), 7.81 (d, J=9.1 Hz, 2H), 7.44 (d, J=9.5 Hz, 1H), 7.36 (s, 2H), 7.30 (d, J=8.7 Hz, 2H), 7.09-7.00 (m, 1H), 5.12 (d, J=3.5 Hz, 1H), 4.52 (d, J=9.5 Hz, 1H), 4.45-4.26 (m, 3H), 4.21 (dd, J=15.8, 5.7 Hz, 1H), 3.98 (d, J=2.3 Hz, 2H), 3.73-3.53 (m, 4H), 3.06 (s, 1H), 2.40 (s, 3H), 2.00 (d, J=8.4 Hz, 1H), 1.94-1.79 (m, 1H), 1.13 (s, 2H), 0.89 (s, 9H).

### N-(4-(chlorodifluoromethoxy)phenyl)-6-((2-(2-(((S)-1-((2S,4R)-4-hydroxy-2-((4-(4-methyl-1,2,3-thiadiazol-5-yl)benzyl)carbamoyl)pyrrolidin-1-yl)-3,3-dimethyl-1-oxobutan-2-yl)amino)-2-oxoethoxy)ethyl)amino)-5-(1-(tetrahydro-2H-pyran-2-yl)-1H-pyrazol-5-yl)nicotinamide

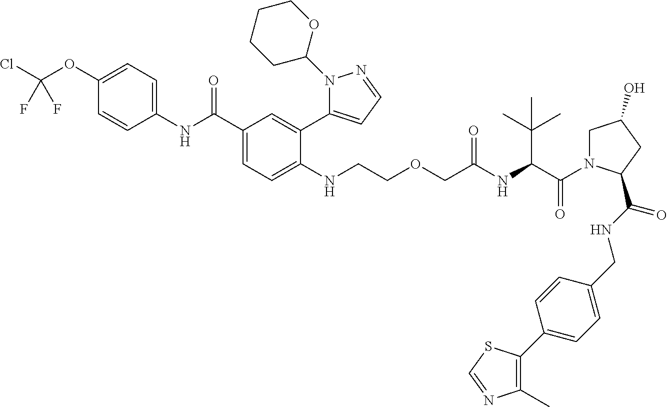 [SMILES: Cc1ncsc1-c1ccc(CNC(=O)[C@@H]2C[C@@H](O)CN2C(=O)[C@@H](NC(=O)COCCNc2ccc(C(=O)Nc3ccc(OC(F)(F)Cl)cc3)cc2-c2ccnn2C2CCCCO2)C(C)(C)C)cc1]

5-Bromo-N-(4-(chlorodifluoromethoxy)phenyl)-6-((2-(2-(((S)-1-((2S,4R)-4-hydroxy-2-((4-(4-methylthiazol-5-yl) benzyl)carbamoyl)pyrrolidin-1-yl)-3,3-dimethyl-1-oxobutan-2-yl)amino)-2-oxoethoxy) ethyl)amino)nicotinamide (18 mg, 0.026 mmol), 1-tetrahydropyran-2-yl-5-(4,4,5,5-tetramethyl-1,3,2-dioxaborolan-2-yl)pyrazole (7.2 mg, 0.026 mmol), tetrakis(triphenylphosphine)palladium(0) (2.3 mg, 10 mol %) and tripotassium phosphate (13 mg, 0.060 mmol) were suspended in toluene (5 ml), placed under a nitrogen atmosphere and heated to 110° C. overnight. Concentrated in vacuo and purified by preparative TLC eluting with 10% methanol/DCM. ¹H NMR (400 MHz, Chloroform-d) δ 8.85 (d, J=16.4 Hz, 1H), 8.76-8.40 (m, 2H), 7.90 (t, J=2.9 Hz, 1H), 7.76-7.65 (m, 2H), 7.63 (d, J=1.7 Hz, 1H), 7.28 (t, J=7.3 Hz, 3H), 7.16 (d, J=8.6 Hz, 2H), 7.07 (dd, J=9.2, 5.7 Hz, 1H), 6.33 (dd, J=7.2, 1.8 Hz, 1H), 5.46 (d, J=6.0 Hz, 1H), 4.97 (ddd, J=10.6, 5.1, 2.4 Hz, 1H), 4.74-4.24 (m, 5H), 4.17-3.85 (m, 4H), 3.80-3.35 (m, 7H), 2.44 (d, J=1.7 Hz, 4H), 2.33-2.21 (m, 1H), 2.21-1.90 (m, 5H), 1.80 (d, J=13.3 Hz, 1H), 1.74-1.41 (m, 3H), 1.18 (t, J=7.0 Hz, 2H), 0.91 (d, J=4.8 Hz, 9H). HRMS: calc. [M+H]⁺ for C₄₇H₅₄ClF₂N₉OS=978.3545; found=978.3815[M+H]⁺.

### N-(4-(chlorodifluoromethoxy)phenyl)-6-((2-(2-(((S)-1-((2S,4R)-4-hydroxy-2-((4-(4-methyl-1,2,3-thiadiazol-5-yl)benzyl)carbamoyl)pyrrolidin-1-yl)-3,3-dimethyl-1-oxobutan-2-yl)amino)-2-oxoethoxy)ethyl)amino)-5-(1H-pyrazol-5-yl)nicotinamide

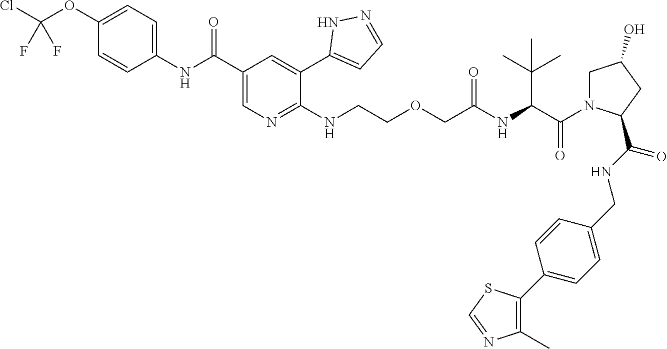 [SMILES: Cc1ncsc1-c1ccc(CNC(=O)[C@@H]2C[C@@H](O)CN2C(=O)[C@@H](NC(=O)COCCNc2ncc(C(=O)Nc3ccc(OC(F)(F)Cl)cc3)cc2-c2ccn[nH]2)C(C)(C)C)cc1]

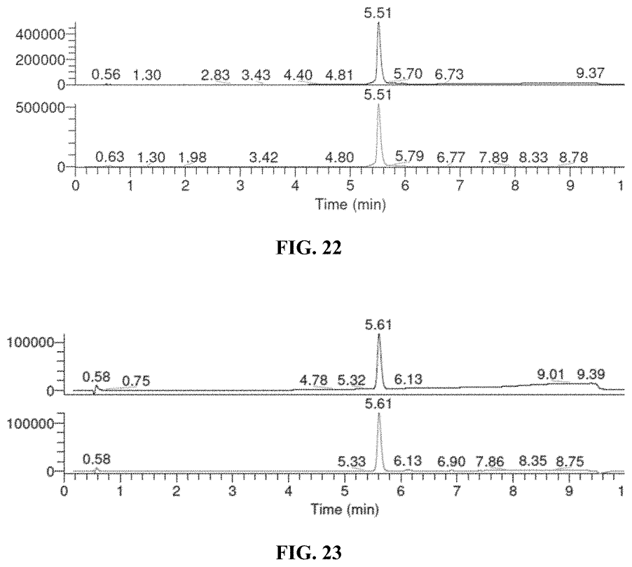

N-(4-(Chlorodifluoromethoxy)phenyl)-6-((2-(2-(((S)-1-((2S,4R)-4-hydroxy-2-((4-(4-methyl-1,2,3-thiadiazol-5-yl)benzyl)carbamoyl)pyrrolidin-1-yl)-3,3-dimethyl-1-oxobutan-2-yl)amino)-2-oxoethoxy)ethyl)amino)-5-(1-(tetrahydro-2H-pyran-2-yl)-1H-pyrazol-5-yl)nicotinamide (14 mg, 0.014 mmol) was dissolved in 20% TFA/DCM and stirred for 2 hours. Concentrated in vacuo and purified by preparative TLC eluting with 10% methanol/DCM. ¹H NMR (400 MHz, Chloroform-d) δ 12.72 (s, 1H), 9.40 (s, 1H), 8.66 (d, J=1.9 Hz, 1H), 8.51 (d, J=2.2 Hz, 1H), 8.34 (s, 1H), 8.17 (d, J=2.3 Hz, 1H), 7.79-7.71 (m, 2H), 7.37 (d, J=8.1 Hz, 2H), 7.32 (d, J=8.2 Hz, 2H), 7.22 (s, 1H), 7.07 (d, J=2.4 Hz, 1H), 6.83 (d, J=7.0 Hz, 1H), 6.55 (d, J=2.4 Hz, 1H), 4.74-4.47 (m, 4H), 4.38-4.28 (m, 2H), 4.01 (s, 2H), 3.79-3.69 (m, 2H), 2.40 (s, 3H), 1.23 (s, 4H), 0.99 (s, 9H), 0.92 (d, J=3.3 Hz, 2H). HRMS: calc. [M+H]⁺ for C₄₂H₄₆ClF₂N₉O₇S=894.2970; found=894.3103[M+H]⁺. See FIG. 22.

### 5-bromo-N-(4-(chlorodifluoromethoxy)phenyl)-6-(3-hydroxypyrrolidin-1-yl)nicotinamide

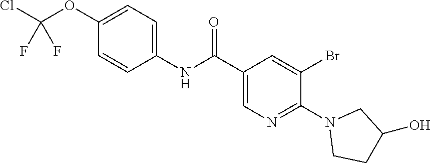 [SMILES: O=C(Nc1ccc(OC(F)(F)Cl)cc1)c1cnc(N2CCC(O)C2)c(Br)c1]

Following a literature procedure, 5-bromo-6-chloro-N-[4-[chloro(difluoro)methoxy]phenyl]pyridine-3-carboxamide (100 mg, 0.243 mmol), (3R)-pyrrolidin-3-ol (25 mg, 0.291 mmol) and triethylamine (75 μl, 0.534 mmol) were heated to 140° C. in isopropanol (5 ml) under microwave conditions for 1 hour. The reaction mixture was allowed to cool to r.t. and poured into water (20 ml). The resulting precipitate was collected by filtration and dried in vacuo to give the title compound (90 mg, 80%). ¹H NMR (400 MHz, DMSO-d₆) δ 10.19 (s, 1H), 8.64 (d, J=2.0 Hz, 1H), 8.31 (d, J=2.1 Hz, 1H), 7.82 (d, J=9.1 Hz, 2H), 7.30 (d, J=8.7 Hz, 2H), 4.95 (d, J=3.3 Hz, 1H), 3.90-3.76 (m, 2H), 3.68 (ddd, J=11.0, 8.0, 3.4 Hz, 1H), 3.53 (d, J=11.5 Hz, 1H), 2.03-1.71 (m, 2H). LC-MS (ESI) m/z: 462,464

### tert-butyl 3-((1-(3-bromo-5-((4-(chlorodifluoromethoxy)phenyl)carbamoyl)pyridin-2-yl)pyrrolidin-3-yl)oxy)propanoate

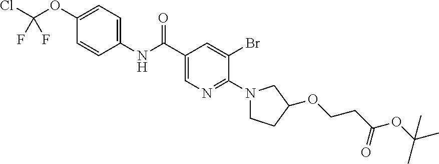 [SMILES: CC(C)(C)OC(=O)CCOC1CCN(c2ncc(C(=O)Nc3ccc(OC(F)(F)Cl)cc3)cc2Br)C1]

5-Bromo-N-[4-[chloro(difluoro)methoxy]phenyl]-6-(3-hydroxypyrrolidin-1-yl)pyridine-3-carboxamide (40 mg, 0.086 mmol) was dissolved in acetonitrile (10 ml) and treated with tert-butyl prop-2-enoate (15 μl, 0.104 mmol) and Triton-B (40% aq., 41 μl, 12 mol %). The reaction mixture was stirred overnight at r.t., diluted with water (10 ml) and extracted with ethyl acetate (3×10 ml). The combined organics were dried over MgSO4 and concentrated in vacuo. The resulting residue was purified by column chromatography eluting with 0-50% ethyl acetate/hexane to yield the title compound (36 mg, 70%). ¹H NMR (400 MHz, Chloroform-d) δ 8.52 (d, J=2.1 Hz, 1H), 8.15 (d, J=2.2 Hz, 1H), 7.64 (d, J=9.0 Hz, 2H), 7.21 (dt, J=9.1, 1.1 Hz, 2H), 4.17-4.07 (m, 1H), 3.99-3.60 (m, 8H), 2.47 (dd, J=7.1, 5.4 Hz, 2H), 1.41 (s, 9H). LC-MS (ESI) m/z: 590/592

### 3-[1-[3-bromo-5-[[4-[chloro(difluoro)methoxy]phenyl]carbamoyl]-2-pyridyl]pyrrolidin-3-yl]oxypropanoic acid

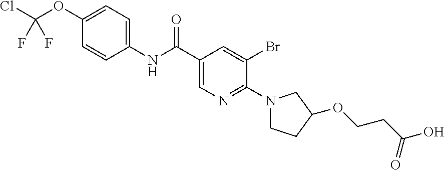 [SMILES: O=C(O)CCOC1CCN(c2ncc(C(=O)Nc3ccc(OC(F)(F)Cl)cc3)cc2Br)C1]

Tert-butyl 3-[1-[3-bromo-5-[[4-[chloro(difluoro)methoxy]phenyl]carbamoyl]-2-pyridyl]pyrrolidin-3-yl]oxypropanoate (36 mg, 0.061 mmol) was dissolved in 20% TFA/DCM and stirred for 2 hours at r.t. The reaction mixture was concentrated in vacuo and used immediately in the next step.

### 5-bromo-N-(4-(chlorodifluoromethoxy)phenyl)-6-(3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)pyrrolidin-1-yl)nicotinamide

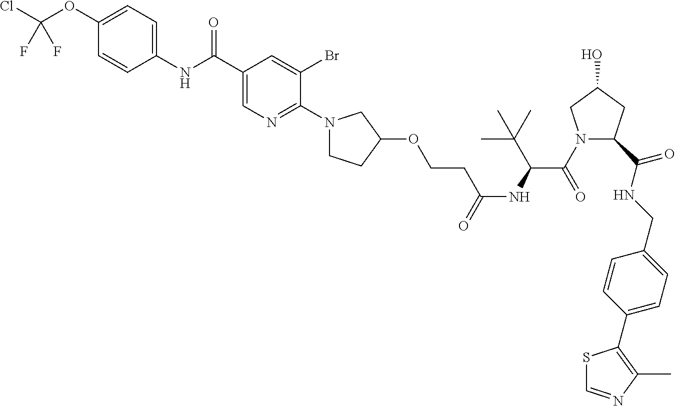 [SMILES: Cc1ncsc1-c1ccc(CNC(=O)[C@@H]2C[C@@H](O)CN2C(=O)[C@@H](NC(=O)CCOC2CCN(c3ncc(C(=O)Nc4ccc(OC(F)(F)Cl)cc4)cc3Br)C2)C(C)(C)C)cc1]

3-[1-[3-Bromo-5-[[4-[chloro(difluoro)methoxy]phenyl]carbamoyl]-2-pyridyl]pyrrolidin-3-yl]oxypropanoic acid (32 mg, 0.061 mmol) was dissolved in DMF (5 ml) and treated sequentially with HATU (25 mg, 0.067 mmol), triethylamine (30 μl, 0.213 mmol) and (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (29 mg, 0.067 mmol) and stirred overnight at r.t. The reaction mixture was diluted with ethyl acetate (15 ml), washed with water (10 ml) and brine (10 ml), dried over MgSO4 and concentrated in vacuo. The residue was purified by column chromatography eluting with 0-10% methanol/DCM to yield the title compound (42 mg, 73%). HRMS: calc. [M+H]⁺ for C₄₂H₄₇ClF₂BrN₇O₇S=946.2170; found=946.2484[M+H]⁺.

### N-(4-(chlorodifluoromethoxy)phenyl)-6-(3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)pyrrolidin-1-yl)-5-(1-(tetrahydro-2H-pyran-2-yl)-1H-pyrazol-5-yl)nicotinamide

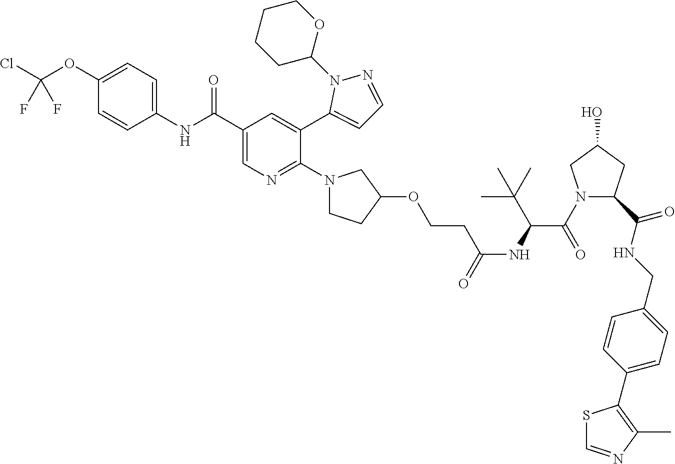 [SMILES: Cc1ncsc1-c1ccc(CNC(=O)[C@@H]2C[C@@H](O)CN2C(=O)[C@@H](NC(=O)CCOC2CCN(c3ncc(C(=O)Nc4ccc(OC(F)(F)Cl)cc4)cc3-c3ccnn3C3CCCCO3)C2)C(C)(C)C)cc1]

5-Bromo-N-(4-(chlorodifluoromethoxy)phenyl)-6-(3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)pyrrolidin-1-yl)nicotinamide (42 mg, 0.043 mol) 1-tetrahydropyran-2-yl-5-(4,4,5,5-tetramethyl-1,3,2-dioxaborolan-2-yl)pyrazole (16 mg, 0.057 mmol), tetrakis(triphenylphosphine)palladium(0) (5 mg, 10 mol %) and tripotassium phosphate (28 mg, 0.133 mmol) were suspended in toluene (5 ml), placed under a nitrogen atmosphere and heated to 110° C. overnight. Concentrated in vacuo and purified by preparative TLC eluting with 10% methanol/DCM. HRMS: calc. [M+H]⁺ for C₅₀H₅₈ClF₂N₉O₈S=1018.3858; found=1018.4096[M+H]⁺.

### N-(4-(chloroodifluoromethoxy)phenyl)-6-(3-(3-(((S)-1-((2S,4R)-4-hydroxy-2-((4-(4-methylthiazol-5-yl) benzyl)carbamoyl) pyrrolidin-1-yl)-3,3-dimethyl-1-oxobutan-2-yl) amino)-3-oxopropoxy) pyrrolidin-1-yl)-5-(1H-pyrazol-5-yl)nicotinamide

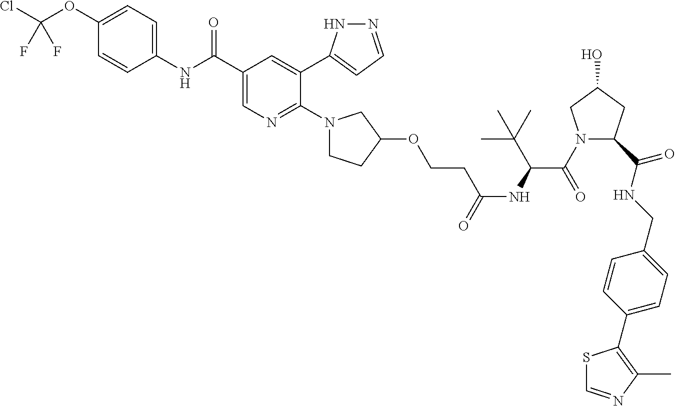 [SMILES: Cc1ncsc1-c1ccc(CNC(=O)[C@@H]2C[C@@H](O)CN2C(=O)[C@@H](NC(=O)CCOC2CCN(c3ncc(C(=O)Nc4ccc(OC(F)(F)Cl)cc4)cc3-c3ccn[nH]3)C2)C(C)(C)C)cc1]

N-(4-(Chlorodifluoromethoxy)phenyl)-6-(3-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)pyrrolidin-1-yl)-5-(1-(tetrahydro-2H-pyran-2-yl)-1H-pyrazol-5-yl)nicotinamide (22 mg, 0.021 mmol) was dissolved in 20% TFA/DCM and stirred for 2 hours. Concentrated in vacuo and purified by preparative TLC eluting with 10% methanol/DCM to yield the title compound (5.5 mg, 27%). HRMS: calc. [M+H]⁺ for C₄₅H₅₀ClF₂N₉O₇S=934.3413; found=934.3506 [M+H]⁺.

### 3-((1-(3-bromo-5-((4-(chlorodifluoromethoxy)phenyl)carbamoyl)pyridin-2-yl)piperidin-4-yl)methoxy)propanoic acid

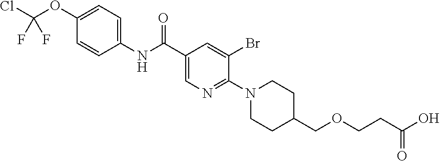 [SMILES: O=C(O)CCOCC1CCN(c2ncc(C(=O)Nc3ccc(OC(F)(F)Cl)cc3)cc2Br)CC1]

5-Bromo-6-chloro-N-[4-[chloro(difluoro)methoxy]phenyl]pyridine-3-carboxamide (50 mg, 0.121 mmol) and tert-butyl 4-[(3-tert-butoxy-3-oxo-propoxy)methyl]piperidine-1-carboxylate (62 mg, 0.182 mmol) and triethylamine (68 μl, 0.485 mmol) were suspended in trifluoroethanol and heated to 150° C. under microwave conditions for 2 hours. The reaction mixture was concentrated in vacuo and purified by column chromatography eluting with 0-15% methanol/DCM to yield the title compound (46 mg, 67%). ¹H NMR (400 MHz, Methanol-d4) δ 8.73 (d, J=2.2 Hz, 1H), 8.38 (d, J=2.2 Hz, 1H), 7.79 (d, J=9.1 Hz, 2H), 7.25 (d, J=8.7 Hz, 2H), 4.03 (dq, J=13.3, 2.3 Hz, 4H), 3.67 (dt, J=15.8, 6.2 Hz, 4H), 3.40-3.32 (m, 1H), 2.92-2.82 (m, 2H), 2.50 (dt, J=12.2, 6.2 Hz, 2H). LC-MS (ESI) m/z: 562,564.

### 5-bromo-N-(4-(chlorodifluoromethoxy)phenyl)-6-(4-((3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)methyl)piperidin-1-yl)nicotinamide

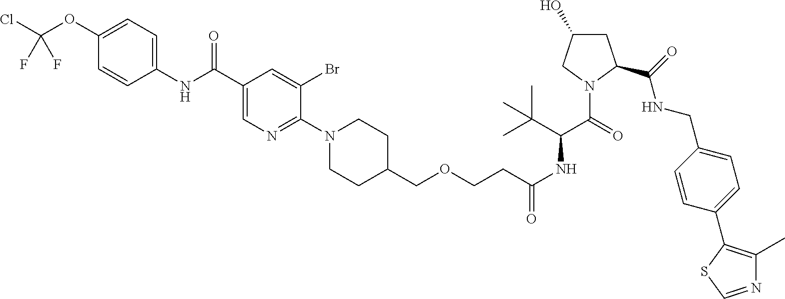 [SMILES: Cc1ncsc1-c1ccc(CNC(=O)[C@@H]2C[C@@H](O)CN2C(=O)[C@@H](NC(=O)CCOCC2CCN(c3ncc(C(=O)Nc4ccc(OC(F)(F)Cl)cc4)cc3Br)CC2)C(C)(C)C)cc1]

3-[[1-[3-Bromo-5-[[4-[chloro(difluoro)methoxy]phenyl]carbamoyl]-2-pyridyl]-4-piperidyl]methoxy]propanoic acid (42 mg, 0.075 mmol) was dissolved in DMF (5 ml) and treated sequentially with HATU (32 mg, 0.085 mmol), triethylamine (35 μl, 0.255 mmol) and (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (37 mg, 0.085 mmol) and stirred overnight at r.t. The reaction mixture was diluted with ethyl acetate (15 ml), washed with water (10 ml) and brine (10 ml), dried over MgSO4 and concentrated in vacuo. The residue was purified by column chromatography eluting with 0-10% methanol/DCM to yield the title compound (43 mg, 59%). HRMS: calc. [M+H]⁺ for C₄₄H₅₁BrClF₂N₇O₇S=974.2483; found=974.2599 [M+H]⁺.

### N-(4-(chlorodifluoromethoxy)phenyl)-6-(4-((3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)methyl)piperidin-1-yl)-5-(1-(tetrahydro-2H-pyran-2-yl)-1H-pyrazol-5-yl)nicotinamide

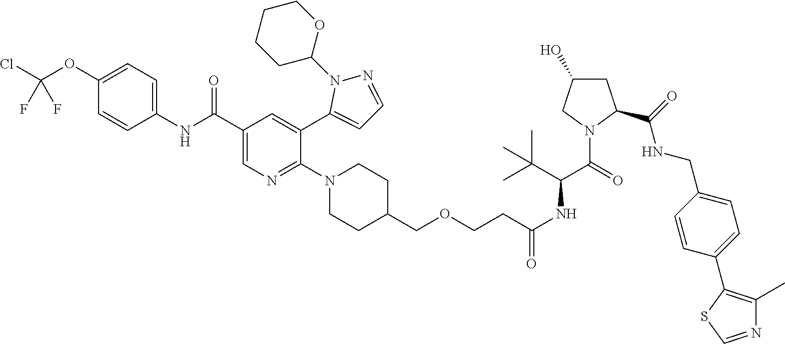 [SMILES: Cc1ncsc1-c1ccc(CNC(=O)[C@@H]2C[C@@H](O)CN2C(=O)[C@@H](NC(=O)CCOCC2CCN(c3ncc(C(=O)Nc4ccc(OC(F)(F)Cl)cc4)cc3-c3ccnn3C3CCCCO3)CC2)C(C)(C)C)cc1]

5-Bromo-N-(4-(chlorodifluoromethoxy)phenyl)-6-(4-((3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)methyl)piperidin-1-yl)nicotinamide (43 mg, 0.044 mol) 1-tetrahydropyran-2-yl-5-(4,4,5,5-tetramethyl-1,3,2-dioxaborolan-2-yl)pyrazole (16 mg, 0.057 mmol), tetrakis(triphenylphosphine)palladium(0) (5 mg, 10 mol %) and tripotassium phosphate (28 mg, 0.133 mmol) were suspended in toluene (5 ml), placed under a nitrogen atmosphere and heated to 110° C. overnight. Concentrated in vacuo and purified by preparative TLC eluting with 10% methanol/DCM. HRMS: calc. [M+H]⁺ for C₅₂H₆₂ClF₂N₉O₈S=1046.4171; found=1045.4429[M+H]⁺.

### N-(4-(chlorodifluoromethoxy)phenyl)-6-(4-((3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)methyl)piperidin-1-yl)-5-(1H-pyrazol-5-yl)nicotinamide

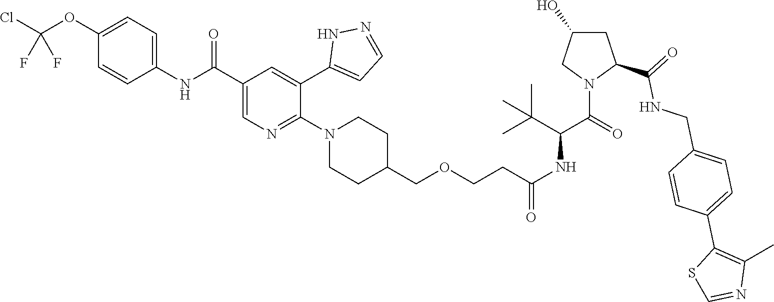 [SMILES: Cc1ncsc1-c1ccc(CNC(=O)[C@@H]2C[C@@H](O)CN2C(=O)[C@@H](NC(=O)CCOCC2CCN(c3ncc(C(=O)Nc4ccc(OC(F)(F)Cl)cc4)cc3-c3ccn[nH]3)CC2)C(C)(C)C)cc1]

N-(4-(Chlorodifluoromethoxy)phenyl)-6-(4-((3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)methyl)piperidin-1-yl)-5-(1-(tetrahydro-2H-pyran-2-yl)-1H-pyrazol-5-yl)nicotinamide (22 mg, 0.021 mmol) was dissolved in 20% TFA/DCM and stirred for 2 hours. Concentrated in vacuo and purified by preparative TLC eluting with 10% methanol/DCM to yield the title compound (7 mg, 34%). HRMS: calc. [M+H]⁺ for C₅₂H₆₂ClF₂N₉O₈S=962.3596; found=962.3867 [M+H]⁺.

### 3-[[(3S)-1-[3-bromo-5-[[4-[chloro(difluoro)methoxy]phenyl]carbamoyl]-2-pyridyl]pyrrolidin-3-yl]methoxy]propanoic acid

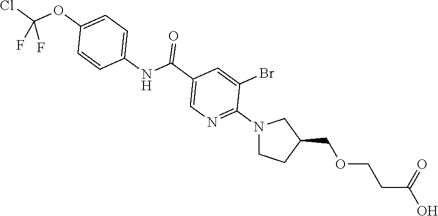 [SMILES: O=C(O)CCOC[C@H]1CCN(c2ncc(C(=O)Nc3ccc(OC(F)(F)Cl)cc3)cc2Br)C1]

5-Bromo-6-chloro-N-[4-[chloro(difluoro)methoxy]phenyl]pyridine-3-carboxamide (50 mg, 0.121 mmol) and tert-butyl (3 S)-3-[(3-tert-butoxy-3-oxo-propoxy)methyl]pyrrolidine-1-carboxylate (60 mg, 0.182 mmol) and triethylamine (68 μl, 0.485 mmol) were suspended in trifluoroethanol and heated to 150° C. under microwave conditions for 2 hours. The reaction mixture was concentrated in vacuo and purified by column chromatography eluting with 0-15% methanol/DCM to yield the title compound (21 mg, 32%). LC-MS (ESI) m/z: 548, 550.

### 5-bromo-N-(4-(chlorodifluoromethoxy)phenyl)-6-(4-((3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)methyl)piperidin-1-yl)nicotinamide

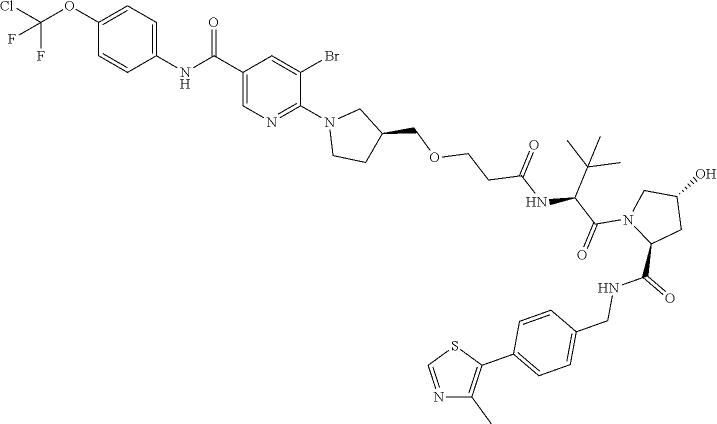 [SMILES: Cc1ncsc1-c1ccc(CNC(=O)[C@@H]2C[C@@H](O)CN2C(=O)[C@@H](NC(=O)CCOC[C@H]2CCN(c3ncc(C(=O)Nc4ccc(OC(F)(F)Cl)cc4)cc3Br)C2)C(C)(C)C)cc1]

3-[[1-[3-Bromo-5-[[4-[chloro(difluoro)methoxy]phenyl]carbamoyl]-2-pyridyl]-4-piperidyl]methoxy]propanoic acid (21 mg, 0.038 mmol) was dissolved in DMF (5 ml) and treated sequentially with HATU (16 mg, 0.043 mmol), triethylamine (18 μl, 0.255 mmol) and (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (18 mg, 0.043 mmol) and stirred overnight at r.t. The reaction mixture was diluted with ethyl acetate (15 ml), washed with water (10 ml) and brine (10 ml), dried over MgSO4 and concentrated in vacuo. The residue was purified by column chromatography eluting with 0-10% methanol/DCM to yield the title compound (35 mg, 93%). HRMS: calc. [M+H]⁺ for C₄₃H₄₉BrClF₂N₇O₇S=960/2563; found=960.2327 [M+H]⁺.

### N-(4-(chlorodifluoromethoxy)phenyl)-6-((S)-3-((3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)methyl)pyrrolidin-1-yl)-5-(1-(tetrahydro-2-pyran-2-yl)-1H-pyrazol-5-yl)nicotinamide

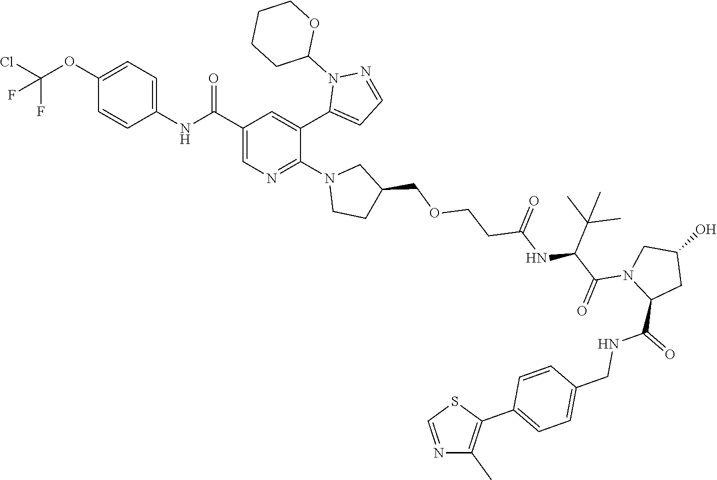 [SMILES: Cc1ncsc1-c1ccc(CNC(=O)[C@@H]2C[C@@H](O)CN2C(=O)[C@@H](NC(=O)CCOC[C@H]2CCN(c3ncc(C(=O)Nc4ccc(OC(F)(F)Cl)cc4)cc3-c3ccnn3C3CCCCO3)C2)C(C)(C)C)cc1]

5-Bromo-N-[4-[chloro(difluoro)methoxy]phenyl]-6-[(3 S)-3-[[3-[[(1 S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-3-oxo-propoxy]methyl]pyrrolidin-1-yl]pyridine-3-carboxamide (35 mg, 0.036 mol) 1-tetrahydropyran-2-yl-5-(4,4,5,5-tetramethyl-1,3,2-dioxaborolan-2-yl)pyrazole (13 mg, 0.047 mmol), tetrakis(triphenylphosphine)palladium(0) (4.2 mg, 10 mol %) and tripotassium phosphate (23 mg, 0.110 mmol) were suspended in toluene (5 ml), placed under a nitrogen atmosphere and heated to 110° C. overnight. Concentrated in vacuo and purified by preparative TLC eluting with 10% methanol/DCM. HRMS: calc. [M+H]⁺ for C₅₁H₆₀ClF₂N₉O₈S=1032.4015; found=1032.4292[M+H]⁺.

### N-(4-(chlorodifluoromethoxy)phenyl)-6-((S)-3-((3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)methyl)pyrrolidin-1-yl)-5-(1H-pyrazol-5-yl)nicotinamide

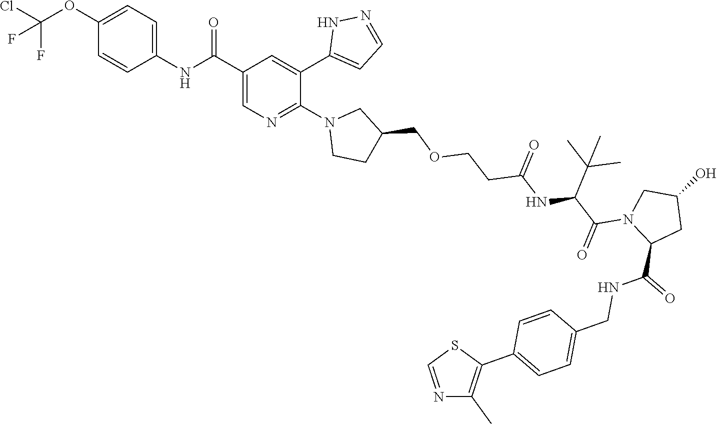 [SMILES: Cc1ncsc1-c1ccc(CNC(=O)[C@@H]2C[C@@H](O)CN2C(=O)[C@@H](NC(=O)CCOC[C@H]2CCN(c3ncc(C(=O)Nc4ccc(OC(F)(F)Cl)cc4)cc3-c3ccn[nH]3)C2)C(C)(C)C)cc1]

N-(4-(Chlorodifluoromethoxy)phenyl)-6-((S)-3-((3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)methyl)pyrrolidin-1-yl)-5-(1-(tetrahydro-2H-pyran-2-yl)-1H-pyrazol-5-yl)nicotinamide (14 mg, 0.021 mmol) was dissolved in 20% TFA/DCM and stirred for 2 hours. Concentrated in vacuo and purified by preparative TLC eluting with 10% methanol/DCM to yield the title compound (3 mg, 23%). HRMS: calc. [M+H]⁺ for C₅₂H₆₂ClF₂N₉O₈S=948.3440; found=948.3391 [M+H]⁺.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

### N-(4-(chlorodifluoromethoxy)phenyl)-6-((2-(2-(((R)-1-((2R,4S)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethyl)amino)-5-(1H-pyrazol-5-yl)nicotinamide

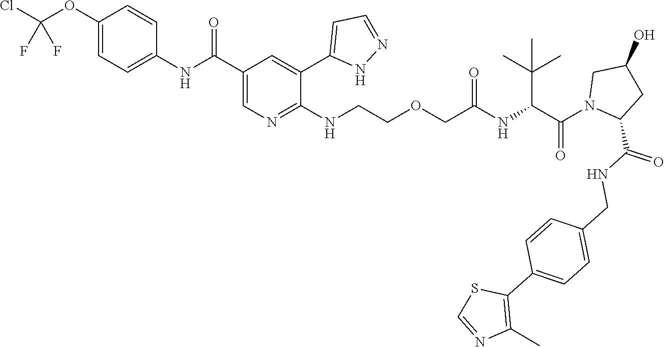 [SMILES: Cc1ncsc1-c1ccc(CNC(=O)[C@H]2C[C@H](O)CN2C(=O)[C@H](NC(=O)COCCNc2ncc(C(=O)Nc3ccc(OC(F)(F)Cl)cc3)cc2-c2ccn[nH]2)C(C)(C)C)cc1]

Prepared as described above but using the diastereomeric VHL ligand. ¹H NMR (400 MHz, DMSO-d₆) δ 13.13 (s, 1H), 10.19 (s, 1H), 8.94 (s, 1H), 8.65-8.59 (m, 2H), 8.28 (s, 1H), 7.85 (dd, J=9.9, 2.7 Hz, 2H), 7.32 (d, J=8.7 Hz, 2H), 6.91 (s, 1H), 6.25 (s, 1H), 5.42 (s, 1H), 4.42-4.13 (m, 4H), 3.99 (s, 2H), 3.91-3.67 (m, 4H), 2.63 (d, J=2.0 Hz, 2H), 2.39 (s, 3H), 2.29 (t, J=1.9 Hz, 2H), 1.71 (dt, J=12.3, 6.1 Hz, 1H), 1.20 (s, 2H), 0.88 (s, 9H).

HRMS: calc. [M+H]⁺ for C₄₂H₄₆ClF₂N₉O₇S=894.2970; found=894.3213 [M+H]⁺. See FIG. 23

### ENUMERATED EMBODIMENTS

The following enumerated embodiments are provided, the order of which is not to be construed as designating levels of importance:

An aspect of the present disclosure provides a compound of Formula (I):

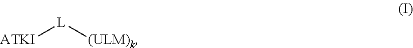

wherein:
ATKI is an allosteric tyrosine kinase inhibitor, L is a linker, each ULM is independently a ubiquitin ligase binder, and k is an integer ranging from 1 to 4, wherein ATKI is covalently linked to L and wherein each ULM is covalently linked to L; or a salt, enantiomer, stereoisomer, solvate, polymorph or N-oxide thereof.

In any aspect or embodiment described herein, ATKI is capable of binding to c-ABL and/or BCR-ABL.

In any aspect or embodiment described herein, upon binding of the compound of Formula (I) simultaneously to a tyrosine kinase and a ubiquitin ligase, the tyrosine kinase is ubiquitinated by the ubiquitin ligase.

In any aspect or embodiment described herein, at least one ULM binds to an E3 ubiquitin ligase.

In any aspect or embodiment described herein, the E3 ubiquitin ligase comprises a Von Hippel Lindau (VHL) E3 ubiquitin ligase, an MDM2 E3 ubiquitin ligase, Inhibitor of Apoptosis Protein (IAP) E3 ubiquitin ligase, or a Cereblon (CRBN) E3 ubiquitin ligase.

In any aspect or embodiment described herein, the ATKI binds to an allosteric site on c-ABL and inhibits c-ABL.

In any aspect or embodiment described herein, the ATKI binds to an allosteric site on BCR-ABL and inhibits BCR-ABL.

In any aspect or embodiment described herein, the ATKI binds to an allosteric site on at least one of c-ABL and BCR-ABL and inhibits at least one of c-ABL and BCR-ABL.

In any aspect or embodiment described herein, the ATKI is selected from the group consisting of GNF-2, GNF-5, asciminib, or any combinations thereof.

In any aspect or embodiment described herein, at least one ULM comprises Formula (XXI):

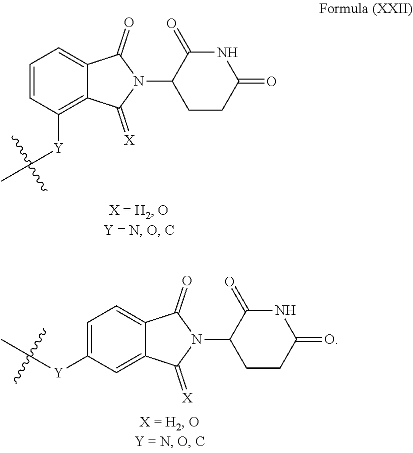

In any aspect or embodiment described herein, at least one ULM comprises Formula (XXIII):

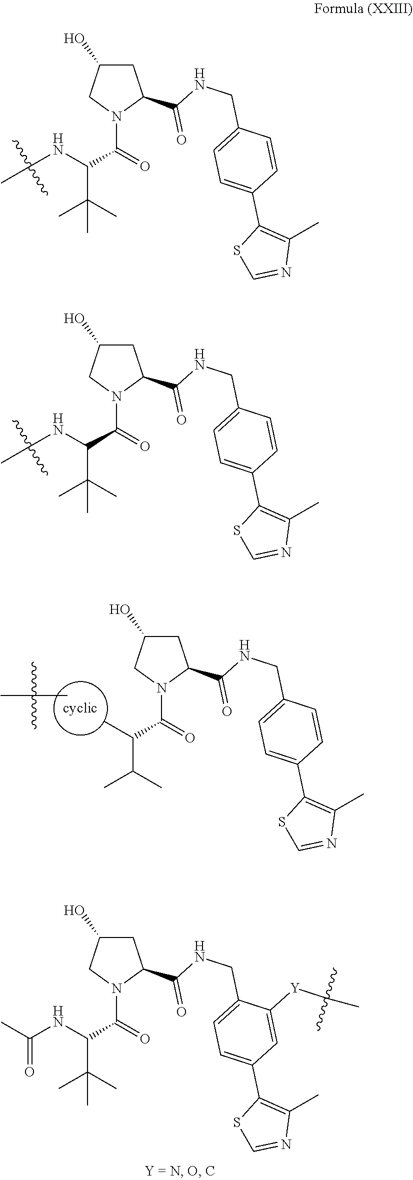

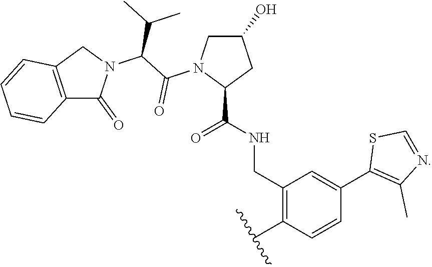 [SMILES: *c1ccc(-c2scnc2C)cc1CNC(=O)[C@@H]1C[C@@H](O)CN1C(=O)[C@H](C(C)C)N1Cc2ccccc2C1=O]

In any aspect or embodiment described herein, k is 1.

In any aspect or embodiment described herein, the linker L has the formula —(CH₂)ₘ₁—X⁴—((CH₂)ₘ₂—X⁵)ₘ₂—(CH₂)ₙ₃—X⁶—, wherein: if m1 is greater than 0 then —(CH₂)ₘ₁ is covalently bonded to the ATKI; if m1 is 0 then X⁴ is covalently bonded to the ATKI; —X⁶ is covalently bonded to the ULM; each m1, m2, m2′, and m3 is independently 0, 1, 2, 3, 4, 5, 6, 7, 8, 9, or 10; each X⁴ and X⁵ independently absent (a bond), O, S, or N—R²⁰; each X⁶ is independently absent (a bond), C(═O), NHC(═O), C(═S), C(═NR²⁰), O, S, or N—R²⁰; and wherein each R²⁰ is independently selected from the group consisting of hydrogen, optionally substituted C₁-C₆ alkyl, optionally substituted aryl, optionally substituted heteroaryl, optionally substituted C₃-C₈ cycloalkyl, and optionally substituted C₃-C₈ cycloheteroalkyl.

In any aspect or embodiment described herein, m1 is 0; m2′ is 2; m2 is 1 or 2; m3 is 1; and X⁴, X⁵, and X⁶ are O.

In any aspect or embodiment described herein, m1 is 2; m2′ is 2; m2 is 1; m3 is 1; and X⁴, X⁵, and X⁶ are O.

In any aspect or embodiment described herein, m1 is 2; m2′ is 2; m2 is 3; m3 is 1; and X⁴, X⁵, and X⁶ are O.

In any aspect or embodiment described herein, the compound of the present disclosure is selected from the group consisting of:

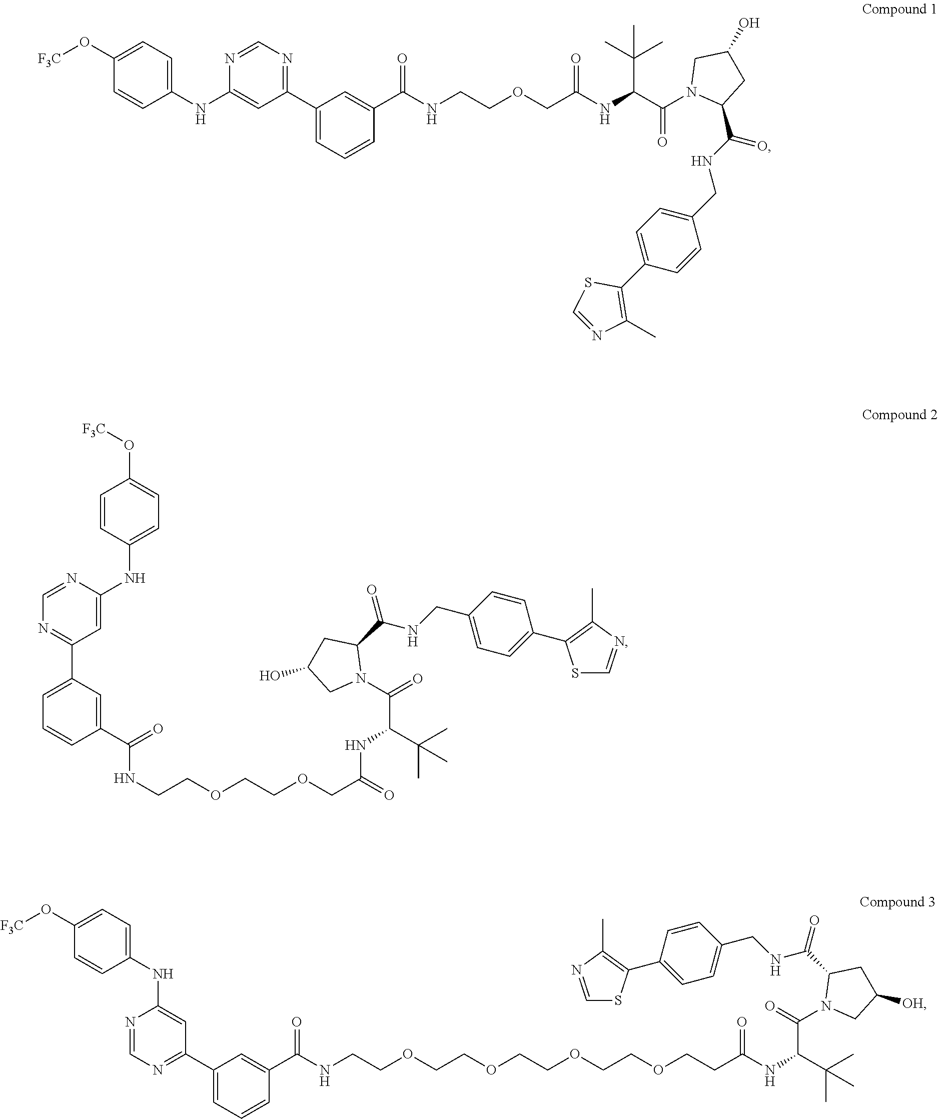 [SMILES: Cc1ncsc1-c1ccc(CNC(=O)[C@@H]2C[C@@H](O)CN2C(=O)[C@@H](NC(=O)COCCNC(=O)c2cccc(-c3cc(Nc4ccc(OC(F)(F)F)cc4)ncn3)c2)C(C)(C)C)cc1 ; Cc1ncsc1-c1ccc(CNC(=O)[C@@H]2C[C@@H](O)CN2C(=O)[C@@H](NC(=O)COCCOCCNC(=O)c2cccc(-c3cc(Nc4ccc(OC(F)(F)F)cc4)ncn3)c2)C(C)(C)C)cc1 ; Cc1ncsc1-c1ccc(CNC(=O)[C@@H]2C[C@@H](O)CN2C(=O)[C@@H](NC(=O)CCOCCOCCOCCOCCNC(=O)c2cccc(-c3cc(Nc4ccc(OC(F)(F)F)cc4)ncn3)c2)C(C)(C)C)cc1]

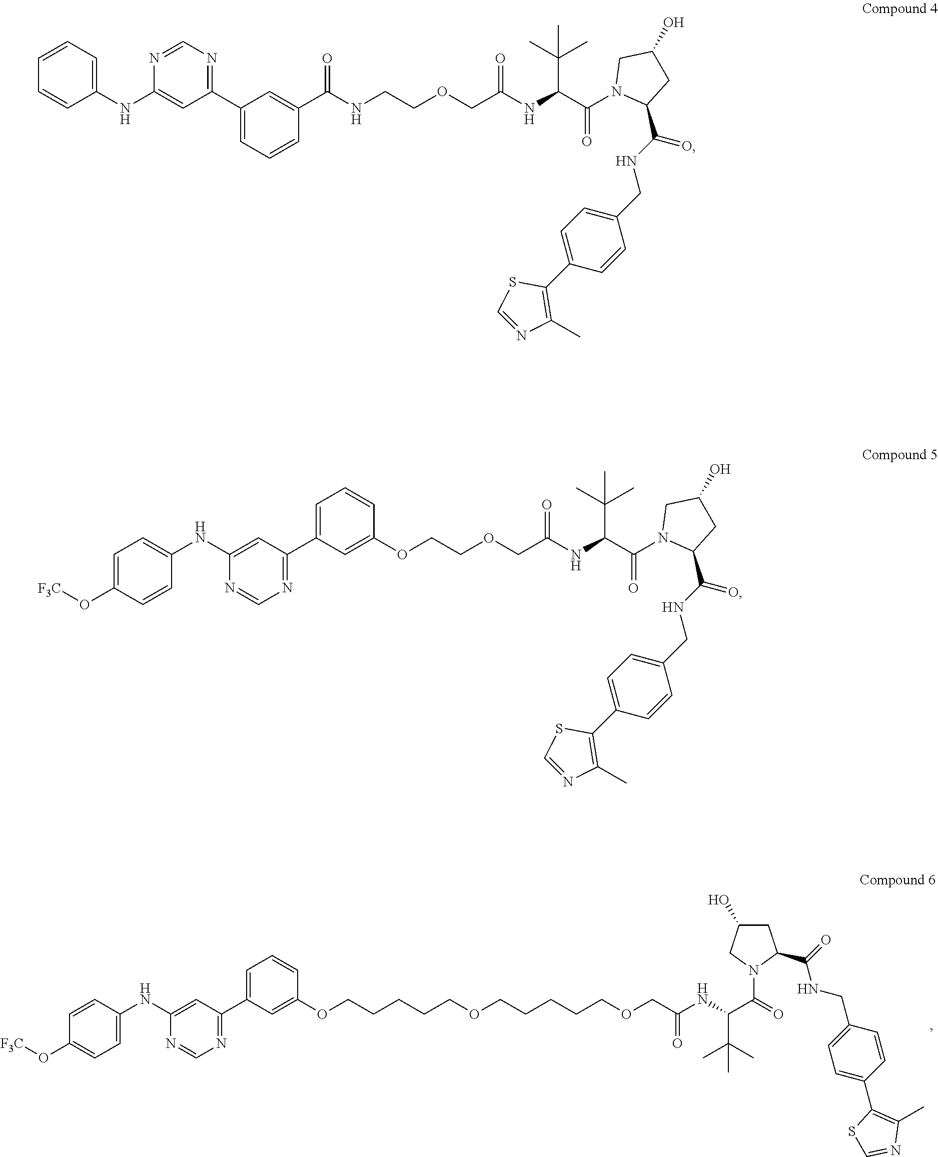 [SMILES: Cc1ncsc1-c1ccc(CNC(=O)[C@@H]2C[C@@H](O)CN2C(=O)[C@@H](NC(=O)COCCNC(=O)c2cccc(-c3cc(Nc4ccccc4)ncn3)c2)C(C)(C)C)cc1 ; Cc1ncsc1-c1ccc(CNC(=O)[C@@H]2C[C@@H](O)CN2C(=O)[C@@H](NC(=O)COCCOc2cccc(-c3cc(Nc4ccc(OC(F)(F)F)cc4)ncn3)c2)C(C)(C)C)cc1 ; Cc1ncsc1-c1ccc(CNC(=O)[C@@H]2C[C@@H](O)CN2C(=O)[C@@H](NC(=O)COCCCCCOCCCCCOc2cccc(-c3cc(Nc4ccc(OC(F)(F)F)cc4)ncn3)c2)C(C)(C)C)cc1]

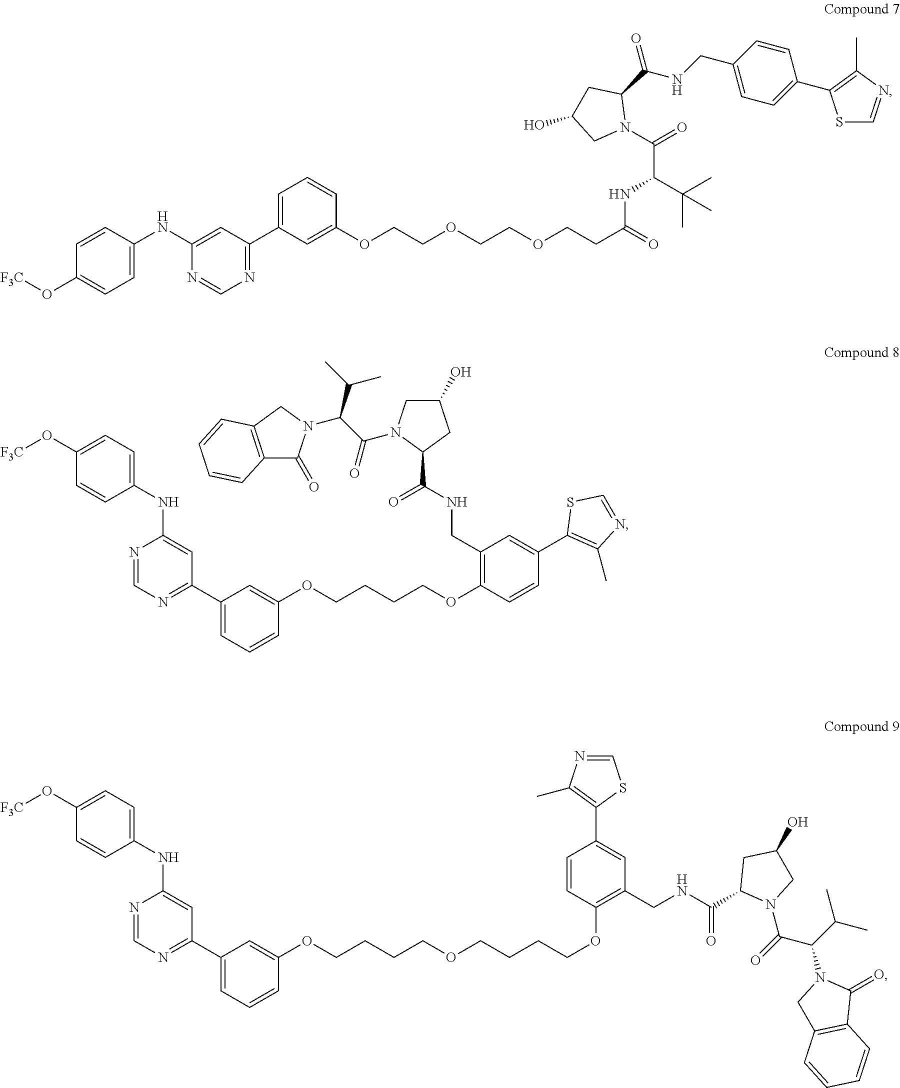 [SMILES: Cc1ncsc1-c1ccc(CNC(=O)[C@@H]2C[C@@H](O)CN2C(=O)[C@@H](NC(=O)CCOCCOCCOc2cccc(-c3cc(Nc4ccc(OC(F)(F)F)cc4)ncn3)c2)C(C)(C)C)cc1 ; Cc1ncsc1-c1ccc(OCCCCOc2cccc(-c3cc(Nc4ccc(OC(F)(F)F)cc4)ncn3)c2)c(CNC(=O)[C@@H]2C[C@@H](O)CN2C(=O)[C@H](C(C)C)N2Cc3ccccc3C2=O)c1 ; Cc1ncsc1-c1ccc(OCCCCOCCCCOc2cccc(-c3cc(Nc4ccc(OC(F)(F)F)cc4)ncn3)c2)c(CNC(=O)[C@@H]2C[C@@H](O)CN2C(=O)[C@H](C(C)C)N2Cc3ccccc3C2=O)c1]

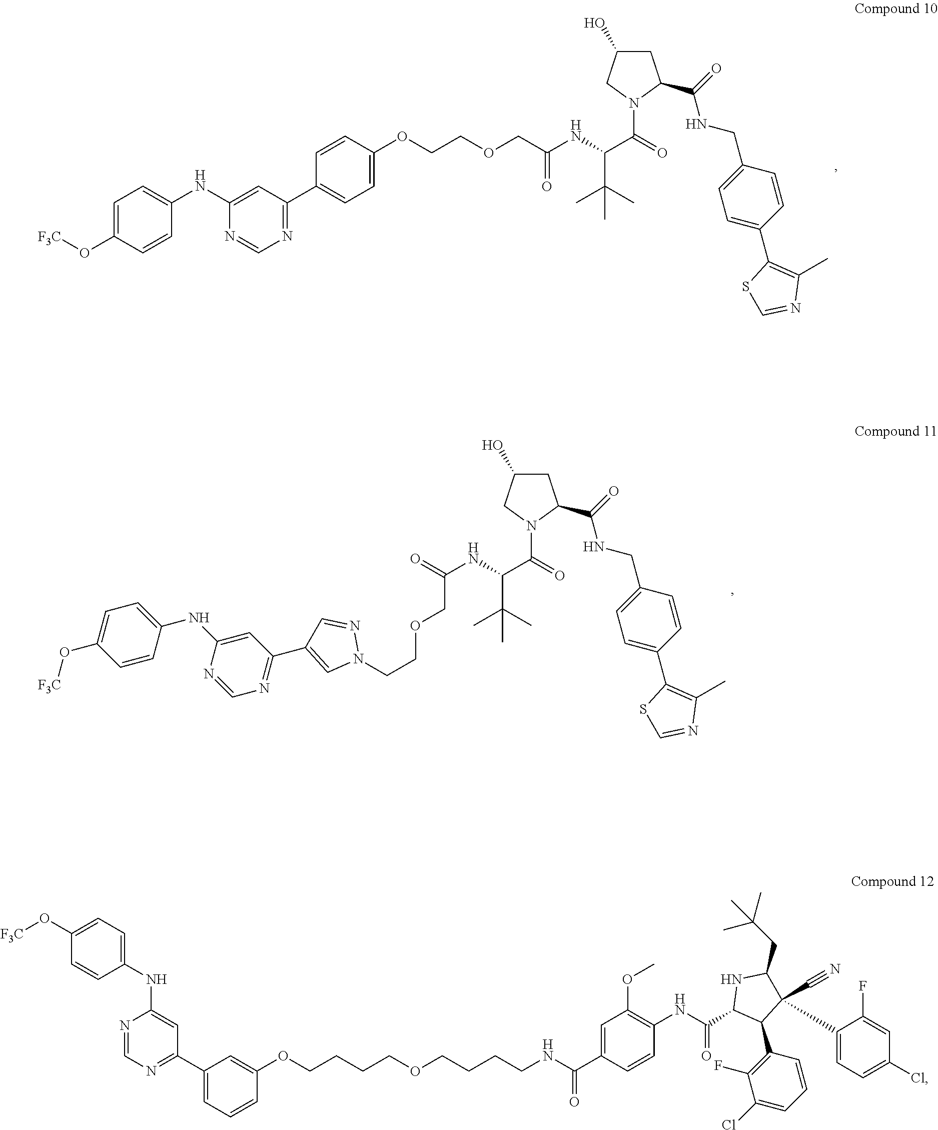 [SMILES: Cc1ncsc1-c1ccc(CNC(=O)[C@@H]2C[C@@H](O)CN2C(=O)[C@@H](NC(=O)COCCOc2ccc(-c3cc(Nc4ccc(OC(F)(F)F)cc4)ncn3)cc2)C(C)(C)C)cc1 ; Cc1ncsc1-c1ccc(CNC(=O)[C@@H]2C[C@@H](O)CN2C(=O)[C@@H](NC(=O)COCCn2cc(-c3cc(Nc4ccc(OC(F)(F)F)cc4)ncn3)cn2)C(C)(C)C)cc1 ; COc1cc(C(=O)NCCCCOCCCCOc2cccc(-c3cc(Nc4ccc(OC(F)(F)F)cc4)ncn3)c2)ccc1NC(=O)[C@@H]1N[C@@H](CC(C)(C)C)[C@](C#N)(c2ccc(Cl)cc2F)[C@H]1c1cccc(Cl)c1F]

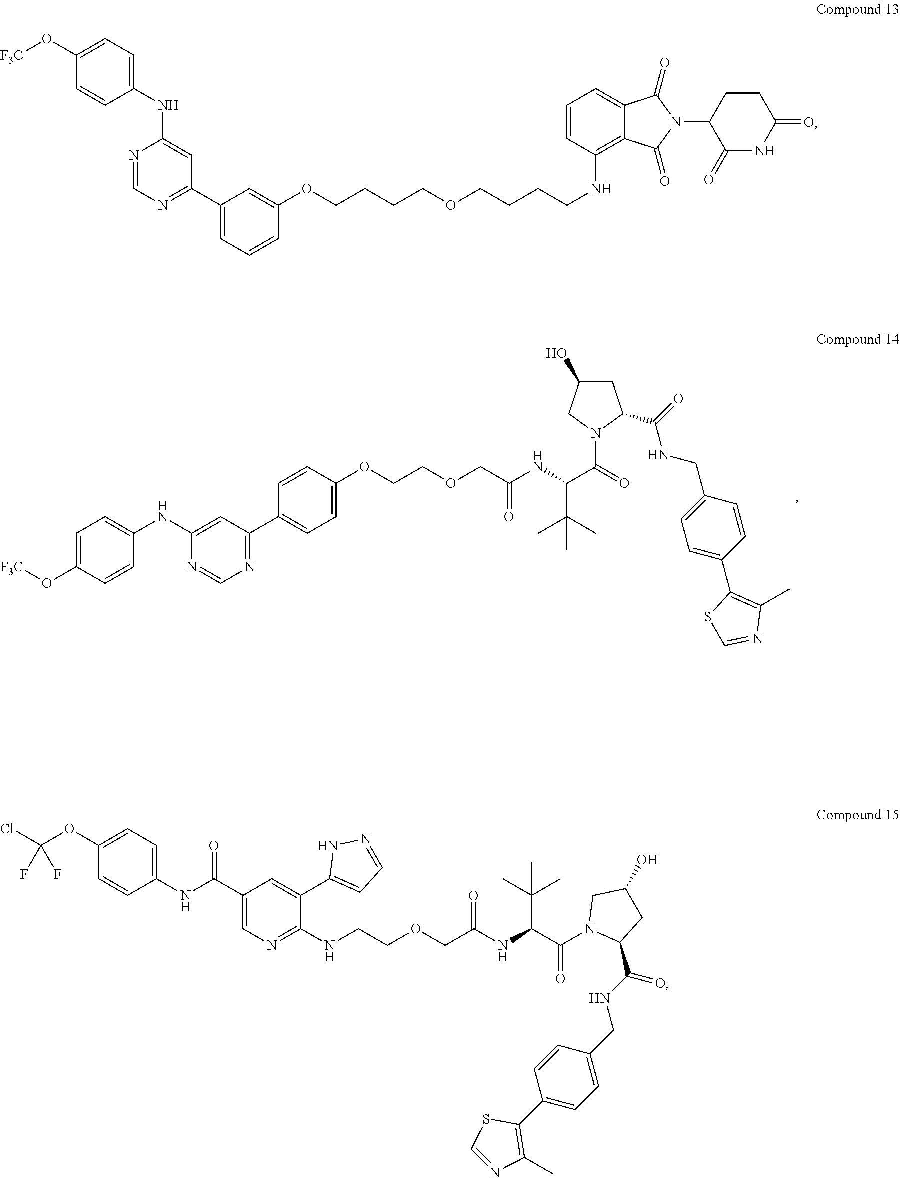 [SMILES: O=C1CCC(N2C(=O)c3cccc(NCCCCOCCCCOc4cccc(-c5cc(Nc6ccc(OC(F)(F)F)cc6)ncn5)c4)c3C2=O)C(=O)N1 ; Cc1ncsc1-c1ccc(CNC(=O)[C@H]2C[C@H](O)CN2C(=O)[C@@H](NC(=O)COCCOc2ccc(-c3cc(Nc4ccc(OC(F)(F)F)cc4)ncn3)cc2)C(C)(C)C)cc1 ; Cc1ncsc1-c1ccc(CNC(=O)[C@@H]2C[C@@H](O)CN2C(=O)[C@@H](NC(=O)COCCNc2ncc(C(=O)Nc3ccc(OC(F)(F)Cl)cc3)cc2-c2ccn[nH]2)C(C)(C)C)cc1]

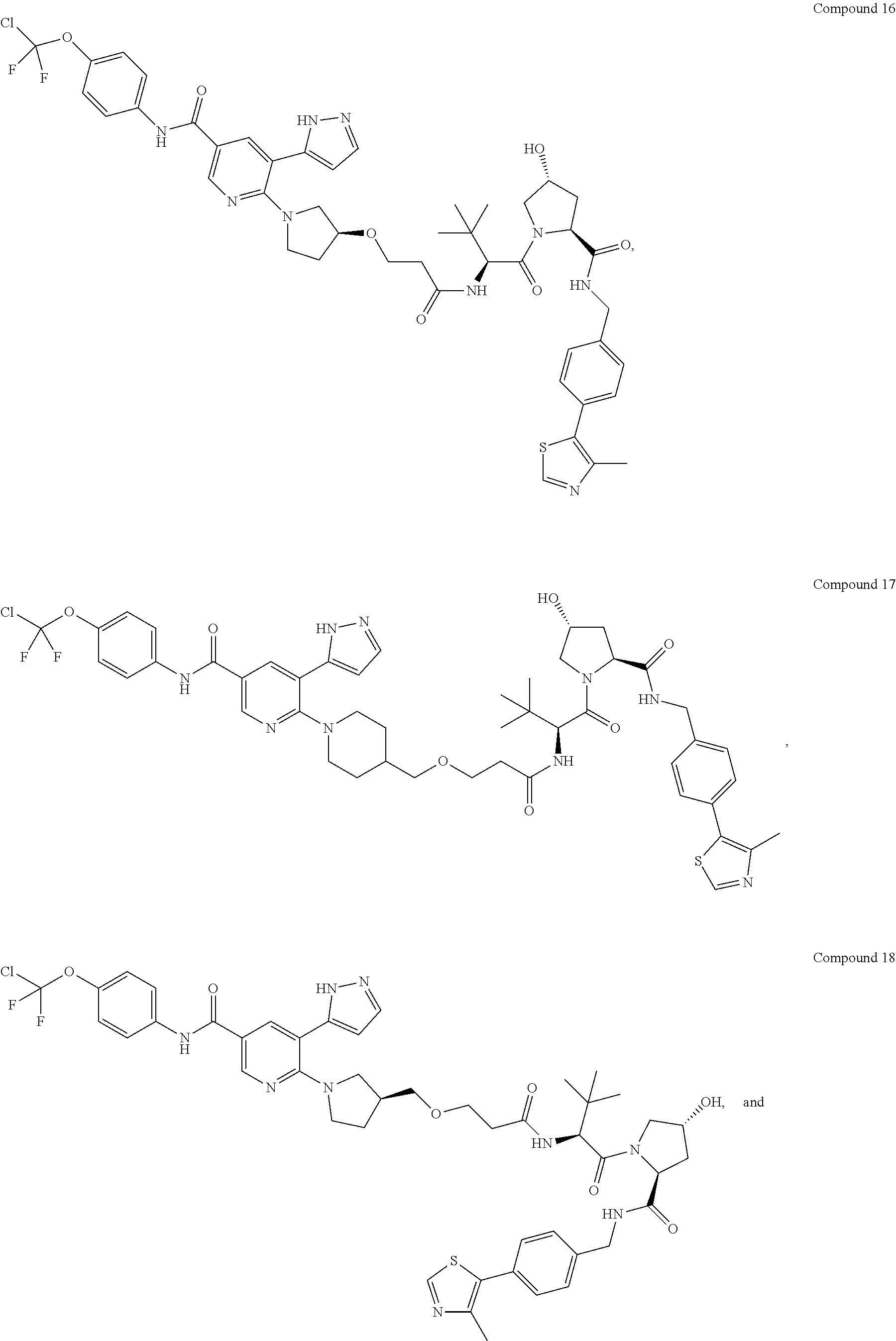 [SMILES: Cc1ncsc1-c1ccc(CNC(=O)[C@@H]2C[C@@H](O)CN2C(=O)[C@@H](NC(=O)CCO[C@H]2CCN(c3ncc(C(=O)Nc4ccc(OC(F)(F)Cl)cc4)cc3-c3ccn[nH]3)C2)C(C)(C)C)cc1 ; Cc1ncsc1-c1ccc(CNC(=O)[C@@H]2C[C@@H](O)CN2C(=O)[C@@H](NC(=O)CCOCC2CCN(c3ncc(C(=O)Nc4ccc(OC(F)(F)Cl)cc4)cc3-c3ccn[nH]3)CC2)C(C)(C)C)cc1 ; Cc1ncsc1-c1ccc(CNC(=O)[C@@H]2C[C@@H](O)CN2C(=O)[C@@H](NC(=O)CCOC[C@H]2CCN(c3ncc(C(=O)Nc4ccc(OC(F)(F)Cl)cc4)cc3-c3ccn[nH]3)C2)C(C)(C)C)cc1]

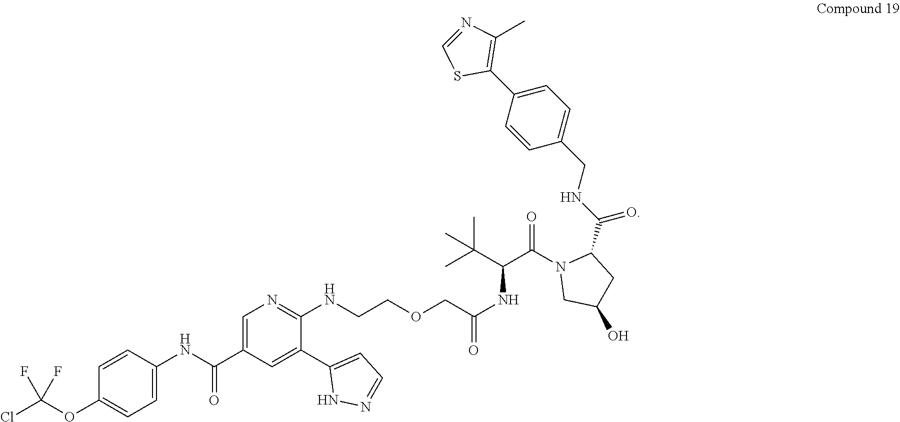 [SMILES: Cc1ncsc1-c1ccc(CNC(=O)[C@@H]2C[C@@H](O)CN2C(=O)[C@@H](NC(=O)COCCNc2ncc(C(=O)Nc3ccc(OC(F)(F)Cl)cc3)cc2-c2ccn[nH]2)C(C)(C)C)cc1]

A further aspect of the present disclosure provides a pharmaceutical composition comprising at least one compound of the present disclosure and at least one pharmaceutically acceptable carrier.

In any aspect or embodiment described herein, the composition further comprises at least one additional therapeutic compound that treats or prevents cancer.

Another aspect of the present disclosure provides a method of treating or preventing a disease or disorder associated with overexpression and/or uncontrolled activation of c-Abl and/or BCR-ABL, the method comprising administering to the subject a therapeutically effective amount of at least one compound of the present disclosure.

In any aspect or embodiment described herein, the disease or disorder comprises cancer.

In any aspect or embodiment described herein, the cancer comprises chronic myelogenous leukemia (CML).

In any aspect or embodiment described herein, the compound is administered to the subject by at least one route selected from the group consisting of nasal, inhalational, topical, oral, buccal, rectal, pleural, peritoneal, vaginal, intramuscular, subcutaneous, transdermal, epidural, intrathecal and intravenous routes.

An additional aspect of the present disclosure provides a method of preventing or treating a tyrosine kinase-dependent cancer in a subject in need thereof, the method comprising administering to the subject a therapeutically effective amount of at least one compound of the present disclosure.

In any aspect or embodiment described herein, the cancer is associated with overexpression and/or uncontrolled activation of the tyrosine kinase.

In any aspect or embodiment described herein, the tyrosine kinase is oncogenic.

In any aspect or embodiment described herein, the subject is a human.

In any aspect or embodiment described herein, the cancer comprises chronic myelogenous leukemia.

In any aspect or embodiment described herein, the compound of the present disclosure is administered to the subject by at least one route selected from the group consisting of nasal, inhalational, topical, oral, buccal, rectal, pleural, peritoneal, vaginal, intramuscular, subcutaneous, transdermal, epidural, intrathecal and intravenous routes.

### ENUMERATED EMBODIMENTS

The following exemplary embodiments are provided, the numbering of which is not to be construed as designating levels of importance:

Embodiment 1 provides a compound of Formula (I):

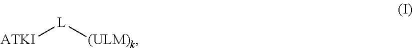

wherein:

ATKI is an allosteric tyrosine kinase inhibitor,

L is a linker,

each ULM is independently a ubiquitin ligase binder, and

k is an integer ranging from 1 to 4,

wherein ATKI is covalently linked to L and wherein each ULM is covalently linked to L; or a salt, enantiomer, stereoisomer, solvate, polymorph or N-oxide thereof.

Embodiment 2 provides the compound of embodiment 1, wherein ATKI is capable of binding to c-ABL and/or BCR-ABL.

Embodiment 3 provides the compound of any one of embodiments 1-2, wherein, upon binding of the compound of Formula (I) simultaneously to a tyrosine kinase and a ubiquitin ligase, the tyrosine kinase is ubiquitinated by the ubiquitin ligase.

Embodiment 4 provides the compound of any one of embodiments 1-3, wherein at least one ULM binds to an E3 ubiquitin ligase.

Embodiment 5 provides the compound of any one of embodiments 1-4, wherein the E3 ubiquitin ligase comprises a Von Hippel Lindau (VHL) E3 ubiquitin ligase, an MDM2 E3 ubiquitin ligase, Inhibitor of Apoptosis Protein (IAP) E3 ubiquitin ligase, or a Cereblon (CRBN) E3 ubiquitin ligase.

Embodiment 6 provides the compound of any one of embodiments 1-5, wherein the ATKI binds to an allosteric site on c-ABL and inhibits c-ABL.

Embodiment 7 provides the compound of any one of embodiments 1-6, wherein the ATKI binds to an allosteric site on BCR-ABL and inhibits BCR-ABL.

Embodiment 8 provides the compound of any one of embodiments 1-7, wherein the ATKI binds to an allosteric site on at least one of c-ABL and BCR-ABL and inhibits at least one of c-ABL and BCR-ABL.

Embodiment 9 provides the compound of any one of embodiments 1-8, wherein the ATKI is selected from the group consisting of GNF-2, GNF-5, asciminib, or any combinations thereof.

Embodiment 10 provides the compound of any one of embodiments 1-9, wherein at least one ULM comprises Formula (XXI):

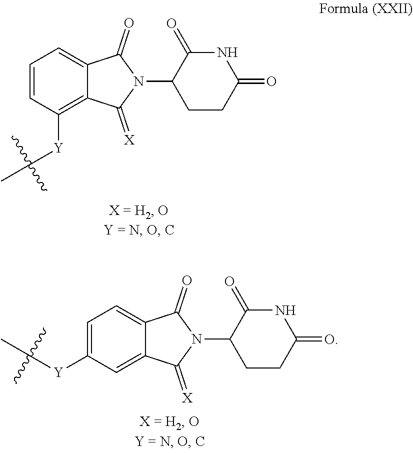

Embodiment 11 provides the compound of any one of embodiments 1-10, wherein at least one ULM comprises Formula (XXIII):

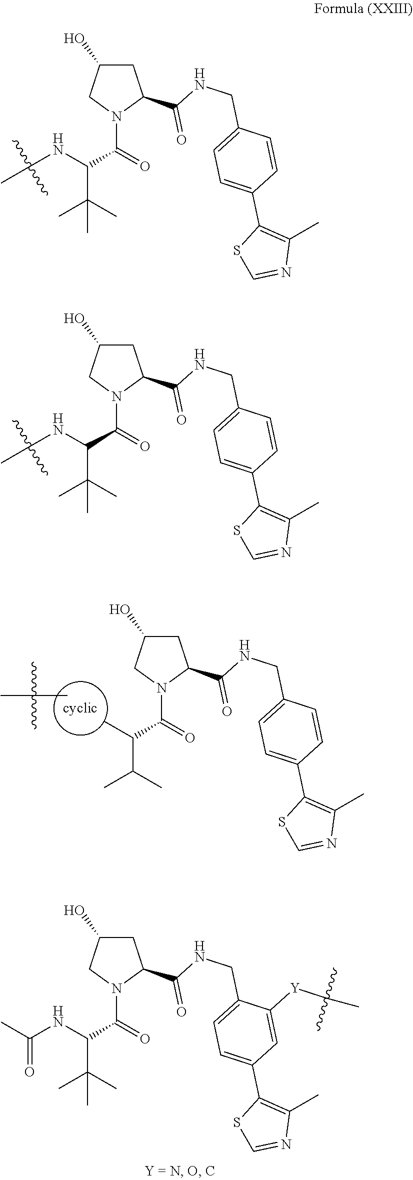

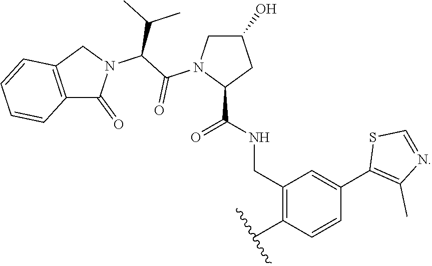 [SMILES: *c1ccc(-c2scnc2C)cc1CNC(=O)[C@@H]1C[C@@H](O)CN1C(=O)[C@H](C(C)C)N1Cc2ccccc2C1=O]

Embodiment 12 provides the compound of any one of embodiments 1-11, wherein k is 1.

Embodiment 13 provides the compound of any one of embodiments 1-12, wherein the linker L has the formula —(CH₂)ₘ₁—X⁴—((CH₂)_{m2′}—X⁵)ₘ₂—(CH₂)ₘ₃—X⁶—, wherein:

if m1 is greater than 0 then —(CH₂)ₘ₁ is covalently bonded to the ATKI;

if m1 is 0 then X⁴ is covalently bonded to the ATKI;

—X⁶ is covalently bonded to the ULM;

each m1, m2, m2′, and m3 is independently 0, 1, 2, 3, 4, 5, 6, 7, 8, 9, or 10;

each X⁴ and X⁵ independently absent (a bond), O, S, or N—R²⁰;

each X⁶ is independently absent (a bond), C(═O), NHC(═O), C(═S), C(═NR²⁰), O, S, or N—R²⁰; and

wherein each R²⁰ is independently selected from the group consisting of hydrogen, optionally substituted C₁-C₆ alkyl, optionally substituted aryl, optionally substituted heteroaryl, optionally substituted C₃-C₈ cycloalkyl, and optionally substituted C₃-C₈ cycloheteroalkyl.

Embodiment 14 provides the compound of any one of embodiments 1-13, wherein m1 is 0; m2′ is 2; m2 is 1 or 2; m3 is 1; and X⁴, X⁵, and X⁶ are O.

Embodiment 15 provides the compound of any one of embodiments 1-14, wherein m1 is 2; m2′ is 2; m2 is 1; m3 is 1; and X⁴, X⁵, and X⁶ are O.

Embodiment 16 provides the compound of any one of embodiments 1-15, wherein m1 is 2; m2′ is 2; m2 is 3; m3 is 1; and X⁴, X⁵, and X⁶ are O.

Embodiment 17 provides the compound of any one of embodiments 1-16, wherein the compound is selected from the group consisting of:

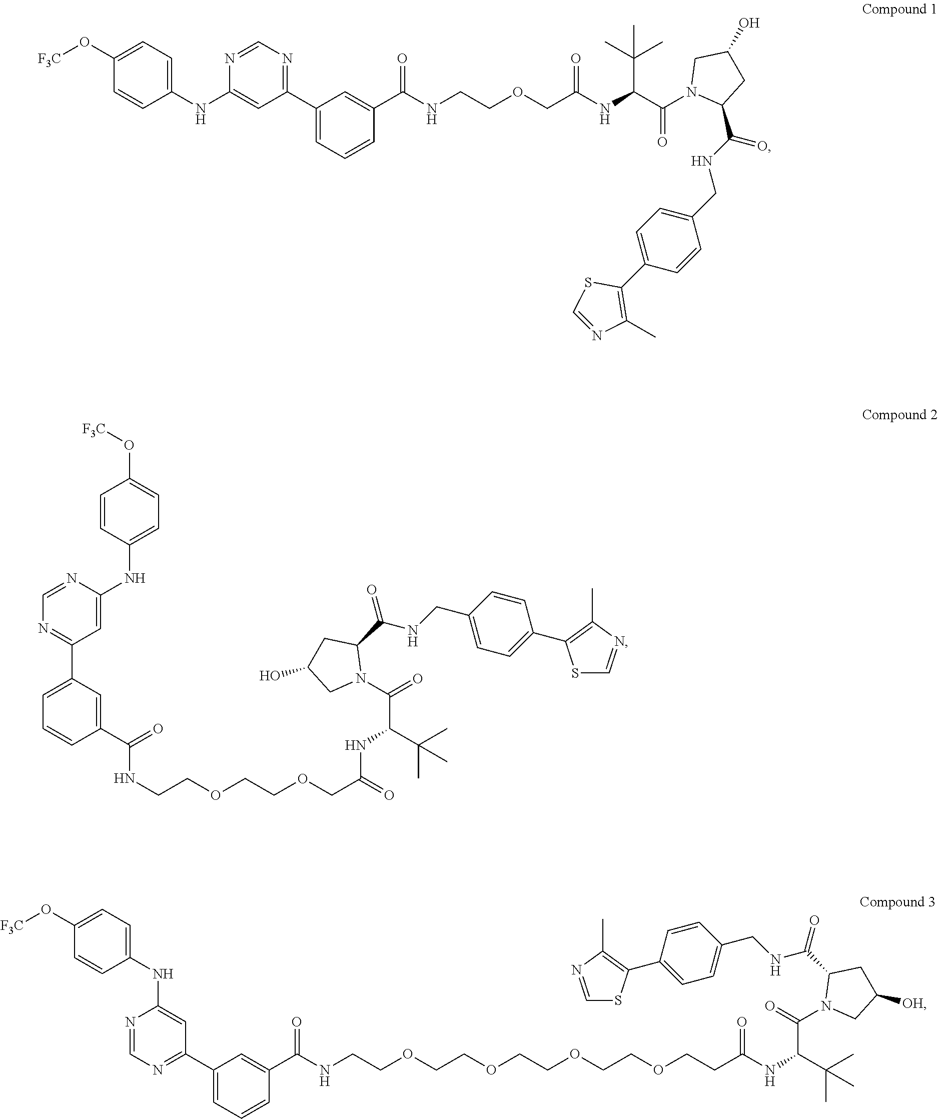 [SMILES: Cc1ncsc1-c1ccc(CNC(=O)[C@@H]2C[C@@H](O)CN2C(=O)[C@@H](NC(=O)COCCNC(=O)c2cccc(-c3cc(Nc4ccc(OC(F)(F)F)cc4)ncn3)c2)C(C)(C)C)cc1 ; Cc1ncsc1-c1ccc(CNC(=O)[C@@H]2C[C@@H](O)CN2C(=O)[C@@H](NC(=O)COCCOCCNC(=O)c2cccc(-c3cc(Nc4ccc(OC(F)(F)F)cc4)ncn3)c2)C(C)(C)C)cc1 ; Cc1ncsc1-c1ccc(CNC(=O)[C@@H]2C[C@@H](O)CN2C(=O)[C@@H](NC(=O)CCOCCOCCOCCOCCNC(=O)c2cccc(-c3cc(Nc4ccc(OC(F)(F)F)cc4)ncn3)c2)C(C)(C)C)cc1]

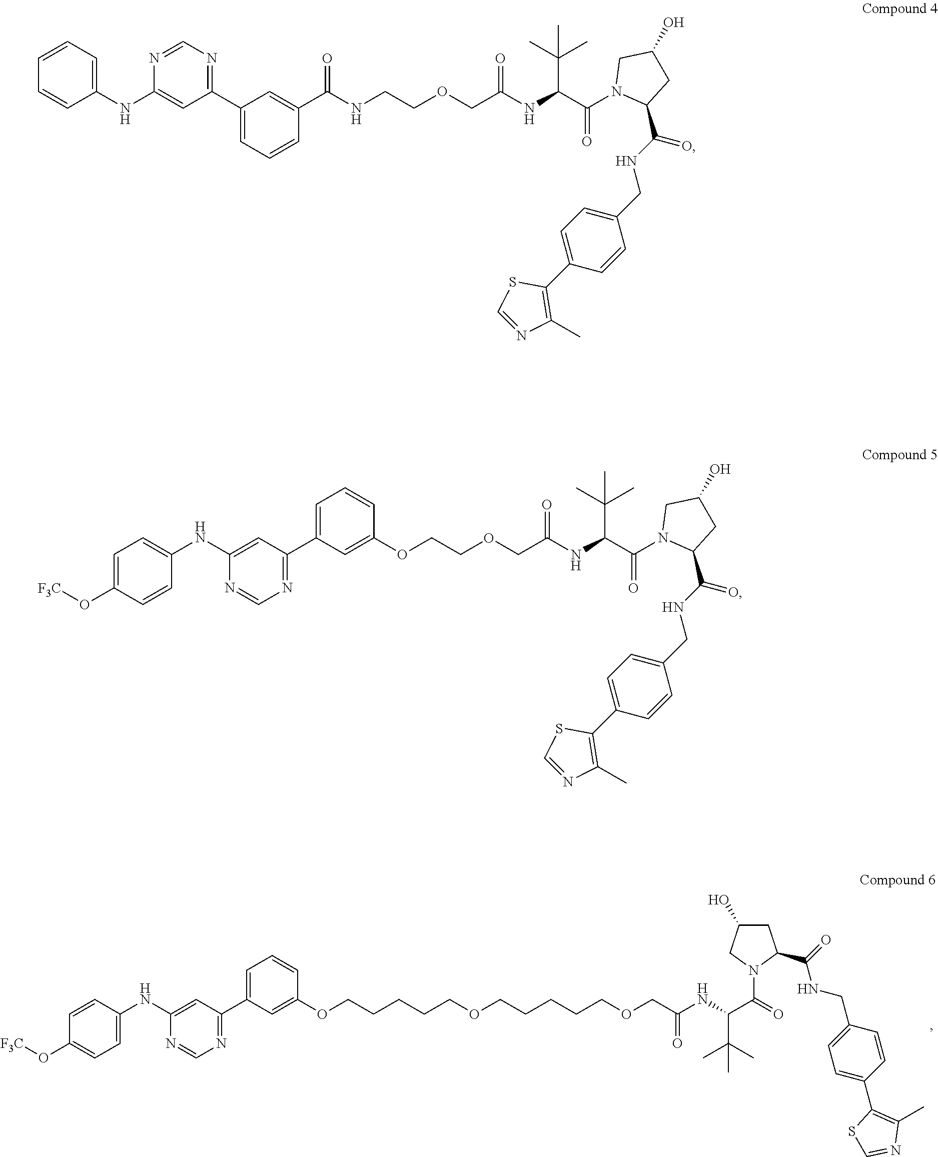 [SMILES: Cc1ncsc1-c1ccc(CNC(=O)[C@@H]2C[C@@H](O)CN2C(=O)[C@@H](NC(=O)COCCNC(=O)c2cccc(-c3cc(Nc4ccccc4)ncn3)c2)C(C)(C)C)cc1 ; Cc1ncsc1-c1ccc(CNC(=O)[C@@H]2C[C@@H](O)CN2C(=O)[C@@H](NC(=O)COCCOc2cccc(-c3cc(Nc4ccc(OC(F)(F)F)cc4)ncn3)c2)C(C)(C)C)cc1 ; Cc1ncsc1-c1ccc(CNC(=O)[C@@H]2C[C@@H](O)CN2C(=O)[C@@H](NC(=O)COCCCCCOCCCCCOc2cccc(-c3cc(Nc4ccc(OC(F)(F)F)cc4)ncn3)c2)C(C)(C)C)cc1]

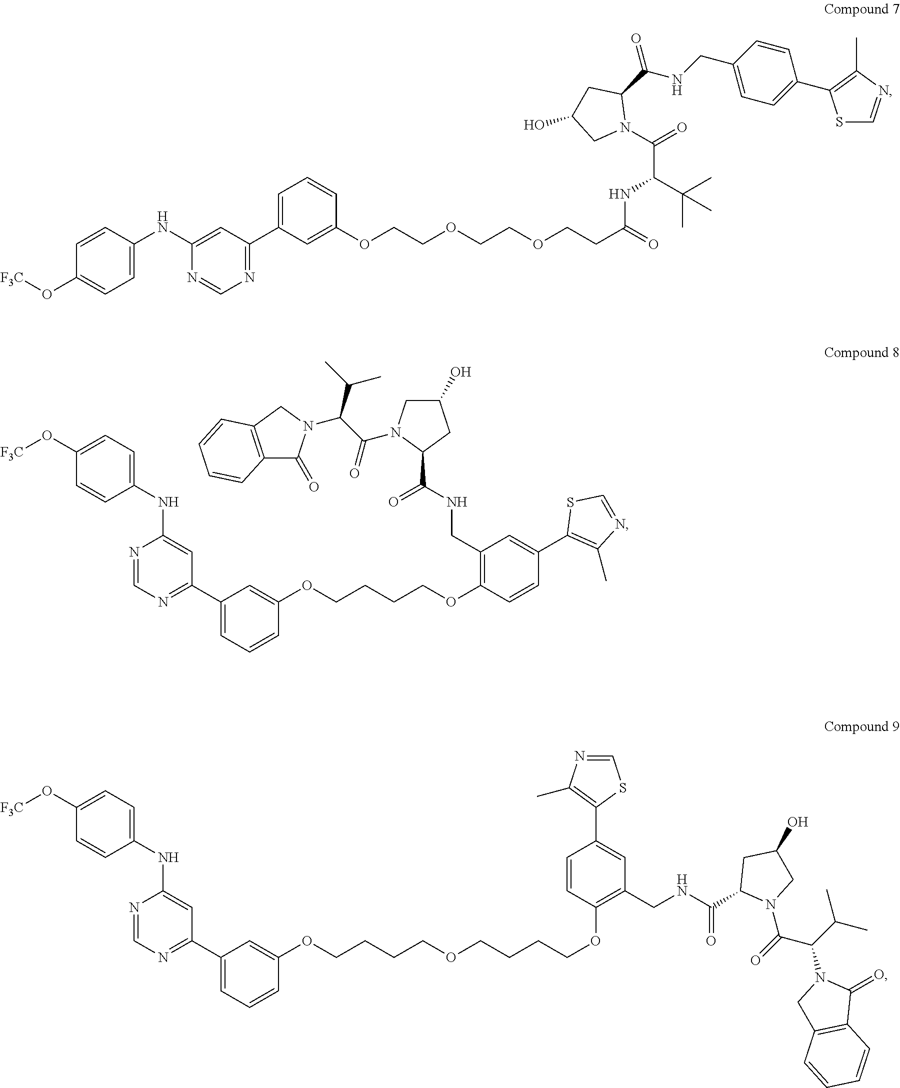 [SMILES: Cc1ncsc1-c1ccc(CNC(=O)[C@@H]2C[C@@H](O)CN2C(=O)[C@@H](NC(=O)CCOCCOCCOc2cccc(-c3cc(Nc4ccc(OC(F)(F)F)cc4)ncn3)c2)C(C)(C)C)cc1 ; Cc1ncsc1-c1ccc(OCCCCOc2cccc(-c3cc(Nc4ccc(OC(F)(F)F)cc4)ncn3)c2)c(CNC(=O)[C@@H]2C[C@@H](O)CN2C(=O)[C@H](C(C)C)N2Cc3ccccc3C2=O)c1 ; Cc1ncsc1-c1ccc(OCCCCOCCCCOc2cccc(-c3cc(Nc4ccc(OC(F)(F)F)cc4)ncn3)c2)c(CNC(=O)[C@@H]2C[C@@H](O)CN2C(=O)[C@H](C(C)C)N2Cc3ccccc3C2=O)c1]

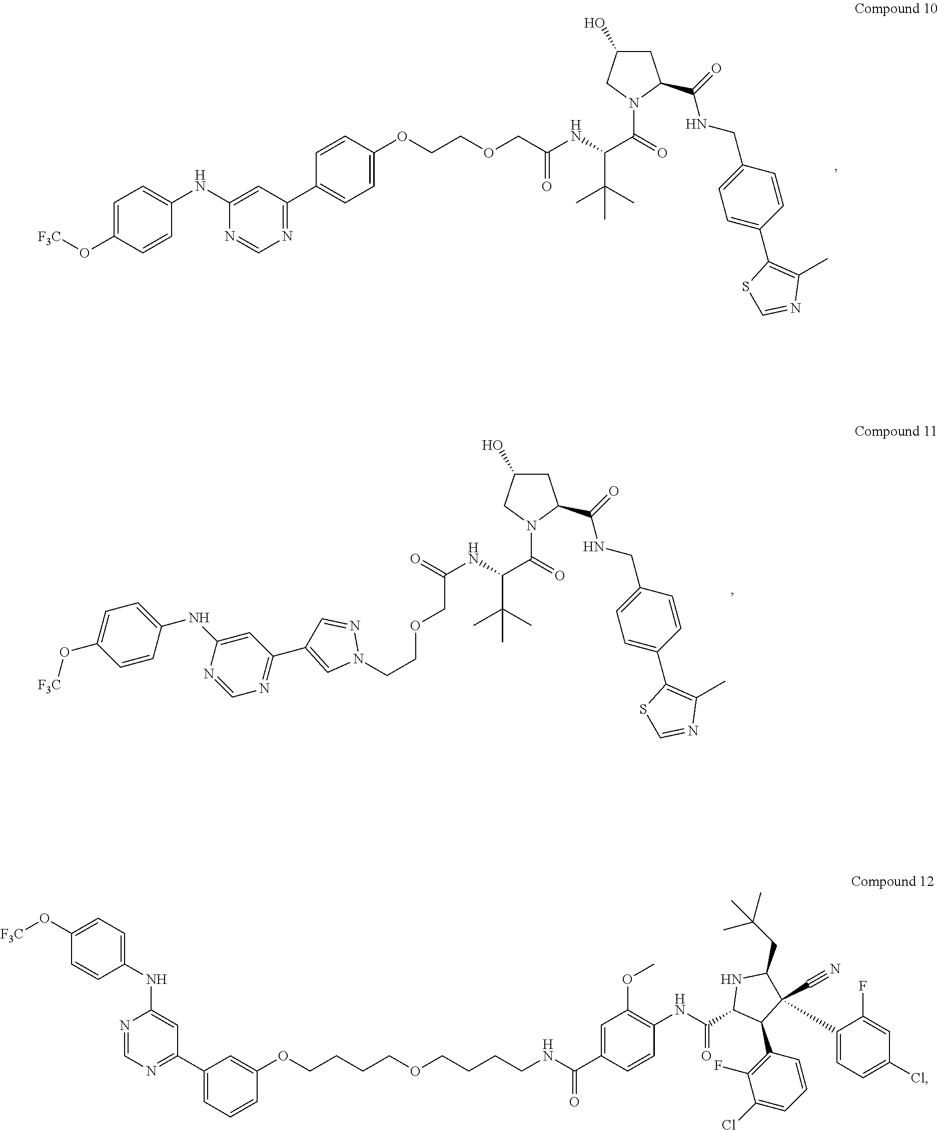 [SMILES: Cc1ncsc1-c1ccc(CNC(=O)[C@@H]2C[C@@H](O)CN2C(=O)[C@@H](NC(=O)COCCOc2ccc(-c3cc(Nc4ccc(OC(F)(F)F)cc4)ncn3)cc2)C(C)(C)C)cc1 ; Cc1ncsc1-c1ccc(CNC(=O)[C@@H]2C[C@@H](O)CN2C(=O)[C@@H](NC(=O)COCCn2cc(-c3cc(Nc4ccc(OC(F)(F)F)cc4)ncn3)cn2)C(C)(C)C)cc1 ; COc1cc(C(=O)NCCCCOCCCCOc2cccc(-c3cc(Nc4ccc(OC(F)(F)F)cc4)ncn3)c2)ccc1NC(=O)[C@@H]1N[C@@H](CC(C)(C)C)[C@](C#N)(c2ccc(Cl)cc2F)[C@H]1c1cccc(Cl)c1F]

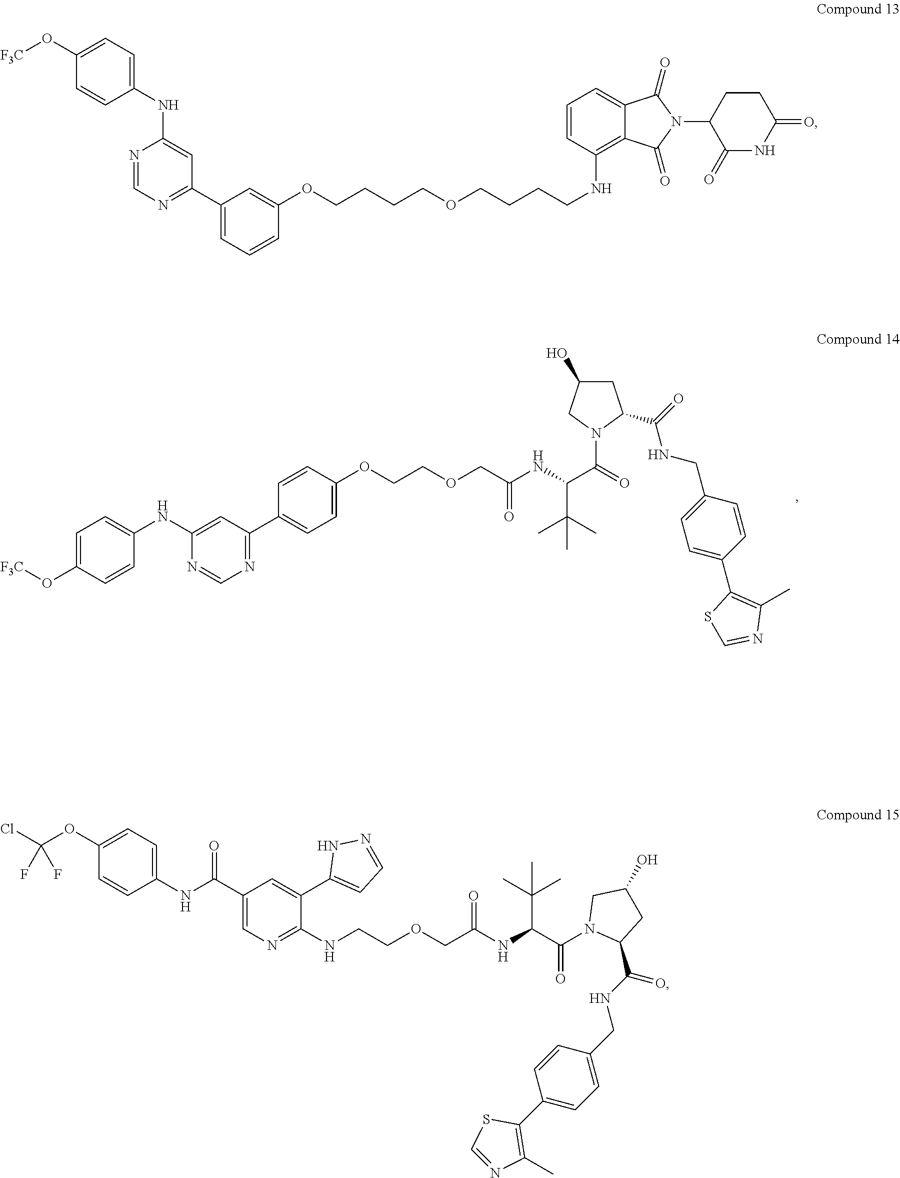 [SMILES: O=C1CCC(N2C(=O)c3cccc(NCCCCOCCCCOc4cccc(-c5cc(Nc6ccc(OC(F)(F)F)cc6)ncn5)c4)c3C2=O)C(=O)N1 ; Cc1ncsc1-c1ccc(CNC(=O)[C@H]2C[C@H](O)CN2C(=O)[C@@H](NC(=O)COCCOc2ccc(-c3cc(Nc4ccc(OC(F)(F)F)cc4)ncn3)cc2)C(C)(C)C)cc1 ; Cc1ncsc1-c1ccc(CNC(=O)[C@@H]2C[C@@H](O)CN2C(=O)[C@@H](NC(=O)COCCNc2ncc(C(=O)Nc3ccc(OC(F)(F)Cl)cc3)cc2-c2ccn[nH]2)C(C)(C)C)cc1]

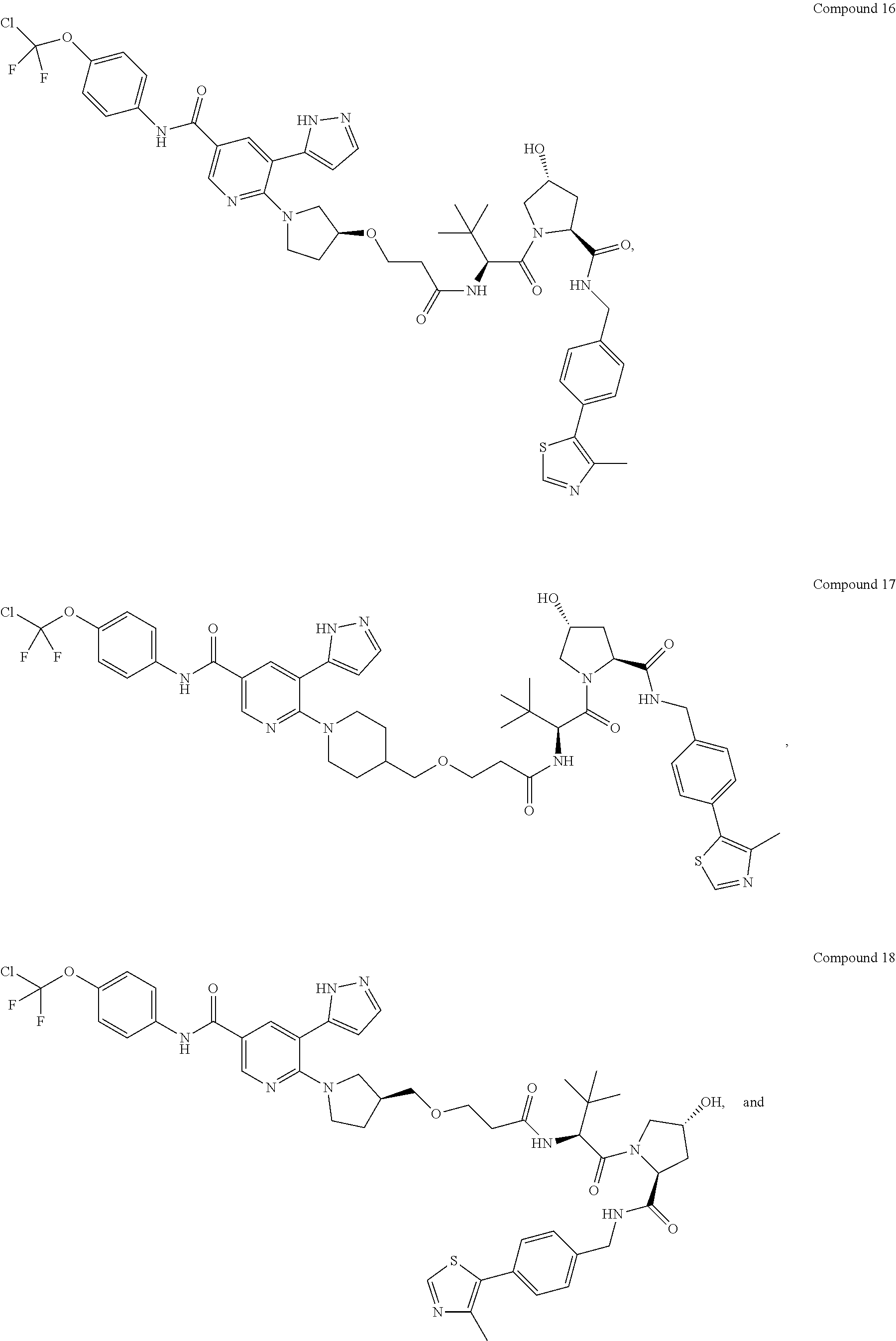 [SMILES: Cc1ncsc1-c1ccc(CNC(=O)[C@@H]2C[C@@H](O)CN2C(=O)[C@@H](NC(=O)CCO[C@H]2CCN(c3ncc(C(=O)Nc4ccc(OC(F)(F)Cl)cc4)cc3-c3ccn[nH]3)C2)C(C)(C)C)cc1 ; Cc1ncsc1-c1ccc(CNC(=O)[C@@H]2C[C@@H](O)CN2C(=O)[C@@H](NC(=O)CCOCC2CCN(c3ncc(C(=O)Nc4ccc(OC(F)(F)Cl)cc4)cc3-c3ccn[nH]3)CC2)C(C)(C)C)cc1 ; Cc1ncsc1-c1ccc(CNC(=O)[C@@H]2C[C@@H](O)CN2C(=O)[C@@H](NC(=O)CCOC[C@H]2CCN(c3ncc(C(=O)Nc4ccc(OC(F)(F)Cl)cc4)cc3-c3ccn[nH]3)C2)C(C)(C)C)cc1]

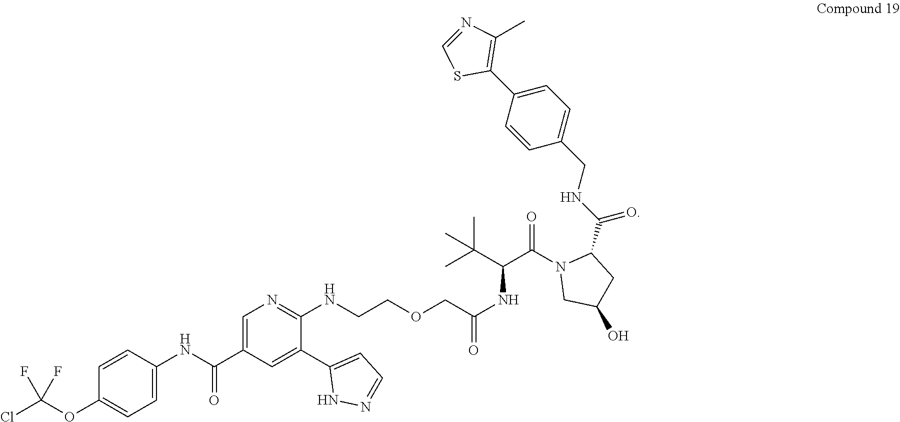 [SMILES: Cc1ncsc1-c1ccc(CNC(=O)[C@@H]2C[C@@H](O)CN2C(=O)[C@@H](NC(=O)COCCNc2ncc(C(=O)Nc3ccc(OC(F)(F)Cl)cc3)cc2-c2ccn[nH]2)C(C)(C)C)cc1]

Embodiment 18. provides a pharmaceutical composition comprising at least one compound of any one of embodiments 1-17 and at least one pharmaceutically acceptable carrier.

Embodiment 19 provides the composition of embodiment 18, further comprising at least one additional therapeutic compound that treats or prevents cancer.

Embodiment 20 provides a method of treating or preventing a disease or disorder associated with overexpression and/or uncontrolled activation of c-Abl and/or BCR-ABL, the method comprising administering to the subject a therapeutically effective amount of at least one compound of claim 2.

Embodiment 21 provides the method of embodiment 20, wherein the disease or disorder comprises cancer.

Embodiment 22 provides the method of any one of embodiments 20-21, wherein the cancer comprises chronic myelogenous leukemia (CML).

Embodiment 23 provides the method of any one of embodiments 20-22, wherein the compound is administered to the subject by at least one route selected from the group consisting of nasal, inhalational, topical, oral, buccal, rectal, pleural, peritoneal, vaginal, intramuscular, subcutaneous, transdermal, epidural, intrathecal and intravenous routes.

Embodiment 24 provides a method of preventing or treating a tyrosine kinase-dependent cancer in a subject in need thereof, the method comprising administering to the subject a therapeutically effective amount of at least one compound of any one of embodiments 1-18.

Embodiment 25 provides the method of embodiment 24, wherein the cancer is associated with overexpression and/or uncontrolled activation of the tyrosine kinase.

Embodiment 26 provides the method of any one of embodiments 24-25, wherein the tyrosine kinase is oncogenic.

Embodiment 27 provides the method of any one of embodiments 24-26, wherein the subject is a human.

Embodiment 28 provides the method of any one of embodiments 24-27, wherein the cancer comprises chronic myelogenous leukemia.

Embodiment 29 provides the method of any one of embodiments 24-28, wherein the compound is administered to the subject by at least one route selected from the group consisting of nasal, inhalational, topical, oral, buccal, rectal, pleural, peritoneal, vaginal, intramuscular, subcutaneous, transdermal, epidural, intrathecal and intravenous routes.

What is claimed is:

1. A compound of Formula (I): 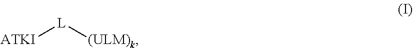
wherein:
ATKI is an allosteric tyrosine kinase inhibitor,
L is a linker,
each ULM is independently a ubiquitin ligase binder, and
k is an integer ranging from 1 to 4,
ATKI is covalently linked to L and wherein each ULM is covalently linked to L;
or a salt, enantiomer, stereoisomer, solvate, polymorph or N-oxide thereof.

2. The compound of claim 1, wherein ATKI is capable of binding to c-ABL and/or BCR-ABL.

3. The compound of claim 1, wherein, upon binding of the compound of Formula (I) simultaneously to a tyrosine kinase and a ubiquitin ligase, the tyrosine kinase is ubiquitinated by the ubiquitin ligase.

4. The compound of claim 1, wherein at least one ULM binds to an E3 ubiquitin ligase.

5. The compound of claim 4, wherein the E3 ubiquitin ligase comprises a Von Hippel Lindau (VHL) E3 ubiquitin ligase, an MDM2 E3 ubiquitin ligase, Inhibitor of Apoptosis Protein (IAP) E3 ubiquitin ligase, or a Cereblon (CRBN) E3 ubiquitin ligase.

6. The compound of claim 2, wherein the ATKI binds to an allosteric site on c-ABL and inhibits c-ABL.

7. The compound of claim 2, wherein the ATKI binds to an allosteric site on BCR-ABL and inhibits BCR-ABL.

8. The compound of claim 2, wherein the ATKI binds to an allosteric site on at least one of c-ABL and BCR-ABL and inhibits at least one of c-ABL and BCR-ABL.

9. The compound of claim 1, wherein the ATKI is selected from the group consisting of GNF-2, GNF-5, asciminib, or any combinations thereof.

10. The compound of claim 1, wherein at least one ULM comprises Formula (XXI):

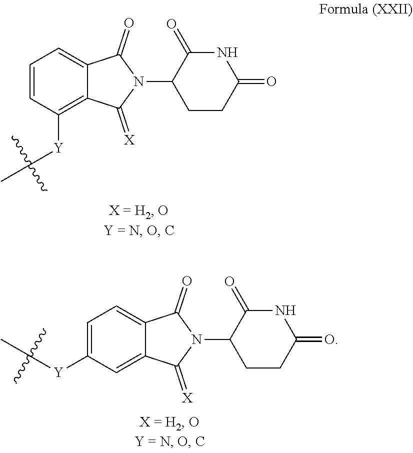

11. The compound of claim 1, wherein at least one ULM comprises Formula (XXIII):

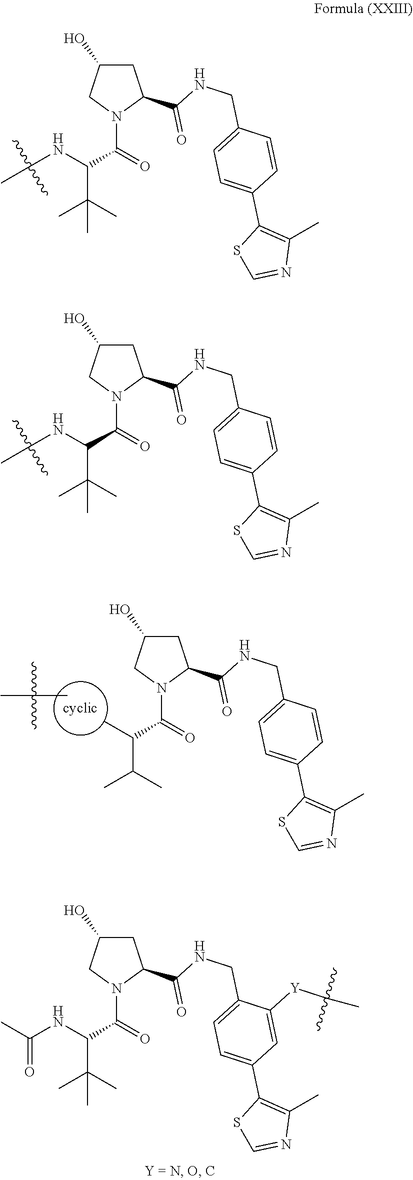

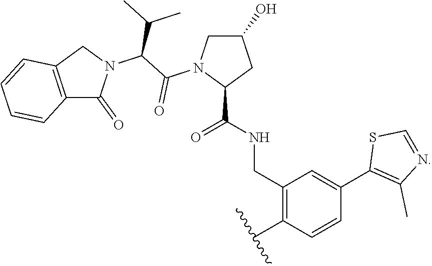 [SMILES: *c1ccc(-c2scnc2C)cc1CNC(=O)[C@@H]1C[C@@H](O)CN1C(=O)[C@H](C(C)C)N1Cc2ccccc2C1=O]

12. The compound of claim 1, wherein k is 1.

13. The compound of claim 1, wherein the linker L has the formula —(CH₂)ₘ₁—X⁴—((CH₂)_{m2′}—X⁵)ₘ(CH₎m2-(CH₂)ₘ₃—X⁶—, wherein:
if m1 is greater than 0 then —(CH₂)ₘ₁ is covalently bonded to the ATKI;
if m1 is 0 then X⁴ is covalently bonded to the ATKI;
—X⁶ is covalently bonded to the ULM;
each m1, m2, m2′, and m3 is independently 0, 1, 2, 3, 4, 5, 6, 7, 8, 9, or 10;
each X⁴ and X⁵ independently absent (a bond), O, S, or N—R²⁰;
each X⁶ is independently absent (a bond), C(═O), NHC(═O), C(═S), C(═NR²⁰), O, S, or N—R²⁰; and
wherein each R²⁰ is independently selected from the group consisting of hydrogen, optionally substituted C₁-C₆ alkyl, optionally substituted aryl, optionally substituted heteroaryl, optionally substituted C₃-C₈ cycloalkyl, and optionally substituted C₃-C₈ cycloheteroalkyl.

14. The compound of claim 13, wherein m1 is 0; m2′ is 2; m2 is 1 or 2; m3 is 1; and X⁴, X⁵, and X⁶ are O.

15. The compound of claim 13, wherein m1 is 2; m2′ is 2; m2 is 1; m3 is 1; and X⁴, X⁵, and X⁶ are O.

16. The compound of claim 13, wherein m1 is 2; m2′ is 2; m2 is 3; m3 is 1; and X⁴, X⁵, and X⁶ are O.

17. The compound of claim 1, wherein the compound is selected from the group consisting of:

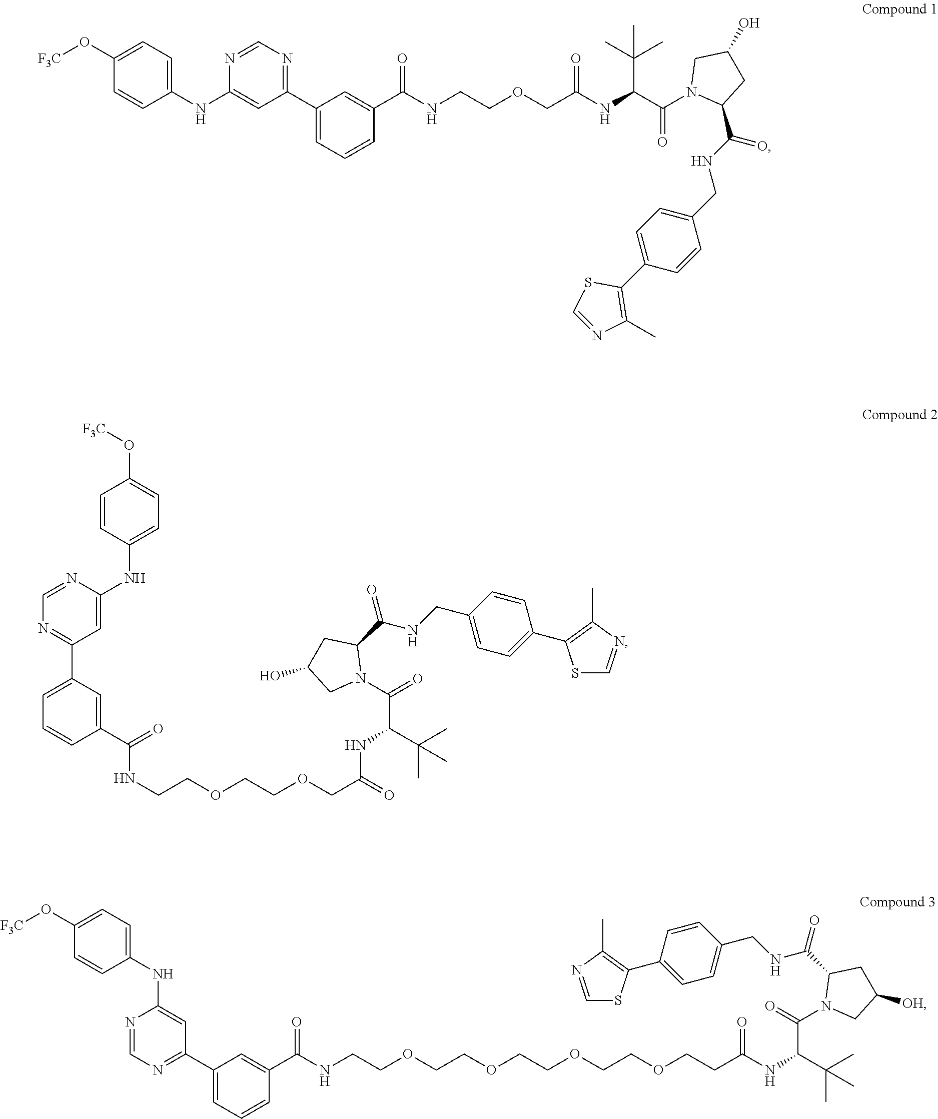 [SMILES: Cc1ncsc1-c1ccc(CNC(=O)[C@@H]2C[C@@H](O)CN2C(=O)[C@@H](NC(=O)COCCNC(=O)c2cccc(-c3cc(Nc4ccc(OC(F)(F)F)cc4)ncn3)c2)C(C)(C)C)cc1 ; Cc1ncsc1-c1ccc(CNC(=O)[C@@H]2C[C@@H](O)CN2C(=O)[C@@H](NC(=O)COCCOCCNC(=O)c2cccc(-c3cc(Nc4ccc(OC(F)(F)F)cc4)ncn3)c2)C(C)(C)C)cc1 ; Cc1ncsc1-c1ccc(CNC(=O)[C@@H]2C[C@@H](O)CN2C(=O)[C@@H](NC(=O)CCOCCOCCOCCOCCNC(=O)c2cccc(-c3cc(Nc4ccc(OC(F)(F)F)cc4)ncn3)c2)C(C)(C)C)cc1]

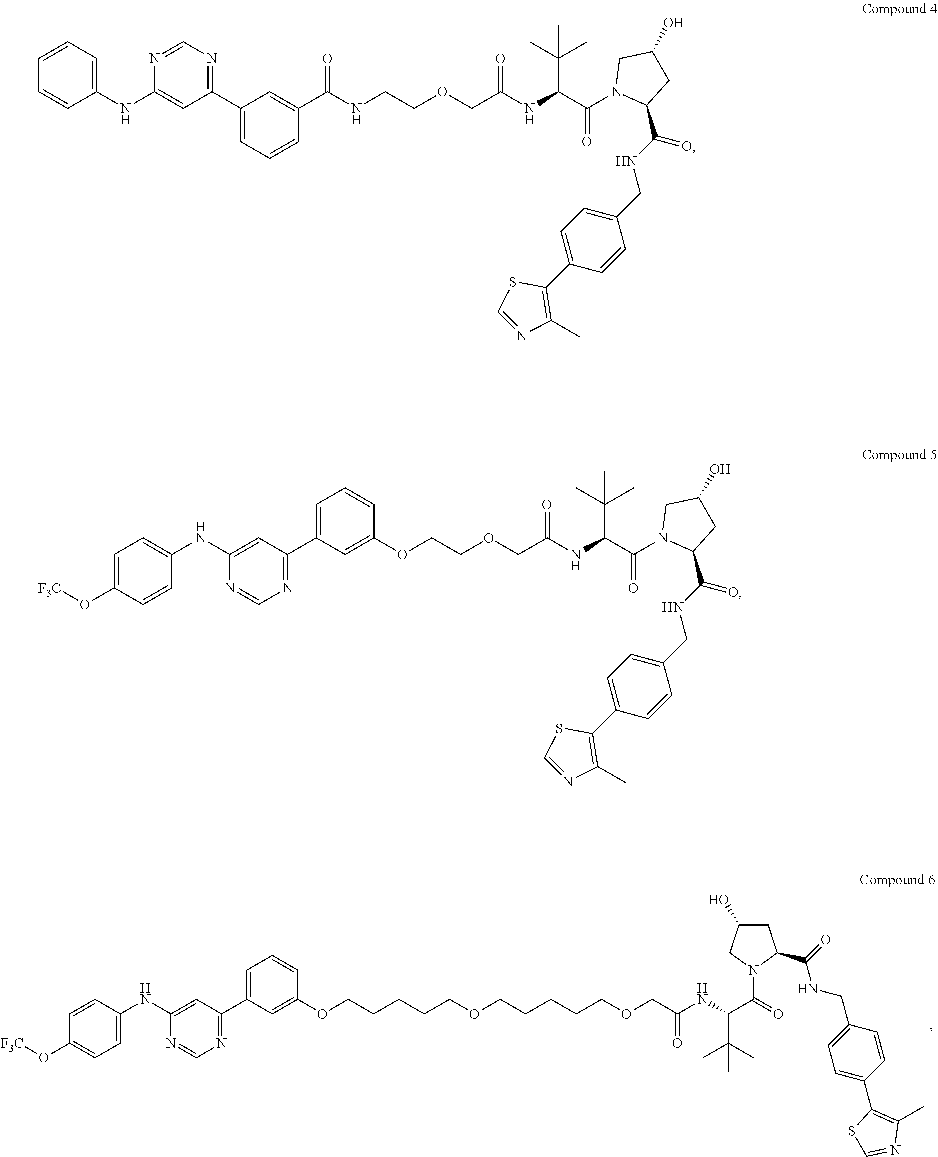 [SMILES: Cc1ncsc1-c1ccc(CNC(=O)[C@@H]2C[C@@H](O)CN2C(=O)[C@@H](NC(=O)COCCNC(=O)c2cccc(-c3cc(Nc4ccccc4)ncn3)c2)C(C)(C)C)cc1 ; Cc1ncsc1-c1ccc(CNC(=O)[C@@H]2C[C@@H](O)CN2C(=O)[C@@H](NC(=O)COCCOc2cccc(-c3cc(Nc4ccc(OC(F)(F)F)cc4)ncn3)c2)C(C)(C)C)cc1 ; Cc1ncsc1-c1ccc(CNC(=O)[C@@H]2C[C@@H](O)CN2C(=O)[C@@H](NC(=O)COCCCCCOCCCCCOc2cccc(-c3cc(Nc4ccc(OC(F)(F)F)cc4)ncn3)c2)C(C)(C)C)cc1]

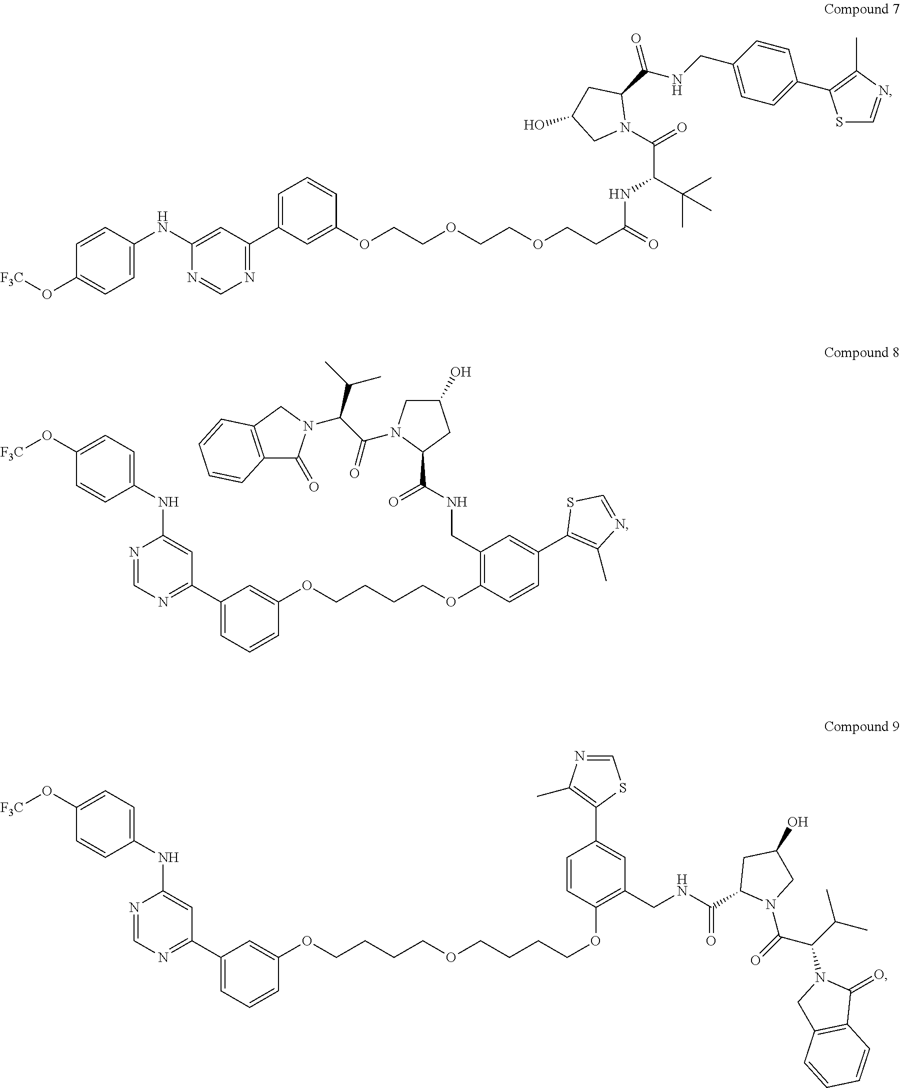 [SMILES: Cc1ncsc1-c1ccc(CNC(=O)[C@@H]2C[C@@H](O)CN2C(=O)[C@@H](NC(=O)CCOCCOCCOc2cccc(-c3cc(Nc4ccc(OC(F)(F)F)cc4)ncn3)c2)C(C)(C)C)cc1 ; Cc1ncsc1-c1ccc(OCCCCOc2cccc(-c3cc(Nc4ccc(OC(F)(F)F)cc4)ncn3)c2)c(CNC(=O)[C@@H]2C[C@@H](O)CN2C(=O)[C@H](C(C)C)N2Cc3ccccc3C2=O)c1 ; Cc1ncsc1-c1ccc(OCCCCOCCCCOc2cccc(-c3cc(Nc4ccc(OC(F)(F)F)cc4)ncn3)c2)c(CNC(=O)[C@@H]2C[C@@H](O)CN2C(=O)[C@H](C(C)C)N2Cc3ccccc3C2=O)c1]

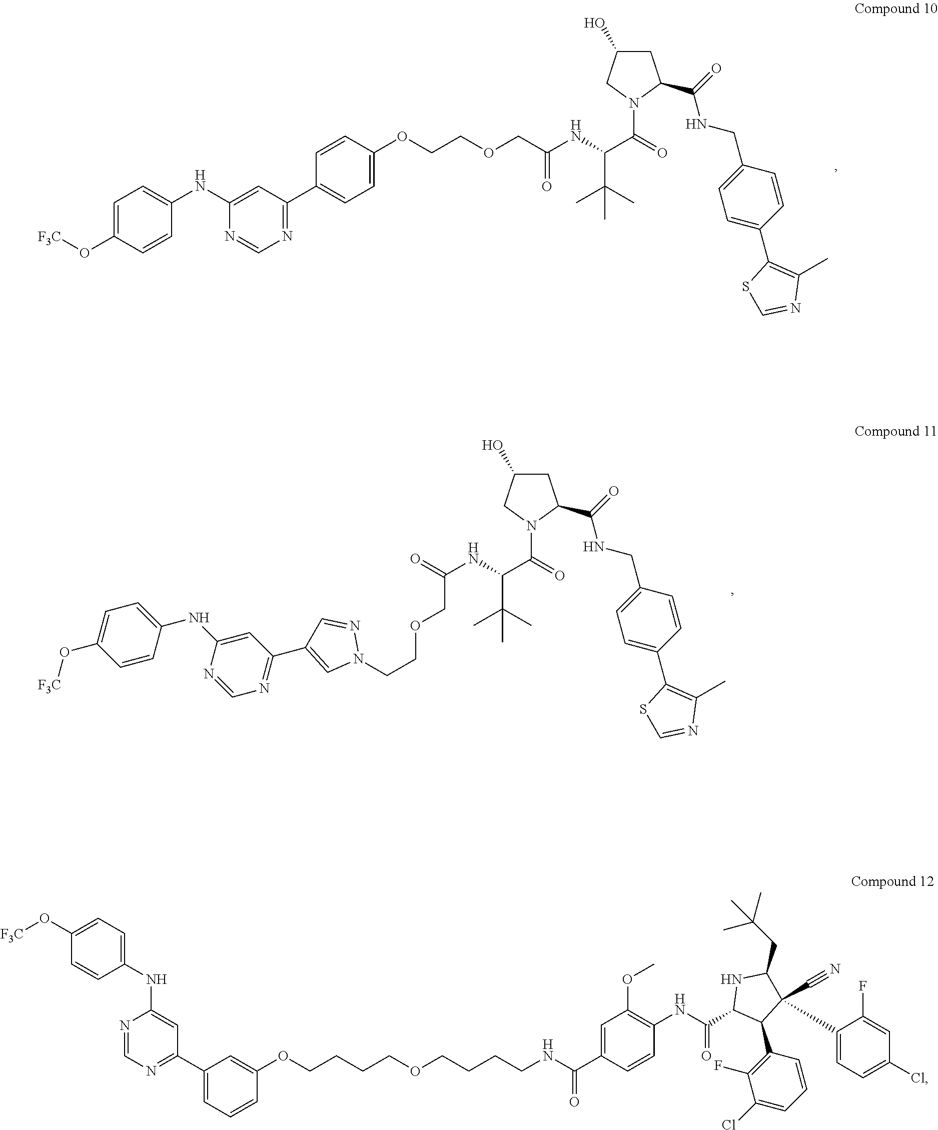 [SMILES: Cc1ncsc1-c1ccc(CNC(=O)[C@@H]2C[C@@H](O)CN2C(=O)[C@@H](NC(=O)COCCOc2ccc(-c3cc(Nc4ccc(OC(F)(F)F)cc4)ncn3)cc2)C(C)(C)C)cc1 ; Cc1ncsc1-c1ccc(CNC(=O)[C@@H]2C[C@@H](O)CN2C(=O)[C@@H](NC(=O)COCCn2cc(-c3cc(Nc4ccc(OC(F)(F)F)cc4)ncn3)cn2)C(C)(C)C)cc1 ; COc1cc(C(=O)NCCCCOCCCCOc2cccc(-c3cc(Nc4ccc(OC(F)(F)F)cc4)ncn3)c2)ccc1NC(=O)[C@@H]1N[C@@H](CC(C)(C)C)[C@](C#N)(c2ccc(Cl)cc2F)[C@H]1c1cccc(Cl)c1F]

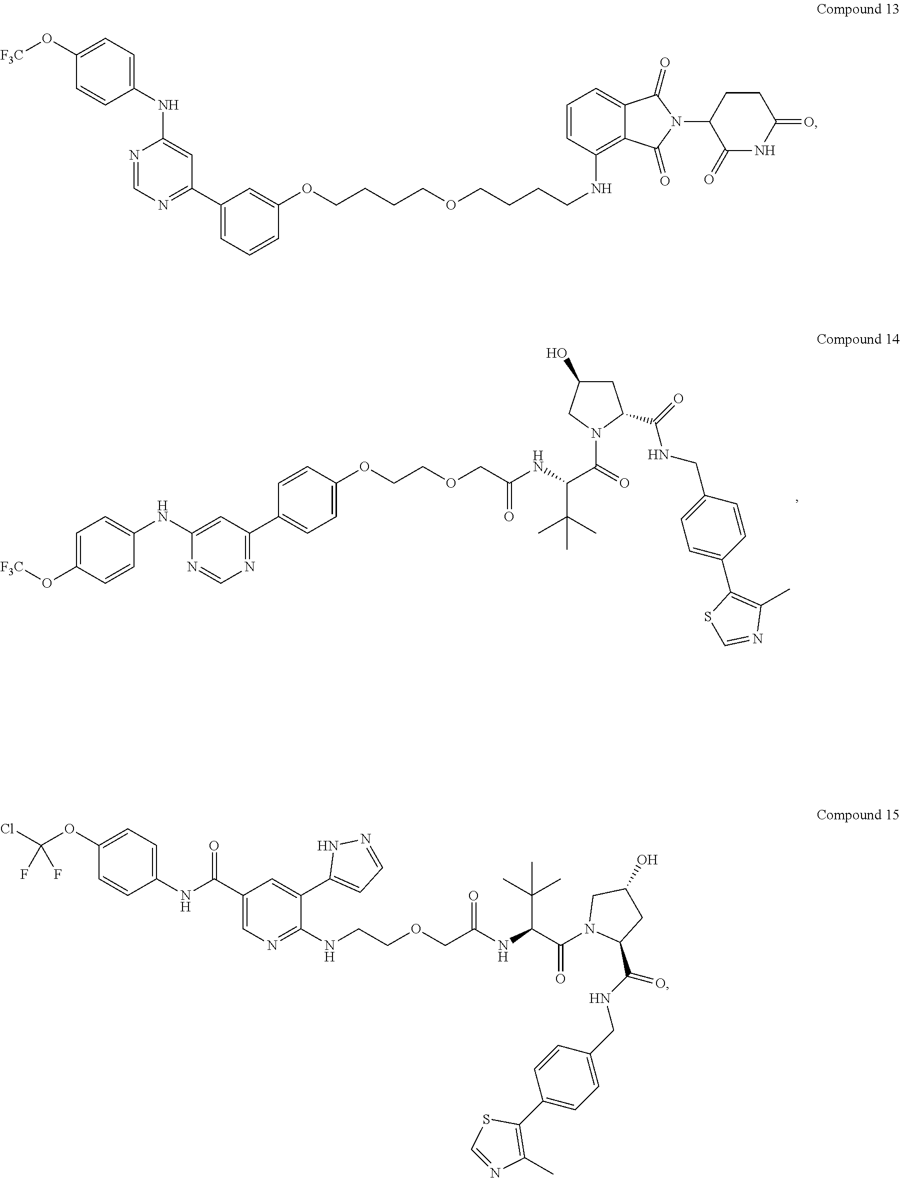 [SMILES: O=C1CCC(N2C(=O)c3cccc(NCCCCOCCCCOc4cccc(-c5cc(Nc6ccc(OC(F)(F)F)cc6)ncn5)c4)c3C2=O)C(=O)N1 ; Cc1ncsc1-c1ccc(CNC(=O)[C@H]2C[C@H](O)CN2C(=O)[C@@H](NC(=O)COCCOc2ccc(-c3cc(Nc4ccc(OC(F)(F)F)cc4)ncn3)cc2)C(C)(C)C)cc1 ; Cc1ncsc1-c1ccc(CNC(=O)[C@@H]2C[C@@H](O)CN2C(=O)[C@@H](NC(=O)COCCNc2ncc(C(=O)Nc3ccc(OC(F)(F)Cl)cc3)cc2-c2ccn[nH]2)C(C)(C)C)cc1]

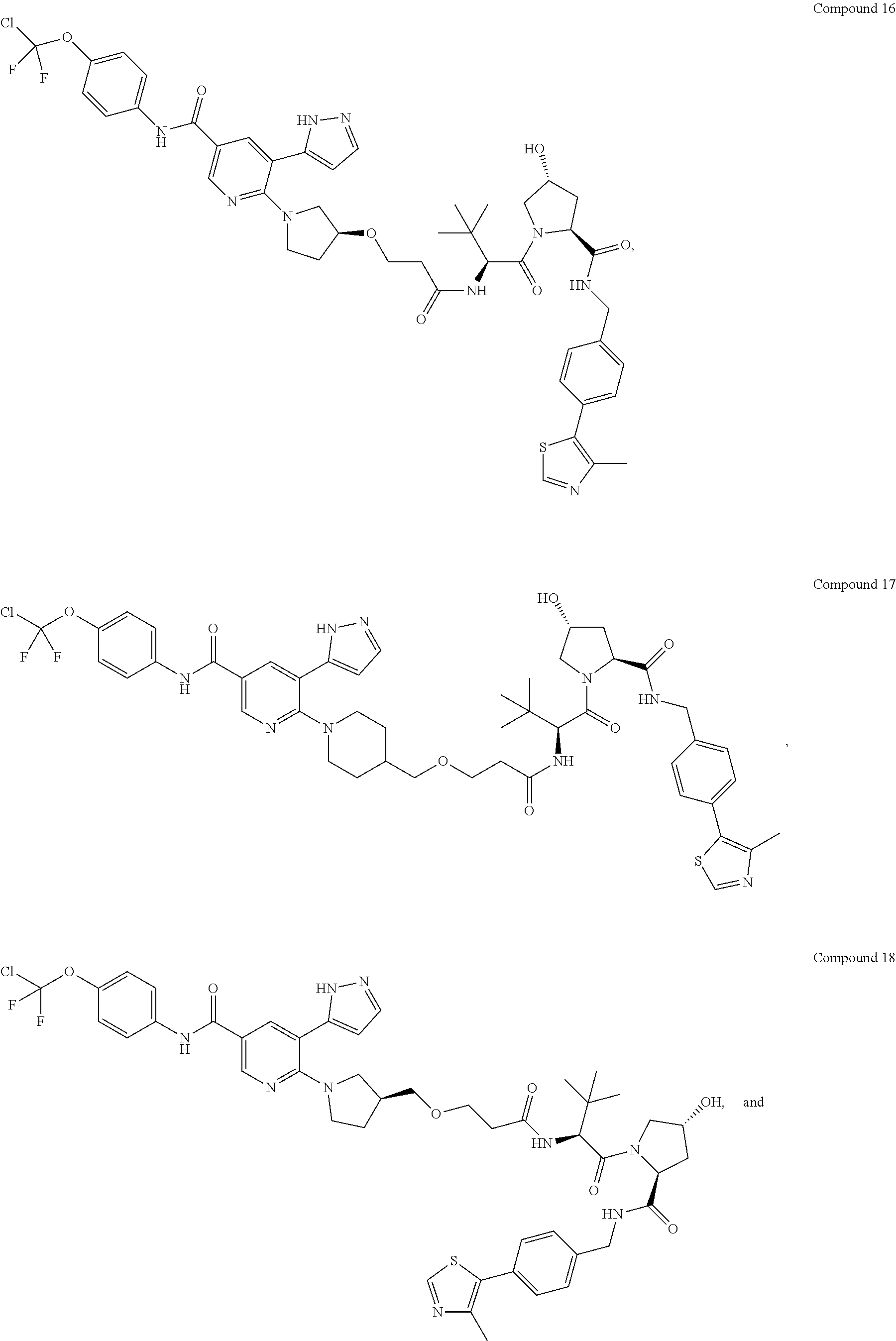 [SMILES: Cc1ncsc1-c1ccc(CNC(=O)[C@@H]2C[C@@H](O)CN2C(=O)[C@@H](NC(=O)CCO[C@H]2CCN(c3ncc(C(=O)Nc4ccc(OC(F)(F)Cl)cc4)cc3-c3ccn[nH]3)C2)C(C)(C)C)cc1 ; Cc1ncsc1-c1ccc(CNC(=O)[C@@H]2C[C@@H](O)CN2C(=O)[C@@H](NC(=O)CCOCC2CCN(c3ncc(C(=O)Nc4ccc(OC(F)(F)Cl)cc4)cc3-c3ccn[nH]3)CC2)C(C)(C)C)cc1 ; Cc1ncsc1-c1ccc(CNC(=O)[C@@H]2C[C@@H](O)CN2C(=O)[C@@H](NC(=O)CCOC[C@H]2CCN(c3ncc(C(=O)Nc4ccc(OC(F)(F)Cl)cc4)cc3-c3ccn[nH]3)C2)C(C)(C)C)cc1]

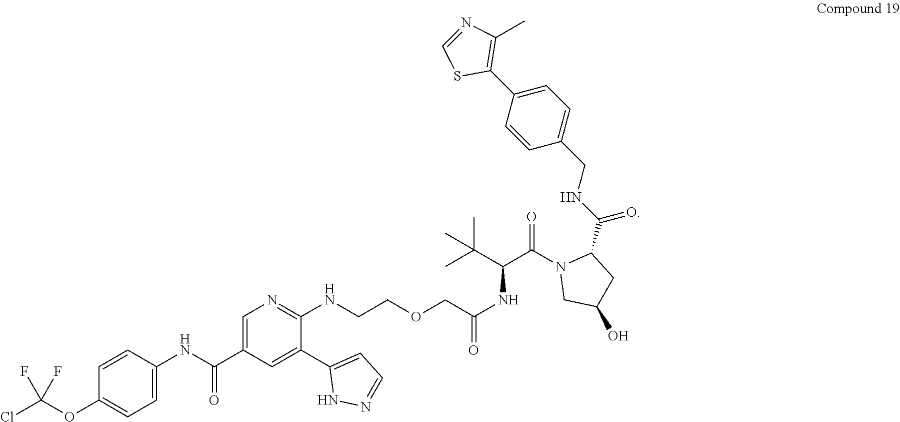 [SMILES: Cc1ncsc1-c1ccc(CNC(=O)[C@@H]2C[C@@H](O)CN2C(=O)[C@@H](NC(=O)COCCNc2ncc(C(=O)Nc3ccc(OC(F)(F)Cl)cc3)cc2-c2ccn[nH]2)C(C)(C)C)cc1]

18. A pharmaceutical composition comprising at least one compound of claim 1 and at least one pharmaceutically acceptable carrier.

19. The composition of claim 18, further comprising at least one additional therapeutic compound that treats or prevents cancer.

20. A method of treating or preventing a disease or disorder associated with overexpression and/or uncontrolled activation of c-Abl and/or BCR-ABL, the method comprising administering to the subject a therapeutically effective amount of at least one compound of claim 2.

21. The method of claim 20, wherein the disease or disorder comprises cancer.

22. The method of claim 21, wherein the cancer comprises chronic myelogenous leukemia (CML).

23. The method of claim 20, wherein the compound is administered to the subject by at least one route selected from the group consisting of nasal, inhalational, topical, oral, buccal, rectal, pleural, peritoneal, vaginal, intramuscular, subcutaneous, transdermal, epidural, intrathecal and intravenous routes.

24. A method of preventing or treating a tyrosine kinase-dependent cancer in a subject in need thereof, the method comprising administering to the subject a therapeutically effective amount of at least one compound of claim 1.

25. The method of claim 24, wherein the cancer is associated with overexpression and/or uncontrolled activation of the tyrosine kinase.

26. The method of claim 24, wherein the tyrosine kinase is oncogenic.

27. The method of claim 24, wherein the subject is a human.

28. The method of claim 24, wherein the cancer comprises chronic myelogenous leukemia.

29. The method of claim 24, wherein the compound is administered to the subject by at least one route selected from the group consisting of nasal, inhalational, topical, oral, buccal, rectal, pleural, peritoneal, vaginal, intramuscular, subcutaneous, transdermal, epidural, intrathecal and intravenous routes.
